SEQUENCE LISTING (147 sequences)

SEQ ID NO: 1 - DNA, 2030 bp, Yarrowia lipolytica
atggccaaggagcgaggtaagactcaattcactgtccgagatgtgaccaacttcctcaatggtggagaagaagagacccagattgtcgagaagatcatgagcagtattgaacgtgatccagtactgtctgtcactgctgactacgactgcaaccttcagcaggcccgaaaacagaccatggagcgggtggctgctctgtcgccttatctggtcaccgatactgagaagctatctctgtggcgtgcgcaactgcatggaatggttgatatgtctactcgtacgcggttgtcgatccacaacaacctgttcattggttccatcaggggatctggtactcctgaacagttcaagtactgggtcaagaagggagcggtggctgttaagcagttctatggatgctttgccatgacagagttgggccatggaagcaacctcaagggactagagacaaccgccacttatgaccaggacagtgaccagttcattatcaacactcctcatattggtgctaccaagtggtggattggcggtgcagcccacacttccacccattgtgtttgtttcgcgaaactgattgtgcatggcaaggactatggtactcgaaactttgtggtacctctccgaaatgtccacgatcacagtctcaaggtcggtgtttcaattggagacattggaaagaagatgggcagagatggtgttgacaatggctggatccagttcaccaatgttcgaatccccagacagaacatgctaatgagatatgccaaggtgtctgatactggagtggtaaccaaacccgctcttgaccaactcacttatggagccctcattcgaggtcgagtgtccatgattgccgactcgttccacgtctccaaacgattcctcacaattgctcttcggtacgcttgtgtccgacgacagtttggaacctctggagacactaaggagaccaagatcatcgactacccttaccaccagcgacgattgctgcctcttctggcctactgctacgctatgaagatgggtgctgatgaggctcagaagacttggattgagaccaccgatcgaattctggctctcaatcccaacgaccccgcccagaagaacgatctggagaaggccgtcaccgacacaaaggagctgtttgctgcgtctgcaggaatgaaggcatttaccacgtggggatgtgccaaaatcattgatgagtgccgacaggcctgtggaggtcatggatactctggatataacggatttggccagggctacgctgactgggttgtccagtgtacctgggaaggagacaacaacgttctgtgtctgtcaatgggccgagggctggttcagtcagctctacagattttggctggaaagcacgtcggtgcttctattcagtacgtaggagacaagtctaaaatctcccagaacggccagggtacccccagagagcaacttctgtcccccgagtttctagtagaagctttcagaacggcttctcgaaacaacattctcagaaccaccgataaataccaagagcttgtcaaaactctcaatcccgaccaggcctttgaggagctgtctcagcagagattccagtgtgctcgaatccacacacgacagcatcttatctcttcattctatgcccgaattgccactgccaaagacgatatcaagccccatctgctgaaactggccaatctgtttgccctctggtcaattgaggaggacactggaatcttcctgcgggagaacatcctcacccctggagacattgacctgatcaacagtcttgtggacgagctctgtgttgcagttcgagatcaggtaattggactcactgatgcctttggtctctctgacttcttcattaacgctcccatcggctcctacgatggtaatgtttacgaaaagtactttgccaaggtcaaccagcaaaaccccgctactaaccctcgtcctccctactacgagtcgactctcaagcccttcttgttccgagaagaggaggacgatgaaatttgcgatctcgatga

SEQ ID NO: 2 - protein, 677 aa, Yarrowia lipolytica
MAKERGKTQFTVRDVTNFLNGGEEETQIVEKIMSSIERDPVLSVTADYDCNLQQARKQTMERVAALSPYLVTDTEKLSLWRAQLHGMVDMSTRTRLSIHNNLFIGSIRGSGTPEQFKYWVKKGAVAVKQFYGCFAMTELGHGSNLKGLETTATYDQDSDQFIINTPHIGATKWWIGGAAHTSTHCVCFAKLIVHGKDYGTRNFVVPLRNVHDHSLKVGVSIGDIGKKMGRDGVDNGWIQFTNVRIPRQNMLMRYAKVSDTGVVTKPALDQLTYGALIRGRVSMIADSFHVSKRFLTIALRYACVRRQFGTSGDTKETKIIDYPYHQRRLLPLLAYCYAMKMGADEAQKTWIETTDRILALNPNDPAQKNDLEKAVTDTKELFAASAGMKAFTTWGCAKIIDECRQACGGHGYSGYNGFGQGYADWVVQCTWEGDNNVLCLSMGRGLVQSALQILAGKHVGASIQYVGDKSKISQNGQGTPREQLLSPEFLVEAFRTASRNNILRTTDKYQELVKTLNPDQAFEELSQQRFQCARIHTRQHLISSFYARIATAKDDIKPHLLKLANLFALWSIEEDTGIFLRENILTPGDIDLINSLVDELCVAVRDQVIGLTDAFGLSDFFINAPIGSYDGNVYEKYFAKVNQQNPATNPRPPYYESTLKPFLFREEEDDEICDLDE

SEQ ID NO: 3 - DNA, 2103 bp, Yarrowia lipolytica
atgaaccccaacaacactggcaccattgaaatcaacggtaaggagtacaacaccttcaccgagccccccgtggccatggctcaggagcgagccaagacctccttccccgtgcgagagatgacctacttcctcgacggtggcgagaagaacaccctcaaaaacgagcagatcatggaggagattgagcgagaccctcttttcaacaacgacaactactacgatctcaacaaggagcagatccgagagctcaccatggagcgagtcgccaagctgtctctgtttgtgcgtgatcagcccgaggacgacatcaagaagcgatttgctctcattggtatcgccgatatgggaacctacacccgacttggtgtccactacggcctcttctttggcgccgtccgaggtaccggaactgccgagcagtttggccactggatctccaagggagccggagacctgcgaaagttctacggatgtttctccatgaccgagctgggccatggctccaacctggctggtctcgagaccaccgccatctacgatgaggagaccgacgagttcatcatcaacacccctcacattgccgccaccaagtggtggattggaggagccgcccacaccgccacccacactgtcgtgttcgcccgactcattgtcaagggcaaggactacggtgtcaagacctttgttgtccagctgcgaaacatcaacgaccacagcctcaaggtcggtatctctattggtgatatcggaaagaagatgggccgagacggtatcgataacggatggatccagttcaccaacgtgcgaatcccccgacagaacctgctcatgaagtacacaaaggtcgaccgagagggtaacgtgacccagcctcctctggctcagcttacctacggttctcttatcactggtcgagtctccatggcctctgattctcaccaggtcggaaagcgattcatcaccattgctctgcgatacgcctgcattcgacgacagttctccaccacccccggccagcccgagaccaagatcatcgactacccctaccatcagcgacgacttctgcctcttctggcctatgtctatgctcttaagatgactgccgatgaggttggagctctcttctcccgaaccatgcttaagatggacgacctcaagcccgacgacaaggccggcctcaatgaggttgtttccgacgtcaaggagctcttctccgtctccgccggtctcaaggccttctccacctgggcttgtgccgacgtcattgacaagacccgacaggcttgcggtggccacggttactctggatacaacggtttcggccaggcctacgccgactgggttgtccagtgcacctgggagggtgacaacaacattctcaccctttctgccggccgagctcttatccagtctgccgttgctctgcgaaagggcgagcctgttggtaacgccgtttcttacctgaagcgatacaaggatctggccaacgctaagctcaatggccgatctctcaccgaccccaaggtcctcgtcgaggcctgggaggttgctgccggtaacatcatcaaccgagccaccgaccagtacgagaagctcattggcgagggtcttaacgccgaccaggcctttgaggttctgtctcagcagcgattccaggccgccaaggtccacacacgacgacacctcattgccgctttcttctcccgaattgacaccgaggctggcgaggccatcaagcagcccctgcttaacctggctctgctgtttgccctgtggtccatcgaagaggactctggtctgttcctgcgagagggcttcctcgagcccaaggatatcgacaccgtcaccgagctcgtcaacaagtactgcaccactgtgcgagaggaggtcattggctacaccgatgccttcaacctgtccgactacttcatcaacgctcctattggatgctacgatggtgacgcttaccgacactacttccagaaggtcaacgagcagaaccctgcccgagacccccgacctccttactacgcctctactctcaagcccttccttttccgagaggaggaggatgatgacatttgcgagcttgatgaggaatag

SEQ ID NO: 4 - protein, 700 aa, Yarrowia lipolytica
MNPNNTGTIEINGKEYNTFTEPPVAMAQERAKTSFPVREMTYFLDGGEKNTLKNEQIMEEIERDPLFNNDNYYDLNKEQIRELTMERVAKLSLFVRDQPEDDIKKRFALIGIADMGTYTRLGVHYGLFFGAVRGTGTAEQFGHWISKGAGDLRKFYGCFSMTELGHGSNLAGLETTAIYDEETDEFIINTPHIAATKWWIGGAAHTATHTVVFARLIVKGKDYGVKTFVVQLRNINDHSLKVGISIGDIGKKMGRDGIDNGWIQFTNVRIPRQNLLMKYTKVDREGNVTQPPLAQLTYGSLITGRVSMASDSHQVGKRFITIALRYACIRRQFSTTPGQPETKIIDYPYHQRRLLPLLAYVYALKMTADEVGALFSRTMLKMDDLKPDDKAGLNEVVSDVKELFSVSAGLKAFSTWACADVIDKTRQACGGHGYSGYNGFGQAYADWVVQCTWEGDNNILTLSAGRALIQSAVALRKGEPVGNAVSYLKRYKDLANAKLNGRSLTDPKVLVEAWEVAAGNIINRATDQYEKLIGEGLNADQAFEVLSQQRFQAAKVHTRRHLIAAFFSRIDTEAGEAIKQPLLNLALLFALWSIEEDSGLFLREGFLEPKDIDTVTELVNKYCTTVREEVIGYTDAFNLSDYFINAPIGCYDGDAYRHYFQKVNEQNPARDPRPPYYASTLKPFLFREEEDDDICELDEE

SEQ ID NO: 5 - DNA, 2102 bp, Yarrowia lipolytica
atgatctcccccaacctcacagctaacgtcgagattgacggcaagcagtacaacaccttcacagagccacccaaggcgctcgccggcgagcgagccaaggtcaagttccccatcaaggacatgacggagtttctgcacggtggcgaggagaacgtgaccatgatcgagcgactgatgacggagctcgagcgagaccccgtgctcaacgtgtcgggcgactacgacatgcccaaggagcagctgcgagagacggccgtggcgcgaattgcggcgctgtccggccactggaagaaggacacagaaaaggaggcgctgctgcggtcccagctgcacggcattgtggacatgggcacccgaatccgactcggtgtgcacacgggcctgttcatgggcgccatccggggttccggcaccaaggagcagtacgactactgggtgcgaaagggcgccgcggacgtcaagggcttctacggctgctttgctatgaccgagctgggccatggctccaacgtggccggtcttgagaccaccgccacctacatccaggacacggacgagttcatcatcaacacccccaacactggagccaccaagtggtggattggaggagccgcccactcggccacccacaccgcctgctttgctcgtctgcttgtcgacggcaaggactacggcgtcaagatctttgttgtccagctgcgagacgtctcttctcactctctcatgcccggcatcgctctcggcgacattggaaagaagatgggccgagacgccatcgacaacggctggatccagttcaccaatgtgcgaatcccccgacagaacatgctcatgaagtacgccaaggtctcgtctaccggcaaggtgtcgcagcctcctctggcccagctcacctacggcgctctcattggcggccgagtcaccatgattgccgactccttctttgtctcccagcgattcatcaccattgctctgcgatacgcctgtgtgcgacgacagtttggcaccacccccggccagcccgagactaagatcatcgactacccctaccatcagcgacgtctgctgcctcttctggccttcacctacgccatgaagatggccgccgaccagtcccagattcagtacgatcagaccaccgatctgctgcagaccatcgaccctaaggacaagggcgctctgggcaaggccattgtcgacctcaaggagctgtttgcctcttctgctggtctcaaggccttcaccacctggacctgtgccaacatcattgaccagtgccgacaggcctgcggtggccacggctactctggctacaacggctttggccaggcctacgccgactgggttgtccagtgcacctgggagggtgacaacaacgtcctgtgtctgtccatgggccgaggtctcatccagtcgtgtctgggccaccgaaagggtaagcctctgggctcttctgtcggctacctggctaacaagggtcttgagcaggctactctgagcggccgagacctcaaggaccccaaggttctcatcgaggcctgggagaaggtcgccaacggcgccatccagcgggccactgacaaatttgtcgagctcaccaagggcggcctctctcctgaccaggcctttgaggagctgtcgcagcagcgattccagtgtgccaagatccacacccgaaagcacctggtgactgccttctacgagcgaatcaacgcctctgcgaaggccgacgtcaagccttacctcatcaacctcgccaacctcttcactctgtggtccattgaggaggactctggtctcttcctgcgagagggtttcctgcagcccaaggacattgaccaggtgactgagctggtgaaccactactgcaaggaggttcgagaccaggttgccggctacaccgatgcctttggtctgtctgactggttcatcaacgctcccattggaaactacatggtgacgtttacaagcattactttgccaaggttaaccagcagaaccctgctcagaacccccgacctccttactatgagagcactcttcgacctttcctgttccgagaggatgaggatgacgacatttgcgagctggacgaggaatag

SEQ ID NO: 6 - protein, 700 aa, Yarrowia lipolytica
MISPNLTANVEIDGKQYNTFTEPPKALAGERAKVKFPIKDMTEFLHGGEENVTMIERLMTELERDPVLNVSGDYDMPKEQLRETAVARIAALSGHWKKDTEKEALLRSQLHGIVDMGTRIRLGVHTGLFMGAIRGSGTKEQYDYWVRKGAADVKGFYGCFAMTELGHGSNVAGLETTATYIQDTDEFIINTPNTGATKWWIGGAAHSATHTACFARLLVDGKDYGVKIFVVQLRDVSSHSLMPGIALGDIGKKMGRDAIDNGWIQFTNVRIPRQNMLMKYAKVSSTGKVSQPPLAQLTYGALIGGRVTMIADSFFVSQRFITIALRYACVRRQFGTTPGQPETKIIDYPYHQRRLLPLLAFTYAMKMAADQSQIQYDQTTDLLQTIDPKDKGALGKAIVDLKELFASSAGLKAFTTWTCANIIDQCRQACGGHGYSGYNGFGQAYADWVVQCTWEGDNNVLCLSMGRGLIQSCLGHRKGKPLGSSVGYLANKGLEQATLSGRDLKDPKVLIEAWEKVANGAIQRATDKFVELTKGGLSPDQAFEELSQQRFQCAKIHTRKHLVTAFYERINASAKADVKPYLINLANLFTLWSIEEDSGLFLREGFLQPKDIDQVTELVNHYCKEVRDQVAGYTDAFGLSDWFINAPIGNYDGDVYKHYFAKVNQQNPAQNPRPPYYESTLRPFLFREDEDDDICELDEE

SEQ ID NO: 7 - DNA, 2105 bp, Yarrowia lipolytica
atgatcaccccaaaccccgctaacgacattgtccatgacggcaagctctacgacaccttcactgagccccccaagctgatggctcaggagcgagctcagctggacttcgaccctagagacatcacctactttctggatggctctaaggaggagaccgagctgctggagtcgctcatgctcatgtacgagcgagaccctctcttcaacaaccagaacgagtacgatgaatcgtttgaaacactgcgagagcgatctgtgaagcgaattttccagtgtccaagtccatcgccatggaccccgagcccatgtctttccgaaagattgggttcctgggtattcttgacatgggaacgtatgctcgactgggagtccactacgcgctcttctgtaactccatccggggccagggaacccccgatcagctcatgtactggctggaccagggagccatggtcatcaagggcttctacggctgttttgccatgaccgaaatgggccatggatctaacctgtcgcgtctggaaaccatcgccactttcgacaaagagaccgacgaatttatcattaacacgccccacgttggagccacaaagtggtggattggaggagccgcccacactgctactcacacacttgcctttgcccgtcttcaagtagacggaaaggactacggtgtgaaatcgtttgtcgtacctctccgaaacctggacgaccattcgctgcgtcctggaatcgccacaggtgatattggtaagaagatgggtcgagatgccgttgacaacggctggattcagttcaccaacgtccgagtgccccgaaactacatgctcatgaagcataccaaggttcttcgagacggtaccgtcaagcagccgcctttggcccaactgacttacggatctctcatcactggacgagtccagatgaccactgactctcacaatgtgtccaaaaagttcctcaccattgccctgagatacgccaccatccgacgacagttctcgtcaactccaggagagcccgaaacccgactaattgactacctgtaccaccaaagacgactcctgcctcttatggcttactcttacgccatgaaactagctggagatcacgtccgagagctgttctttgcatcccaggagaaggctgagagcctcaaggaggacgacaaagccggagttgagtcttacgtccaggatatcaaggagctcttctctgtttctgctggtctcaaggctgccactacatgggcttgtgctgacatcattgacaaggcccgacaggcgtgtggaggccacggatactctgcctacaacggctttggacaggccttccaggactgggttgtccagtgcacttgggagggtgacaatactgttctgactctatctgccggccgagctctgatccaatctgctctcgtctaccgaaaggagggcaaactaggtaacgccacgaagtacctctctcggtccaaggagcttgccaacgccaagagaaacggacgatccctggaagaccccaagctgctcgtggaggcatgggaggctgtctctgccggtgctatcaacgctgctactgacgcttacgaggagctctccaagcagggagtttctgttgacgagtgctttgagcaggtgtcccaggagcgattccaggctgcccgaatccacactcgacgagctcttatcgaggccttctactcacgaatcgccactgctgatgagaaggtgaagcctcatctgatccctctggccaacctgtttgccctgtggtccattgaggaggactctgctctgttcctggctgagggctactttgagcctgaggatatcattgaggtgacttctcttgtcaacaagtactgcggaattgttcgaaagaacgttattggatacaccgatgccttcaacctgtccgactacttcatcaacgctgccattggacgatacgacggagacgtgtacaagaactactttgagaaggtcaaacagcagtaccctcctgagggtggcaagcctcactactacgaggatgtcatgaagcccttcctgcatcgagagcgaattcccgatgtccccatggagcccgaggatattcagtaa

SEQ ID NO: 8 - protein, 701 aa, Yarrowia lipolytica
MITPNPANDIVHDGKLYDTFTEPPKLMAQERAQLDFDPRDITYFLDGSKEETELLESLMLMYERDPLFNNQNEYDESFETLRERSVKRIFQLSKSIAMDPEPMSFRKIGFLGILDMGTYARLGVHYALFCNSIRGQGTPDQLMYWLDQGAMVIKGFYGCFAMTEMGHGSNLSRLETIATFDKETDEFIINTPHVGATKWWIGGAAHTATHTLAFARLQVDGKDYGVKSFVVPLRNLDDHSLRPGIATGDIGKKMGRDAVDNGWIQFTNVRVPRNYMLMKHTKVLRDGTVKQPPLAQLTYGSLITGRVQMTTDSHNVSKKFLTIALRYATIRRQFSSTPGEPETRLIDYLYHQRRLLPLMAYSYAMKLAGDHVRELFFASQEKAESLKEDDKAGVESYVQDIKELFSVSAGLKAATTWACADIIDKARQACGGHGYSAYNGFGQAFQDWVVQCTWEGDNTVLTLSAGRALIQSALVYRKEGKLGNATKYLSRSKELANAKRNGRSLEDPKLLVEAWEAVSAGAINAATDAYEELSKQGVSVDECFEQVSQERFQAARIHTRRALIEAFYSRIATADEKVKPHLIPLANLFALWSIEEDSALFLAEGYFEPEDIIEVTSLVNKYCGIVRKNVIGYTDAFNLSDYFINAAIGRYDGDVYKNYFEKVKQQYPPEGGKPHYYEDVMKPFLHRERIPDVPMEPEDIQ

SEQ ID NO: 9 - DNA, 2100 bp, Yarrowia lipolytica
atgaacaacaaccccaccaacgtgatccttggaggcaaggagtacgacaccttcaccgagcctccggcccagatggagctggagcgagccaagacacaattcaaggtccgagacgtgaccaacttcctcacaggcagcgagcaggagacactgctgaccgagcgaatcatgcgggagattgagcgagatcccgttctcaacgtcgccggcgactacgacgccgatcttcccaccaagcgacgacaagctgttgagcgaatcggggctctggcccgatacctgcccaaggattccgagaaggaggccattttgcgaggccagctgcatggtattgtggacatgggtacccgaacccgaatcgccgttcactacggtctgtttatgggcgccattcgtggctcaggaaccaaggagcagtacgattactgggtcgccaagggcgccgctactctgcacaaattctatggctgctttgccatgactgagctgggtcacggatctaacgtggccggtctcgagaccaccgccacccttgataaggacaccgacgagttcatcatcaacacccccaactcgggagccacaaagtggtggattggaggagctgcccactctgctacccacacggcttgtcttgcccgactcattgttgatggcaaggactatggtgttaagatcttcattgttcagctgcgagacctcaactcccactctctactcaacggtattgccattggagatatcggcaagaagatgggccgagatgccattgataatggttggatccagttcacagacgtccgaattccccgacagaacatgctcatgcgatacgaccgggtgtctcgagacggcgaggttaccacctccgagcttgcccagctcacctacggagcacttctgtctggccgagtgaccatgattgccgagtctcacctcctgtctgctcggttcctcaccattgctcttcggtacgcctgtatccgtcgacagttcggagctgtgcctgacaagcccgagactaagctcatcgactacccctaccaccaacgacgtctgctgcctcttctggcctacacctacgccatgaagatgggcgccgacgaggcccagcagcagtacaactcctcctttggcgctcttctcaagctcaaccccgtcaaggacgctgagaagtttgctgtcgccactgccgacctcaaggctctgtttgcctcttctgccggaatgaaggccttcaccacctgggctgccgccaagatcattgacgagtgccgacaggcctgtggtggccatggctactccggctacaacggtttcggtcaggcttacgccgactgggtcgtccaatgcacttgggagggtgacaacaacgtgctgtgtctgtccatgggtcgatcgctcatccagtcgtgcattgccatgagaaagaagaagggccatgtcggcaagtcggtcgagtacctgcagcgacgagacgagctgcagaatgcccgagttgacaacaagcctctcactgaccctgctgtgctcatcactgcatgggagaaggttgcctgcgaggccatcaacagagccactgactccttcatcaagctcacccaggagggtctgtctcctgaccaggcctttgaggagctgtctcaacagagatttgagtgtgcgcgaatccacacccgaaagcatctgatcacctcgttctacgctcgaatctccaaggccaaggcccgagtcaagccccaccttactgttcttgccaacctctttgccgtctggtccatcgaggaggactctggtctcttccttcgggagggctgcttcgagcctgccgagatggacgagatcaccgctctggtcgacgagctgtgctgcgaggctcgagagcaggtcattggattcaccgacgccttcaacctgtccgacttcttcattaacgcccccattggccgattcgacggagacgcctacaagcactacatggacgaggtcaaggctgccaacaaccctcgtaacacccatgctccttactacgagaccaagctgcgacccttcctgttccgacccgatgaggacgaggagatttgcgacctggacgagtag

SEQ ID NO: 10 - protein, 699 aa, Yarrowia lipolytica
MNNNPTNVILGGKEYDTFTEPPAQMELERAKTQFKVRDVTNFLTGSEQETLLTERIMREIERDPVLNVAGDYDADLPTKRRQAVERIGALARYLPKDSEKEAILRGQLHGIVDMGTRTRIAVHYGLFMGAIRGSGTKEQYDYWVAKGAATLHKFYGCFAMTELGHGSNVAGLETTATLDKDTDEFIINTPNSGATKWWIGGAAHSATHTACLARLIVDGKDYGVKIFIVQLRDLNSHSLLNGIAIGDIGKKMGRDAIDNGWIQFTDVRIPRQNMLMRYDRVSRDGEVTTSELAQLTYGALLSGRVTMIAESHLLSARFLTIALRYACIRRQFGAVPDKPETKLIDYPYHQRRLLPLLAYTYAMKMGADEAQQQYNSSFGALLKLNPVKDAEKFAVATADLKALFASSAGMKAFTTWAAAKIIDECRQACGGHGYSGYNGFGQAYADWVVQCTWEGDNNVLCLSMGRSLIQSCIAMRKKKGHVGKSVEYLQRRDELQNARVDNKPLTDPAVLITAWEKVACEAINRATDSFIKLTQEGLSPDQAFEELSQQRFECARIHTRKHLITSFYARISKAKARVKPHLTVLANLFAVWSIEEDSGLFLREGCFEPAEMDEITALVDELCCEAREQVIGFTDAFNLSDFFINAPIGRFDGDAYKHYMDEVKAANNPRNTHAPYYETKLRPFLFRPDEDEEICDLDE

SEQ ID NO: 11 - DNA, 2070 bp, Yarrowia lipolytica
atgctctctcaacagtccctcaacacgtttaccgagcccccggtcgaaatggcccgggagcgaaaccagacttccttcaacccgcgtctgctgacgtactttctggacggaggcgaaaagaacactctgcttatggaccgactgatgcaagagtacgagcgagaccctgtgtttcgaaacgagggcgactacgatattaccgatgtggcccagtcgcgagagctggccttcaagcgaatcgccaagctcatcgagtatgtgcacaccgacgacgaggagacgtatctgtaccgatgcatgcttctgggccaaatcgatatgggagcctttgcccggtacgccatccaccacggagtctggggcggtgccattcgaggtgcaggaacgcctgagcagtacgaattctgggtcaagaaaggatctctgtcggttaagaagttctatggatccttctccatgaccgagctgggccacggcagtaacttggtgggtctggagaccaccgccaccctggacaagaacgcagacgagttcgtgatcaacactcccaacgttgctgccactaaatggtggatcggaggagccgccgataccgccactcacacagctgtgtttgcacgtctcattgtcgacggagaggaccacggtgtcaagacgtttgtggtgcagctgcgagacgtggagactcacaacctgatgcctggtattgctatcggagactgcggcaagaagatgggacgtcagggaaccgacaacggctggatccagttcacccatgtgcgaattccccgacagaacatgctcatgcgatactgtcacgtggacagcgacggaaatgttaccgagcccatgatggctcagatggcctacggagctcttctggctggccgagtcggaatggccatggacagttatttcacctcgcgaaagttccttaccattgctcttcgatatgccaccattcgacgagcttttgctgccggaggaggtcaggagaccaagctgatcgactacccttaccaccagcgacgtctgctccccctcatggcccagacatatgccatcaagtgcaccgccgataaggtcagagatcagttcgtcaaggtcaccgacatgctcctaaacctcgatgtttctgaccaagaggccgtgcccaaggccattgccgaggctaaggagctcttctctgtttctgctggtgtcaaggctaccacaacttgggcttgcgcacacaccattgaccagtgcagacaggcgtgtggaggccacggatactctgcttacaacggttttggacgtgcttactccgattgggtgatccagtgcacctgggagggagacaataacattctgtgtctgtcagctggcagagctctggtccagtctaaccgagctgtccgggctggcaagcccattggaggtcctaccgcctacctggctgctcccgctggttcccccaagctcgctggtcgaaacttgtacgaccccaaggtcatgattggggcctgggagactgtttcccgagctctgatcaaccgaaccaccgatgagtttgaggtgctggccaagaagggtctgtctactgcccaggcctacgaggagctgtcccagcaacgattcctgtgtactcgaatccacacccgtctgtacatggtcaagaacttctacgagcgaattgccgaggagggcaccgagttcaccaaggagcctcttaccagacttgccaacctgtacgccttctggtccgtcgaagaggaggctggaatcttcctccgagagggctacatcactccccaggagctcaagtacatcagtgccgagatccgaaagcagctcttggaggtgcgaaaggacgtcattggctacaccgatgccttcaacgtgcctgattttttcctcaactctgccattggacgagctgacggagatgtctacaagaactacttcaaggtggtcaacactcagaaccctccccaagaccctcgacctccttattacgagtctgtcattagacccttcctgttccgaaaggacgaggatgaggaaatttgctctcttgaggatgagtag

SEQ ID NO: 12 - protein, 689 aa, Yarrowia lipolytica
MLSQQSLNTFTEPPVEMARERNQTSFNPRLLTYFLDGGEKNTLLMDRLMQEYERDPVFRNEGDYDITDVAQSRELAFKRIAKLIEYVHTDDEETYLYRCMLLGQIDMGAFARYAIHHGVWGGAIRGAGTPEQYEFWVKKGSLSVKKFYGSFSMTELGHGSNLVGLETTATLDKNADEFVINTPNVAATKWWIGGAADTATHTAVFARLIVDGEDHGVKTFVVQLRDVETHNLMPGIAIGDCGKKMGRQGTDNGWIQFTHVRIPRQNMLMRYCHVDSDGNVTEPMMAQMAYGALLAGRVGMAMDSYFTSRKFLTIALRYATIRRAFAAGGGQETKLIDYPYHQRRLLPLMAQTYAIKCTADKVRDQFVKVTDMLLNLDVSDQEAVPKAIAEAKELFSVSAGVKATTTWACAHTIDQCRQACGGHGYSAYNGFGRAYSDWVIQCTWEGDNNILCLSAGRALVQSNRAVRAGKPIGGPTAYLAAPAGSPKLAGRNLYDPKVMIGAWETVSRALINRTTDEFEVLAKKGLSTAQAYEELSQQRFLCTRIHTRLYMVKNFYERIAEEGTEFTKEPLTRLANLYAFWSVEEEAGIFLREGYITPQELKYISAEIRKQLLEVRKDVIGYTDAFNVPDFFLNSAIGRADGDVYKNYFKVVNTQNPPQDPRPPYYESVIRPFLFRKDEDEEICSLEDE

SEQ ID NO: 13 - DNA, 2088 bp, Agrotis segetum
atgcccattttcatctgtatcatcacctcgcaagccatcatccgatcgaacgtggaacgagtggcagtcattcttaacatcaacatgggtaaggtgaacgaggacctggttcgagagcgtgcaaagtgtaccttcaacattgaggagctcacctactttcttgacggaggaaaagataaaaccttggaaagaaaggagaccgaacgagctatgttgaccaagcgagaagagctctttggaggcgttcccgacgaatacctgtcacacaaggagaagtacgagaactccatgcgaaaggctgttattctcttcggaatccttagaaagatccaaaaggataacaacaccgacctcaccaactaccggaatctgctgagcggagtgctgtccgtgtccatctcccaggatggctctcctttcggcctccactacatcatgttcatgcctgtgctgctgtctcaggctgatgaaaaacaacaagagaagtggctgaagcgagccatgaactgtgaaatcatcggctcttacgcccagaccgagctgggccacggcactttcatccgaggccttgagactactgccacttacgatcccgccacccaggagttcgtgctgcactctcctgctctttcgtcctacaagtggtggcctggaggcctcggaaacactgtcaattactgcatcgttattgcccagctgtactctaagggcgtctgtcacggcattcattccttcatcgtgcaggtgcgagacgaggacactcacatgccgttgcccggcatcaaggtcggagagatcggtgtgaagatgggtctgaactctgtcaacaacggattcctgggattcgagaacgtccgaatcccccgagtgaacatgcttatgaagcacgccaagatcctggaggatggaacttacgttaagtcgaagaacaacaagctcatctacggagcaatggtgttcgtccgagtggtgatcgtgttcgactctgtgaactacctggccaaggccatcaccattggcgcacgatactccctggtgcggcgacagtcgcaattaaaagctggagaacctgagcgacagatcctcgactatgtcacccagcagcacaagattctgcccgctatcgctggatgctacgccatgaagatgaacgcttggaggttgtgggacaccttcaacctgatcaacggacaactgcatcagggcaacatggaacggctgggcgagctgcatgccctcgcgtgctgcctcaaggctatctctaccaccgacgcggctatgttcacctctctgtgtcgactcggatgcggaggtcacggttacatgacttcttccaacctccctcccacatacgctttgacttctgcctcgtgcacttacgagggagacaacaccgttctgctgctgcagaccgctcgatttcttctcaagacctggcgacagattgacacccaccctctgactagaaccgtggcctacctgaagaccgtgtctgcccctggattctctgacagatgggagtcttccgtggagggcatcattcgaggcttccagaccgtcgctatgaaaaagatttcttcctgtctggacatcatgacttctaaggtgatgtctggaatgtcccaggaggatgcatggaacgctatttctatccagctggtttcggccgctgaatcccattctcgaggcaccgtgatctctacgttttacgaagacatgtctaaggccatgcgatccatgactgctcccttggcgaaggtgatgggtcagctggttgagctgtacgcggtttattggactctagagcgactgggagacatgttgcagtacaccagtatttctcacaccgacgttgttgacctccgatcctggtacgaagagctcctccgaaagatccgacctaacactatcggactggtggacgcgtttgatattattgatgaactgctccagtccaccctgggtgcttatgacggtcgtgtttacgaacgactgatggaagaagctttgaagtctcccctgaacgctgagcccgtgaaccagtccttccacaagtacctcaagccctttatgcagtctaagctgtaa

SEQ ID NO: 14 - protein, 695 aa, Agrotis segetum
MPIFICIITSQAIIRSNVERVAVILNINMGKVNEDLVRERAKCTFNIEELTYFLDGGKDKTLERKETERAMLTKREELFGGVPDEYLSHKEKYENSMRKAVILFGILRKIQKDNNTDLTNYRNLLSGVLSVSISQDGSPFGLHYIMFMPVLLSQADEKQQEKWLKRAMNCEIIGSYAQTELGHGTFIRGLETTATYDPATQEFVLHSPALSSYKWWPGGLGNTVNYCIVIAQLYSKGVCHGIHSFIVQVRDEDTHMPLPGIKVGEIGVKMGLNSVNNGFLGFENVRIPRVNMLMKHAKILEDGTYVKSKNNKLIYGAMVFVRVVIVFDSVNYLAKAITIGARYSLVRRQSQLKAGEPERQILDYVTQQHKILPAIAGCYAMKMNAWRLWDTFNLINGQLHQGNMERLGELHALACCLKAISTTDAAMFTSLCRLGCGGHGYMTSSNLPPTYALTSASCTYEGDNTVLLLQTARFLLKTWRQIDTHPLTRTVAYLKTVSAPGFSDRWESSVEGIIRGFQTVAMKKISSCLDIMTSKVMSGMSQEDAWNAISIQLVSAAESHSRGTVISTFYEDMSKAMRSMTAPLAKVMGQLVELYAVYWTLERLGDMLQYTSISHTDVVDLRSWYEELLRKIRPNTIGLVDAFDIIDELLQSTLGAYDGRVYERLMEEALKSPLNAEPVNQSFHKYLKPFMQSKL

SEQ ID NO: 15 - DNA, 2031 bp, Arabidopsis thaliana
atggaaggcatcgaccacctggccgacgagcgaaacaaggccgagttcgacgttgaggacatgaagatcgtgtgggccggctctcgacacgccttcgaggtgtctgaccgaatcgcccgactggtggcttctgaccccgtgttcgagaagtctaaccgagccagactgtctcgaaaggaactgttcaagtctaccctgcgaaagtgcgcccacgccttcaagcgaatcatcgagctgcgactgaacgaggaagaggccggacgactgcgacacttcattgaccagcctgcctacgtggacctgcactggggcatgttcgtgcccgccatcaagggccagggcaccgaggaacagcagaagaagtggctgtctctggccaacaagatgcagatcatcggctgctacgcccagaccgagcttggccacggctctaacgtgcagggcctcgagactaccgccactttcgaccccaagaccgacgagttcgtgattcacacccctactcagaccgcctctaagtggtggcccggtggcctcggcaaggtgtctacccacgccgtggtgtacgctcgactgatcaccaacggcaaggactacggcatccacggcttcatcgtgcagctgcgatctctcgaggaccactctcctctgcctaacatcaccgtgggcgacatcggcaccaagatgggcaacggcgcctacaactctatggacaacggcttcctgatgttcgaccacgtgcgaattccccgagatcagatgctgatgcgactgtctaaggtgacccgagagggcgagtacgtgccctctgacgtgcccaagcagctggtgtacggaaccatggtgtacgtgcgacagaccatcgtggccgacgcttctaacgccctgtctcgagccgtgtgtatcgctacccgatactctgccgtgcgacgacagttcggcgcccacaacggcggcatcgagactcaggtgatcgactacaagacccagcagaaccgactgttccctctgctggcctccgcctacgccttccgattcgtcggcgagtggctgaagtggctctacaccgacgtgaccgagcgactggccgcctctgacttcgccactctgcccgaggctcacgcctgcaccgccggactgaagtctctgaccaccaccgccaccgctgacggcatcgaagagtgccgaaagctgtgtggcggccacggatacctgtggtgctctggactgcccgagctgttcgccgtgtacgtccccgcctgtacctacgagggcgacaacgtggtgctgcagctccaggtggcccgattcctgatgaagaccgtcgctcagctcggctctggcaaggtgcccgtgggaaccaccgcctacatgggccgagccgctcacctcctgcagtgccgatctggcgtgcagaaggccgaggactggctgaaccccgacgtcgtgctcgaggctttcgaggcccgagcactgcgaatggccgtgacctgcgccaagaacctgtctaagttcgagaaccaggaacagggcttccaagagctgctggccgacctggtcgaggccgctatcgcccactgccagctgatcgtggtgtccaagttcattgctaagctcgagcaggacatcggcggcaagggcgtgaagaagcagctgaacaacctgtgctacatctacgccctgtacctgctgcacaagcacctgggcgactttctgtctaccaactgcattacccctaagcaggcctctctggctaacgaccagctccgatcgctgtacacccaggtgcgacccaacgctgtggccctggtggacgctttcaactacactgaccactacctgaactctgtgctgggccgatacgacggcaacgtgtaccccaagctgttcgaagaggccctgaaggaccctctgaacgactctgtggtgcccgacggctaccaagagtacctgcgacctgtcctgcagcagcagctccgaaccgctcgactcgaccagattacctctgtgggatcttcttcgaagctgtag

SEQ ID NO: 16 - protein, 676 aa, Arabidopsis thaliana
MEGIDHLADERNKAEFDVEDMKIVWAGSRHAFEVSDRIARLVASDPVFEKSNRARLSRKELFKSTLRKCAHAFKRIIELRLNEEEAGRLRHFIDQPAYVDLHWGMFVPAIKGQGTEEQQKKWLSLANKMQIIGCYAQTELGHGSNVQGLETTATFDPKTDEFVIHTPTQTASKWWPGGLGKVSTHAVVYARLITNGKDYGIHGFIVQLRSLEDHSPLPNITVGDIGTKMGNGAYNSMDNGFLMFDHVRIPRDQMLMRLSKVTREGEYVPSDVPKQLVYGTMVYVRQTIVADASNALSRAVCIATRYSAVRRQFGAHNGGIETQVIDYKTQQNRLFPLLASAYAFRFVGEWLKWLYTDVTERLAASDFATLPEAHACTAGLKSLTTTATADGIEECRKLCGGHGYLWCSGLPELFAVYVPACTYEGDNVVLQLQVARFLMKTVAQLGSGKVPVGTTAYMGRAAHLLQCRSGVQKAEDWLNPDVVLEAFEARALRMAVTCAKNLSKFENQEQGFQELLADLVEAAIAHCQLIVVSKFIAKLEQDIGGKGVKKQLNNLCYIYALYLLHKHLGDFLSTNCITPKQASLANDQLRSLYTQVRPNAVALVDAFNYTDHYLNSVLGRYDGNVYPKLFEEALKDPLNDSVVPDGYQEYLRPVLQQQLRTARLDQITSVGSSSKL

SEQ ID NO: 17 - DNA, 2115 bp, Arabidopsis thaliana
atggaatctcgacgagagaagaaccccatgaccgaggaagagtctgacggcctgatcgccgctcgacgaatccagcgactgtctctgcacctgtcgccttctctgaccccttctccttcgctgcccctggtgcagaccgagacttgctctgcccgatctaagaagctggacgtcaacggcgaggccctgtctctgtacatgcgaggcaagcacatcgacatccaagagaagattttcgacttcttcaactctcgacccgacctgcagacccctatcgagatctctaaggacgaccaccgagagctgtgcatgaaccagctgatcggcctggtgcgagaggccggcgtgcgacccttccgatacgtggctgacgaccctgagaagtactttgccatcatggaagccgtgggctctgtggacatgtctctgggcatcaagatgggcgtgcagtactctctgtggggcggctctgtgatcaacctgggcaccaagaagcaccgagacaagtacttcgacggcatcgacaacctggactacaccggctgcttcgctatgaccgagctgcaccacggctctaacgtgcagggactgcagaccaccgccactttcgaccctctgaaggacgagttcgtgatcgacacccctaacgacggcgccatcaagtggtggatcggcaacgccgccgtccacggcaagttcgccaccgtgttcgcccgactgattctgcccactcacgactctaagggcgtgtctgacatgggagtgcacgccttcatcgtgcccatccgagacatgaagacccaccagactctgcccggcgtcgagatccaggactgcggccacaaggtgggcctgaacggcgtggacaacggcgccctgcgattccgatctgtgcgaattccccgagacaacctgctgaaccgattcggcgacgtgtctcgagatggcacctacacctcttctctgcccaccatcaacaagcgattcggagctaccctgggcgagctggtcggcggacgagtcggcctggcctacgcctctgtgggcgtgctgaagatctccgccactatcgccatccgatactccctgctgcgacagcagttcggccctcctaagcagcccgaggtgtctattctggactaccagtctcagcagcacaagctgatgcccatgctggcctctacctacgcctaccacttcgctaccgtgtacctggtggaaaagtactctgagatgaagaagactcacgacgagcagctggtggccgacgtgcacgccctgtctgccggactgaagtcttacgtgacctcttacaccgccaaggctctgtctgtgtgccgagaggcctgtggcggccacggctacgccgctgtcaaccgatttggctctctgcgaaacgaccacgacatcttccagaccttcgagggcgacaacaccgtgctgctccagcaggtcgccgccgacctcctgaagcgatacaaggaaaagttccaaggcggcaccctgaccgtcacctggtcttacctgcgagagtctatgaacacctacctctcgcagcccaaccctgtgaccgctcgatgggaaggcgaggaccatctgcgagatcccaagtttcagctggacgctttccgataccgaacctctcgactgctgcagaacgtggctgcccgactgcagaagcactctaagaccctcggcggcttcggcgcctggaaccgatgcctgaaccatctgctgaccctggccgagtctcacatcgagactgtgatcctggccaagttcatcgaggccgtgaagaactgccccgatccttctgccaaggccgctctgaagctggcctgcgacctgtacgccctggaccgaatctggaaggacatcggcacctaccgaaacgtggactacgtggctcccaacaaggccaaggccatccacaagctcaccgagtacctgtctttccaggtgcgaaacgtcgccaaggaactggtggacgccttcgagctgcctgaccacgtgactcgagcccctattgccatgcagtctgacgcctactctcagtacacccaggtggtgggcttcgaccagattacctccgtgggatcttcttcgaagctgtag

SEQ ID NO: 18 - protein, 704 aa, Arabidopsis thaliana
MESRREKNPMTEEESDGLIAARRIQRLSLHLSPSLTPSPSLPLVQTETCSARSKKLDVNGEALSLYMRGKHIDIQEKIFDFFNSRPDLQTPIEISKDDHRELCMNQLIGLVREAGVRPFRYVADDPEKYFAIMEAVGSVDMSLGIKMGVQYSLWGGSVINLGTKKHRDKYFDGIDNLDYTGCFAMTELHHGSNVQGLQTTATFDPLKDEFVIDTPNDGAIKWWIGNAAVHGKFATVFARLILPTHDSKGVSDMGVHAFIVPIRDMKTHQTLPGVEIQDCGHKVGLNGVDNGALRFRSVRIPRDNLLNRFGDVSRDGTYTSSLPTINKRFGATLGELVGGRVGLAYASVGVLKISATIAIRYSLLRQQFGPPKQPEVSILDYQSQQHKLMPMLASTYAYHFATVYLVEKYSEMKKTHDEQLVADVHALSAGLKSYVTSYTAKALSVCREACGGHGYAAVNRFGSLRNDHDIFQTFEGDNTVLLQQVAADLLKRYKEKFQGGTLTVTWSYLRESMNTYLSQPNPVTARWEGEDHLRDPKFQLDAFRYRTSRLLQNVAARLQKHSKTLGGFGAWNRCLNHLLTLAESHIETVILAKFIEAVKNCPDPSAKAALKLACDLYALDRIWKDIGTYRNVDYVAPNKAKAIHKLTEYLSFQVRNVAKELVDAFELPDHVTRAPIAMQSDAYSQYTQVVGFDQITSVGSSSKL

SEQ ID NO: 19 - DNA, 2091 bp, Aspergillus nidulans
atgcccaaccctccgcctgcctgggtgcaagctctgaagcccgcttcgccccagggcaccgagctgctgacccaagagcgagcccagtctaacatcgacgtggacaccctgggcgacctgctgcacaccaaggaagccctgaagaagcaggacgagatcctgtctgtgctgaagtctgagaaggtgttcgacaagtctcgaaaccacgtgctgggccgaaccgagaagatccagctggccctggctcgaggcaagcgactgcagcagctgaagaaggcccacaactggtctgacgaggacgtccacgtggccaacgacctggtgtctgagcccactccttacggcctgcacgcctctatgtttctggtgaccctgcgagagcagggcacccctgagcagcacaagctgttctacgaacgagcccgaaactacgagatcatcggctgctacgcccagaccgagcttggccacggctctaacgtgcgaggactcgagactaccgccacttgggacccctctgaccagaccttcatcattcactctcccactctgaccgcctctaagtggtggatcggctctctgggacgaaccgccaaccacgccgtggtgatggcccagctgtacatcggcggcaagaactacggaccccatcctttcgtggtgcagatccgagacatggaaacccaccagcctctcgagaacgtgtacgtgggcgacatcggccccaagttcggctacaacaccatggacaacggctttctgctgttcaacaagctgaagattccccacgtgaacatgctggcccgattcgcccaggtggacaaggccaccaacaagtacattcgacccgcttctccctctctgatgtacggcaccatgacctgggtgcgatccaacatcgtgctgcaggctggcggcgtgctcgcccgaggcgtgaccattgccgtgcgatactgcgccgtgcgacgacagttccaggaccgagatgccaaggccaacgccgaagagaaccaggtgctgaactacaagatggtccagattcgactgctgcccctgctggccgccatgtacgccctgcacttcaccggccgaggcatgatgcgactgtacgaagaaaaccaagaacgaatgaaggctgccgctcaggccgaccaagagaagcgaggcgctggccccgagcagctgcgagccggatctgacctgctggctgacctgcacgctacctcttgcggcctgaaggccctggcctctaccaccgctggcgagggcctcgaggtgtgccgacgagcctgtggcggccacggatactctaactactctggcatcggaccctggtacgccgactacctgcctactctgacctgggagggcgacaactacatgctgactcagcaggttgcccgatacctgctcaagtctgcccgagccgtgctggccggcaagggcaccgccaacgacacctctcgaatcctgcaggcctacctcgctcgacgagacaagggcgcctctttcgacatcctgggcaacgacgccgacattgtggccgccttcgcctggcgaaccgctcacctgaccttcgagactctgaagtaccgagatgtcgagaagcgatcttggaactctctgctgatcaacttctggcgactgtctaccgctctgtctcagtacctggtggtgaagaacttctacgaggccgtgaactctcccgagatccgatcttctctggacaaggacactgcttctaccctgcgatctctgttccgactgcacgctctgcacaccctggaccgagaggcctccgagttcttctcttctgccgccgtgaccgtgcgacagatcggactgacccagacctctgaggtgcccaagctgctggacgagattcgaccccacgccgtccgactggtggactcttggaagatccccgactggcagctggactctgccctgggccgatctgacggcgacgtgtaccccgacctgttcaagcgagcctctatgcagaaccccgtgaacgacctcgtgttcgacccctatccttggaacgagaacgtcctgaagaacgccggtgagatcaagtctaagctgtag

SEQ ID NO: 20 - protein, 696 aa, Aspergillus nidulans
MPNPPPAWVQALKPASPQGTELLTQERAQSNIDVDTLGDLLHTKEALKKQDEILSVLKSEKVFDKSRNHVLGRTEKIQLALARGKRLQQLKKAHNWSDEDVHVANDLVSEPTPYGLHASMFLVTLREQGTPEQHKLFYERARNYEIIGCYAQTELGHGSNVRGLETTATWDPSDQTFIIHSPTLTASKWWIGSLGRTANHAVVMAQLYIGGKNYGPHPFVVQIRDMETHQPLENVYVGDIGPKFGYNTMDNGFLLFNKLKIPHVNMLARFAQVDKATNKYIRPASPSLMYGTMTWVRSNIVLQAGGVLARGVTIAVRYCAVRRQFQDRDAKANAEENQVLNYKMVQIRLLPLLAAMYALHFTGRGMMRLYEENQERMKAAAQADQEKRGAGPEQLRAGSDLLADLHATSCGLKALASTTAGEGLEVCRRACGGHGYSNYSGIGPWYADYLPTLTWEGDNYMLTQQVARYLLKSARAVLAGKGTANDTSRILQAYLARRDKGASFDILGNDADIVAAFAWRTAHLTFETLKYRDVEKRSWNSLLINFWRLSTALSQYLVVKNFYEAVNSPEIRSSLDKDTASTLRSLFRLHALHTLDREASEFFSSAAVTVRQIGLTQTSEVPKLLDEIRPHAVRLVDSWKIPDWQLDSALGRSDGDVYPDLFKRASMQNPVNDLVFDPYPWNENVLKNAGEIKSKL

SEQ ID NO: 21 - DNA, 1977 bp, Cucurbita maxima
atggccgctggcaaggccaaggctaagatcgaggtggacatgggatctctgtctctgtacatgcgaggcaagcaccgagagatccaagagcgagtgttcgagtacttcaactctcgacccgagctgcagacccctgtgggcatctctatggccgaccaccgagagctgtgcatgaagcagctggtcggcctggtgcgagaggccggcattcgacccttccgattcgtgaacgaggaccccgccaagtacttcgccatcatggaagccgtgggctctgtggacgtgtctctggccatcaagatgggcgtgcagttctctctgtggggcggctctgtgatcaacctgggcaccaagaagcaccgggaccgattcttcgacggcatcgacaacgtggactaccccggctgcttcgccatgactgagctgcaccacggctctaacgtgcagggcctgcagaccaccgccactttcgaccccatcaccgacgagttcatcatcaacacccctaacgacggcgccatcaagtggtggatcggcaacgccgccgtccacggcaagttcgccaccgtgttcgccaagctggtgctgcccactcacgactctcgaaagaccgccgacatgggagtgcacgccttcatcgtgcccatccgagatctgaagtctcacaagaccctgcctggcatcgagatccacgactgcggccacaaggtgggcctgaacggcgtggacaacggcgccctgcgattccgatctgtgcgaattccccgagacaacctgctgaaccgattcggcgaggtgtctcgagatggcaagtacaagtcctctctgccctctatcaacaagcgattcgccgccactctgggcgagctggttggcggccgagtcggactggcctactcttctgcctctgtgctgaagatcgcctctactatcgccatccgatactccctgctgcgacagcagttcggccctcctaagcagcccgaggtgtccatcctggactaccagtctcagcagcacaagctgatgcccatgctggcctctacctacgccttccacttctctaccatgcagctcgtcgagaagtacgcccagatgaagaagacccacgacgaggaactggtgggcgacgtgcacgccctgtctgccggcctgaaggcctacgtgacctcttacaccgccaagtctctgtctacctgccgagaggcctgtggcggccacggatacgccgtggtcaaccgatttggcaccctgcgaaacgaccacgacatcttccagaccttcgagggcgacaacaccgtgctgctccagcaggtcgccgcctacctgctcaagcagtaccaagagaagttccaaggcggcaccctggccgtgacctggaactacctgcgagaatctatgaacacctacctctcgcagcccaaccctgtgaccgctcgatgggagtctgccgaccatctgcgagatcccaagtttcagctggacgctttccagtaccgaacctctcgactgctgcagtctgtggccgtgcgactgcgaaagcacaccaagaacctgggatctttcggcgcctggaaccgatgcctgaaccatctgctgaccctggctgagtctcacatcgagtctgtgattctggcccagttcatcgagtccgtgcagagatgtcccaacgctaacacccaggctaccctgaagctggtgtgcgacctgtacgctctggaccgaatctggaacgacatcggcacctaccgaaacgtcgactacgtggctcccaacaaggcaaaggccatccacaagctcaccgagtacctgtgcttccaggtgcgaaacattgcccaagagctggtggacgccttcgacctgcctgaccacgtgactcgagcccctattgccatgaagtctaacgcctactctcagtacacccagtacatcggcttcgaccagattacctctgtgggatcttcgtctaagctgtag

SEQ ID NO: 22 - protein, 658 aa, Cucurbita maxima
MAAGKAKAKIEVDMGSLSLYMRGKHREIQERVFEYFNSRPELQTPVGISMADHRELCMKQLVGLVREAGIRPFRFVNEDPAKYFAIMEAVGSVDVSLAIKMGVQFSLWGGSVINLGTKKHRDRFFDGIDNVDYPGCFAMTELHHGSNVQGLQTTATFDPITDEFIINTPNDGAIKWWIGNAAVHGKFATVFAKLVLPTHDSRKTADMGVHAFIVPIRDLKSHKTLPGIEIHDCGHKVGLNGVDNGALRFRSVRIPRDNLLNRFGEVSRDGKYKSSLPSINKRFAATLGELVGGRVGLAYSSASVLKIASTIAIRYSLLRQQFGPPKQPEVSILDYQSQQHKLMPMLASTYAFHFSTMQLVEKYAQMKKTHDEELVGDVHALSAGLKAYVTSYTAKSLSTCREACGGHGYAVVNRFGTLRNDHDIFQTFEGDNTVLLQQVAAYLLKQYQEKFQGGTLAVTWNYLRESMNTYLSQPNPVTARWESADHLRDPKFQLDAFQYRTSRLLQSVAVRLRKHTKNLGSFGAWNRCLNHLLTLAESHIESVILAQFIESVQRCPNANTQATLKLVCDLYALDRIWNDIGTYRNVDYVAPNKAKAIHKLTEYLCFQVRNIAQELVDAFDLPDHVTRAPIAMKSNAYSQYTQYIGFDQITSVGSSSKL

SEQ ID NO: 23 - DNA, 2019 bp, Homo sapiens
atgaaccccgacctgcgacgagagcgagactctgcctctttcaaccccgagctgctgacccacatcctggacggctctcccgaaaagacccgacgacgacgagaaatcgagaacatgatcctgaacgaccccgacttccagcacgaggacctgaactttctgacccgatctcagcgatacgaggtggccgtgcgaaagtctgccatcatggtgaagaagatgcgagagttcggaatcgctgaccccgacgagatcatgtggttcaagaacttcgtgcaccgaggacgacccgagcctctggacctgcacctgggcatgtttctgcccactctgctgcaccaggccaccgccgagcagcaagagcgattcttcatgcccgcctggaacctcgagatcatcggcacctacgctcagaccgagatgggccacggcacccacctccgaggactcgagactaccgccacctacgatcccgagactcaagagttcatcctgaactctcccaccgtgacctctatcaagtggtggcccggtggactgggcaagacctctaaccacgccatcgtgctggcccagctgatcaccaagggcaagtgctacggcctgcacgccttcatcgtgcccatccgagagatcggaacccacaagcctctgcctggcatcaccgtgggcgacatcggccccaagttcggctacgacgagattgacaacggctacctgaagatggacaaccaccgaattcctcgagagaacatgctgatgaagtacgcccaggtgaagcccgacggaacctacgtgaagcccctgtctaacaagctgacctacggaaccatggtgttcgtgcgatctttcctggtcggcgaggccgctcgagccctgtccaaggcctgcaccattgccatccgatactctgccgtgcgacaccagtctgagatcaagcccggcgagcctgagcctcagatcctggactttcagacccagcagtacaagctgttccctctgctggccaccgcttacgccttccagttcgtgggcgcctacatgaaggaaacctaccatcgaatcaacgaaggcatcggccagggcgacctgtctgagctgcccgaactgcacgccctgaccgccggactgaaggctttcacctcttggaccgccaacaccggcatcgaggcctgccgaatggcctgtggcggccacggctactctcactgctctggactgcccaacatctacgtgaacttcaccccttcgtgtaccttcgagggcgagaacaccgtgatgatgctgcagaccgctcgattcctcatgaagtcttacgaccaggtgcactctggcaagctggtgtgcggcatggtgtcttacctgaacgatctgccctctcagcgaattcagcctcagcaggttgccgtgtggcccaccatggtcgacatcaactctcccgagtctctgaccgaggcctacaagctgcgagctgctcgactggtcgagatcgccgccaagaacctgcagaaggaagtcatccaccgaaagtctaaggaagtggcttggaacctgacctctgtggacctggtgcgagcttctgaggcccactgccactacgtggtggtgaagctgttctctgagaagctgctgaagatccaggacaaggccatccaggccgtgctgcgatctctgtgcctgctgtactctctgtacggcatctctcagaacgccggcgacttcctgcagggctctatcatgactgagccccagattacccaggtcaaccagcgagtgaaggaactgctcaccctgatccgatctgacgccgtggctctggtggacgccttcgactttcaggacgtgaccctgggctctgtgctgggccgatacgacggcaacgtgtacgagaacctgttcgagtgggccaagaactcgcccctgaacaaggccgaggtgcacgagtcttacaagcacctgaagtctctgcagtctaagctggaccagattacttctgtgggatcttcttcgaagctgtag

SEQ ID NO: 24 - protein, 672 aa, Homo sapiens
MNPDLRRERDSASFNPELLTHILDGSPEKTRRRREIENMILNDPDFQHEDLNFLTRSQRYEVAVRKSAIMVKKMREFGIADPDEIMWFKNFVHRGRPEPLDLHLGMFLPTLLHQATAEQQERFFMPAWNLEIIGTYAQTEMGHGTHLRGLETTATYDPETQEFILNSPTVTSIKWWPGGLGKTSNHAIVLAQLITKGKCYGLHAFIVPIREIGTHKPLPGITVGDIGPKFGYDEIDNGYLKMDNHRIPRENMLMKYAQVKPDGTYVKPLSNKLTYGTMVFVRSFLVGEAARALSKACTIAIRYSAVRHQSEIKPGEPEPQILDFQTQQYKLFPLLATAYAFQFVGAYMKETYHRINEGIGQGDLSELPELHALTAGLKAFTSWTANTGIEACRMACGGHGYSHCSGLPNIYVNFTPSCTFEGENTVMMLQTARFLMKSYDQVHSGKLVCGMVSYLNDLPSQRIQPQQVAVWPTMVDINSPESLTEAYKLRAARLVEIAAKNLQKEVIHRKSKEVAWNLTSVDLVRASEAHCHYVVVKLFSEKLLKIQDKAIQAVLRSLCLLYSLYGISQNAGDFLQGSIMTEPQITQVNQRVKELLTLIRSDAVALVDAFDFQDVTLGSVLGRYDGNVYENLFEWAKNSPLNKAEVHESYKHLKSLQSKLDQITSVGSSSKL

SEQ ID NO: 25 - DNA, 2148 bp, Paenarthrobacter ureafaciens
atgaccgaggtggtggaccgagcctcttctcccgcctctcctggctctaccaccgccgctgccgacggcgccaaggtggccgtcgagcctcgagtggacgtggccgctctgggcgagcagctcctcggccgatgggccgacatccgactgcacgcccgagatctggccggacgagaggtggtgcagaaggtcgagggactgacccacaccgagcaccgatctcgagtgttcggccagctgaagtacctggtggacaacaacgccgtgcaccgagctttcccttctcgactcggcggatctgacgaccacggcggcaacattgccggcttcgaggaactggtgactgctgacccctcgctgcagatcaaggccggcgtccagtggggcctgttcggctctgccgtgatgcacctgggcacccgagagcaccacgacaagtggctgcccggcatcatgtctctcgagatccccggctgcttcgccatgaccgagactggccacggctctgacgtggcctctatcgccaccaccgccacctacgacgaagagactcaagagttcgtgatcgacacacccttccgagccgcctggaaggactacatcggcaacgccgccaacgacggcctggccgccgtcgtgttcgctcagctgatcacccgaaaggtgaaccacggcgtccacgccttctacgtggacctgcgagatcccgccaccggcgactttctgcccggaatcggcggcgaggacgacggcatcaagggcggcctgaacggcatcgacaacggacgactgcacttcaccaacgtgcgaattccccgaactaacctgctgaaccgatacggcgacgtggctgtggacggcacctactcttctaccatcgagtctcccggccgacgattcttcaccatgctgggaaccctggtgcagggccgagtgtctctggacggcgctgccgtggccgcctctaaggtggccctgcagtctgccatccactacgccgccgagcgacgacagttcaacgccacctctcctaccgaggaagaggtgctgctggactaccagcgacatcagcgacgactgtttacccgactggctactacctacgctgcctctttcgcccacgaacagctgctgcaaaagttcgacgacgtgttctctggcgcccacgacaccgacgccgaccgacaggacctcgagactctggctgccgctctgaagcccctgtctacctggcacgccctggacaccctgcaagagtgccgagaggcctgcggcggagccggcttcctgatcgagaaccgattcgcctctctgcgagctgacctggacgtgtacgtgaccttcgagggcgacaacaccgtgctgctgcagctggtggccaagcgactgctggccgactacgccaaggaattccgaggcgccaacttcggcgtgctggcccgatacgtcgtggaccaggccgctggcgtggctctgcaccgaaccggcctgcgacaggtggcccagttcgtggccgactccggctctgtgcagaagtctgccctggctctgcgagatgaggaaggccagcgaaccctgctgaccgaccgagtgcagtctatggtggccgaggtgggcgctgccctgaagggcgctggcaagctgccccagcaccaggctgctgccctgttcaaccagcatcagaacgagctgatcgaggccgctcaggcccacgccgagctgctccagtgggaagccttcaccgaggctctggccaaggtcgacgacgccggcaccaaggaagtgctgacccgactgcgggacctgttcggactgtctctgattgagaagcacctgtcttggtatctgatgaacggccgactgtctatgcagcggggacgaaccgtgggcacctacatcaaccgactgctcgtgaagattcgaccccacgctctggacctggtcgacgccttcggctacggcgctgagcatctgcgagccgccattgccaccggtgccgaggccactcgacaggacgaggcccgaacctacttccgacagcagcgagcctctggatctgcccctgccgacgaaaagaccctgctggccattaaggccggcaagtcccgagatcagattacctctgtgggatcttcttcgaagctgtag

SEQ ID NO: 26 - protein, 715 aa, Paenarthrobacter ureafaciens
MTEVVDRASSPASPGSTTAAADGAKVAVEPRVDVAALGEQLLGRWADIRLHARDLAGREVVQKVEGLTHTEHRSRVFGQLKYLVDNNAVHRAFPSRLGGSDDHGGNIAGFEELVTADPSLQIKAGVQWGLFGSAVMHLGTREHHDKWLPGIMSLEIPGCFAMTETGHGSDVASIATTATYDEETQEFVIDTPFRAAWKDYIGNAANDGLAAVVFAQLITRKVNHGVHAFYVDLRDPATGDFLPGIGGEDDGIKGGLNGIDNGRLHFTNVRIPRTNLLNRYGDVAVDGTYSSTIESPGRRFFTMLGTLVQGRVSLDGAAVAASKVALQSAIHYAAERRQFNATSPTEEEVLLDYQRHQRRLFTRLATTYAASFAHEQLLQKFDDVFSGAHDTDADRQDLETLAAALKPLSTWHALDTLQECREACGGAGFLIENRFASLRADLDVYVTFEGDNTVLLQLVAKRLLADYAKEFRGANFGVLARYVVDQAAGVALHRTGLRQVAQFVADSGSVQKSALALRDEEGQRTLLTDRVQSMVAEVGAALKGAGKLPQHQAAALFNQHQNELIEAAQAHAELLQWEAFTEALAKVDDAGTKEVLTRLRDLFGLSLIEKHLSWYLMNGRLSMQRGRTVGTYINRLLVKIRPHALDLVDAFGYGAEHLRAAIATGAEATRQDEARTYFRQQRASGSAPADEKTLLAIKAGKSRDQITSVGSSSKL

SEQ ID NO: 27 - DNA, 2022 bp, Rattus norvegicus
atgaaccccgacctgcgaaaggaacgagcctctgccactttcaaccccgagctgatcacccacatcctggacggctctcccgagaacacccgacgacgacgagaaatcgagaacctgatcctgaacgaccccgacttccagcacgaggactacaactttctgacccgatctcagcgatacgaggtggccgtgaagaagtctgccaccatggtcaagaagatgcgagagtacggcatctctgaccccgaagagatcatgtggttcaagaactctgtgcaccgaggacaccctgagcctctggacctgcacctgggcatgtttctgcccactctgctgcaccaggctaccgccgagcagcaagagcgattcttcatgcccgcctggaacctcgagatcaccggcacctacgctcagaccgagatgggccacggcacccacctccgaggactcgagactaccgccacttacgaccccaagactcaagagttcatcctgaactctcccaccgtgacctctatcaagtggtggcccggtggcctgggcaagacctctaaccacgccatcgtgctggcccagctgattacccagggcgagtgctacggcctgcacgccttcgtggtgcccatccgagagatcggaacccacaagccactgcctggcatcaccgtgggcgacatcggccccaagttcggctacgaggaaatggacaacggctacctgaagatggacaactaccgaattcctcgagagaacatgctgatgaagtacgcccaggtgaagcccgacggaacctacgtgaagcccctgtctaacaagctgacctacggaaccatggtgttcgtgcgatctttcctggtgggcaacgccgctcagtctctgtctaaggcctgcaccattgccatccgatactctgccgtgcgacgacagtctgagatcaagcagtctgagcccgagcctcagatcctggactttcagacccagcagtacaagctgttccctctgctggccaccgcctacgccttccacttcgtgggccgatatatgaaggaaacctacctgcgaatcaacgagtctatcggccagggcgacctgtctgagctgcccgagctgcacgccctgaccgccggactgaaggctttcaccacctggaccgccaacgccggcatcgaggaatgccgaatggcctgtggcggccacggctactctcactcctctggcatccccaacatctacgtgaccttcactcccgcctgcaccttcgagggtgagaacaccgtgatgatgctgcagaccgctcgattcctgatgaagatctacgaccaggtgcgatctggcaagctggtcggcggcatggtgtcttacctgaacgatctgccctctcagcgaattcagcctcagcaggttgccgtgtggcccactatggtggacatcaactcgctcgagggcctgaccgaggcctacaagctgcgagccgctcgactggtcgagatcgccgccaagaacctgcagacccacgtgtctcaccgaaagtctaaggaagtggcttggaacctgacctctgtggacctggtgcgagcttctgaggcccactgccactacgtggtggtgaaggtgttctctgacaagctgcccaagatccaggacaaggctgtccaggccgtgctgcgaaacctgtgcctgctgtactctctgtacggaatctctcagaagggcggcgacttcctcgagggctctatcatcaccggcgctcagctgtctcaggtcaacgctcgaatcctcgagctgctgaccctgattcgacccaacgccgtggctctggtggacgctttcgacttcaaggacatgaccctgggctctgtgctgggacgatacgacggcaacgtgtacgagaacctcttcgagtgggccaagaagtctcccctgaacaagaccgaggtgcacgagtcttaccacaagcacctgaagcctctgcagtctaagctggaccagattacctccgtgggatcttcttcgaagctgtag

SEQ ID NO: 28 - protein, 673 aa, Rattus norvegicus
MNPDLRKERASATFNPELITHILDGSPENTRRRREIENLILNDPDFQHEDYNFLTRSQRYEVAVKKSATMVKKMREYGISDPEEIMWFKNSVHRGHPEPLDLHLGMFLPTLLHQATAEQQERFFMPAWNLEITGTYAQTEMGHGTHLRGLETTATYDPKTQEFILNSPTVTSIKWWPGGLGKTSNHAIVLAQLITQGECYGLHAFVVPIREIGTHKPLPGITVGDIGPKFGYEEMDNGYLKMDNYRIPRENMLMKYAQVKPDGTYVKPLSNKLTYGTMVFVRSFLVGNAAQSLSKACTIAIRYSAVRRQSEIKQSEPEPQILDFQTQQYKLFPLLATAYAFHFVGRYMKETYLRINESIGQGDLSELPELHALTAGLKAFTTWTANAGIEECRMACGGHGYSHSSGIPNIYVTFTPACTFEGENTVMMLQTARFLMKIYDQVRSGKLVGGMVSYLNDLPSQRIQPQQVAVWPTMVDINSLEGLTEAYKLRAARLVEIAAKNLQTHVSHRKSKEVAWNLTSVDLVRASEAHCHYVVVKVFSDKLPKIQDKAVQAVLRNLCLLYSLYGISQKGGDFLEGSIITGAQLSQVNARILELLTLIRPNAVALVDAFDFKDMTLGSVLGRYDGNVYENLFEWAKKSPLNKTEVHESYHKHLKPLQSKLDQITSVGSSSKL

SEQ ID NO: 29 - DNA, 1533 bp, Saccharomyces cerevisiae
atgcccacctctggcaccaccatcgagctgattgacgatcagttccccaaggacgactctgcctcttctggcatcgtggacgaggtggacctgaccgaggccaacatcctggccaccggcctgaacaagaaggctccccgaatcgtcaacggcttcggctctctgatgggctctaaggaaatggtgtctgtcgagttcgacaagaagggcaacgagaagaagtctaacctggaccgactgctcgagaaggacaaccaggaaaaggaagaggccaagaccaagatccacatctctgagcagccctggactctgaacaactggcaccagcacctgaactggctgaacatggtgctggtctgcggcatgcccatgatcggctggtacttcgccctgtctggcaaggtgcccctgcacctcaacgtgtttctgttctctgtgttctactacgccgtcggcggcgtgtctatcaccgccggctaccaccgactgtggtcccaccgatcttactctgctcactggcccctgcgactgttctacgccatcttcggctgcgcctctgtcgagggctctgccaagtggtggggccactctcaccgaatccaccaccgatacaccgacactctgcgagatccctacgacgcccgacgaggcctgtggtactctcacatgggctggatgctgctgaagcccaaccctaagtacaaggcccgagccgacatcaccgacatgactgacgactggaccatccgattccagcaccgacattacatcctgctgatgctgctcaccgccttcgtgatccccactctgatctgcggctacttcttcaacgactacatgggcggcctgatctacgccggcttcatccgagtgttcgtgattcagcaggctaccttctgtatcaactctctggcccactacatcggcacccagcctttcgacgaccgacgaacccctcgagacaactggatcaccgccattgtgaccttcggcgagggctaccacaacttccaccacgagttccccaccgactaccgaaacgccatcaagtggtatcagtacgaccccaccaaggtgattatctacctgacctctctggtcggcctggcctacgacctgaagaagttctctcagaacgccatcgaggaagccctgatccagcaagagcagaagaagatcaacaagaagaaggcaaagatcaactggggacccgtgctgaccgacctgcctatgtgggacaagcagaccttcctggccaagtccaaggaaaacaagggcctcgtcatcatctctggcattgtgcacgacgtgtctggctacatttctgagcaccccggtggcgagactctgatcaagaccgctctcggcaaggacgccaccaaggccttctctggcggagtgtaccgacattctaacgccgctcagaacgtgctggccgacatgcgagtggccgtgatcaaggaatctaagaactctgccatccgaatggcctctaagcgaggcgagatctacgagactggcaagttcttttag

SEQ ID NO: 30 - protein, 510 aa, Saccharomyces cerevisiae
MPTSGTTIELIDDQFPKDDSASSGIVDEVDLTEANILATGLNKKAPRIVNGFGSLMGSKEMVSVEFDKKGNEKKSNLDRLLEKDNQEKEEAKTKIHISEQPWTLNNWHQHLNWLNMVLVCGMPMIGWYFALSGKVPLHLNVFLFSVFYYAVGGVSITAGYHRLWSHRSYSAHWPLRLFYAIFGCASVEGSAKWWGHSHRIHHRYTDTLRDPYDARRGLWYSHMGWMLLKPNPKYKARADITDMTDDWTIRFQHRHYILLMLLTAFVIPTLICGYFFNDYMGGLIYAGFIRVFVIQQATFCINSLAHYIGTQPFDDRRTPRDNWITAIVTFGEGYHNFHHEFPTDYRNAIKWYQYDPTKVIIYLTSLVGLAYDLKKFSQNAIEEALIQQEQKKINKKKAKINWGPVLTDLPMWDKQTFLAKSKENKGLVIISGIVHDVSGYISEHPGGETLIKTALGKDATKAFSGGVYRHSNAAQNVLADMRVAVIKESKNSAIRMASKRGEIYETGKFF

SEQ ID NO: 31 - DNA, 1449 bp, Yarrowia lipolytica
atggtgaaaaacgtggaccaagtggatctctcgcaggtcgacaccattgcctccggccgagatgtcaactacaaggtcaagtacacctccggcgttaagatgagccagggcgcctacgacgacaagggccgccacatttccgagcagcccttcacctgggccaactggcaccagcacatcaactggctcaacttcattctggtgattgcgctgcctctgtcgtcctttgctgccgctcccttcgtctccttcaactggaagaccgccgcgtttgctgtcggctattacatgtgcaccggtctcggtatcaccgccggctaccaccgaatgtgggcccatcgagcctacaaggccgctctgcccgttcgaatcatccttgctctgtttggaggaggagctgtcgagggctccatccgatggtgggcctcgtctcaccgagtccaccaccgatggaccgactccaacaaggacccttacgacgcccgaaagggattctggttctcccactttggctggatgctgcttgtgcccaaccccaagaacaagggccgaactgacatttctgacctcaacaacgactgggttgtccgactccagcacaagtactacgtttacgttctcgtcttcatggccattgttctgcccaccctcgtctgtggctttggctggggcgactggaagggaggtcttgtctacgccggtatcatgcgatacacctttgtgcagcaggtgactttctgtgtcaactcccttgcccactggattggagagcagcccttcgacgaccgacgaactccccgagaccacgctcttaccgccctggtcacctttggagagggctaccacaacttccaccacgagttcccctcggactaccgaaacgccctcatctggtaccagtacgaccccaccaagtggctcatctggaccctcaagcaggttggtctcgcctgggacctccagaccttctcccagaacgccatcgagcagggtctcgtgcagcagcgacagaagaagctggacaagtggcgaaacaacctcaactggggtatccccattgagcagctgcctgtcattgagtttgaggagttccaagagcaggccaagacccgagatctggttctcatttctggcattgtccacgacgtgtctgcctttgtcgagcaccaccctggtggaaaggccctcattatgagcgccgtcggcaaggacggtaccgctgtcttcaacggaggtgtctaccgacactccaacgctggccacaacctgcttgccaccatgcgagtttcggtcattcgaggcggcatggaggttgaggtgtggaagactgcccagaacgaaaagaaggaccagaacattgtctccgatgagagtggaaaccgaatccaccgagctggtctccaggccacccgggtcgagaaccccggtatgtctggcatggctgcttag

SEQ ID NO: 32 - protein, 482 aa, Yarrowia lipolytica
MVKNVDQVDLSQVDTIASGRDVNYKVKYTSGVKMSQGAYDDKGRHISEQPFTWANWHQHINWLNFILVIALPLSSFAAAPFVSFNWKTAAFAVGYYMCTGLGITAGYHRMWAHRAYKAALPVRIILALFGGGAVEGSIRWWASSHRVHHRWTDSNKDPYDARKGFWFSHFGWMLLVPNPKNKGRTDISDLNNDWVVRLQHKYYVYVLVFMAIVLPTLVCGFGWGDWKGGLVYAGIMRYTFVQQVTFCVNSLAHWIGEQPFDDRRTPRDHALTALVTFGEGYHNFHHEFPSDYRNALIWYQYDPTKWLIWTLKQVGLAWDLQTFSQNAIEQGLVQQRQKKLDKWRNNLNWGIPIEQLPVIEFEEFQEQAKTRDLVLISGIVHDVSAFVEHHPGGKALIMSAVGKDGTAVFNGGVYRHSNAGHNLLATMRVSVIRGGMEVEVWKTAQNEKKDQNIVSDESGNRIHRAGLQATRVENPGMSGMAA

SEQ ID NO: 33 - DNA, 1086 bp, Drosophila melanogaster
atggcaccctattcccgtatctaccatcaggataagtcttccagagagaccggtgtcctttttgaggacgatgctcagacagtggacagcgacctgactaccgatcggttccaactcaaacgagcagaaaagagacggctccctctggtctggcgaaacatcattctttttgctctggtccaccttgccgccctgtatggtctgcacagcatctttacccgggcaaaacttgcaactaccctctttgccgcaggactttatatcatcggaatgctcggtgtgactgccggtgctcaccgtctttgggctcatcgaacctataaagccaagtggcctctgcgacttctccttgtgatcttcaatactattgcctttcaagacgcagtctatcactgggcacgggaccatcgagtgcatcataaatactccgagacggacgctgaccctcataacgcaactcgtggattttttttctcgcacgttggctggctcctttgtaagaaacaccctgacatcaaagagaagggacgaggtctcgatctgagcgacctgagagctgatcctattctgatgtttcagcgaaaacattattatattctgatgcctctggcatgtttcgtcctgcctacggtgatccctatggtgtattggaacgagactcttgccagcagctggttcgtcgcaactatgttccggtggtgcttccagcttaacatgacctggcttgtcaattcggctgcccacaaatttggaaaccggccctacgataaaactatgaaccctactcaaaacgcattcgtttctgctttcacctttggtgaaggctggcataactaccaccacgcatttccttgggattataaaaccgctgagtggggctgctacagcctgaacattacaacggcatttattgatctttttgcaaagatcggatgggcctatgacctgaagaccgtggcacccgacgtcatccaacggcgggtcctccgaaccggagacggttcgcatgaactgtggggatggggcgacaaagatcttactgctgaggacgctcgtaatgtcctcctcgtggataaaagccgataa

SEQ ID NO: 34 - DNA, 1086 bp, Drosophila melanogaster
atggctccctactctcgaatctaccaccaggacaagtcgtcccgagagactggcgtgctgttcgaggacgacgcccagaccgtggactctgacctgaccaccgaccgattccagctgaagcgagccgagaagcgacgactgcccctggtgtggcgaaacatcatcctgttcgccctggtgcacctggccgctctgtacggcctgcactctatcttcacccgagccaagctggccaccactctgttcgctgccggcctgtacatcatcggcatgctgggcgtgaccgctggcgcccaccgactgtgggctcaccgaacctacaaggccaagtggcccctgcgactgctgctggtgatcttcaacaccattgccttccaggacgccgtgtaccactgggcccgagatcaccgagtgcaccacaagtactctgagactgacgctgaccctcacaacgctacccgaggcttcttcttctctcacgtcggctggctgctgtgcaagaagcaccccgacatcaaggaaaagggccgaggcctggacctgtctgacctgcgagctgaccccatcctgatgttccagcgaaagcactactacattctgatgcccctggcctgcttcgtgctgcccaccgtgattcccatggtgtactggaacgagactctggcctcttcctggttcgtggccaccatgttccgatggtgcttccagctcaacatgacctggctggtgaactctgccgctcacaagttcggcaaccgaccttacgacaagactatgaaccccactcagaacgccttcgtgtctgccttcaccttcggcgaaggctggcacaactaccaccacgcattcccttgggactacaagaccgccgagtggggctgctactctctgaacatcaccaccgccttcatcgacctgttcgctaagatcggctgggcctacgacctcaagaccgtggctcccgacgtgatccagcgacgagtgctgcgaaccggcgacggctctcacgagctgtggggctggggcgacaaggacctgaccgctgaggacgcccgaaacgtcctgctggtggacaagtctcgataa

SEQ ID NO: 35 - protein, 361 aa, Drosophila melanogaster
MAPYSRIYHQDKSSRETGVLFEDDAQTVDSDLTTDRFQLKRAEKRRLPLVWRNIILFALVHLAALYGLHSIFTRAKLATTLFAAGLYIIGMLGVTAGAHRLWAHRTYKAKWPLRLLLVIFNTIAFQDAVYHWARDHRVHHKYSETDADPHNATRGFFFSHVGWLLCKKHPDIKEKGRGLDLSDLRADPILMFQRKHYYILMPLACFVLPTVIPMVYWNETLASSWFVATMFRWCFQLNMTWLVNSAAHKFGNRPYDKTMNPTQNAFVSAFTFGEGWHNYHHAFPWDYKTAEWGCYSLNITTAFIDLFAKIGWAYDLKTVAPDVIQRRVLRTGDGSHELWGWGDKDLTAEDARNVLLVDKSR

SEQ ID NO: 36 - DNA, 981 bp, Amyelois transitella
atggtgcccaataagggatctagcgacgttctgtcggaacatagcgaaccccaatttacaaagctgattgcacctcaggcaggtcctcgaaaatacaagattgtgtatagaaaccttctgacgtttggttattggcacctttccgctgtgtatggactctacctgtgtttcacctgcgcaaagtgggccactattctgtttgcatttttcctctacgttattgcagagattggaattactggcggtgcccacagactgtgggcccatcggacgtacaaagcaaaactgcctctggaaattctcctgctcatcatgaatagcatcgcttttcaagacactgcatttacatgggcccgagatcaccgtcttcatcacaagtacagcgacacggatgcagatccccacaacgcaacgagaggtttcttctactcccatgtgggatggctgctggtgaaaaagcaccccgaagtcaaagcacggggaaaatacctttctctggacgaccttaaaaataatcctctcctcaagtttcaaaagaaatatgcaattcttgtgattggtacactctgtttcctcatgcctacattcgttcccgtttatttctggggtgagggtattagcactgcatggaatatcaatctgcttcgttacgtgatgaacctcaatatgacctttctcgtgaatagcgcagctcacatcttcggaaacaagccctacgataagtccattgctagcgttcaaaacattagcgtgtcccttgctactttcggagagggattccacaattatcatcacacctatccctgggattaccgggccgcagagctgggaaacaaccggctcaatatgacgacagcttttattgatttcttcgcctggatcggctgggcttatgatctcaaatctgttccccaagaagccattgccaaacggtgcgccaagacaggagatggaactgacatgtggggtcgtaagagataa

SEQ ID NO: 37 - DNA, 981 bp, Amyelois transitella
atggtgcccaacaagggttcttccgacgtcctgtctgagcactccgagccccagttcaccaagctgattgctccccaggctggcccccgaaagtacaagatcgtgtaccgaaacctgctgaccttcggatactggcacctgtctgccgtctacggtctgtacctgtgtttcacctgcgccaagtgggctaccattctgttcgccttcttcctgtacgtgatcgctgagatcggcattaccggcggagcccaccgactgtgggctcaccgaacctacaaggccaagctgcccctggagatcctgctgctgattatgaactctatcgctttccaggacaccgccttcacctgggctcgagatcaccgactgcaccacaagtactctgacaccgacgctgaccctcacaacgctacccgaggtttcttctactcccacgtgggctggctgctggtcaagaagcaccccgaggtgaaggcccggggaaagtacctgtccctggacgacctgaagaacaaccccctgctgaagttccagaagaagtacgctatcctggtcattggcaccctgtgtttcctgatgcccaccttcgtgcccgtctacttctggggtgagggcatttctaccgcctggaacatcaacctgctgcgatacgtgatgaacctgaacatgaccttcctggtcaactccgccgctcacattttcggcaacaagccctacgacaagtctattgcctccgtgcagaacatctctgtctccctggctaccttcggagagggtttccacaactaccaccacacctacccttgggactaccgagctgctgagctgggcaacaaccgactgaacatgaccaccgccttcattgacttcttcgcctggatcggatgggcttacgacctgaagtccgtcccccaggaagccatcgctaagcgatgcgctaagaccggcgacggaaccgacatgtggggacgaaagcgatag

SEQ ID NO: 38 - protein, 326 aa, Amyelois transitella
MVPNKGSSDVLSEHSEPQFTKLIAPQAGPRKYKIVYRNLLTFGYWHLSAVYGLYLCFTCAKWATILFAFFLYVIAEIGITGGAHRLWAHRTYKAKLPLEILLLIMNSIAFQDTAFTWARDHRLHHKYSDTDADPHNATRGFFYSHVGWLLVKKHPEVKARGKYLSLDDLKNNPLLKFQKKYAILVIGTLCFLMPTFVPVYFWGEGISTAWNINLLRYVMNLNMTFLVNSAAHIFGNKPYDKSIASVQNISVSLATFGEGFHNYHHTYPWDYRAAELGNNRLNMTTAFIDFFAWIGWAYDLKSVPQEAIAKRCAKTGDGTDMWGRKR

SEQ ID NO: 39 - DNA, 1008 bp, Choristoneura rosaceana
atggctcccaacgtcgaggacatggaatctgacctgcctgagtctgaggaaaagctcgagaagctggtggctccccaggctgctccccgaaagtaccagatcatctacaccaacctgctgaccttcggctactggcacattgccggcctgtacggactgtacctgtgcttcacctctgccaagtggcagaccatcattctggccctgatcctgaacgagatggccattctgggcatcaccgctggcgcccaccgactgtgggctcaccgatcttacaaggccaccgtgcctctgcagatcatcctgatcatcttcaactccctgtctttccagaactctgccatccactggatccgagatcaccgaatgcaccacaagtactctgacaccgacggcgaccctcacaacgcctctcgaggcttcttctactctcacgtcggctggctgctggtgaagaagcaccccgaggtcaagaagcgagccaagaccatcgacatgtctgacatctactctaaccccatcctgcgattccagaagaagtacgctatccccttcatcggcatgatctgcttcgtgctgcccactattatccctatgtacttctggggcgagactctgtctaacgcctggcacatcaccatgctgcgatacgtgttctctctgaactctatcttcctggtgaactccgccgctcacctgtacggctaccgaccttacgacaagaacattctgcccgccgagaacaagatgaccttcattgcctgcctgggcgagaacttccacaactaccaccacgtgttcccttgggactaccgagcctctgagctgggcaacatcggaatgaactggaccgccaagttcatcgactttttcgcctggatcggctgggcctacgacctcaagaccgcctctgacgagaacatcaagtctcgaatgaagcgaaccggcgacggcaccgacgtgtctggacagaagtactcttgcgagtcctctgaggtgctgcagtaa

SEQ ID NO: 40 - protein, 335 aa, Choristoneura rosaceana
MAPNVEDMESDLPESEEKLEKLVAPQAAPRKYQIIYTNLLTFGYWHIAGLYGLYLCFTSAKWQTIILALILNEMAILGITAGAHRLWAHRSYKATVPLQIILIIFNSLSFQNSAIHWIRDHRMHHKYSDTDGDPHNASRGFFYSHVGWLLVKKHPEVKKRAKTIDMSDIYSNPILRFQKKYAIPFIGMICFVLPTIIPMYFWGETLSNAWHITMLRYVFSLNSIFLVNSAAHLYGYRPYDKNILPAENKMTFIACLGENFHNYHHVFPWDYRASELGNIGMNWTAKFIDFFAWIGWAYDLKTASDENIKSRMKRTGDGTDVSGQKYSCESSEVLQ

SEQ ID NO: 41 - DNA, 990 bp, Ostrinia nubilalis
atggtcccctacgccactaccgccgacggccaccccgaaaaggacgagtgcttcgaggacaacgagatcaagtctaattctctacctaagctggagattctgtactttaacgttatgaccttcaccttcctgcacctgtctgctctgtacggtctgtacctgggcttcacctctgtgaagtgggctaccatcggactgggcatcattttctacttcttcgccgagatcggaattaccgccggagcccatcgactgtggtctcaccgatcttacaaggccaagttgcccttggagatcctgctgatggtgttcaactccatggccttccagaacaccgccctgtcttgggctcgagaccatcgagttcaccacaagtgccccgacaccaacggagaccctcataacgctaatcgaggattcttctactcccacgttggctggttgatgaccaagaagtctgacgaggtgattaagcagggaaagctgtgtgacgtggctgacctgtattctaaccccgttctgcgattccagaagaagtacgctgttcctttcattggaactctgtgcttcgtcctgcccaccctcatccccatgtacttctggggagagaccctgaacaatgcttggcatttcaacatgttccgatacgtgattaacctgaacgccaccttctgcgtgaactctgtcgtgcacaagtggggatacaagccctacgacaagaacatctgtcccacccagaacgtgctgctgaacttggctgttctgggtgaggcctttcataactaccaccatgtgttcccttgggactaccgagccgctgagctgggtaatcagaagatgaaccccactactctgttcattgatttctttgcctggattggctgggcctacgaccttaagactgcctccaaggagatgatcaagtctcgatctgaacgaaccggtgacggaaccgacctgtggggtcactcggccgacaagctgaagtaa

SEQ ID NO: 42 - protein, 329 aa, Ostrinia nubilalis
MVPYATTADGHPEKDECFEDNEIKSNSLPKLEILYFNVMTFTFLHLSALYGLYLGFTSVKWATIGLGIIFYFFAEIGITAGAHRLWSHRSYKAKLPLEILLMVFNSMAFQNTALSWARDHRVHHKCPDTNGDPHNANRGFFYSHVGWLMTKKSDEVIKQGKLCDVADLYSNPVLRFQKKYAVPFIGTLCFVLPTLIPMYFWGETLNNAWHFNMFRYVINLNATFCVNSVVHKWGYKPYDKNICPTQNVLLNLAVLGEAFHNYHHVFPWDYRAAELGNQKMNPTTLFIDFFAWIGWAYDLKTASKEMIKSRSERTGDGTDLWGHSADKLK

SEQ ID NO: 43 - DNA, 1044 bp, Thaumetopoea pityocampa
atggcccccaacacccgagagaacgagactatctacgatgaggtggaacacaagctggagaagctggtgcctccccaggccggcccgtggaactacaagatcgtgtacctcaacctgttaaccttctcctactggctgatcgctggcgcttacggcctgtacctgtgtttcacctctgccaagtgggccaccatcatctttgagttcatcctgttcttcttcgctgagatgggcatcaccgccggtgctcaccgactgtggacccacaagtcctacaaggccaagctgcccctggagatttttctgatggtgctcaactctgtggcttttcagaacactgctactgattgggtgcgagaccaccgactccatcacaagtattcggacactgacgccgacccccacaacgccgcccgaggactgttcttctctcacgtgggatggctgcttgtccgaaaacacgacgaggtcaagaagcgaggcaagttcaccgacatgtctgacatctacaacaaccctgtgctgaagttccagaagaagtacgccatcccctttatcggagccgtgtgtttcatcctgcccaccgtgattcctatgtacttctggggcgagtctctgaacaacgcttggcatatttgtatcttgcgatacgccatgaacttaaacgttaccttctctgtgaactctctagcccatatctggggtaacaagccctacgacaaggacatcaagcccgcacagaacttcggcgtgaccctggccacctttggtgagggcttccacaactaccaccacgtgtttccctgggactatagaacctctgagctcggcgacaacaagtttaacttcaccaccaagttcatcaatttcttcgagagaattggactggcctacgacctgaagactgtgtccgacgacgtgatcgcccagcgagctaagcgaactggagatggcacccacctgtgggactgtgccgataagaacaacaacgacgtggtgcaaaccaaggcccagattgacaccctctgtaccaagcacgagtaa

SEQ ID NO: 44 - protein, 347 aa, Thaumetopoea pityocampa
MAPNTRENETIYDEVEHKLEKLVPPQAGPWNYKIVYLNLLTFSYWLIAGAYGLYLCFTSAKWATIIFEFILFFFAEMGITAGAHRLWTHKSYKAKLPLEIFLMVLNSVAFQNTATDWVRDHRLHHKYSDTDADPHNAARGLFFSHVGWLLVRKHDEVKKRGKFTDMSDIYNNPVLKFQKKYAIPFIGAVCFILPTVIPMYFWGESLNNAWHICILRYAMNLNVTFSVNSLAHIWGNKPYDKDIKPAQNFGVTLATFGEGFHNYHHVFPWDYRTSELGDNKFNFTTKFINFFERIGLAYDLKTVSDDVIAQRAKRTGDGTHLWDCADKNNNDVVQTKAQIDTLCTKHE

SEQ ID NO: 45 - DNA, 1059 bp, Dendrophilus punctatus
atggctcccaaggaagccgacgtcaacggcgtgctgttcgagtctgacgccaccactcctgacatggctctgcccactactcccgtgcagcaggccgacaactaccccaagaagctggtctggcgaaacatcctgctgttcgcctacctgcacctggccgctctgtacggcggctacctgttcctgttctctgccaagtggcagaccgacatcttcgcctacatcctgtacatcatgtctggcctgggcatcaccgctggcgcccaccgactgtgggctcacaagtcttacaaggctaagtggcccctgcgactgatcctggtgctgttcaacaccctggccttccaggactctgccatcgactgggcccgagatcaccgaatgcaccacaagtactctgagactgacgctgaccctcacaacgctacccgaggcttcttcttctctcacatcggctggctgctggtgcgaaagcaccccgagcttaagaagaagggcaagggcctcgacatctctgacctgtacgctgatcccatcctgcgattccagaagaagtactacctgctgctgatgcccctgggctgcttcatcctgcctaccgtgattcccgtgtacctgtggaacgagacttggtctaacgccttcctggtggctgccctgttccgatacgccgtgatcctgaacgtgacctggctggtgaactctgctgcccacaagtggggcgacaagccctacgacaagtctatcaagccctctgagaacctgtctgtggccctgttcgccctcggcgagggattccacaactaccaccacacattcccttgggactacaagaccgccgagctgggcaacaaccgactgaacttcactactaccttcatcaactttttcgccaagatcggctgggcctacgacatgaagaccgtgtctgacgagatcatccagaaccgagtgaagcgaaccggcgacggctctcaccacctgtggggctggggagacaaggaccactctaaggaagagatcaacgccgccatccgaatcaaccccaaggacgactag

SEQ ID NO: 46 - protein, 352 aa, Dendrophilus punctatus
MAPKEADVNGVLFESDATTPDMALPTTPVQQADNYPKKLVWRNILLFAYLHLAALYGGYLFLFSAKWQTDIFAYILYIMSGLGITAGAHRLWAHKSYKAKWPLRLILVLFNTLAFQDSAIDWARDHRMHHKYSETDADPHNATRGFFFSHIGWLLVRKHPELKKKGKGLDISDLYADPILRFQKKYYLLLMPLGCFILPTVIPVYLWNETWSNAFLVAALFRYAVILNVTWLVNSAAHKWGDKPYDKSIKPSENLSVALFALGEGFHNYHHTFPWDYKTAELGNNRLNFTTTFINFFAKIGWAYDMKTVSDEIIQNRVKRTGDGSHHLWGWGDKDHSKEEINAAIRINPKDD

SEQ ID NO: 47 - DNA, 1041 bp, Grapholita molesta
atgcctcccgagtccaagaacgtgcccatccagcagaactttcgaaagccccttgagttcctgccccgaaagtacgacgtcgtgtacgagaacgtgttcctgcacatcgccggccatatctcggccgcctacggactgtacctgtgctttaccgttgctaagtggcagactatcgccctggccttcgtgtggtaccatctgggaaagatcggaatcatctgcggagctcacagactgtggtctcaccgatgctacaaggccaagatgcctctgcacatcatcctgatgatctgcaactgtattggatttgaaaacaccgccatcaattgggttcgaaaccaccgaatgcaccacaagcactctgacaccgacggcgatccccacaactctaaccgaggtgccttcttctcccacatcggctggctgtgcgtgcgaaagcatcctgaaactcgaaactgtaaggtggacatgtctgatatttactctaaccccgtgctggtgttccagaagcgctacaagtatcccctggtgggcttcctgtgctatggacttcccaccttcatccccatgtacttctggggcgaaactctggtgaccgcttggcacgtgaacatcctgcggtacttcctgtcgatgaacgccgtcttcctggtgaattcccttgcccatctgtacggcaacaagccctacgacatttccatctgcccccggcagtctcccttcgtgtctctcctgaccatcggagagggctttcacaactaccaccacaccttcccttgggactaccgagccgccgagctgggcaataactaccttaacgtgggaaagtgggtgatcgacttcttcgccatgattggatgggcctacgacctgaagactgtgcccgatgagaccattaagcgacgaatgaagcgaactggagacggaaccaactgctggggatggggcgacaaggacatgactcgagaggaccgagacatcgccaaaattatttaccccgagtctatttctaaggaagagcgagacattatcgccatgtaa

SEQ ID NO: 48 - protein, 346 aa, Grapholita molesta
MPPESKNVPIQQNFRKPLEFLPRKYDVVYENVFLHIAGHISAAYGLYLCFTVAKWQTIALAFVWYHLGKIGIICGAHRLWSHRCYKAKMPLHIILMICNCIGFENTAINWVRNHRMHHKHSDTDGDPHNSNRGAFFSHIGWLCVRKHPETRNCKVDMSDIYSNPVLVFQKRYKYPLVGFLCYGLPTFIPMYFWGETLVTAWHVNILRYFLSMNAVFLVNSLAHLYGNKPYDISICPRQSPFVSLLTIGEGFHNYHHTFPWDYRAAELGNNYLNVGKWVIDFFAMIGWAYDLKTVPDETIKRRMKRTGDGTNCWGWGDKDMTREDRDIAKIIYPESISKEERDIIAM

SEQ ID NO: 49 - DNA, 1047 bp, Cydia pomonella
atgcctccccgagagtctaagaaggtggccctgcgatcttacgagacccctgtcgcttctctccctcctcgaaagtacgagattatctacctcaacctcttcctgcacatcgctggacatatctccgccgtctacggcctgtatctgtgcttcaccgccgcccagtggaagaccatcttctttgcctacctgtggctgttgatgggcgagctcggcgtggtgtgtggcgctcacagattgtggtctcaccgttctttcaaggtgaagcctcctctcgagatcatgctgatgctgttcaactgtattggattccagaacaccgccactgactgggtccgaaaccaccggctccatcacaagcactctgacactgacgccgacccccataactctaaccgaggaatgctgttctcccacattggctggctgtgtgtgcgaaagcacccagatgttaaagaacgaggcaagaccaccgacatgtctgacatctactctaaccccgtgctccgattccagaagaagcacaaggtaccccttttcggcgccatgtgtttcggcctgcccacccttattcccaccctgtggggagaggacatcgtcaccgcttggcacgtcaacctgctgcgattcgttcttaatctgaactctatcctgctggtcaactccattgctcataagtacggcacccgaccctacgatcgaaccatctgccctcgacaaaacaccacctgtaacatgatgactcttggagagggcttccacaactaccaccacacctttccttgggactaccgatctgccgagctgggaaagaactacctgaacttcaccaagtggttcatcgacttcttcgccctgattggatgggcctacgacctgaagaccgttcctgacgatatgatccagcgacgaatgaaaagaaccggagacggatccaactcgtggggatggggagacaaggacatgactaaggaggagcgagactctgctactatcatttatcccgagaagaaggatgatattaagatgatctccaaaaagaactaa

SEQ ID NO: 50 - protein, 348 aa, Cydia pomonella
MPPRESKKVALRSYETPVASLPPRKYEIIYLNLFLHIAGHISAVYGLYLCFTAAQWKTIFFAYLWLLMGELGVVCGAHRLWSHRSFKVKPPLEIMLMLFNCIGFQNTATDWVRNHRLHHKHSDTDADPHNSNRGMLFSHIGWLCVRKHPDVKERGKTTDMSDIYSNPVLRFQKKHKVPLFGAMCFGLPTLIPTLWGEDIVTAWHVNLLRFVLNLNSILLVNSIAHKYGTRPYDRTICPRQNTTCNMMTLGEGFHNYHHTFPWDYRSAELGKNYLNFTKWFIDFFALIGWAYDLKTVPDDMIQRRMKRTGDGSNSWGWGDKDMTKEERDSATIIYPEKKDDIKMISKKN

SEQ ID NO: 51 - DNA, 999 bp, Epiphyas postvittana
atggcccccaacgtggaggagatcgagactgacctcaccgagactgaggagaaatgggagaagctggtggctccccaggccgcccctagaaagcacgagatcctgtacaccaacctgctgattttcggctacggacacctggccggactgtatggactgtacctgtgtttcacctctgcccgacttcagaccatcatcctggccttcattctgcacgccatggccatcctgggaattactgccggcgctcaccgactgtggactcaccgttcttacaaggccaccatgcccctgcagattatcctgattatcttcaactctctgtcctttcaaaactccgccatcaactgggtccgagatcaccgatctcaccacaagtactgtgacaccgacgcagatcctcataacgccgctcgaggcctgttctacagccatatcggatggctgctggtgaagaagcaccccgaggtcaagaagcgaggaaagatgaccgacatgtctgatgtgtatcgaaaccccgtgctgcgattccagaagaagtacgctgtgccttttatcggaaccatctgcttcgtcctgcctaccatcattcccatgtacttctggggcgagtccctgaacaacgcctggcatatcaccctcctgcgatacattttctccatgcacactattttcctggtgaactctgtggcccatctgtggggcaaccgaccctacgacaagaacattcttcccgctgacaaccgaaccctgtctatcgccaccctcggggaggcctcccacaactaccaccacaccttcccttgggattaccggtccactgagctgggatacctgcccacgaactttaccaccaactttatcgacttcttcgcctggatcggctgggcttacgacctgaagaccacctctggagagatcatcaattctagaatccagcgaaccggcgatggaacccattctcgatctaagaagaacatctccacccaggacgagtaa

SEQ ID NO: 52 - protein, 332 aa, Epiphyas postvittana
MAPNVEEIETDLTETEEKWEKLVAPQAAPRKHEILYTNLLIFGYGHLAGLYGLYLCFTSARLQTIILAFILHAMAILGITAGAHRLWTHRSYKATMPLQIILIIFNSLSFQNSAINWVRDHRSHHKYCDTDADPHNAARGLFYSHIGWLLVKKHPEVKKRGKMTDMSDVYRNPVLRFQKKYAVPFIGTICFVLPTIIPMYFWGESLNNAWHITLLRYIFSMHTIFLVNSVAHLWGNRPYDKNILPADNRTLSIATLGEASHNYHHTFPWDYRSTELGYLPTNFTTNFIDFFAWIGWAYDLKTTSGEIINSRIQRTGDGTHSRSKKNISTQDE

SEQ ID NO: 53 - DNA, 1017 bp, Spodoptera littoralis
atggcccagtgcgtccagaccaccaccatcctcgagcagaaggaagagaaaaccgtgaccctcctggtgccccaggccggaaagcgaaagttcgagatcgtgtacttcaacattattaccttcgcttactggcacattgccggactttacggcctgtacctgtgcttcacctctaccaagtgggccaccgttcttttctctttcttcctgttcgtggtggctgaggttggagttaccgccggctctcaccgactttggtctcataagacctacaaggccaagctccccctgcagattcttcttatggtgatgaactctctggcttttcaaaatactgttatcgactgggtgcgagaccatcggctgcaccataagtactctgacaccgacgctgatccccacaacgcttctcgaggattcttctactctcacgtgggatggcttctggtgcgaaagcaccccgacgtcaagaagcgaggaaaggagatcgacatttctgacatctacaacaatcccgtgcttcgattccagaagaagtacgctatccccttcattggcgccgtgtgtttcgtgctgcccaccctcatccctgtttacggctggggagagacctggaccaacgcctggcatgtggccatgctgcgatacattatgaacctgaacgtgactttcctggtgaactctgccgctcacatttacggcaagcgaccctacgataagaagatccttccctctcagaacatcgccgtctctatcgccaccttcggagagggatttcacaactaccaccacgtgttcccctgggattaccgagctgctgagctgggaaacaactctctgaacttccctaccaagtttattgacttcttcgcctggattggatgggcctacgacctgaagaccgtgtctaaggagatgatcaagcagcgatcgaagcgaaccggcgacggcacaaacctgtggggactggaggacgtcgacactcccgaggaccttaagaacaccaagggcgagtaa

SEQ ID NO: 54 - protein, 338 aa, Spodoptera littoralis
MAQCVQTTTILEQKEEKTVTLLVPQAGKRKFEIVYFNIITFAYWHIAGLYGLYLCFTSTKWATVLFSFFLFVVAEVGVTAGSHRLWSHKTYKAKLPLQILLMVMNSLAFQNTVIDWVRDHRLHHKYSDTDADPHNASRGFFYSHVGWLLVRKHPDVKKRGKEIDISDIYNNPVLRFQKKYAIPFIGAVCFVLPTLIPVYGWGETWTNAWHVAMLRYIMNLNVTFLVNSAAHIYGKRPYDKKILPSQNIAVSIATFGEGFHNYHHVFPWDYRAAELGNNSLNFPTKFIDFFAWIGWAYDLKTVSKEMIKQRSKRTGDGTNLWGLEDVDTPEDLKNTKGE

SEQ ID NO: 55 - DNA, 1005 bp, Choristoneura parallela
atggctcccaacgtcgaggacatggaatctgacatgcccgagtctgagaagtgggagaagctggtggctccccaggctgctccccgaaagtacgagatcatctacaccaacctgctgaccttcggctacggccacattgccggcctgtacggactgtacctgtgcttcacctctgccaagtggcagaccgtgatcctggccatcatcctgaacgagatggccattctgggcatcaccgctggcgcccaccgactgtggtcccaccgatcttacaaggccgctgtgcccctgcagatcattctgatgatcttcaactctctggccttccagaactctgccatcaactgggtgcgagatcaccgaatgcaccacaagtactctgacaccgacggcgaccctcacaacgcctctcgaggcttcttctactctcacgtcggctggctgctggtgaagaagcaccccgaggtcaaaaagcgaggcaagatgatcgacatgagcgacatctactctaaccccgtgctgcgattccagaagaagtacgctatccccttcatcggcatgatctgcttcgtgctgcccactattatccctatgtacttctggggcgagactctgtctaacgcctggcacatcaccatgctgcgatacgtgttctctctgaactctatcttcctggtgaactccgccgctcacctgtacggctaccgaccttacgacaagaacattctgcccgccgagaacaagatcgccctgatcgcctgcctgggcgactctttccacaactaccaccacgtgttcccttgggactaccgagcctctgagctgggcaacatcggaatgaactggaccgctcagttcatcgactttttcgcctggatcggctgggcctacgacctcaagaccgcctctgacgagaacatcaactctcgaatgaagcgaaccggcgacggcaccgacatctctggacagaagtactcttgcgagtcctctgaggtgctgcagtaa

SEQ ID NO: 56 - protein, 334 aa, Choristoneura parallela
MAPNVEDMESDMPESEKWEKLVAPQAAPRKYEIIYTNLLTFGYGHIAGLYGLYLCFTSAKWQTVILAIILNEMAILGITAGAHRLWSHRSYKAAVPLQIILMIFNSLAFQNSAINWVRDHRMHHKYSDTDGDPHNASRGFFYSHVGWLLVKKHPEVKKRGKMIDMSDIYSNPVLRFQKKYAIPFIGMICFVLPTIIPMYFWGETLSNAWHITMLRYVFSLNSIFLVNSAAHLYGYRPYDKNILPAENKIALIACLGDSFHNYHHVFPWDYRASELGNIGMNWTAQFIDFFAWIGWAYDLKTASDENINSRMKRTGDGTDISGQKYSCESSEVLQ

SEQ ID NO: 57 - DNA, 1368 bp, Helicoverpa armigera
atggtcgttctcacatcgaaagaaaccaaaccctccgtcgccgagttctacgctggcaaaagcgtcttcattacgggaggcacgggttttctcggcaaagtgtttatcgagaaacttctttactcctgccccgacattggaaacatttatatgctgattagagaaaaaaaaggtctcagcgtgtcggagagaatcaagcactttctggacgaccccctcttcacacggctgaaggagaaacgtcctgcagatctcgagaagattgttctgatccctggtgacatcacagcacctgaccttggaatcacttcggaaaatgaaaaaatgctgattgagaaggtgagcgttattatccattccgccgctacagttaaattcaatgagcctcttcctaccgcctggaagattaacgtcgagggtactcggatgatgctcgcactcagcagaagaatgaaacgaatcgaagtctttattcatatcagcacagcatatacaaataccaatagagaggtggttgacgagattctctatcctgcccctgcagacatcgaccaggtccaccgatacgttaaagatggtatctcggaagaagaaactgaaaaaattctcaatggacgacccaatacttacacttttaccaaagcccttacagagcatctcgtggcagagaaccaggcatacgttcctacgatcattgtgagaccctccgttgtcgcagcaattaaggatgagcctatcaagggatggcttggcaattggtatggtgcaactggactcacggtcttcactgctaagggtctgaatagagtcatttacggtcacagctctaacatcgttgacctcatccccgtggattatgtcgcaaatctggtgattgcagcaggagccaaatctagcaagagcacagagcttaaggtttacaattgttgctcgtccgcctgcaaccctatcacgattggcaagctcatgtccatgttcgcagaagacgcaatcaaacagaaatcgtatgcaatgcctcttcctggctggtatattttcacaaaatacaaatggctggttcttcttctcacaatcctgtttcaggtgattcctgcttatattacagacctctaccgacaccttattggcaagaaccctcgatacattaagctgcagtctctggtgaatcagaccagatcctcgattgacttcttcacttcgcactcctgggttatgaaggcagatcgagtgcgagaactttttgcttccctgtctcctgccgataagtatctctttccttgcgatcccactgatatcaattggacgcactatatccaagattattgctggggcgttagacatttccttgagaaaaagtcttacgaataa

SEQ ID NO: 58 - DNA, 1368 bp, Helicoverpa armigera
atggtggtcctgacctctaaggagactaagccctccgtggccgagttctacgctggcaagtctgtcttcatcaccggcggaaccggtttcctgggcaaggtcttcattgagaagctgctgtactcctgtcccgacatcggcaacatctacatgctgatccgagagaagaagggactgtctgtgtccgagcgaattaagcacttcctggacgaccccctgttcacccgactgaaggagaagcgacccgccgacctggagaagatcgtgctgattcccggagacatcaccgctcccgacctgggtattacctctgagaacgagaagatgctgatcgagaaggtgtctgtcatcattcactccgccgctaccgtcaagttcaacgagcccctgcccaccgcctggaagatcaacgtggagggaacccgaatgatgctggctctgtctcgacgaatgaagcgaattgaggtcttcatccacatttccaccgcctacaccaacaccaaccgagaggtggtggacgagatcctgtaccctgctcctgctgacattgaccaggtgcaccgatacgtcaaggacggtatctctgaggaagagactgagaagattctgaacggccgacccaacacctacaccttcaccaaggccctgaccgagcacctggtggctgagaaccaggcttacgtgcccaccatcattgtccgaccctccgtggtcgccgctatcaaggacgagcccattaagggatggctgggtaactggtacggagctaccggactgaccgtgttcaccgctaagggtctgaaccgagtcatctacggccactcttccaacatcgtggacctgattcccgtggactacgtcgccaacctggtcattgccgctggcgctaagtcttccaagtccaccgagctgaaggtgtacaactgttgctcttccgcctgcaaccccatcaccattggaaagctgatgtctatgttcgccgaggacgctatcaagcagaagtcctacgctatgcccctgcccggttggtacatcttcaccaagtacaagtggctggtcctgctgctgaccattctgttccaggtcatccccgcctacattaccgacctgtaccgacacctgatcggcaagaacccccgatacattaagctgcagtctctggtcaaccagacccgatcttccattgacttcttcacctctcactcctgggtcatgaaggctgaccgagtccgagagctgttcgcctctctgtcccccgctgacaagtacctgttcccctgtgaccccaccgacatcaactggacccactacattcaggactactgctggggagtgcgacacttcctggagaagaagtcctacgagtag

SEQ ID NO: 59 - protein, 455 aa, Helicoverpa armigera
MVVLTSKETKPSVAEFYAGKSVFITGGTGFLGKVFIEKLLYSCPDIGNIYMLIREKKGLSVSERIKHFLDDPLFTRLKEKRPADLEKIVLIPGDITAPDLGITSENEKMLIEKVSVIIHSAATVKFNEPLPTAWKINVEGTRMMLALSRRMKRIEVFIHISTAYTNTNREVVDEILYPAPADIDQVHRYVKDGISEEETEKILNGRPNTYTFTKALTEHLVAENQAYVPTIIVRPSVVAAIKDEPIKGWLGNWYGATGLTVFTAKGLNRVIYGHSSNIVDLIPVDYVANLVIAAGAKSSKSTELKVYNCCSSACNPITIGKLMSMFAEDAIKQKSYAMPLPGWYIFTKYKWLVLLLTILFQVIPAYITDLYRHLIGKNPRYIKLQSLVNQTRSSIDFFTSHSWVMKADRVRELFASLSPADKYLFPCDPTDINWTHYIQDYCWGVRHFLEKKSYE

SEQ ID NO: 60 - DNA, 1578 bp, Saccharomyces cerevisiae
atgaacgagatcgacgagaagaaccaggctcctgtgcagcaagagtgcctgaaggaaatgatccagaacggacacgcccgacgaatgggctctgtcgaggacctgtacgtggccctgaaccgacagaacctgtaccgaaacttctgcacctacggcgagctgtctgactactgcacccgagatcagctgaccctggctctgcgagagatctgcctgaagaaccctactctgctgcatatcgtgctgcccactcgatggcccaaccacgagaactactaccgatcttctgagtactactctcgaccccatcctgtgcacgactacatctccgtgctgcaagagctgaagctgtctggcgtggtgctgaacgagcagcccgagtactctgccgtgatgaagcagatcctggaagagttcaagaactctaagggctcttacaccgccaagatcttcaagctgactactaccctgaccattccttacttcggccccactggaccctcttggcgactgatctgtctgcccgaggaacacaccgagaagtggaagaagttcatcttcgtttctaaccactgcatgtctgacggacgatcctctatccacttctttcacgacctgcgagatgagctgaacaacatcaagacccctccaaagaagctggactacattttcaagtacgaagaggactaccagctgctgcgaaagctgcccgagcctatcgagaaggtgatcgacttccgacctccttacctgttcatccccaagtctctgctgtctggattcatctacaaccacctccgattctcttcgaagggcgtgtgcatgcgaatggacgacgtggaaaagaccgacgacgttgtgaccgagatcatcaacatctctcccaccgagttccaggccatcaaggccaacattaagtctaacatccagggcaagtgtaccatcactccctttctgcacgtgtgctggttcgtgtctctgcacaagtggggcaagttctttaagcccctgaacttcgagtggctgaccgacatcttcatccccgccgactgccgatctcagctgcctgacgacgacgagatgcgacagatgtaccgatacggcgccaacgtgggcttcatcgacttcaccccttggatctctgagttcgacatgaacgacaacaaggaaaacttctggcccctgatcgagcactaccacgaggtgatttctgaggccctgcgaaacaagaagcacctccacggcctgggcttcaacattcagggcttcgtccagaagtacgtcaacattgacaaggtgatgtgcgaccgagccatcggcaagcgacgaggcggcaccctgctgtctaacgtgggcctgttcaaccagctcgaggaacccgacgccaagtactctatctgcgacctggccttcggccagttccaaggctcttggcaccaggctttctccctgggcgtgtgttctaccaacgtgaagggcatgaacatcgtggtggcctctaccaagaacgtggtgggctctcaagagtctctggaagaactgtgctctatctacaaggccctgctgctgggcccctaa

SEQ ID NO: 61 - protein, 525 aa, Saccharomyces cerevisiae
MNEIDEKNQAPVQQECLKEMIQNGHARRMGSVEDLYVALNRQNLYRNFCTYGELSDYCTRDQLTLALREICLKNPTLLHIVLPTRWPNHENYYRSSEYYSRPHPVHDYISVLQELKLSGVVLNEQPEYSAVMKQILEEFKNSKGSYTAKIFKLTTTLTIPYFGPTGPSWRLICLPEEHTEKWKKFIFVSNHCMSDGRSSIHFFHDLRDELNNIKTPPKKLDYIFKYEEDYQLLRKLPEPIEKVIDFRPPYLFIPKSLLSGFIYNHLRFSSKGVCMRMDDVEKTDDVVTEIINISPTEFQAIKANIKSNIQGKCTITPFLHVCWFVSLHKWGKFFKPLNFEWLTDIFIPADCRSQLPDDDEMRQMYRYGANVGFIDFTPWISEFDMNDNKENFWPLIEHYHEVISEALRNKKHLHGLGFNIQGFVQKYVNIDKVMCDRAIGKRRGGTLLSNVGLFNQLEEPDAKYSICDLAFGQFQGSWHQAFSLGVCSTNVKGMNIVVASTKNVVGSQESLEELCSIYKALLLGP

SEQ ID NO: 62 - DNA, 406 bp, Yarrowia lipolytica
agagaccgggttggcggcgcatttgtgtcccaaaaaacagccccaattgccccaattgaccccaaattgacccagtagcgggcccaaccccggcgagagcccccttctccccacatatcaaacctcccccggttcccacacttgccgttaagggcgtagggtactgcagtctggaatctacgcttgttcagactttgtactagtttctttgtctggccatccgggtaacccatgccggacgcaaaatagactactgaaaatttttttgctttgtggttgggactttagccaagggtataaaagaccaccgtccccgaattacctttcctcttcttttctctctctccttgtcaactcacacccgaaatcgttaagcatttccttctgagtataagaatcattcaaa

SEQ ID NO: 63 - DNA, 531 bp, Yarrowia lipolytica
agagaccgggttggcggcgcatttgtgtcccaaaaaacagccccaattgccccaattgaccccaaattgacccagtagcgggcccaaccccggcgagagcccccttctccccacatatcaaacctcccccggttcccacacttgccgttaagggcgtagggtactgcagtctggaatctacgcttgttcagactttgtactagtttctttgtctggccatccgggtaacccatgccggacgcaaaatagactactgaaaatttttttgctttgtggttgggactttagccaagggtataaaagaccaccgtccccgaattacctttcctcttcttttctctctctccttgtcaactcacacccgaaatcgttaagcatttccttctgagtataagaatcattcaaaatggtgagtttcagaggcagcagcaattgccacgggctttgagcacacggccgggtgtggtcccattcccatcgacacaagacgccacgtcatccgaccagcactttttgcagtactaaccgcag

SEQ ID NO: 64 - DNA, 1614 bp, Artificial sequence
  Biobricks
  misc_feature (1)..(1614): BB8251
agagaccgggttggcggcgcatttgtgtcccaaaaaacagccccaattgccccaattgaccccaaattgacccagtagcgggcccaaccccggcgagagcccccttctccccacatatcaaacctcccccggttcccacacttgccgttaagggcgtagggtactgcagtctggaatctacgcttgttcagactttgtactagtttctttgtctggccatccgggtaacccatgccggacgcaaaatagactactgaaaatttttttgctttgtggttgggactttagccaagggtataaaagaccaccgtccccgaattacctttcctcttcttttctctctctccttgtcaactcacacccgaaatcgttaagcatttccttctgagtataagaatcattcaaaatggtgagtttcagaggcagcagcaattgccacgggctttgagcacacggccgggtgtggtcccattcccatcgacacaagacgccacgtcatccgaccagcactttttgcagtactaaccgcaggctccctactctcgaatctaccaccaggacaagtcgtcccgagagactggcgtgctgttcgaggacgacgcccagaccgtggactctgacctgaccaccgaccgattccagctgaagcgagccgagaagcgacgactgcccctggtgtggcgaaacatcatcctgttcgccctggtgcacctggccgctctgtacggcctgcactctatcttcacccgagccaagctggccaccactctgttcgctgccggcctgtacatcatcggcatgctgggcgtgaccgctggcgcccaccgactgtgggctcaccgaacctacaaggccaagtggcccctgcgactgctgctggtgatcttcaacaccattgccttccaggacgccgtgtaccactgggcccgagatcaccgagtgcaccacaagtactctgagactgacgctgaccctcacaacgctacccgaggcttcttcttctctcacgtcggctggctgctgtgcaagaagcaccccgacatcaaggaaaagggccgaggcctggacctgtctgacctgcgagctgaccccatcctgatgttccagcgaaagcactactacattctgatgcccctggcctgcttcgtgctgcccaccgtgattcccatggtgtactggaacgagactctggcctcttcctggttcgtggccaccatgttccgatggtgcttccagctcaacatgacctggctggtgaactctgccgctcacaagttcggcaaccgaccttacgacaagactatgaaccccactcagaacgccttcgtgtctgccttcaccttcggcgaaggctggcacaactaccaccacgcattcccttgggactacaagaccgccgagtggggctgctactctctgaacatcaccaccgccttcatcgacctgttcgctaagatcggctgggcctacgacctcaagaccgtggctcccgacgtgatccagcgacgagtgctgcgaaccggcgacggctctcacgagctgtggggctggggcgacaaggacctgaccgctgaggacgcccgaaacgtcctgctggtggacaagtctcgataa

SEQ ID NO: 65 - DNA, 1509 bp, Artificial sequence
  Biobricks
  misc_feature (1)..(1509): BB8248
agagaccgggttggcggcgcatttgtgtcccaaaaaacagccccaattgccccaattgaccccaaattgacccagtagcgggcccaaccccggcgagagcccccttctccccacatatcaaacctcccccggttcccacacttgccgttaagggcgtagggtactgcagtctggaatctacgcttgttcagactttgtactagtttctttgtctggccatccgggtaacccatgccggacgcaaaatagactactgaaaatttttttgctttgtggttgggactttagccaagggtataaaagaccaccgtccccgaattacctttcctcttcttttctctctctccttgtcaactcacacccgaaatcgttaagcatttccttctgagtataagaatcattcaaaatggtgagtttcagaggcagcagcaattgccacgggctttgagcacacggccgggtgtggtcccattcccatcgacacaagacgccacgtcatccgaccagcactttttgcagtactaaccgcaggtgcccaacaagggttcttccgacgtcctgtctgagcactccgagccccagttcaccaagctgattgctccccaggctggcccccgaaagtacaagatcgtgtaccgaaacctgctgaccttcggatactggcacctgtctgccgtctacggtctgtacctgtgtttcacctgcgccaagtgggctaccattctgttcgccttcttcctgtacgtgatcgctgagatcggcattaccggcggagcccaccgactgtgggctcaccgaacctacaaggccaagctgcccctggagatcctgctgctgattatgaactctatcgctttccaggacaccgccttcacctgggctcgagatcaccgactgcaccacaagtactctgacaccgacgctgaccctcacaacgctacccgaggtttcttctactcccacgtgggctggctgctggtcaagaagcaccccgaggtgaaggcccggggaaagtacctgtccctggacgacctgaagaacaaccccctgctgaagttccagaagaagtacgctatcctggtcattggcaccctgtgtttcctgatgcccaccttcgtgcccgtctacttctggggtgagggcatttctaccgcctggaacatcaacctgctgcgatacgtgatgaacctgaacatgaccttcctggtcaactccgccgctcacattttcggcaacaagccctacgacaagtctattgcctccgtgcagaacatctctgtctccctggctaccttcggagagggtttccacaactaccaccacacctacccttgggactaccgagctgctgagctgggcaacaaccgactgaacatgaccaccgccttcattgacttcttcgcctggatcggatgggcttacgacctgaagtccgtcccccaggaagccatcgctaagcgatgcgctaagaccggcgacggaaccgacatgtggggacgaaagcgatag

SEQ ID NO: 66 - DNA, 1134 bp, Yarrowia lipolytica
atgtggggaagttcacatgcattcgctggtgaatctgatctgacactacaactacacaccaggtccaacatgagcgacaatacgacaatcaaaaagccgatccgacccaaaccgatccggacggaacgcctgccttacgctggggccgcagaaatcatccgagccaaccagaaagaccactactttgagtccgtgcttgaacagcatctcgtcacgtttctgcagaaatggaagggagtacgatttatccaccagtacaaggaggagctggagacggcgtccaagtttgcatatctcggtttgtgtacgcttgtgggctccaagactctcggagaagagtacaccaatctcatgtacactatcagagaccgaacagctctaccgggggtggtgagacggtttggctacgtgctttccaacactctgtttccatacctgtttgtgcgctacatgggcaagttgcgcgccaaactgatgcgcgagtatccccatctggtggagtacgacgaagatgagcctgtgcccagcccggaaacatggaaggagcgggtcatcaagacgtttgtgaacaagtttgacaagttcacggcgctggaggggtttaccgcgatccacttggcgattttctacgtctacggctcgtactaccagctcagtaagcggatctggggcatgcgttatgtatttggacaccgactggacaagaatgagcctcgaatcggttacgagatgctcggtctgctgattttcgcccggtttgccacgtcatttgtgcagacgggaagagagtacctcggagcgctgctggaaaagagcgtggagaaagaggcaggggagaaggaagatgaaaaggaagcggttgtgccgaaaaagaagtcgtcaattccgttcattgaggatacagaaggggagacggaagacaagatcgatctggaggaccctcgacagctcaagttcattcctgaggcgtccagagcgtgcactctgtgtctgtcatacattagtgcgccggcatgtacgccatgtggacactttttctgttgggactgtatttccgaatgggtgagagagaagcccgagtgtcccttgtgtcggcagggtgtgagagagcagaacttgttgcctatcagataa

SEQ ID NO: 67 - protein, 377 aa, Yarrowia lipolytica
MWGSSHAFAGESDLTLQLHTRSNMSDNTTIKKPIRPKPIRTERLPYAGAAEIIRANQKDHYFESVLEQHLVTFLQKWKGVRFIHQYKEELETASKFAYLGLCTLVGSKTLGEEYTNLMYTIRDRTALPGVVRRFGYVLSNTLFPYLFVRYMGKLRAKLMREYPHLVEYDEDEPVPSPETWKERVIKTFVNKFDKFTALEGFTAIHLAIFYVYGSYYQLSKRIWGMRYVFGHRLDKNEPRIGYEMLGLLIFARFATSFVQTGREYLGALLEKSVEKEAGEKEDEKEAVVPKKKSSIPFIEDTEGETEDKIDLEDPRQLKFIPEASRACTLCLSYISAPACTPCGHFFCWDCISEWVREKPECPLCRQGVREQNLLPIR

SEQ ID NO: 68 - DNA, 6337 bp, Yarrowia lipolytica
atggtgagtatcgaccgaagcaggatgatctctacatgagatatgcaacgcgtacgtgattcaattgatcctaacacagtaccctaccacaggtgtcaacaccccccagagcgccgcctcattaagaccactggtgctatcgcacggccaaactgagcactcgctgctggtgcccacctctctgtacatcaactgcaccacgctccgagaccagttctacgcctctctacctccagccactgaagacaaggccgacgatgatgagccctcctcctccacagagcttctagctgccttcctgggatttactgccaagaccgtcgaggaagagcccggaccatacgacgacgttctctctctcgtgcttaacgagtttgagacccggtacttgcgaggtaacgacatccacgctgtggcctcctccttgttacaagacgaggacgtgcctaccaccgttggtaagatcaagagggtgattcgagcctactacgccgcacgaattgcctgcaaccggcccatcaaggcccactcgtcggctctgttccgagccgcatctgaagactcggacaacgtctctctgtacgccatcttcggtggccagggaaacaccgaggactactttgaggaactgcgggagatttacgacatctaccaggggctggtcggcgacttcattcgggaatgtggagcccagcttctggcgctgtctcgagatcacattgctgctgagaaaatttataccaagggctttgatatcgtcaagtggctggaacaccccgagaccatccccgactttgagtacctaatttctgctcccatctctgtacccatcatcggtgttatccagctggcacactacgctgtcacctgtcgagttttgggtcttaatcctggccaggtccgagacaacctcaagggtgccactggccattctcagggtctgatcaccgcaattgccatctctgcctccgactcgtgggacgagttctataactctgcctctcgaattctcaagatcttcttcttcatcggtgtccgtgtccaacaggcttacccctccactttcctgcctccctccactctggaagacagtgtcaagcagggtgagggcaagcccactcccatgctgtccatccgagacctgtctctcaaccaggttcaggagttcgtcgatgccaccaacttgcatttgcccgaagataagcagatcgtcgtgtctctgatcaatggtcctcgaaacgttgtcgttactggccccccccagtctctgtatggtctgtgtcttgtgcttcgaaaacagaaggccgagaccggtctggaccaaagccgagtgccccacagtcagcgaaagctcaaattcacacatcgtttcctgcccatcacctctcctttccactcgtacctgctggagaagagcacggatctgatcatcaacgacctggagtcttccggtgtggagtttgtgtcctccgagctcaaggtgcctgtttacgacacctttgatggctccgtgctgtctcagctacccaagggtatcgtcagccgtctggtcaacctcatcactcatctgcccgtcaagtgggagaaggccactcagtttcaggcctcccacattgtggactttggtcccggtggcgcttctggtcttggtctgttgacccacaagaacaaggatggaactggagtgcgaactattcttgctggtgtcattgaccagcccctcgagttcggcttcaagcaggagctgtttgaccgacaggagtcgtccattgtttttgctcaaaactgggccaaggagttttctcccaagctcgtcaagatctcctccaccaacgaggtctatgtcgacaccaaattctctcgtctgactggccgagcccccatcatggtcgctggtatgacccctaccactgtcaaccccaaatttgtggctgccactatgaactccggctaccacatcgagcttggtggtggaggctactttgcccccggtatgatgaccaaggcccttgaacacattgagaagaacactcctcccggatccggtatcaccatcaacctgatctacgtcaaccctcgactgattcaatggggtattcctctgattcaggagcttcgacagaagggtttccccattgaaggtctcaccattggtgccggtgtgccctctctggaggttgctaacgagtggattcaggatctgggcgtcaagcacatcgccttcaagcctggatccatcgaggccatctcctcggtgattcgaatcgccaaggccaacccagactttcctatcatccttcagtggaccggaggtcgaggaggaggacatcattcgtttgaggacttccacgctcccattctgcagatgtactccaagatccgacgatgcagcaacattgtgctgattgccggatctggtttcggtgcttctaccgactcctacccatacctcaccggttcatggtcccgagactttgactaccctcccatgccctttgacggtatcctggttggttctcgagtcatggttgccaaggaggctttcacttctctgggagccaagcagctcattgttgactctccgggtgttgaggattctgagtgggagaaaacctacgacaagcccactggtggcgtcatcaccgttctctccgagatgggtgagcctatccacaagctcgccactcgaggtgtgctcttctggcacgagatggacaagaccgtgttctccctgcccaagaagaagcgtctggaagtgctcaagtccaagcgagcctacatcatcaagcgtctcaacgacgacttccagaagacttggtttgccaagaacgcccagggacaggtgtgtgatctcgaagacctcacctacgcggaggtcatccagcgacttgttgacctcatgtacgtgaagaaggaaagccgatggatcgatgtcactctccgaaatcttgccggcactttcattcgacgagttgaggagcgattctccaccgagacaggtgcctcttctgtgttgcagagcttttccgagctggattccgagcccgagaaggttgtcgagcgggtgtttgagctcttccctgcctctactacccagatcatcaacgctcaagacaaggaccacttcctcatgctgtgtctcaaccccatgcagaagcccgtgcccttcatccctgttctggatgacaactttgagttcttcttcaagaaggactctctgtggcagtgcgaggacctcgcagctgttgtggacgaagacgttggacgaatctgtattcttcagggtcccgttgctgtcaagcactccaagattgtcaacgagcccgtcaaggagattctcgactccatgcacgaaggtcacatcaagcagctgcttgaggatggcgagtacgctggcaacatggccaacatcccccaggtcgaatgctttggtggaaagcctgctcagaacttcggtgacgttgctctcgactctgtcatggttcttgatgacctcaacaagaccgtgttcaagattgagaccggcacctctgctctgccttctgctgcagattggttctctctgctggccggtgacaagaactcttggcgacaggtcttcctgtccactgacaccattgtgcagaccaccaagatgatctccaaccctctgcatcgacttctggagcccatcgcaggtttgcaggttgagattgagcaccctgatgagcccgagaacaccgtcatctctgctttcgagcccatcaacggcaaggtcaccaaggtgctggagctgcgaaagggtgccggagacgtcatttcgctgcagctgatcgaagcgcgtggcgttgaccgagtccccgttgctcttcctctggaattcaagtaccagccccagattggctacgctcccattgttgaggttatgaccgacaggaacacccgaatcaaggagttctactggaagctgtggtttggccaggactccaagtttgagattgacaccgacatcaccgaggaaatcattggcgatgacgttaccatctctggcaaggccattgccgactttgtccacgctgttggcaacaagggcgaggcctttgttggtcgatctacctctgctggtactgtcttcgctcccatggactttgccattgttttgggctggaaggccattatcaaggcaatctttccccgagcaattgatgctgacattctgcgtctggtacatctgtccaacggcttcaagatgatgcctggcgccgaccctctgcagatgggtgatgttgtttccgccactgccaagatcgacactgtcaagaactccgctaccggcaagactgttgctgttcgaggtcttctcacccgagacggcaagcctgtcatggaggttgtttccgaattcttctaccgaggcgaattctccgacttccagaacacttttgagcgacgagaggaggtacccatgcaactgaccctcaaggacgccaaggccgtggccattctctgctccaaggagtggtttgagtacaatggcgacgataccaaggacctcgagggcaagaccattgtgttccgaaactcgtcattcatcaagtacaagaatgagaccgtcttctcttctgtgcacaccaccggtaaggtattgatggagctgccctccaaggaggtcattgagattgccactgttaactaccaggctggcgagtctcatggcaatcccgtcattgattacctggagcgaaatggaaccaccattgagcagcctgttgagtttgagaagcccatccctctgtccaaggcagatgatcttctctccttcaaggctccttcttccaacgagccctacgctggtgtgtccggtgactacaatcccatccacgtgtctcgagcctttgcttcctatgcatcccttcctggaaccatcacccacggtatgtactcttctgctgctgttcgatctctgattgaggtctgggctgccgagaacaatgtgtctcgagttcgagccttctcctgtcagttccagggcatggttttgcccaacgacgagattgtgactcgactggagcacgttggcatgatcaacggtcgaaagatcatcaaggttacctccaccaaccgggagaccgaggctgttgttctgtctggcgaggctgaggtcgagcagcccatctccacctttgtctttactggccagggctctcaggagcagggcatgggtatggacctgtacgcctcttccgaggtggccaagaaggtctgggacaaggctgacgagcacttcttgcagaactacggtttctccatcatcaagatcgttgtggagaaccccaaggagctggatattcattttggaggccccaagggtaagaagatccgagacaactatatctctatgatgttcgagaccattgatgagaagaccggcaacctcatttccgagaagatcttcaaggagattgacgagaccaccgactctttcaccttcaagtcccccaccggtctgctttctgctacccagttcactcagcccgctctgaccctcatggagaaggcgtcctttgaggacatgaaggctaagggtcttgtccccgtggatgcaacctttgctggtcactcccttggtgagtactccgctcttgcttctcttggtgatgtcatgcccatcgagtctcttgttgatgtcgtcttctaccgaggtatgactatgcaggttgctgttccccgagatgcccagggtcggtccaattacggtatgtgcgctgtcaacccctctcgaatctctaccaccttcaacgacgctgctcttcggtttgtcgttgaccacatctccgagcagaccaagtggctgcttgagattgtcaactacaacgttgagaactctcagtacgtgactgccggtgacctgcgagctctcgacaccctcaccaatgtgctcaacgtgctcaaactcgagaagatcaacattgacaagctgctcgagtctctgcctctggagaaggtcaaggagcacctttctgagatcgtcaccgaggtggccaagaagtccgttgctaagcctcagcccattgagctggaacgaggctttgccgtgatccctctcaagggcatctctgtgcctttccactcttcgtacctgcgaaatggtgtcaagcccttccaaaacttcctggtgaagaaggtgcccaagaacgctgtcaaacctgccaacctcattggcaagtacatccccaacctcactgccaagccctttgagatcaccaaggagtactttgaagaggtttacaagctcaccggttccgagaaggtcaagagcatcatcaacaactgggagtcttatgagtccaagcagtaa

SEQ ID NO: 69 - protein, 2086 aa, Yarrowia lipolytica
MYPTTGVNTPQSAASLRPLVLSHGQTEHSLLVPTSLYINCTTLRDQFYASLPPATEDKADDDEPSSSTELLAAFLGFTAKTVEEEPGPYDDVLSLVLNEFETRYLRGNDIHAVASSLLQDEDVPTTVGKIKRVIRAYYAARIACNRPIKAHSSALFRAASEDSDNVSLYAIFGGQGNTEDYFEELREIYDIYQGLVGDFIRECGAQLLALSRDHIAAEKIYTKGFDIVKWLEHPETIPDFEYLISAPISVPIIGVIQLAHYAVTCRVLGLNPGQVRDNLKGATGHSQGLITAIAISASDSWDEFYNSASRILKIFFFIGVRVQQAYPSTFLPPSTLEDSVKQGEGKPTPMLSIRDLSLNQVQEFVDATNLHLPEDKQIVVSLINGPRNVVVTGPPQSLYGLCLVLRKQKAETGLDQSRVPHSQRKLKFTHRFLPITSPFHSYLLEKSTDLIINDLESSGVEFVSSELKVPVYDTFDGSVLSQLPKGIVSRLVNLITHLPVKWEKATQFQASHIVDFGPGGASGLGLLTHKNKDGTGVRTILAGVIDQPLEFGFKQELFDRQESSIVFAQNWAKEFSPKLVKISSTNEVYVDTKFSRLTGRAPIMVAGMTPTTVNPKFVAATMNSGYHIELGGGGYFAPGMMTKALEHIEKNTPPGSGITINLIYVNPRLIQWGIPLIQELRQKGFPIEGLTIGAGVPSLEVANEWIQDLGVKHIAFKPGSIEAISSVIRIAKANPDFPIILQWTGGRGGGHHSFEDFHAPILQMYSKIRRCSNIVLIAGSGFGASTDSYPYLTGSWSRDFDYPPMPFDGILVGSRVMVAKEAFTSLGAKQLIVDSPGVEDSEWEKTYDKPTGGVITVLSEMGEPIHKLATRGVLFWHEMDKTVFSLPKKKRLEVLKSKRAYIIKRLNDDFQKTWFAKNAQGQVCDLEDLTYAEVIQRLVDLMYVKKESRWIDVTLRNLAGTFIRRVEERFSTETGASSVLQSFSELDSEPEKVVERVFELFPASTTQIINAQDKDHFLMLCLNPMQKPVPFIPVLDDNFEFFFKKDSLWQCEDLAAVVDEDVGRICILQGPVAVKHSKIVNEPVKEILDSMHEGHIKQLLEDGEYAGNMANIPQVECFGGKPAQNFGDVALDSVMVLDDLNKTVFKIETGTSALPSAADWFSLLAGDKNSWRQVFLSTDTIVQTTKMISNPLHRLLEPIAGLQVEIEHPDEPENTVISAFEPINGKVTKVLELRKGAGDVISLQLIEARGVDRVPVALPLEFKYQPQIGYAPIVEVMTDRNTRIKEFYWKLWFGQDSKFEIDTDITEEIIGDDVTISGKAIADFVHAVGNKGEAFVGRSTSAGTVFAPMDFAIVLGWKAIIKAIFPRAIDADILRLVHLSNGFKMMPGADPLQMGDVVSATAKIDTVKNSATGKTVAVRGLLTRDGKPVMEVVSEFFYRGEFSDFQNTFERREEVPMQLTLKDAKAVAILCSKEWFEYNGDDTKDLEGKTIVFRNSSFIKYKNETVFSSVHTTGKVLMELPSKEVIEIATVNYQAGESHGNPVIDYLERNGTTIEQPVEFEKPIPLSKADDLLSFKAPSSNEPYAGVSGDYNPIHVSRAFASYASLPGTITHGMYSSAAVRSLIEVWAAENNVSRVRAFSCQFQGMVLPNDEIVTRLEHVGMINGRKIIKVTSTNRETEAVVLSGEAEVEQPISTFVFTGQGSQEQGMGMDLYASSEVAKKVWDKADEHFLQNYGFSIIKIVVENPKELDIHFGGPKGKKIRDNYISMMFETIDEKTGNLISEKIFKEIDETTDSFTFKSPTGLLSATQFTQPALTLMEKASFEDMKAKGLVPVDATFAGHSLGEYSALASLGDVMPIESLVDVVFYRGMTMQVAVPRDAQGRSNYGMCAVNPSRISTTFNDAALRFVVDHISEQTKWLLEIVNYNVENSQYVTAGDLRALDTLTNVLNVLKLEKINIDKLLESLPLEKVKEHLSEIVTEVAKKSVAKPQPIELERGFAVIPLKGISVPFHSSYLRNGVKPFQNFLVKKVPKNAVKPANLIGKYIPNLTAKPFEITKEYFEEVYKLTGSEKVKSIINNWESYESKQ

SEQ ID NO: 70 - DNA, 5947 bp, Yarrowia lipolytica
agtgagtcttgaaatatgggatatgaggaggggtttgaagaggttgcaatcgataactcacgacacggacgaaaaagaataaggaccaacacgatctccagacaaccacagatcagcagtcgaacccccctcaacagcagacaaatgatgttgtggaattgcagtagatgatttcttctgcgacgctagtattggctgtcggcgacactattctctgacagtgcccaatggtctttttattgtgcaccaaccgctgatttgtggctcaggttttgtgacggcgagagtcattctcgtgatgcatgggatgattggtctctttgaagccgacagatcgacatatttccacacacagcaacgacaatgttatcttatccattgccattctaacccagtgcaccccgaagtcgaacaagaactcgcccacgtgctcctgacggagctgctggcctaccaatttgcctcgcccgtgcgatggatcgagacccaggacgtgctgttcaagcagttcaatgtcgagcgagtcgtcgaagtcggcccatccccaactctcgccggcatggcccagcgaacccttaagtccaagtacgagtcatacgacgctgctctgtctctgcagcgagagatcctgtgttactccaaggaccagaaggacatctactaccttgccgatgaggccgatgaagcccctgcccccgctgctggtggtgatgcccccgctgctcctgccgctgccgctcctgccgccgctgccgctcctgctgccgctgccgccccctctggccccgttgccaaggttgaggacgcccccgtcaaggcccaggagattctccacgccctggtcgcccataagctcaagaagacccccgagcaggtgcccctgtccaaggccatcaaagaccttgttggtggtaagtctaccatccagaacgagattctcggtgatctcggaaaggaatttggtgccacccctgagaagcccgaggatactccccttggcgagctggctgagtccttccaggcctcctttgacggcaagctcggtaagcagtcttcttctctcattgcccgactcatgtcctccaagatgcccggagggttctctctcacctctgctcgatcctacctcgacagcagatggggcctggctgctggccgacaggactccgttctgcttgttgctctgatgaacgaacccaagaaccgacttggctctgaagccgaggccaaggcctacctcgacgagcagacccagaagtatgctgcttctgccggtcttaacctgtctgcccccgctggtggtgccgagggtggcaatggcggtggcgccgtcattgactccgctgcctttgacgctctcaccaaggaccagcgatacctggtccagcagcaactcgagttgtttgccaactacctgaagcaggatctgcgacagggctccaaggtggctgctgcccagaaggaggccatggatattctgcaagctgaactggatctttggaactccgagcacggcgaggtctacgctgagggcatcaagcccgccttctctgccctgaaggcccgtgtctacgactcgtactggaactgggctcgacaggactcgctctccatgtactttgacattgttttcggtcgtctctccaccgttgaccgagagattatggctaagtgtatccacctgatgaaccgaaccaaccacaacctgatcgactacatgcagtaccacatggaccacgtccccgttcacaagggagccacctacgagcttgccaagcagctcggtctgcagctcctcgagaactgtaaggagactctcaccgaggcccccgtctacaaggatgtctcttaccccactggaccccagaccaccattgatgtcaagggtaacattgtttacaacgaggtgccccgacccaatgtccgaaagctcgagcagtatgtccacgagatggcctgtggtggtgagctgaccaaggacccctcttttgttggagaaggtgtccagggcgagctcaagaagctgtactctcagatctctgctcttgccaagacccagaccggctctaccctcgacatcgaggctctgtactccgacctggtcgctaagatctcccaggccgaggacgcgtccaagcctgtcgttgagaacaaggctgtttctgcctccatcactcccggcactctcccttttctccacatcaagaagaagaccgaacttggtgcctggaattacgacagcgagaccaccgccacctacctcgatggtcttgaggttgctgcccgtgatggtctcactttccagggcaagactgctctgatcaccggtgctggtgctggctccattggtgcctcaatcctccagggtctcatttccggaggctgcaaagtcattgtcacaacctctcgatactcccgaaaggtgaccgagtactaccagtccctctacaccaagttcggtgctaagggttccactctgattgttgtccccttcaaccaaggctccaagaaggacgtggacgagctggtgtcgttcatctacaacgaccccaagaacggcggtcttggctgggatctggactttgttgttccctttgctgctctgcccgagaacggtattgagctggagcacattgactcaaagtccgagcttgcccatcgaatcatgctcaccaacctcctgcgtctgcttggtaacgtcaagaagcagaaagtggcccattcctacgagactcgacccgcccaggtcatgctgcccctgtcgcccaaccatggcaacttcggctccgatggtctgtactccgagtccaagatctctctcgagactctgttcaaccggtggcacaccgagtcctggggctcttatctcaccattgttggtgtggtgattggctggacccgaggtaccggtctgatgagcgccaacaacatcaccgccgagggtctggagcagctcggcgtccgaaccttctcccagactgagatggccttttccatcatgggtctcatgaccaaggacattgtgcgactggcccagaactcccccgtgtgggccgatctcaacggtggcttccagtacattcccgacctcaagggagttgttggaaagatccgacgagacattgtggagacctccgagatccgacgggctgtggctcaggagactgccattgaacagaaggtggtcaacggcccccacgccgatcttccttaccagaaggtcgaggtcaagccccgagccaacctcaagtttgacttccccaccctcaaatcctacgccgaggtcaaggagctgtctcctgctggtgatgctctggagggtcttctggatctctcttccgtcattgttgtcactggtttcgccgaggtcggtccttggggtaacgcccgaacccgatgggacatggaggccaacggtgtcttctcccttgagggtgccattgagatggcctggatcatgggtctgatcaagcaccacaatggtcccctgcccggcatgcctcagtactctggctggatcgataccaagaccaagcagcccgtcgatgaccgagatatcaagaccaagtacgaggactacctgcttgagcacgccggtatccgactcattgagcctgagctgttccacggctacaaccccaagaagaagaccttcctccaggaggttattgtggagcacgatctcgagccctttgaggcctccaaggagtctgctgagcaatttgctctcgagcagggcgcgaacgttgagatcttcgccgtccccgagtccgaccagtggactgtgcgacttctcaagggcgccaagctcctcattcccaaggccctcaagtttgaccgacttgtggccggccagattcccactggatgggatgcccgacgatacggtattcccgaggacatttgtgaccaggttgaccccatcactctgtacgctcttgtctccactgttgaggctctgttggcctccggtattaccgacccctacgagttctacaagtacgtccacgtgtccgaggtcggtaactgttccggttccggtatgggtggtatcaccgccctgcgaggcatgttcaaggaccggttcatggacaagcctgttcagaacgatattctccaggagtccttcatcaacaccatgtctgcctgggtcaacatgttgctgctctcctcttccggtcccatcaagacccccgttggagcttgtgccactgctgtcgagtctgtggacattggttgcgaaaccattctgtccggcaaggccagaatctgtctggtcggtggttacgatgatttccaggaggagtcttctcaggagtttgcaaacatgaacgcaacatccaacgctgagaccgagatcactcacggccgaactccggccgagatgtctcgacccatcacttccacacgagccggtttcatggaggctcagggtgctggaacccaggtgctgatggccgccgacctcgccatcgccatgggtgtgcccatctactgtatcgttggttacgtcaacactgccaccgacaagattggccgatctgtgcctgctcccggtaagggtatcctgaccactgctcgagagcaccagactctcaaacacgccaaccctctcctcaacatcaagtaccgaaagcgacagctcgattctcgactccgagacattaagcgatgggctgagggcgaaatggaggctattgacattgagcttgacgacgtgtctgacgccgacaaggagtccttcatccaggagcgatctgcccacatccagtctcagtccgatcgaatgatccgagaggctaagaactcttggggtaacgcctttttcaagcaggacgcccgaatctcccccatccgaggagcgctggcaacctacggtctcaccattgatgacatctccgtcgcttctttccatggtacatccaccaaggccaacgagaagaacgagaccaccaccgtcaacgccatgctggagcatctcggcagaacccggggtaaccctgtctacggtatcttccagaagtaccttactggtcaccccaagggagctgctggtgcctggatgctcaacggagccatccaatgcctcaactctggtatcatccctggtaaccgaaacgccgataacgtggatgcctactttgagcagtgccagcacgtggtgttcccctcgcgatctctgcagaccgatggcctcaaggctgcttccgtgacctcctttggtttcggtcagaagggtgcccaggccattgtcatccaccccgactacctgtacgctgccctgacaccctccgagtactccgagtacaccacccgagtcgcccagcgatacaagaaggcttaccgatactaccacaacgccattgccgaggagtccatgttccaggccaaggacaaggctccctactctgctgagctggagcaggaggtctacctggatcctcttgtgcgagtccaccagaacgaggacaccgagcagtactccttcaacgccaaggacctcgctgcctccgcctttgtcaagaactcccacaaggacaccgccaaggtgcttgccaacctcacctcccaggtgtccggttctggtaagaacgttggtgtcgacgttgaggccatctccgccatcaacattgataacgacaccttccttgaccgaaacttcaccgccaacgagcaggcctactgcttcaaggccccctccccccagtcttctttcgctggcacttggtctgccaaggaggctgttttcaagtctctgggcgtcaagtcccagggcggaggagctgagctcaagtccattgagatcactcgagatggcaacggagctcccgtcgtggttcttcacggagctgccaaggacgctgctgcttctaagggtatctccaccgtcaaggtgtccatttcccatgacgactctcaggccgtggctgttgctgttgccgagtag

SEQ ID NO: 71 - protein, 1850 aa, Yarrowia lipolytica
MHPEVEQELAHVLLTELLAYQFASPVRWIETQDVLFKQFNVERVVEVGPSPTLAGMAQRTLKSKYESYDAALSLQREILCYSKDQKDIYYLADEADEAPAPAAGGDAPAAPAAAAPAAAAAPAAAAAPSGPVAKVEDAPVKAQEILHALVAHKLKKTPEQVPLSKAIKDLVGGKSTIQNEILGDLGKEFGATPEKPEDTPLGELAESFQASFDGKLGKQSSSLIARLMSSKMPGGFSLTSARSYLDSRWGLAAGRQDSVLLVALMNEPKNRLGSEAEAKAYLDEQTQKYAASAGLNLSAPAGGAEGGNGGGAVIDSAAFDALTKDQRYLVQQQLELFANYLKQDLRQGSKVAAAQKEAMDILQAELDLWNSEHGEVYAEGIKPAFSALKARVYDSYWNWARQDSLSMYFDIVFGRLSTVDREIMAKCIHLMNRTNHNLIDYMQYHMDHVPVHKGATYELAKQLGLQLLENCKETLTEAPVYKDVSYPTGPQTTIDVKGNIVYNEVPRPNVRKLEQYVHEMACGGELTKDPSFVGEGVQGELKKLYSQISALAKTQTGSTLDIEALYSDLVAKISQAEDASKPVVENKAVSASITPGTLPFLHIKKKTELGAWNYDSETTATYLDGLEVAARDGLTFQGKTALITGAGAGSIGASILQGLISGGCKVIVTTSRYSRKVTEYYQSLYTKFGAKGSTLIVVPFNQGSKKDVDELVSFIYNDPKNGGLGWDLDFVVPFAALPENGIELEHIDSKSELAHRIMLTNLLRLLGNVKKQKVAHSYETRPAQVMLPLSPNHGNFGSDGLYSESKISLETLFNRWHTESWGSYLTIVGVVIGWTRGTGLMSANNITAEGLEQLGVRTFSQTEMAFSIMGLMTKDIVRLAQNSPVWADLNGGFQYIPDLKGVVGKIRRDIVETSEIRRAVAQETAIEQKVVNGPHADLPYQKVEVKPRANLKFDFPTLKSYAEVKELSPAGDALEGLLDLSSVIVVTGFAEVGPWGNARTRWDMEANGVFSLEGAIEMAWIMGLIKHHNGPLPGMPQYSGWIDTKTKQPVDDRDIKTKYEDYLLEHAGIRLIEPELFHGYNPKKKTFLQEVIVEHDLEPFEASKESAEQFALEQGANVEIFAVPESDQWTVRLLKGAKLLIPKALKFDRLVAGQIPTGWDARRYGIPEDICDQVDPITLYALVSTVEALLASGITDPYEFYKYVHVSEVGNCSGSGMGGITALRGMFKDRFMDKPVQNDILQESFINTMSAWVNMLLLSSSGPIKTPVGACATAVESVDIGCETILSGKARICLVGGYDDFQEESSQEFANMNATSNAETEITHGRTPAEMSRPITSTRAGFMEAQGAGTQVLMAADLAIAMGVPIYCIVGYVNTATDKIGRSVPAPGKGILTTAREHQTLKHANPLLNIKYRKRQLDSRLRDIKRWAEGEMEAIDIELDDVSDADKESFIQERSAHIQSQSDRMIREAKNSWGNAFFKQDARISPIRGALATYGLTIDDISVASFHGTSTKANEKNETTTVNAMLEHLGRTRGNPVYGIFQKYLTGHPKGAAGAWMLNGAIQCLNSGIIPGNRNADNVDAYFEQCQHVVFPSRSLQTDGLKAASVTSFGFGQKGAQAIVIHPDYLYAALTPSEYSEYTTRVAQRYKKAYRYYHNAIAEESMFQAKDKAPYSAELEQEVYLDPLVRVHQNEDTEQYSFNAKDLAASAFVKNSHKDTAKVLANLTSQVSGSGKNVGVDVEAISAINIDNDTFLDRNFTANEQAYCFKAPSPQSSFAGTWSAKEAVFKSLGVKSQGGGAELKSIEITRDGNGAPVVVLHGAAKDAAASKGISTVKVSISHDDSQAVAVAVAE

SEQ ID NO: 72 - DNA, 1362 bp, Heliothis subflexa
atggttgtcttgacctccaaagaaactaagccatctgttgctgaattttacgctggtaagtctgttttcattactggtggtactggtttcttgggtaaggttttcattgaaaagttgttgtactcctgcccagatatcggtaatatctacatgttgatcagagaaaagaagggtttgtccgtttccgaaagaatcaagcactttttggatgatcctttgttcaccagattgaaagaaaaaagaccagccgacttggaaaagatcgttttgattccaggtgatattactgctccagatttgggtattacctccgaaaacgaaaagatgttgatcgaaaaggtcagtgtcattattcattctgctgctaccgttaagttcaacgaaccattgccaactgcttggaagattaacgttgaaggtactagaatgatgttggccttgtctagaagaatgaagagaatcgaagttttcatccatatctctaccgcttacactaacaccaacagagaagttgttgacgaaatcttgtatccagctccagctgatattgatcaagttcaccaatatgttaaggacggtatctctgaagaagaaactgaaaaaatcttgaacggtagaccaaacacttacactttcactaaggctttgaccgaacatttggttgctgaaaatcaagcttacgttccaaccattatcgttagaccatcagttgttgctgccattaaggatgaacctattaagggttggttgggtaattggtatggtgctacaggtttgactgtttttactgctaagggtttgaacagagttatctacggtcactcttctaacatcgttgatttgatcccagttgattacgttgccaacttggttattgctgctggtgctaaatcttctaagtctactgaattgaaggtctacaactgctgttcttctgcttgtaacccaattactatcggtaagttgatgtccatgtttgctgaagatgctatcaagcaaaagtcttacgctatgccattgccaggttggtacatttttactaagtacaagtggttggtcttgttgttgaccattttgttccaagttattccagcctacattaccgacttgtacagacatttgattggtaagaacccaagatatatcaagttgcaatccttggtcaatcaaaccagatcctccattgatttcttcaccaaccattcttgggttatgaaggctgatagagtcagagaattattcgcttctttgtctccagcagataagtacttgtttccatgtgatccagtcaacatcaattggagacaatatatccaagattactgctggggtgttagacatttcttggaaaaaaagacttaa

SEQ ID NO: 73 - protein, 453 aa, Heliothis subflexa
MVVLTSKETKPSVAEFYAGKSVFITGGTGFLGKVFIEKLLYSCPDIGNIYMLIREKKGLSVSERIKHFLDDPLFTRLKEKRPADLEKIVLIPGDITAPDLGITSENEKMLIEKVSVIIHSAATVKFNEPLPTAWKINVEGTRMMLALSRRMKRIEVFIHISTAYTNTNREVVDEILYPAPADIDQVHQYVKDGISEEETEKILNGRPNTYTFTKALTEHLVAENQAYVPTIIVRPSVVAAIKDEPIKGWLGNWYGATGLTVFTAKGLNRVIYGHSSNIVDLIPVDYVANLVIAAGAKSSKSTELKVYNCCSSACNPITIGKLMSMFAEDAIKQKSYAMPLPGWYIFTKYKWLVLLLTILFQVIPAYITDLYRHLIGKNPRYIKLQSLVNQTRSSIDFFTNHSWVMKADRVRELFASLSPADKYLFPCDPVNINWRQYIQDYCWGVRHFLEKKT

SEQ ID NO: 74 - DNA, 1371 bp, Helicoverpa assulta
atggttgtcttgacctccaaagaaactaagccatctgttgctgaattttacgctggtaagtctgttttcattactggtggtactggtttcttgggtaagatcttcattgaaaagttgttgtactcctgcccagatatcggtaatatctacatgttgatcagagaaaagaagggtttgtccgtttccgaaagaatcaagcaatttttggatgaccctttgttcaccagattgaaagaaaaaagaccagccgacttggaaaagatcgttttgattccaggtgatattactgctccagatttgggtattacctccgaaaacgaaaagatgttgatcgaaaaggtcagtgtcattattcattctgctgctaccgttaagttcaacgaaccattgccaactgcttggaagattaacgttgaaggtactagaatgatgttggccttgtctagaagaatgaagagaatcgaagttttcatccatatctctaccgcttacactaacaccaacagagaagttgttgacgaaatcttgtatccagctccagctgatattgatcaagttcaccaatatgttaaggacggtatctctgaagaagaaactgaaaaaatcttgaacggtagaccaaacacttacactttcactaaggctttgaccgaacatttggttgctgaaaatcaagcttacgttccaaccattatcgttagaccatcagttgttgctgccattaaggatgaacctattaagggttggttgggtaattggtatggtgctacaggtttgactgtttttactgctaagggtttgaacagagttatctacggtcattcctcttacatcgttgatttgatcccagttgattacgttgccaacttggttattgctgctggtgctaaatcttctaagtctactgaattgaaggtctacaactgctgttcttctgcttgtaacccaattactatcggtaagttgatgtccatgtttgctgaagatgctatcaagcaaaagtcttacgctatgccattgccaggttggtatgtttttacaaagtacaagtggttggtcttgttgttgaccattttgttccaagttattccagcctacattaccgacttgtacagacatttgattggtaagaacccaagatatatcaagttgcaatccttggtcaatcaaaccagatcctccattgatttcttcacctctcattcttgggttatgaaggctgatagagtcagagaattattcgcttctttgtctccagcagataagtacttgtttccatgtgatccaaccgatattaactggacccattacattcaagattactgctggggtgttagacacttcttggaaaaaaagactaccaacaagtaa

SEQ ID NO: 75 - protein, 456 aa, Helicoverpa assulta
MVVLTSKETKPSVAEFYAGKSVFITGGTGFLGKIFIEKLLYSCPDIGNIYMLIREKKGLSVSERIKQFLDDPLFTRLKEKRPADLEKIVLIPGDITAPDLGITSENEKMLIEKVSVIIHSAATVKFNEPLPTAWKINVEGTRMMLALSRRMKRIEVFIHISTAYTNTNREVVDEILYPAPADIDQVHQYVKDGISEEETEKILNGRPNTYTFTKALTEHLVAENQAYVPTIIVRPSVVAAIKDEPIKGWLGNWYGATGLTVFTAKGLNRVIYGHSSYIVDLIPVDYVANLVIAAGAKSSKSTELKVYNCCSSACNPITIGKLMSMFAEDAIKQKSYAMPLPGWYVFTKYKWLVLLLTILFQVIPAYITDLYRHLIGKNPRYIKLQSLVNQTRSSIDFFTSHSWVMKADRVRELFASLSPADKYLFPCDPTDINWTHYIQDYCWGVRHFLEKKTTNK

SEQ ID NO: 76 - DNA, 1437 bp, Bicyclus anynana
atgtaccgaaacgtgaacaacaactacaagggccactctgtgtacaccaacggcgctggctctcgagtgaagtctctgctgtcctctgccaccgacaacaccaacgagtaccagtctatcgccgagtgctacaagggacagtccgtgttcatcaccggcggcaccggcttcgtcggcaaggtgctgctcgagaagctgctgtactcttgccccggcatcgacaaggtgtacctgctggtgcgagagactcagggcgccaccgctcaccagcgaatgcagaagctcctggaagaacccgccttctcgcgaatcaaggaagagaaccctcaggccttcgagaaggtgatccccatcgtgggcgacatcacccagcctcagctgggcatcatggccgagaacgaggaactgctgattaaggaagtgtctttcgtctaccacgtggccgccaccaccaagttcaacgagactctggacattgccatgaacgtgaacgtggccggaaccggacgagtgctggacctgtctaagcgaatggaaaacatcaaggccttcgtgtacgtgtctaccgcctactctaacaccgaccgagaggtggtggaagaggtgctgtaccccgctcctgtgtctctgaacgaggtgcacaagctgctgaagatcggcatcaccgacgctcaggtgaaggaactgatcaagggacgacccaacacctacaccttcaccaaggctctggctgagaacctggtggccgacaaccacggacacgtgcccgccatcatcgtgcgaccctctatcgtgtcctcgtctaagaaggaacccatcaccggatggatcgactcttggtacggcgccaccttcctggccaccgtgaccatgaagggcttcaaccgagtgttcgtgtcgtcttacgagtacaacctggacttcatccccgtggactacgtgtccaacctgatcatcgtggccgctgctcgatgcaagtgctctgacaaggtggacgtgtacaactcttctacctccggcgaaaaccctctgaagattggcgccttcttcgacgacatcattgcctactcttgcaagcacaagttctacgacatccctctgcctatggcctacctgactcgataccgatgggtcatgttcctgatcaccctgctgctgcagaccctgcctgcctatatcgccgacctgttcctgctgatcgtgggcaagaagccccgatacgtcaagctggcctctaagatctctgccgctcacgaggtcctggactacttcccctctcgaacctggtctatgtctgcccgacagaccaccgctctgttccagtctctgtctccctcggaccgagatcagtttccttgcgaccccaccgacatcgactggaaggagtacatcgtcacctactgccagggaatccgacagttcctgtgcaagtcttaa

SEQ ID NO: 77 - protein, 478 aa, Bicyclus anynana
MYRNVNNNYKGHSVYTNGAGSRVKSLLSSATDNTNEYQSIAECYKGQSVFITGGTGFVGKVLLEKLLYSCPGIDKVYLLVRETQGATAHQRMQKLLEEPAFSRIKEENPQAFEKVIPIVGDITQPQLGIMAENEELLIKEVSFVYHVAATTKFNETLDIAMNVNVAGTGRVLDLSKRMENIKAFVYVSTAYSNTDREVVEEVLYPAPVSLNEVHKLLKIGITDAQVKELIKGRPNTYTFTKALAENLVADNHGHVPAIIVRPSIVSSSKKEPITGWIDSWYGATFLATVTMKGFNRVFVSSYEYNLDFIPVDYVSNLIIVAAARCKCSDKVDVYNSSTSGENPLKIGAFFDDIIAYSCKHKFYDIPLPMAYLTRYRWVMFLITLLLQTLPAYIADLFLLIVGKKPRYVKLASKISAAHEVLDYFPSRTWSMSARQTTALFQSLSPSDRDQFPCDPTDIDWKEYIVTYCQGIRQFLCKS

SEQ ID NO: 78 - DNA, 1014 bp, Lobesia botrana
atggtgcctcgagccgcttcggaggagaccgaccttaaggaggctacccagcttgagccccgaaagtacgagatcgtgtacactaacgtgatctacttcacctattggcatatcgccggactgtacggtctgtacctgtgttttacctccgctaaatgggagaccatcgtgttcgcttgggcttggtatgtgctcggagagctgggagtgattgccggcgctcatagattgtgggcccaccgaacctacaaggcaaagatgcccctgcagatcatcctgatgctgtttaattgtatcggttttcagaacaccgctaccgattgggttcgagatcaccgagtgcatcacaagcactctgacaccgacgccgacccccataactctcagcgaggcttctttttctctcacgtgggctggctgctgacccgaaagcatcggctggtgaaggagaagggagaagctgttgacatgactgatatctactctaaccctgttttaagattccagaagaagtactctctccccctgatcggcactctttgctttgctctgcccaccctgctgcccgcttactgctggggagaggccgtcggcaccgcttggaacattaacctgctgcgatactgtcttaacctgaacggaaccttcctgactaactccgccgctcacaagtttggctctaagccctatgacaagaccattcctcccacccagaacttgctggtgtctttcatgactctgggagaaggatttcataattaccaccacgtcttctcgtgggactaccgagctgctgagcttggcaacacctacctgaacatgaccactatctttatcgacttcttcgctcttattggatgggcctacgacctgaagaccgttcctgaggatgttattaaaaagcgaatggcccgaactggagatggtactaacctgtggggttggggagacaaggacatgaccaaggaggacgtggtggacaccgagatacgattccacgctaagaaggagtaa

SEQ ID NO: 79 - protein, 337 aa, Lobesia botrana
MVPRAASEETDLKEATQLEPRKYEIVYTNVIYFTYWHIAGLYGLYLCFTSAKWETIVFAWAWYVLGELGVIAGAHRLWAHRTYKAKMPLQIILMLFNCIGFQNTATDWVRDHRVHHKHSDTDADPHNSQRGFFFSHVGWLLTRKHRLVKEKGEAVDMTDIYSNPVLRFQKKYSLPLIGTLCFALPTLLPAYCWGEAVGTAWNINLLRYCLNLNGTFLTNSAAHKFGSKPYDKTIPPTQNLLVSFMTLGEGFHNYHHVFSWDYRAAELGNTYLNMTTIFIDFFALIGWAYDLKTVPEDVIKKRMARTGDGTNLWGWGDKDMTKEDVVDTEIRFHAKKE

SEQ ID NO: 80 - DNA, 2013 bp, Lobesia botrana
atgactgaggtgaccaaggtgaaccccgacctccagcgagaacgagacaactgtacattcaacgttaccgagctgactaacctgatcgacggcggtgtgcagaagaccgaggagcgacggaagcgagaggagatggtgctgaaagaaggtatccacctggacgaggttccctcagagtacctgtcccacaaggagaagtacgagctcgctgtgaagaaggcatgctacctgttcaagatgatccgacgactgcaggaggaagagaacactggaatggagaattaccaggaggtgctgggcggatccctgggatccgctatcctgcgagacggctcccctctcaccctgcattacgtgatgttcatccctaccatcatgggccaggctacggttgagcagcaagtgctttggatcggccgagccttcaattgcgatattatcggaacttacgcccagactgagcttggtcacggaaccttcattcgaggactggaaaccaccgccacttacgatccctctacaaaggagttcgtcctgcactcccccactttgacttcctacaagtggtggcccggaggccttgctcacacggccaattactgtatcgtggtggctcaactttacacacaaggcaagtgccatggcattcacccctttatcgtccagctacgagacgaagagacccacatgcccctgcccggcatcaaggtgggcgaaatcggcgctaaactgggaatgaacggcaccaacaacggcttccttggattcgataaggtgcgaatcccccgagagcacatgctgatgaagaacgccaaggttctcgaggacggtacctatgtgcgagcccccagcagtaagctgacatacggtactatgatgtttgtgcgagttgtgctggtgaacgacgtgtgctcgtatatggcaaaggccgttaccatcgctactagatactctgctgtgcgacaccagtctcagcctaagcccgacgagcccgagccccaaatcctggagtacgtgactcaacagcacaagcttgccatcggcattgcaaccgtgcacgcctttcgactatccgcctcttggctgtggaacatgtacaacaacgtgacagccgagctggacgccggcgacttagagcgacttccagaactgcacgctctgtcttgctgtctgaaggccgtttcgactgctgatgcctctgaatgcgtcgagcgatgccgacttgcttgtggaggccacggatacatgttgtcctctaacttccctctgatgtacggcatggtgaccgccgcttgtacttacgaaggcgagaacaccgtgatgcacctgcagaccgcccggtatctggtcaaggcctggcagcaggcccagggtggcaaccccctgacgcctactgttgcctacatcggatctattggcgccggacgacgaagcccaccttgggataacaccgtggagggcattatcctgggcttccagcgcgtggctgccggtaagattgcccagtgtgttgctaatatcgaaaaacgacagcgaaccggaatgtcttacgaggatgcttggaacatgacctcagtgcagcttgtgtctgctagcgaggcccactgtcgagctttcatcctccggacctacttcgaggagaccgaaaaccaaatcggttccgtctctcccgctctgcgagcagtgctgctgcaactggtcgatttgtacgttgtgttttgggctctgcagcgtgtcggagaccttctgagattcacctctatctctgagcgagacatcgagcagcttcagtcttggtacgaggatcttctgatcaagctgcgagtcaacgctgtcggactggtcgacgctttcgacatccgagacgagatcttgaactctgctctcggagcctacgacgggcgagcttacgagcggcttatggatgaagcccttaagtctcccctgaacgctgaacctgtgaaccagtctttccataagtatctgaagcctttcatgcagggtaagctgtaa

SEQ ID NO: 81 - protein, 670 aa, Lobesia botrana
MTEVTKVNPDLQRERDNCTFNVTELTNLIDGGVQKTEERRKREEMVLKEGIHLDEVPSEYLSHKEKYELAVKKACYLFKMIRRLQEEENTGMENYQEVLGGSLGSAILRDGSPLTLHYVMFIPTIMGQATVEQQVLWIGRAFNCDIIGTYAQTELGHGTFIRGLETTATYDPSTKEFVLHSPTLTSYKWWPGGLAHTANYCIVVAQLYTQGKCHGIHPFIVQLRDEETHMPLPGIKVGEIGAKLGMNGTNNGFLGFDKVRIPREHMLMKNAKVLEDGTYVRAPSSKLTYGTMMFVRVVLVNDVCSYMAKAVTIATRYSAVRHQSQPKPDEPEPQILEYVTQQHKLAIGIATVHAFRLSASWLWNMYNNVTAELDAGDLERLPELHALSCCLKAVSTADASECVERCRLACGGHGYMLSSNFPLMYGMVTAACTYEGENTVMHLQTARYLVKAWQQAQGGNPLTPTVAYIGSIGAGRRSPPWDNTVEGIILGFQRVAAGKIAQCVANIEKRQRTGMSYEDAWNMTSVQLVSASEAHCRAFILRTYFEETENQIGSVSPALRAVLLQLVDLYVVFWALQRVGDLLRFTSISERDIEQLQSWYEDLLIKLRVNAVGLVDAFDIRDEILNSALGAYDGRAYERLMDEALKSPLNAEPVNQSFHKYLKPFMQGKL

SEQ ID NO: 82 - DNA, 2094 bp, Lobesia botrana
atggagtctaaggactttgaccctgtgtccgatgctgagttgaaagactacttccccgacctcccttctggtcctctggataagttccgaaagaaggccaccttcgactggagacgaatgaagctagtgtacgactctaagcagtctattgagaccaaggataaggtgtggaagttcatgcttgctcaccctctgttcaagcactctgtcgccacccccaccctggatgagcagcgacagattgctaccaaacgtatgtatctgctgcataacgctgacctggtccctctggaggaaatcgctatgcatccccgactctttcagtcgttgactgaggccattttcatgttcgactcttcggtggccgttaagctgtctcttaccttccgaatgttcaccaacacaattcgcggatcgggcagacagcatcactaccacttgattgaggactgcgacaacggcaagatcggcggatgcttcgccctgactgagatcgcccacggatctaatgctaagggcatgcgaaccaccgcaacctacgacgtggagggccgatgtttcgtcatgcacacccccgactttgaagctgccaagtgttgggttggctccctgggtaagtgtgctacccacgccattgtctacgccatgctgatctctaagggcaaaaaccatggtcttcattccttcgtggtgcccatccgagatcctaagacactccgaccttttgcaggcgtgactgtcggagacatcggcgagaagatcggactgaacggagtggacaacggtttcctgatgttcaacaagtaccgactgcccaaggaggccgctctcgacaagctgggtggagttgacgaaaacggcgactacaagactcctttcagagatccttccaagcggttcggtgcttccctgggcattctgtctggcggccgagtgcacattacctctatctctaccaactacctccagaaggctgtggtgatcgctgtgcgatactctgccgtgcgccgacagtttggccccgagaacgccgtcgaggagactcctgtccttgagtaccagcaacagcagattcgacttctgccctacctggccgctactttcgccatgcgagtgttctgtaactggttcggcgaagtgcacgtgcagatgaccatcgataacctggttggtgccgacgagcaggctgcggcccgaggtatcgagatgcatgccctgtcttccgccgtgaagcccgtgtgcggttggaccgcccgagacggactgcagaactgtagagaatgttgcggtggtcatggatacctccgagcctccgccattggcgacctgcgaaacgacaacgacgcaaactgtacctacgagggcgagaactctatgctcctccagcagacttctaactggctgctgggcgtctgggctagacgacgacagcccggtttcgccgacacacctttcggatctatccaattcctggatggcgctcaggccctgctggacgagtcttgtcagtggaactccgctgaagagatcgtgcagccttccaacgtcgtccacatgtacaagtggctaaccacctacatgctcaagctcaccgctgataaggtgtcccagcttcgatctcgaggttcgtctaactacgaggtccgaaacgattcgcagtcctacaacgctgtgactctgtctgtggtgtatggagagcactttattgtgaatcacttctacaagactgcactgcagttccaggacgccgcctgtagagctgtcctgctgaagctggtggccctgtacggagccttccttctcgagaagcacatggctaccctttatatcggaggattctttaccaagcagcagggactgctcctgcgcgaaggagttctctctctctgcagcgccctgtcaccccaggccgtgtcgctggccgacaccctcgctccccctgattggtgtctgaactctgtgctgggatgcgctgacggcgaggcgtacaaacatatccaggattctattatgacttaccctggctcgatgacccggcctgagtggtggcgagatgttgcccactgggagacttacgtgcccgccaagctgtaa

SEQ ID NO: 83 - protein, 697 aa, Lobesia botrana
MESKDFDPVSDAELKDYFPDLPSGPLDKFRKKATFDWRRMKLVYDSKQSIETKDKVWKFMLAHPLFKHSVATPTLDEQRQIATKRMYLLHNADLVPLEEIAMHPRLFQSLTEAIFMFDSSVAVKLSLTFRMFTNTIRGSGRQHHYHLIEDCDNGKIGGCFALTEIAHGSNAKGMRTTATYDVEGRCFVMHTPDFEAAKCWVGSLGKCATHAIVYAMLISKGKNHGLHSFVVPIRDPKTLRPFAGVTVGDIGEKIGLNGVDNGFLMFNKYRLPKEAALDKLGGVDENGDYKTPFRDPSKRFGASLGILSGGRVHITSISTNYLQKAVVIAVRYSAVRRQFGPENAVEETPVLEYQQQQIRLLPYLAATFAMRVFCNWFGEVHVQMTIDNLVGADEQAAARGIEMHALSSAVKPVCGWTARDGLQNCRECCGGHGYLRASAIGDLRNDNDANCTYEGENSMLLQQTSNWLLGVWARRRQPGFADTPFGSIQFLDGAQALLDESCQWNSAEEIVQPSNVVHMYKWLTTYMLKLTADKVSQLRSRGSSNYEVRNDSQSYNAVTLSVVYGEHFIVNHFYKTALQFQDAACRAVLLKLVALYGAFLLEKHMATLYIGGFFTKQQGLLLREGVLSLCSALSPQAVSLADTLAPPDWCLNSVLGCADGEAYKHIQDSIMTYPGSMTRPEWWRDVAHWETYVPAKL

SEQ ID NO: 84 - DNA, 2064 bp, Lobesia botrana
atggaggacctctccttcattcccgacctgccgtccggacctctggacgagtaccgatcctctgcatcctttgactggaagcgactgaagcttgccctcgagggtgatatcgagctcctgaaactgaagtacaaggtctggcatactctcgaaaaggatcccctgttcgcccaccccgccattacgcccacggtcgaggagcagaagcggattgcccagctgcaggttaaccggattaaccagtacaacttcctgtctggtgacatgctgagatctggctactccaagaagacccgagtgattatgaccatcaacgaggctgtgcaatctctgaatccctctgtgtcagtgaagatggctatcggaatttacctaccctccaacgctcttctgtccctgggcacagaacggcactacaagttctacgaggccactctgatcaagcgcgagattctgaccgctttcgctcttactgaggttgcccatggatctgacgcacgattgatgaggaccactgctacctacgacgctcaggcccgagagtttgtgctgcactctccggacttcgaggccgccaaatgttgggtgggtaatctgggccgaacctgcacccatgcccttgtgttcgctcagctgatcactcccgatggcgccagccatggacttcacggcttcgttgtgcccatccgagaccctgacacccttgagacctaccctggccttctcgtgggcgacatgggtgagaaaatcggagtgaacggaattgataacggatttatgatgttcaaccagtaccgaatccctcgagagaacctgctgaaccgaactgctgatgttactgaggagggtgtgtacgagtccactttttcggagccctctaagatccttggagctgctctggagaacctgtcagctggacgaatgggcattatgcaagagtcttgcaattctatcgctatgtgcgcctctatcgccgtgcgatatgccgctactagacgacagttcggatccgccgcccaggaaattcctctgttggagtacgagctccaccagtggcgattgtttggatttgtggccgctgccgtcgtattcaaaatttacattgagtctttcactcgagtctacctgaagatcgttgagtcgtctaacgccggacagaaggtggacaacctttctgagtccgtggccgagatccatgccatggtctcctgttccaagcccatgaccacctggaccgttctccgagccgcgcagcagtgccgtgaggcttgtggcggccatggcttcatgaagcgaagcaacctcggcgacatcagatctaaccatgaggccaccgtcacctacgagggcgataactctgtgctgtcccagcaagccggcaattggctgcttcgacagtttgagtgtgctcgagccggcgccgttgactcgccccttggtaccgtgaccttcctgaaccacgctgaggacatcctgcgacgacgattcgcctgcgctgagactcaccagctgaagtccgacgagttcatcttatctacttacaagtggttgatctgttggctgctaaaggagactcaggataagtacgaatcgctgatggcttctggactgaccaagttccaggccaaggctaagtcccaggtgtacaagtggcaaaagctgaccgaggtctacgccgagtacctagccctgttattctcctgggaatctattcagcgaaaggagaaggagctgcagcccgtgctcaagaacctcttcctgctctacggattgtggtccatggaccgacatctggttgagctgtaccagggcggtttcgctacgggcgcttccctggctaccctcgttagggctgccgtgctggagctgtgcgccgccctgaagcatgaggtggtgtctgttgtcgacgccttagcccccaccgacttcgtcttgaactctgtcctgggcaagtctgacggcaagctttaccagaacctccaaaaggagctgttccacaccccaggtggcatgcagcgggccgcttggtggcgagacattctgctcagaccctctaagctttaa

SEQ ID NO: 85 - protein, 687 aa, Lobesia botrana
MEDLSFIPDLPSGPLDEYRSSASFDWKRLKLALEGDIELLKLKYKVWHTLEKDPLFAHPAITPTVEEQKRIAQLQVNRINQYNFLSGDMLRSGYSKKTRVIMTINEAVQSLNPSVSVKMAIGIYLPSNALLSLGTERHYKFYEATLIKREILTAFALTEVAHGSDARLMRTTATYDAQAREFVLHSPDFEAAKCWVGNLGRTCTHALVFAQLITPDGASHGLHGFVVPIRDPDTLETYPGLLVGDMGEKIGVNGIDNGFMMFNQYRIPRENLLNRTADVTEEGVYESTFSEPSKILGAALENLSAGRMGIMQESCNSIAMCASIAVRYAATRRQFGSAAQEIPLLEYELHQWRLFGFVAAAVVFKIYIESFTRVYLKIVESSNAGQKVDNLSESVAEIHAMVSCSKPMTTWTVLRAAQQCREACGGHGFMKRSNLGDIRSNHEATVTYEGDNSVLSQQAGNWLLRQFECARAGAVDSPLGTVTFLNHAEDILRRRFACAETHQLKSDEFILSTYKWLICWLLKETQDKYESLMASGLTKFQAKAKSQVYKWQKLTEVYAEYLALLFSWESIQRKEKELQPVLKNLFLLYGLWSMDRHLVELYQGGFATGASLATLVRAAVLELCAALKHEVVSVVDALAPTDFVLNSVLGKSDGKLYQNLQKELFHTPGGMQRAAWWRDILLRPSKL

SEQ ID NO: 86 - DNA, 1017 bp, Spodoptera litura
atggctcagaccatccagaccaccaccatcctcgagcagaaggaggagaagaccgtgaccctgctggtccctcaggctggtaagagaaagttcgagatcgtctatttcaacctggtctcttttgcctactggcatatcgctggcctgtacggtctttacctgtgcttcacctccgccaagtgggccaccattctgttctccttcttcctgttcgtcgtcgccgaggttggagttaccgccggagctcaccgactgtggtcccacaagacttacaaggccaagctcccccttcagatccttctgatggtcatgaactccctggccttccagaataccgccattgactgggtccgagaccaccggcttcaccataagtactccgacaccgatgccgaccctcataacgcctcccgaggcttcttctactcccacatcggctggctgttcgtgcgaaagcaccccgatgtcaagaagcgaggtaaggagattgacatctccgacatctacaataaccccgtcctgagattccagaagaagtacgccatccctttcattggcgctgtctgcttcgccctgcccacccttatccccgtctatggttggggcgagacctggaccaacgcctggcacgtcgccatgctgcgatacatcatgaacctcaatgttaccttcctggtcaactctgccgcccacatctacggaaagcgaccctacgacaagaagatccttccctctcagaacatcgccgtctccattgccacctttggtgagggtttccacaactaccaccacgtgttcccttgggattaccgagctgccgagctgggcaacaactgcctgaacttcaccaccaagttcattgacttcttcgcttggatcggatgggcctacgacctcaagacggtctccaaggagatgatcaagcagcgatccaagcgaactggcgacggaactaacctgtggggcctggaggacgtcgatactcctgaggacctgaagaacaccaagggcgagtaa

SEQ ID NO: 87 - protein, 338 aa, Spodoptera litura
MAQTIQTTTILEQKEEKTVTLLVPQAGKRKFEIVYFNLVSFAYWHIAGLYGLYLCFTSAKWATILFSFFLFVVAEVGVTAGAHRLWSHKTYKAKLPLQILLMVMNSLAFQNTAIDWVRDHRLHHKYSDTDADPHNASRGFFYSHIGWLFVRKHPDVKKRGKEIDISDIYNNPVLRFQKKYAIPFIGAVCFALPTLIPVYGWGETWTNAWHVAMLRYIMNLNVTFLVNSAAHIYGKRPYDKKILPSQNIAVSIATFGEGFHNYHHVFPWDYRAAELGNNCLNFTTKFIDFFAWIGWAYDLKTVSKEMIKQRSKRTGDGTNLWGLEDVDTPEDLKNTKGE

SEQ ID NO: 88 - DNA, 1059 bp, Lobesia botrana
atgcctccaaacgtgaccgacgtgaacggcgtgctgttcgagtctgacgccgctactcccgacctggctctggctacccctcctgtgcagaaggccgacaacacccctcgagtctacgtgtggcgaaacatcatcctgttcggctacctgcacattgccgctctgtacggcggatacctgttcctggtgtctgctaagtggcagaccgacatcttcgcctacctgctgtacgtggcctctggcctgggcatcaccgctggcgcccaccgactgtgggctcacaagtcttacaaggccaagtggcccctgcgagtgatcctgaccatcttcaacaccattgccttccaggactctgccatcgactgggcccgagatcaccgaatgcaccacaagtactctgagactgacgctgaccctcacaacgctacccgaggcttcttcttctctcacatcggctggctgctggtgcgaaagcaccccgagctgaagcgaaagggcaagggcctcgacctgtctgacctgtacgctgatcccatcctgcgattccagaagaagtactacctgattctgatgcccctggcctgcttcatcatgcccactctgatccccgtgtacttctggggcgagacttggaccaacgccttcttcgtggctgccctgttccgatacgccttcatcctgaacatcacctggctggtgaactctgctgcccacaagtggggcgacaagccctacgacaagaccatcaagccctctgagaacctgtctgtgtctctgttcgccttcggcgagggcttccacaactaccaccacacattcccttgggactacaagaccgccgagctgggcaaccaccgactcaacttcactaccaagttcatcaactttttcgccaagctcggctgggcctacgacatgaagaccgtgtctgacgagatcgtgcagaagcgagtccagcgaaccggcgacggctctcaccacctgtggggctggggagacaaggaccagtctaaggacgaggtggacgccgccatccgaatcaaccccaaggacgactag

SEQ ID NO: 89 - protein, 352 aa, Lobesia botrana
MPPNVTDVNGVLFESDAATPDLALATPPVQKADNTPRVYVWRNIILFGYLHIAALYGGYLFLVSAKWQTDIFAYLLYVASGLGITAGAHRLWAHKSYKAKWPLRVILTIFNTIAFQDSAIDWARDHRMHHKYSETDADPHNATRGFFFSHIGWLLVRKHPELKRKGKGLDLSDLYADPILRFQKKYYLILMPLACFIMPTLIPVYFWGETWTNAFFVAALFRYAFILNITWLVNSAAHKWGDKPYDKTIKPSENLSVSLFAFGEGFHNYHHTFPWDYKTAELGNHRLNFTTKFINFFAKLGWAYDMKTVSDEIVQKRVQRTGDGSHHLWGWGDKDQSKDEVDAAIRINPKDD

SEQ ID NO: 90 - DNA, 1116 bp, Chilo suppressalis
atggacttcgagtctgagctgcagaactgctctaacaagtctcaaaagcgaaacatcatggctcccaacgcctctgacgtgaacggcgtgctgttcgaggacgacgccgctactcccgacatggccctgtctaacacccctgtgcagaaggccgacaactaccccaagaagctggtctggcgaaacattatcgccttcgcctacctgcacattgccgccgtgtacggcggatacctgttcctgttcgccgccaagtggcagaccgacattttcgcctacatcctgtacgtgatgtctggcctgggcatcaccgctggcgcccaccgactgtgggctcacaagtcttacaaggctaagtggcccctgcgactgatcctggtgttcttcaacaccctggccttccaggactctgccatcgactgggcccgagatcaccgaatgcaccacaagtactctgagactgacgctgaccctcacaacgctacccgaggcttcttcttctctcacatcggctggctgctggtgcgaaagcaccccgagctgaagcagaagggcaagggcctcgacctgtctgacctgtacgctgatcccatcctgcgattccagaagaagtactacctgattctgatgcccattgcctgcttcattctgcccaccgtgattcctctgtactactggggcgagaactgggtgaccgccttcttcgtggccgctctgttccgatacgcctttatcctgaacgtgacctggctggtgaactctgctgcccacaagtggggcgacaagccctacgacaagaacatcaagccctctgagaacatctctgtgtctatcttcgccctcggcgagggattccacaactaccaccacacattcccttgggactacaagaccgccgagctgggcaacaaccgactgaacttcactaccaacttcatcaactttttcgccaagatcggctgggcctacgacatgaagaccgtgtctgacgagatcgtgcagtctcgagtgaagcgaaccggcgacggctctcaccacctgtggggctggggagacaaggaccactgcaaggaagaggtggccgctgccatccgaatcaaccccaaggacgactag

SEQ ID NO: 91 - protein, 371 aa, Chilo suppressalis
MDFESELQNCSNKSQKRNIMAPNASDVNGVLFEDDAATPDMALSNTPVQKADNYPKKLVWRNIIAFAYLHIAAVYGGYLFLFAAKWQTDIFAYILYVMSGLGITAGAHRLWAHKSYKAKWPLRLILVFFNTLAFQDSAIDWARDHRMHHKYSETDADPHNATRGFFFSHIGWLLVRKHPELKQKGKGLDLSDLYADPILRFQKKYYLILMPIACFILPTVIPLYYWGENWVTAFFVAALFRYAFILNVTWLVNSAAHKWGDKPYDKNIKPSENISVSIFALGEGFHNYHHTFPWDYKTAELGNNRLNFTTNFINFFAKIGWAYDMKTVSDEIVQSRVKRTGDGSHHLWGWGDKDHCKEEVAAAIRINPKDD

SEQ ID NO: 92 - DNA, 1377 bp, Agrotis segetum
atgcccgtgctgacctcgcgagaggacgagaagctgtctgtgcccgagttctacgccggcaagtctatcttcgtgaccggcggcaccggattcctcggcaaggtgttcattgagaagctgctctactgctgccccgacatcgacaagatctacatgctgatccgagagaagaagaacctgtctatcgacgagcgaatgtctaagttcctggacgaccctctgttctctcgactgaaggaagaacgacccggcgacctcgagaagatcgtgctgatccccggcgacatcaccgctcctaacctgggcctgtctgccgagaacgaacgaatcctgctcgagaaggtgtccgtgatcatcaactctgccgccaccgtgaagttcaacgagcccctgcctatcgcctggaagatcaacgtcgagggcacccgaatgctgctggccctgtctcgacgaatgaagcgaatcgaggtgtttatccacatctctaccgcctactctaacgcctcttctgaccgaatcgtggtggacgagattctgtaccccgctcctgccgacatggaccaggtgtaccagctcgtgaaggacggcgtgaccgaggaagagactgagcgactgctgaacggactgcccaacacctacaccttcaccaaggctctgaccgagcacctggtggccgagcaccagacctacgtgcccaccatcatcattcgaccctccgtggtggcctctatcaaggacgagcccatccgaggctggctgtgcaactggttcggcgccaccggcatctctgtgttcaccgccaagggcctgaaccgagtgctgctcggaaaggcctctaacatcgtggacgtgatccccgtggactacgtggccaacctggtgatcgtggctggcgccaagtctggcggccagaagtctgacgagctgaagatctataactgctgttcttctgactgcaaccccgtgactctgaagaagatcatcaaggaattcaccgaggacaccatcaagaacaagtctcacatcatgcctctgcctggctggttcgtgttcaccaagtacaagtggctgctgaccctcctgaccatcatcttccagatgctgcccatgtacctggccgacgtgtaccgagtcctgaccggcaagattccccggtacatgaagctgcaccacctggtcattcagacccgactgggaatcgacttcttcacctctcactcttgggtgatgaagaccgaccgagtgcgagagctgttcggctctctgtctctggccgagaagcacatgttcccttgcgacccctcttccatcgactggaccgactacctgcagtcttactgctacggcgtgcgacgattcctggaaaagaagaagtag

SEQ ID NO: 93 - protein, 458 aa, Agrotis segetum
MPVLTSREDEKLSVPEFYAGKSIFVTGGTGFLGKVFIEKLLYCCPDIDKIYMLIREKKNLSIDERMSKFLDDPLFSRLKEERPGDLEKIVLIPGDITAPNLGLSAENERILLEKVSVIINSAATVKFNEPLPIAWKINVEGTRMLLALSRRMKRIEVFIHISTAYSNASSDRIVVDEILYPAPADMDQVYQLVKDGVTEEETERLLNGLPNTYTFTKALTEHLVAEHQTYVPTIIIRPSVVASIKDEPIRGWLCNWFGATGISVFTAKGLNRVLLGKASNIVDVIPVDYVANLVIVAGAKSGGQKSDELKIYNCCSSDCNPVTLKKIIKEFTEDTIKNKSHIMPLPGWFVFTKYKWLLTLLTIIFQMLPMYLADVYRVLTGKIPRYMKLHHLVIQTRLGIDFFTSHSWVMKTDRVRELFGSLSLAEKHMFPCDPSSIDWTDYLQSYCYGVRRFLEKKK

SEQ ID NO: 94 - DNA, 24 bp, Artificial sequence
  Primer sequence
  misc_feature: PR-22847
gtgcaggugccacaatgaccgagg

SEQ ID NO: 95 - DNA, 25 bp, Artificial sequence
  Primer sequence
  misc_feature: PR-22848
cgtgcgauctacagcttcgaagaag

SEQ ID NO: 96 - DNA, 1026 bp, Grapholita molesta
  misc_feature (1)..(1026): codon-optimised for Yarrowia lipolytica
atgacccctgacgctcgagaggaaaccctgctcgaggacgacggctacgtgcgactggtggctccccaggcctcttctggaaagcaagaggcctcctacctgaacattgccatgtactcgttcttccacaccgctggcgcctacggcctgtacctggccgccaccgaggccaagtgggctaccatcttcctggccttcatcctgcacgaggccgccatcctgggcgtgaccgccggtgctcaccgactgtgggctcaccgaacctacaaggccaagctgcccctgcagatcttcctgatgctgctgcactctttcgcctgccgatactctgccttccactgggcccgagatcaccgactccaccacaagtactctgacaccgacggcgaccctcacaacgcctctcgaggcctgttcttctctcacatcggctggctgatgttcaagaagcaccccgaggctaagaagcgaatgggcaagatcgacgtgtctgacctgaaggccaacaaggtcctgatgtttcagaagaagtactctatccccttcatcggcaccatctgcttcattctgcccactctgatccctatgtacttctgggacgagactttctcttctgccttcttcgtgaccgtgctgcgagtgatcgtgtctctgcacgtgacctttctggtgaactccttcgctcacgccttcggcaaccgaccttacgaccggtacatcaagccctctcagtctatccccatctctctggctaccctcggcgagggctaccacaactaccaccacgcattcccttgggactacaaggcttctgagttcggcaacaccatcggcaacttcactactatggtgatcaacttcttcgcctggctcggctgggcttacgacctgaagtctgtgggcgccgacatgatcgccaagaagaaggaaaagaccggcgacggcaccaacctgtggggctggggcgacaaggacatgtctaaggaacacaaggaaatcgccaacgtcacttacaaggaaaagtag

SEQ ID NO: 97 - protein, 341 aa, Grapholita molesta
MTPDAREETLLEDDGYVRLVAPQASSGKQEASYLNIAMYSFFHTAGAYGLYLAATEAKWATIFLAFILHEAAILGVTAGAHRLWAHRTYKAKLPLQIFLMLLHSFACRYSAFHWARDHRLHHKYSDTDGDPHNASRGLFFSHIGWLMFKKHPEAKKRMGKIDVSDLKANKVLMFQKKYSIPFIGTICFILPTLIPMYFWDETFSSAFFVTVLRVIVSLHVTFLVNSFAHAFGNRPYDRYIKPSQSIPISLATLGEGYHNYHHAFPWDYKASEFGNTIGNFTTMVINFFAWLGWAYDLKSVGADMIAKKKEKTGDGTNLWGWGDKDMSKEHKEIANVTYKEK

SEQ ID NO: 98 - DNA, 1047 bp, Cydia pomonella; identical to SEQ ID NO: 49

SEQ ID NO: 99 - protein, 348 aa, Cydia pomonella; identical to SEQ ID NO: 50

SEQ ID NO: 100 - DNA, 984 bp, Pectinophora gossypiella
atggctaaccccgactctgaggctgtgcccttccgacacaccctggtgaagcacaacgtgatcaagttcggctacatgcacctggccgctctgtacggcgtgtacctgtgtttcacctctgccaagtggcctactctgctgtgggccttcctgctgctcgagtgcgccaagatcggcatcaccgctggcgcccaccgactgtggtgccaccgatcttacaaggccaagctgcccctcgagattctgctgctgatcttcaactctatcgcctacatgaacaccgccacctactgggtgcgagatcaccgagtgcaccacaagttcgccgacaccgacgctgaccctcacaacgtcaaccgaggcttctggttctctcagatcggctggctgttcgtgcgaaagcaccccgacgtgctgaacaagggccagaccgtgtacatggaagatatccacaagaaccctctgctgcgattccagaagaagtacgccttcttcgtcatcggcctgtgggcttacgtgatccccaccgtggtgcctatgtacttctggggcgagtctttcaacaactcttggcacatctgcaccatgctgcgacacgtgctgactatcaaccagatcttcctggtgaactccatcggccactcttggggcaacaagccctacgacaagaacatccgagccgtcgagaacattgccgtgtctctgatgtctaccggcgagtgcttccacaactaccaccacgtgttcccattcgactacaaggcctctgagctgggcatgaccaagttcaacgccgctactatgttcatcaactttttcgcctggctcggctgggcctacgacctcaagaccattcctgacgagctgatcattgcccgatctaagcgaaccggcgacggccgaaacctgtggggctggggagacaccgaccagaccgaggaagagactaacggcgtcaagtacctgtaccagaagggcgagtaa

SEQ ID NO: 101 - protein, 327 aa, Pectinophora gossypiella
MANPDSEAVPFRHTLVKHNVIKFGYMHLAALYGVYLCFTSAKWPTLLWAFLLLECAKIGITAGAHRLWCHRSYKAKLPLEILLLIFNSIAYMNTATYWVRDHRVHHKFADTDADPHNVNRGFWFSQIGWLFVRKHPDVLNKGQTVYMEDIHKNPLLRFQKKYAFFVIGLWAYVIPTVVPMYFWGESFNNSWHICTMLRHVLTINQIFLVNSIGHSWGNKPYDKNIRAVENIAVSLMSTGECFHNYHHVFPFDYKASELGMTKFNAATMFINFFAWLGWAYDLKTIPDELIIARSKRTGDGRNLWGWGDTDQTEEETNGVKYLYQKGE

SEQ ID NO: 102 - DNA, 28 bp, Artificial sequence
  Primer sequence
  misc_feature: PR-21661
ataattacuctccacaggaggatcggtg

SEQ ID NO: 103 - DNA, 25 bp, Artificial sequence
  Primer sequence
  misc_feature: PR-21662
agtaattaugagatacattctttgc

SEQ ID NO: 104 - DNA, 20 bp, Artificial sequence
  Primer sequence
  misc_feature: PR-21673
aaagagcaugccactgcaac

SEQ ID NO: 105 - DNA, 31 bp, Artificial sequence
  Primer sequence
  misc_feature: PR-21674
atgctcttutgattttgtaggttttagtaag

SEQ ID NO: 106 - DNA, 29 bp, Artificial sequence
  Primer sequence
  misc_feature: PR-18977 (PrTef->_fw)
agctactgauagagaccgggttggcggcg

SEQ ID NO: 107 - DNA, 41 bp, Artificial sequence
  Primer sequence
  misc_feature: PR10604
cgtgcgauaccgtacccacacaaaaaaagcaccaccgactc

SEQ ID NO: 108 - DNA, 33 bp, Artificial sequence
  Primer sequence
  misc_feature: PR10655
acccattgcugtagatatgtcttgtgtgtaagg

SEQ ID NO: 109 - DNA, 32 bp, Artificial sequence
  Primer sequence
  misc_feature: PR10656
atcatgtaautagttatgtcacgcttacattc

SEQ ID NO: 110 - DNA, 31 bp, Artificial sequence
  Primer sequence
  misc_feature: PR11110
atcgcgtgcattcgcggccgcatttaaatcc

SEQ ID NO: 111 - DNA, 37 bp, Artificial sequence
  Primer sequence
  misc_feature: PR11111
atcgcacgcattcgcggccgcaaatttaaataaaatg

SEQ ID NO: 112 - DNA, 30 bp, Artificial sequence
  Primer sequence
  misc_feature: PR11138
agcaatggguaaaaagcctgaactcaccgc

SEQ ID NO: 113 - DNA, 30 bp, Artificial sequence
  Primer sequence
  misc_feature: PR11139
attacatgautattcctttgccctcggacg

SEQ ID NO: 114 - DNA, 31 bp, Artificial sequence
  Primer sequence
  misc_feature: PR14148
acgtgcaacgctuacgcaactaacatgaatg

SEQ ID NO: 115 - DNA, 30 bp, Artificial sequence
  Primer sequence
  misc_feature: PR14149
agctgttcucagatgcattcttgggcggtc

SEQ ID NO: 116 - DNA, 27 bp, Artificial sequence
  Primer sequence
  misc_feature: PR14588
acgcgauaattattgcacaggacacac

SEQ ID NO: 117 - DNA, 28 bp, Artificial sequence
  Primer sequence
  misc_feature: PR15522
atgacagautgctgtagatatgtcttgt

SEQ ID NO: 118 - DNA, 27 bp, Artificial sequence
  Primer sequence
  misc_feature: PR15781
aggccacucagatgcattcttgggcgg

SEQ ID NO: 119 - DNA, 30 bp, Artificial sequence
  Primer sequence
  misc_feature: PR15788
taaccaaccugcgccgacccggaatcgaac

SEQ ID NO: 120 - DNA, 33 bp, Artificial sequence
  Primer sequence
  misc_feature: PR15789
gttttagagcuagaaatagcaagttaaaataag

SEQ ID NO: 121 - DNA, 30 bp, Artificial sequence
  Primer sequence
  misc_feature: PR15930
acctgcggtuagtactgcaaaaagtgctgg

SEQ ID NO: 122 - DNA, 25 bp, Artificial sequence
  Primer sequence
  misc_feature: PR20763
agggcggugaaaccacccataccgg

SEQ ID NO: 123 - DNA, 28 bp, Artificial sequence
  Primer sequence
  misc_feature: PR20764
accgcccugcgaggcatgttcaaggacc

SEQ ID NO: 124 - DNA, 29 bp, Artificial sequence
  Primer sequence
  misc_feature: PR21771
agctactgauaaggagtttggcgcccgtt

SEQ ID NO: 125 - DNA, 29 bp, Artificial sequence
  Primer sequence
  misc_feature: PR21925
aaccgaggutgctgtagatatgtcttgtg

SEQ ID NO: 126 - DNA, 29 bp, Artificial sequence
  Primer sequence
  misc_feature: PR22075
agaacagcuagagaccgggttggcggcgc

SEQ ID NO: 127 - DNA, 36 bp, Artificial sequence
  Primer sequence
  misc_feature: PR22213
atgacagautgttgatgtgtgtttaattcaagaatg

SEQ ID NO: 128 - DNA, 1224 bp, Cydia pomonella
  misc_feature (1)..(1224): mRNA-coding sequence, codon-optimized for Y. lipolytica
atgcctccccgagagtctaagaaggtggccctgcgatcttacgagacccctgtcgcttctctccctcctcgaaagtacgagattatctacctcaacctcttcctgcacatcgctggacatatctccgccgtctacggcctgtatctgtgcttcaccgccgcccagtggaagaccatcttctttgcctacctgtggctgttgatgggcgagctcggcgtggtgtgtggcgctcacagattgtggtctcaccgttctttcaaggtgaagcctcctctcgagatcatgctgatgctgttcaactgtattggattccagaacaccgccactgactgggtccgaaaccaccggctccatcacaagcactctgacactgacgccgacccccataactctaaccgaggaatgctgttctcccacattggctggctgtgtgtgcgaaagcacccagatgttaaagaacgaggcaagaccaccgacatgtctgacatctactctaaccccgtgctccgattccagaagaagcacaaggtaccccttttcggcgccatgtgtttcggcctgcccacccttattcccaccctgtggggagaggacatcgtcaccgcttggcacgtcaacctgctgcgattcgttcttaatctgaactctatcctgctggtcaactccattgctcataagtacggcacccgaccctacgatcgaaccatctgccctcgacaaaacaccacctgtaacatgatgactcttggagagggcttccacaactaccaccacacctttccttgggactaccgatttgtgttctctaagcagactgacccctacatctcttctgagtgctggcccactcgaccccgacctcgaggccccggaaacctggccatggtgctccgatggcgatctcctccaccacctcctcagtctgccgagctgggaaagaactacctgaacttcaccaagtggttcatcgacttcttcgccctgattggatgggcctacgacctgaagaccgttcctgacgatatgatccagcgacgaatgaaaagaaccggagacggatccaactcgtggggatggggagacaaggacatgactaaggaggagcgagactctgctactatcatttatcccgagaagaaggatgatattaagatgatctccaaaaagaacgactactccaagatcttctctcacgactacttccgactgttcgacgagctgtaa

SEQ ID NO: 129 - protein, 407 aa, Cydia pomonella
MPPRESKKVALRSYETPVASLPPRKYEIIYLNLFLHIAGHISAVYGLYLCFTAAQWKTIFFAYLWLLMGELGVVCGAHRLWSHRSFKVKPPLEIMLMLFNCIGFQNTATDWVRNHRLHHKHSDTDADPHNSNRGMLFSHIGWLCVRKHPDVKERGKTTDMSDIYSNPVLRFQKKHKVPLFGAMCFGLPTLIPTLWGEDIVTAWHVNLLRFVLNLNSILLVNSIAHKYGTRPYDRTICPRQNTTCNMMTLGEGFHNYHHTFPWDYRFVFSKQTDPYISSECWPTRPRPRGPGNLAMVLRWRSPPPPPQSAELGKNYLNFTKWFIDFFALIGWAYDLKTVPDDMIQRRMKRTGDGSNSWGWGDKDMTKEERDSATIIYPEKKDDIKMISKKNDYSKIFSHDYFRLFDEL

SEQ ID NO: 130 - DNA, 1224 bp, Cydia pomonella
  misc_feature (1)..(1224): mRNA-coding sequence, codon-optimized for Y. lipolytica
atgcctccccgagagtctaagaaggtggccctgcgatcttacgagacccctgtcgcttctctccctcctcgaaagtacgagattatctacctcaacctcttcctgcacatcgctggacatatctccgccgtctacggcctgtatctgtgcttcaccgccgcccagtggaagaccatcttctttgcctacctgtggctgttgatgggcgagctcggcgtggtgtgtggcgctcacagattgtggtctcaccgttctttcaaggtgaagcctcctctcgagatcatgctgatgctgttcaactgtattggattccagaacaccgccactgactgggtccgaaaccaccggctccatcacaagcactctgacactgacgccgacccccataactctaaccgaggaatgctgttctcccacattggctggctgtgtgtgcgaaagcacccagatgttaaagaacgaggcaagaccaccgacatgtctgacatctactctaaccccgtgctccgattccagaagaagcacaaggtaccccttttcggcgccatgtgtttcggcctgcccacccttattcccaccctgtggggagaggacatcgtcaccgcttggcacgtcaacctgctgcgattcgttcttaatctgaactctatcctgctggtcaactccattgctcataagtacggcacccgaccctacgatcgaaccatctgccctcgacaaaacaccacctgtaacatgatgactcttggagagggcttccacaactaccaccacacctttccttgggactaccgatttgtgttctctaagcagactgacccctacatctcttctgagtgctggcccactcgaccccgacctcgaggccccggaaacctggccatggtgctccgatggcgatctcctccaccacctcctcagtctgccgagctgggaaagaactacctgaacttcaccaagtggttcatcgacttcttcgccctgattggatgggcctacgacctgaagaccgttcctgacgatatgatccagcgacgaatgaaaagaaccggagacggatccaactcgtggggatggggagacaaggacatgactaaggaggagcgagactctgctactatcatttatcccgagaagaaggatgatattaagatgatctccaaaaagatcgactactccaagatcttctctcacgactacttccgactgttcgacgagctgtaa

SEQ ID NO: 131 - protein, 407 aa, Cydia pomonella
MPPRESKKVALRSYETPVASLPPRKYEIIYLNLFLHIAGHISAVYGLYLCFTAAQWKTIFFAYLWLLMGELGVVCGAHRLWSHRSFKVKPPLEIMLMLFNCIGFQNTATDWVRNHRLHHKHSDTDADPHNSNRGMLFSHIGWLCVRKHPDVKERGKTTDMSDIYSNPVLRFQKKHKVPLFGAMCFGLPTLIPTLWGEDIVTAWHVNLLRFVLNLNSILLVNSIAHKYGTRPYDRTICPRQNTTCNMMTLGEGFHNYHHTFPWDYRFVFSKQTDPYISSECWPTRPRPRGPGNLAMVLRWRSPPPPPQSAELGKNYLNFTKWFIDFFALIGWAYDLKTVPDDMIQRRMKRTGDGSNSWGWGDKDMTKEERDSATIIYPEKKDDIKMISKKIDYSKIFSHDYFRLFDEL

SEQ ID NO: 132 - DNA, 1098 bp, Cydia pomonella
  misc_feature (1)..(1098): mRNA-coding sequence, codon-optimized for Y. lipolytica
atgcctccccgagagtctaagaaggtggccctgcgatcttacgagacccctgtcgcttctctccctcctcgaaagtacgagattatctacctcaacctcttcctgcacatcgctggacatatctccgccgtctacggcctgtatctgtgcttcaccgccgcccagtggaagaccatcttctttgcctacctgtggctgttgatgggcgagctcggcgtggtgtgtggcgctcacagattgtggtctcaccgttctttcaaggtgaagcctcctctcgagatcatgctgatgctgttcaactgtattggattccagaacaccgccactgactgggtccgaaaccaccggctccatcacaagcactctgacactgacgccgacccccataactctaaccgaggaatgctgttctcccacattggctggctgtgtgtgcgaaagcacccagatgttaaagaacgaggcaagaccaccgacatgtctgacatctactctaaccccgtgctccgattccagaagaagcacaaggtaccccttttcggcgccatgtgtttcggcctgcccacccttattcccaccctgtggggagaggacatcgtcaccgcttggcacgtcaacctgctgcgattcgttcttaatctgaactctatcctgctggtcaactccattgctcataagtacggcacccgaccctacgatcgaaccatctgccctcgacaaaacaccacctgtaacatgatgactcttggagagggcttccacaactaccaccacacctttccttgggactaccgatctgccgagctgggaaagaactacctgaacttcaccaagtggttcatcgacttcttcgccctgattggatgggcctacgacctgaagaccgttcctgacgatatgatccagcgacgaatgaaaagaaccggagacggatccaactcgtggggatggggagacaaggacatgactaaggaggagcgagactctgctactatcatttatcccgagaagaaggatgatattaagatgatctccaaaaagaacgactactccaagatcttctctcacgactacttccgactgttcgacgagctgtaa

SEQ ID NO: 133 - protein, 365 aa, Cydia pomonella
MPPRESKKVALRSYETPVASLPPRKYEIIYLNLFLHIAGHISAVYGLYLCFTAAQWKTIFFAYLWLLMGELGVVCGAHRLWSHRSFKVKPPLEIMLMLFNCIGFQNTATDWVRNHRLHHKHSDTDADPHNSNRGMLFSHIGWLCVRKHPDVKERGKTTDMSDIYSNPVLRFQKKHKVPLFGAMCFGLPTLIPTLWGEDIVTAWHVNLLRFVLNLNSILLVNSIAHKYGTRPYDRTICPRQNTTCNMMTLGEGFHNYHHTFPWDYRSAELGKNYLNFTKWFIDFFALIGWAYDLKTVPDDMIQRRMKRTGDGSNSWGWGDKDMTKEERDSATIIYPEKKDDIKMISKKNDYSKIFSHDYFRLFDEL

SEQ ID NO: 134 - DNA, 1098 bp, Cydia pomonella
  misc_feature (1)..(1098): mRNA-coding sequence, codon-optimized for Y. lipolytica
atgcctccccgagagtctaagaaggtggccctgcgatcttacgagacccctgtcgcttctctccctcctcgaaagtacgagattatctacctcaacctcttcctgcacatcgctggacatatctccgccgtctacggcctgtatctgtgcttcaccgccgcccagtggaagaccatcttctttgcctacctgtggctgttgatgggcgagctcggcgtggtgtgtggcgctcacagattgtggtctcaccgttctttcaaggtgaagcctcctctcgagatcatgctgatgctgttcaactgtattggattccagaacaccgccactgactgggtccgaaaccaccggctccatcacaagcactctgacactgacgccgacccccataactctaaccgaggaatgctgttctcccacattggctggctgtgtgtgcgaaagcacccagatgttaaagaacgaggcaagaccaccgacatgtctgacatctactctaaccccgtgctccgattccagaagaagcacaaggtaccccttttcggcgccatgtgtttcggcctgcccacccttattcccaccctgtggggagaggacatcgtcaccgcttggcacgtcaacctgctgcgattcgttcttaatctgaactctatcctgctggtcaactccattgctcataagtacggcacccgaccctacgatcgaaccatctgccctcgacaaaacaccacctgtaacatgatgactcttggagagggcttccacaactaccaccacacctttccttgggactaccgatctgccgagctgggaaagaactacctgaacttcaccaagtggttcatcgacttcttcgccctgattggatgggcctacgacctgaagaccgttcctgacgatatgatccagcgacgaatgaaaagaaccggagacggatccaactcgtggggatggggagacaaggacatgactaaggaggagcgagactctgctactatcatttatcccgagaagaaggatgatattaagatgatctccaaaaagatcgactactccaagatcttctctcacgactacttccgactgttcgacgagctgtaa

SEQ ID NO: 135 - protein, 365 aa, Cydia pomonella
MPPRESKKVALRSYETPVASLPPRKYEIIYLNLFLHIAGHISAVYGLYLCFTAAQWKTIFFAYLWLLMGELGVVCGAHRLWSHRSFKVKPPLEIMLMLFNCIGFQNTATDWVRNHRLHHKHSDTDADPHNSNRGMLFSHIGWLCVRKHPDVKERGKTTDMSDIYSNPVLRFQKKHKVPLFGAMCFGLPTLIPTLWGEDIVTAWHVNLLRFVLNLNSILLVNSIAHKYGTRPYDRTICPRQNTTCNMMTLGEGFHNYHHTFPWDYRSAELGKNYLNFTKWFIDFFALIGWAYDLKTVPDDMIQRRMKRTGDGSNSWGWGDKDMTKEERDSATIIYPEKKDDIKMISKKIDYSKIFSHDYFRLFDEL

SEQ ID NO: 136 - DNA, 1224 bp, Cydia pomonella
  misc_feature (1)..(1224): mRNA-coding sequence, codon-optimized for Y. lipolytica
atgcctccacgagagtctaagaaggtgaccctgcgatcttacgagactcccgaggcctctctgcctcctcgaaagtacgagattatctacctgaacctgttcctgcacattgccggccacatctctgccgtgtacggcctgtacctgtgcttcaccgccgctcagtggaagaccatcttcttcgcctacctgtggctgctgatgggcgagctgggcgtcgtgtgtggcgcccaccgactgtggtcccaccgatctttcaaggtgaagcctcctctggaaatcatgctgatgctgttcaactgcatcggcttccagaacaccgctaccgactgggtgcgaaaccaccgactccaccacaagcactctgacaccgacgctgaccctcacaactctaaccgaggcatgctgttctctcacatcggctggctgtgcgtgcgaaagcaccccgacgtgaaggaacgaggcaagaccaccgacatgtctgacatctactctaaccccgtgctgcgattccaaaagaagcacaaggtccctctgttcggcgccatgtgcttcggactgcccactctgatccccactctgtggggcgaagatatcgtgaccgcctggcacgtgaacctgctccgattcgtgctgaacctgaactctatcctgctggtcaactctatcgcccacaagtacggcacccgaccttacgaccgaaccatctgtccccgacagaacaccacctgtaacatgatgaccctcggcgagggcttccacaactaccaccacactttcccttgggactaccgatttgtgttctctaagcagactgacccctacatctcttctgagtgctggcccactcgaccccgacctcgaggccccggaaacctggccatggtgctccgatggcgatctcctccaccacctcctcagtctgccgagctgggaaagaactacctcaacttcaccaagtggttcatcgactttttcgccctgatcggctgggcctacgacctcaagaccgtgcctgacgacatgatccagcgacgaatgaagcgaaccggcgacggctctaactcttggggctggggcgacaaggacatgaccaaggaagaacgagactctgccaccatcatctaccccgagaagaaggacgacatcaagatgatctctaagaagaacgactactccaagatcttctctcacgactacttccgactgttcgacgagctgtag

SEQ ID NO: 137 - protein, 407 aa, Cydia pomonella
MPPRESKKVTLRSYETPEASLPPRKYEIIYLNLFLHIAGHISAVYGLYLCFTAAQWKTIFFAYLWLLMGELGVVCGAHRLWSHRSFKVKPPLEIMLMLFNCIGFQNTATDWVRNHRLHHKHSDTDADPHNSNRGMLFSHIGWLCVRKHPDVKERGKTTDMSDIYSNPVLRFQKKHKVPLFGAMCFGLPTLIPTLWGEDIVTAWHVNLLRFVLNLNSILLVNSIAHKYGTRPYDRTICPRQNTTCNMMTLGEGFHNYHHTFPWDYRFVFSKQTDPYISSECWPTRPRPRGPGNLAMVLRWRSPPPPPQSAELGKNYLNFTKWFIDFFALIGWAYDLKTVPDDMIQRRMKRTGDGSNSWGWGDKDMTKEERDSATIIYPEKKDDIKMISKKNDYSKIFSHDYFRLFDEL

SEQ ID NO: 138 - DNA, 1224 bp, Cydia pomonella
  misc_feature (1)..(1224): mRNA-coding sequence, codon-optimized for Y. lipolytica
atgcctccacgagagtctaagaaggtgaccctgcgatcttacgagactcccgaggcctctctgcctcctcgaaagtacgagattatctacctgaacctgttcctgcacattgccggccacatctctgccgtgtacggcctgtacctgtgcttcaccgccgctcagtggaagaccatcttcttcgcctacctgtggctgctgatgggcgagctgggcgtcgtgtgtggcgcccaccgactgtggtcccaccgatctttcaaggtgaagcctcctctggaaatcatgctgatgctgttcaactgcatcggcttccagaacaccgctaccgactgggtgcgaaaccaccgactccaccacaagcactctgacaccgacgctgaccctcacaactctaaccgaggcatgctgttctctcacatcggctggctgtgcgtgcgaaagcaccccgacgtgaaggaacgaggcaagaccaccgacatgtctgacatctactctaaccccgtgctgcgattccaaaagaagcacaaggtccctctgttcggcgccatgtgcttcggactgcccactctgatccccactctgtggggcgaagatatcgtgaccgcctggcacgtgaacctgctccgattcgtgctgaacctgaactctatcctgctggtcaactctatcgcccacaagtacggcacccgaccttacgaccgaaccatctgtccccgacagaacaccacctgtaacatgatgaccctcggcgagggcttccacaactaccaccacactttcccttgggactaccgatttgtgttctctaagcagactgacccctacatctcttctgagtgctggcccactcgaccccgacctcgaggccccggaaacctggccatggtgctccgatggcgatctcctccaccacctcctcagtctgccgagctgggaaagaactacctcaacttcaccaagtggttcatcgactttttcgccctgatcggctgggcctacgacctcaagaccgtgcctgacgacatgatccagcgacgaatgaagcgaaccggcgacggctctaactcttggggctggggcgacaaggacatgaccaaggaagaacgagactctgccaccatcatctaccccgagaagaaggacgacatcaagatgatctctaagaagatcgactactccaagatcttctctcacgactacttccgactgttcgacgagctgtag

SEQ ID NO: 139 - protein, 407 aa, Cydia pomonella
MPPRESKKVTLRSYETPEASLPPRKYEIIYLNLFLHIAGHISAVYGLYLCFTAAQWKTIFFAYLWLLMGELGVVCGAHRLWSHRSFKVKPPLEIMLMLFNCIGFQNTATDWVRNHRLHHKHSDTDADPHNSNRGMLFSHIGWLCVRKHPDVKERGKTTDMSDIYSNPVLRFQKKHKVPLFGAMCFGLPTLIPTLWGEDIVTAWHVNLLRFVLNLNSILLVNSIAHKYGTRPYDRTICPRQNTTCNMMTLGEGFHNYHHTFPWDYRFVFSKQTDPYISSECWPTRPRPRGPGNLAMVLRWRSPPPPPQSAELGKNYLNFTKWFIDFFALIGWAYDLKTVPDDMIQRRMKRTGDGSNSWGWGDKDMTKEERDSATIIYPEKKDDIKMISKKIDYSKIFSHDYFRLFDEL

SEQ ID NO: 140 - DNA, 1173 bp, Cydia pomonella
  misc_feature (1)..(1173): mRNA-coding sequence, codon-optimized for Y. lipolytica
atgcctccacgagagtctaagaaggtgaccctgcgatcttacgagactcccgaggcctctctgcctcctcgaaagtacgagattatctacctgaacctgttcctgcacattgccggccacatctctgccgtgtacggcctgtacctgtgcttcaccgccgctcagtggaagaccatcttcttcgcctacctgtggctgctgatgggcgagctgggcgtcgtgtgtggcgcccaccgactgtggtcccaccgatctttcaaggtgaagcctcctctggaaatcatgctgatgctgttcaactgcatcggcttccagaacaccgctaccgactgggtgcgaaaccaccgactccaccacaagcactctgacaccgacgctgaccctcacaactctaaccgaggcatgctgttctctcacatcggctggctgtgcgtgcgaaagcaccccgacgtgaaggaacgaggcaagaccaccgacatgtctgacatctactctaaccccgtgctgcgattccaaaagaagcacaaggtccctctgttcggcgccatgtgcttcggactgcccactctgatccccactctgtggggcgaagatatcgtgaccgcctggcacgtgaacctgctccgattcgtgctgaacctgaactctatcctgctggtcaactctatcgcccacaagtacggcacccgaccttacgaccgaaccatctgtccccgacagaacaccacctgtaacatgatgaccctcggcgagggcttccacaactaccaccacactttcccttgggactaccgatttgtgttctctaagcagactgacccctacatctcttctgagtgctggcccactcgaccccgacctcgaggccccggaaacctggccatggtgctccgatggcgatctcctccaccacctcctcagtctgccgagctgggaaagaactacctcaacttcaccaagtggttcatcgactttttcgccctgatcggctgggcctacgacctcaagaccgtgcctgacgacatgatccagcgacgaatgaagcgaaccggcgacggctctaactcttggggctggggcgacaaggacatgaccaaggaagaacgagactctgccaccatcatctaccccgagaagaaggacgacatcaagatgatctctaagaagaactag

SEQ ID NO: 141 - protein, 390 aa, Cydia pomonella
MPPRESKKVTLRSYETPEASLPPRKYEIIYLNLFLHIAGHISAVYGLYLCFTAAQWKTIFFAYLWLLMGELGVVCGAHRLWSHRSFKVKPPLEIMLMLFNCIGFQNTATDWVRNHRLHHKHSDTDADPHNSNRGMLFSHIGWLCVRKHPDVKERGKTTDMSDIYSNPVLRFQKKHKVPLFGAMCFGLPTLIPTLWGEDIVTAWHVNLLRFVLNLNSILLVNSIAHKYGTRPYDRTICPRQNTTCNMMTLGEGFHNYHHTFPWDYRFVFSKQTDPYISSECWPTRPRPRGPGNLAMVLRWRSPPPPPQSAELGKNYLNFTKWFIDFFALIGWAYDLKTVPDDMIQRRMKRTGDGSNSWGWGDKDMTKEERDSATIIYPEKKDDIKMISKKN

SEQ ID NO: 142 - DNA, 1173 bp, Cydia pomonella
  misc_feature (1)..(1173): mRNA-coding sequence, codon-optimized for Y. lipolytica
atgcctccccgagagtctaagaaggtggccctgcgatcttacgagacccctgtcgcttctctccctcctcgaaagtacgagattatctacctcaacctcttcctgcacatcgctggacatatctccgccgtctacggcctgtatctgtgcttcaccgccgcccagtggaagaccatcttctttgcctacctgtggctgttgatgggcgagctcggcgtggtgtgtggcgctcacagattgtggtctcaccgttctttcaaggtgaagcctcctctcgagatcatgctgatgctgttcaactgtattggattccagaacaccgccactgactgggtccgaaaccaccggctccatcacaagcactctgacactgacgccgacccccataactctaaccgaggaatgctgttctcccacattggctggctgtgtgtgcgaaagcacccagatgttaaagaacgaggcaagaccaccgacatgtctgacatctactctaaccccgtgctccgattccagaagaagcacaaggtaccccttttcggcgccatgtgtttcggcctgcccacccttattcccaccctgtggggagaggacatcgtcaccgcttggcacgtcaacctgctgcgattcgttcttaatctgaactctatcctgctggtcaactccattgctcataagtacggcacccgaccctacgatcgaaccatctgccctcgacaaaacaccacctgtaacatgatgactcttggagagggcttccacaactaccaccacacctttccttgggactaccgatttgtgttctctaagcagactgacccctacatctcttctgagtgctggcccactcgaccccgacctcgaggccccggaaacctggccatggtgctccgatggcgatctcctccaccacctcctcagtctgccgagctgggaaagaactacctgaacttcaccaagtggttcatcgacttcttcgccctgattggatgggcctacgacctgaagaccgttcctgacgatatgatccagcgacgaatgaaaagaaccggagacggatccaactcgtggggatggggagacaaggacatgactaaggaggagcgagactctgctactatcatttatcccgagaagaaggatgatattaagatgatctccaaaaagatctaa

SEQ ID NO: 143 - protein, 390 aa, Cydia pomonella
MPPRESKKVTLRSYETPEASLPPRKYEIIYLNLFLHIAGHISAVYGLYLCFTAAQWKTIFFAYLWLLMGELGVVCGAHRLWSHRSFKVKPPLEIMLMLFNCIGFQNTATDWVRNHRLHHKHSDTDADPHNSNRGMLFSHIGWLCVRKHPDVKERGKTTDMSDIYSNPVLRFQKKHKVPLFGAMCFGLPTLIPTLWGEDIVTAWHVNLLRFVLNLNSILLVNSIAHKYGTRPYDRTICPRQNTTCNMMTLGEGFHNYHHTFPWDYRFVFSKQTDPYISSECWPTRPRPRGPGNLAMVLRWRSPPPPPQSAELGKNYLNFTKWFIDFFALIGWAYDLKTVPDDMIQRRMKRTGDGSNSWGWGDKDMTKEERDSATIIYPEKKDDIKMISKKI

SEQ ID NO: 144 - DNA, 1098 bp, Cydia pomonella; identical to SEQ ID NO: 132
  misc_feature (1)..(1098): mRNA-coding sequence, codon-optimized for Y. lipolytica

SEQ ID NO: 145 - protein, 365 aa, Cydia pomonella
MPPRESKKVTLRSYETPEASLPPRKYEIIYLNLFLHIAGHISAVYGLYLCFTAAQWKTIFFAYLWLLMGELGVVCGAHRLWSHRSFKVKPPLEIMLMLFNCIGFQNTATDWVRNHRLHHKHSDTDADPHNSNRGMLFSHIGWLCVRKHPDVKERGKTTDMSDIYSNPVLRFQKKHKVPLFGAMCFGLPTLIPTLWGEDIVTAWHVNLLRFVLNLNSILLVNSIAHKYGTRPYDRTICPRQNTTCNMMTLGEGFHNYHHTFPWDYRSAELGKNYLNFTKWFIDFFALIGWAYDLKTVPDDMIQRRMKRTGDGSNSWGWGDKDMTKEERDSATIIYPEKKDDIKMISKKNDYSKIFSHDYFRLFDEL

SEQ ID NO: 146 - DNA, 1098 bp, Cydia pomonella; identical to SEQ ID NO: 134

SEQ ID NO: 147 - protein, 365 aa, Cydia pomonella
MPPRESKKVTLRSYETPEASLPPRKYEIIYLNLFLHIAGHISAVYGLYLCFTAAQWKTIFFAYLWLLMGELGVVCGAHRLWSHRSFKVKPPLEIMLMLFNCIGFQNTATDWVRNHRLHHKHSDTDADPHNSNRGMLFSHIGWLCVRKHPDVKERGKTTDMSDIYSNPVLRFQKKHKVPLFGAMCFGLPTLIPTLWGEDIVTAWHVNLLRFVLNLNSILLVNSIAHKYGTRPYDRTICPRQNTTCNMMTLGEGFHNYHHTFPWDYRSAELGKNYLNFTKWFIDFFALIGWAYDLKTVPDDMIQRRMKRTGDGSNSWGWGDKDMTKEERDSATIIYPEKKDDIKMISKKIDYSKIFSHDYFRLFDEL

(19) United States
(12) Patent Application Publication
(10) Pub. No.: US 2023/0332096 A1
(43) Pub. Date: Oct. 19, 2023

(54) METHODS AND CELL FACTORIES FOR PRODUCING INSECT PHEROMONES

(71) Applicant: BioPhero ApS, København Ø (DK)

(72) Inventors: Irina Borodina, Kongens Lyngby (DK); Leonie Wenning, Kongens Lyngby (DK); Carina Holkenbrink, Kongens Lyngby (DK); Kanchana Rueksomtawin Kildegaard, Kongens Lyngby (DK); Baojian Ding, Södra Sandby (SE); Christer Löfstedt, Lund (SE); Karolis Petkevicius, Kongens Lyngby (DK)

(73) Assignee: BioPhero ApS, København Ø (DK)

(21) Appl. No.: 17/432,215

(22) PCT Filed: Feb. 10, 2020
(86) PCT No.: PCT/EP2020/053306

§ 371 Date: Aug. 19, 2021

(30) Foreign Application Priority Data
Feb. 19, 2019 (EP) 19157910.0

(51) Int. Cl. C12N 1/16; C12N 9/02; C12N 15/81

(52) U.S. Cl. CPC C12N 1/16; C12N 9/001; C12N 15/81; C12Y 102/01084; C12Y 103/03006

(57) ABSTRACT

The present invention relates to pheromone production, desaturated fatty alcohols or aldehydes or acetates using recombinant yeasts expressing acyl-CoA oxidase to shorten fatty acyl-CoA by two carbons, deleting endogenous acyl-CoA oxidase, express desaturase to introduce at least one double bond, acyl-CoA-reductase to convert fatty acyl-CoA into fatty alcohol. Further, acetyltransferase can be used to convert to desaturated acyl fatty acetate, alcohol dehydrogenase or fatty acyl oxidase to convert to desaturated fatty aldehyde.

### TECHNICAL FIELD

The present invention relates to microbial cell factories and methods for producing insect pheromones.

### BACKGROUND

Pheromones constitute a group of diverse chemicals that insects use to communicate with individuals of the same species in various contexts, including mate attraction, alarm, trail marking and aggregation. Insect sex pheromones associated with long-range mate finding are already used in agriculture and forestry applications for monitoring and control of pests, as a safe and environmentally friendly alternative to pesticides. Application of insect pheromones for pest control became possible only after industrial-scale synthesis of pheromones started at the end of the 1980s. Nevertheless, the prices for chemically synthesized pheromones remain high and as of today, they present a major barrier for expanding pheromone usage. While the known sex pheromones of e.g. Lepidoptera exceed 3,000 compounds, this large diversity results from the action of a limited number of enzymes, namely elongases, desaturases, fatty acid reductases, acetyl transferases, fatty alcohol oxidases, and presumably chain shortening enzymes that act on fatty acyl-CoA. The resulting pheromones are mainly fatty alcohols, fatty aldehydes or fatty alcohol acetates with a chain length of 10-18 carbons and one or several double bonds in the molecule (www.pherobase.com). The stereochemistry of the compound is often critical for activity [1]. These biosynthetic pathways can be reconstituted in microbial platform strains, optimized for biosynthesis of fatty alcohols and their derivatives, resulting in cell factories that produce pheromones via fermentation [2-4]. Biological production of pheromones by fermentation enables lower production costs by eliminating the need for expensive catalysts, specialty precursors, and extensive purification for removing the side-products of chemical catalysis.

There remains a need for microbial cell factories capable of producing insect pheromones such as fatty alcohols and derivatives thereof with a specific chain length, as well as methods for producing such compounds.

### SUMMARY

The invention is as defined in the claims.

The present disclosure provides microbial cells capable of producing a large diversity of pheromones (FIG. 1), and methods for producing a large diversity of pheromones.

This is achieved by reducing the activity of native acyl-CoA oxidases in a microbial production cell and by expressing specific acyl-CoA oxidases, desaturases, reductases, and acetyltransferases. In short, the present yeast cells are capable of shortening fatty acyl-CoAs, following which the yeast cells are capable of converting the shortened fatty acyl-CoA to desaturated fatty alcohols, fatty acyl acetates or fatty aldehydes.

Herein is provided a yeast cell capable of producing a desaturated fatty alcohol and optionally a desaturated fatty acyl acetate and/or a desaturated fatty aldehyde, said yeast cell:
i) having one or more mutations resulting in reduced activity of one or more native acyl-CoA oxidases; and
ii) expressing at least one first group of enzymes comprising at least one acyl-CoA oxidase capable of oxidising a fatty acyl-CoA, wherein the first group of enzymes is capable of shortening a fatty acyl-CoA of a first carbon chain length X to a shortened fatty acyl-CoA having a second carbon chain length X′, wherein X′ ≤ X-2; and
iii) expressing at least one heterologous desaturase capable of introducing at least one double bond in said fatty acyl-CoA and/or in said shortened fatty acyl-CoA; and
iv) expressing at least one heterologous fatty acyl-CoA reductase, capable of converting at least part of said desaturated fatty acyl-CoA to a desaturated fatty alcohol; and
v) optionally expressing at least one acetyltransferase capable of converting at least part of said desaturated fatty alcohol to a desaturated fatty acyl acetate, and/or at least one alcohol dehydrogenase and/or fatty alcohol oxidase capable of converting at least part of said desaturated fatty alcohol to a desaturated fatty aldehyde.

Also provided is a method for producing a desaturated fatty alcohol and optionally a desaturated fatty acyl acetate and/or a fatty aldehyde of carbon chain length X′, comprising the steps of providing a yeast cell capable of converting a fatty acyl-CoA of a first carbon chain length X to a desaturated fatty alcohol and optionally a desaturated fatty acyl acetate and/or a fatty aldehyde of carbon chain length X′ and incubating said yeast cell in a medium, wherein the yeast cell is as described herein and wherein X′ ≤ X-2.

Also provided is a nucleic acid construct for modifying a yeast cell, said construct comprising at least one first group of polynucleotides encoding at least one first group of enzymes comprising at least one acyl-CoA oxidase capable of oxidising a fatty acyl-CoA, wherein the first group of enzymes is capable of shortening a fatty acyl-CoA of a first carbon chain length X to a shortened fatty acyl-CoA having a second carbon chain length X′, wherein X′ ≤ X-2.

Also provided is a kit of parts comprising:
a) a yeast cell as described herein; and/or
b) a nucleic acid construct as described herein; and/or
c) a set of primers for introducing one or more mutations resulting in reduced activity of one or more acyl-CoA oxidases;
and optionally the yeast cell to be modified.

Also provided is a desaturated fatty alcohol, a desaturated fatty acyl acetate or a desaturated fatty aldehyde obtainable by the methods described herein.

Also provided is the use of a desaturated fatty alcohol, a desaturated fatty acyl acetate or a desaturated fatty aldehyde.

### DESCRIPTION OF THE DRAWINGS

FIG. 1. A. Method for obtaining a desaturated fatty acyl-CoA precursor of desired length (Δ_{z/En}-X:CoA), where X is carbon chain length, e.g., 10,12,14,16, etc. Each chain shortening step reduces the carbon chain by two carbons (-2C), which are removed from the carboxy-end. One, two or more double bonds can be introduced at desired positions (n) and in desired confirmation (cis or trans or mixed) (A_{Z/E}). B. Desaturated fatty acyl-CoA precursors (Δ_{Z/En}-X:CoA) are reduced into corresponding desaturated fatty alcohols (Δ_{Z/En}-X:OH) by fatty-acyl-CoA reductase (FAR). Desaturated fatty alcohols (Δ_{Z/En}-X:OH) can be further converted into corresponding desaturated fatty aldehydes (Δ_{Z/En}-X:Ald) or desaturated fatty alcohols acetates (Δ_{Z/En}-X:OAc) by enzymatic or chemical conversion.

FIG. 2. Application of the method to produce Δ_{Z5}-12:OAc by expression of a Δ_{Z9}-16-desaturase, a fatty acyl-CoA reductase, and an acetyltransferase in a background strain that has fatty acyl-CoA carbon chain shortening to 12:CoA implemented.

FIG. 3. A. Application of the method to produce Δ_{Z7}-12:OAc by expression of a Δ_{Z9}-14-desaturase, a fatty acyl-CoA reductase, and an acetyltransferase in a background strain that has fatty acyl-CoA carbon chain shortening to 12:CoA implemented. B. Application of the method to produce Δ_{Z7}-12:OAc by expression of a Δ_{Z11}-16-desaturase, a fatty acyl-CoA reductase, and an acetyltransferase in a background strain that has fatty acyl-CoA carbon chain shortening to 12:CoA implemented.

FIG. 4. Application of the method to produce Δ_{E7}Δ_{Z9}-12:OAc by expression of a Δ_{Z11}-14-desaturase, a Δ_{E9}-14-desaturase, a that acyl-CoA reductase, and an acetyltransferase in a background strain which has fatty acyl-CoA carbon chain shortening to 12:CoA implemented.

FIG. 5. Application of the method to produce A_{Z8}-12:OAc by expression of a Δ_{Z/E10}-14-desaturase, a fatty acyl-CoA reductase, and an acetyltransferase in a background strain that has fatty acyl-CoA carbon chain shortening to 12:CoA implemented.

FIG. 6. Application of the method to produce Δ_{E8}Δ_{E10}-12:OH by expression of a Δ_{Z/E9}-12-desaturase, a Δ_{E8}Δ_{E10}-12-desaturase, and a fatty acyl-CoA reductase in a background strain that has fatty acyl-CoA carbon chain shortening to 12:CoA implemented.

FIG. 7. Application of the method to produce Δ_{Z9}-12:OAc by expression of a Δ_{Z11}-14-desaturase, a fatty acyl-CoA reductase, and an acetyltransferase in a background strain that has fatty acyl-CoA carbon chain shortening to 12:CoA implemented.

FIG. 8. Application of the method to produce Δ_{Z7}-14:OH by expression of a Δ_{Z9}-16-desaturase a fatty acyl-CoA reductase in a background strain that has fatty acyl-CoA carbon chain shortening to 14:CoA implemented.

FIG. 9. Application of the method to produce Δ_{Z9}-14:OAc by expression of a Δ_{Z11}-16-desaturase, a fatty acyl-CoA reductase, and an acetyltransferase in a background strain that has fatty acyl-CoA carbon chain shortening to 14:CoA implemented.

FIG. 10. Application of the method to produce Δ_{E11}-14:OAc by expression of a Δ_{E11}-14-desaturase, a fatty acyl-CoA reductase, and an acetyltransferase in a background strain that has fatty acyl-CoA carbon chain shortening to 14:CoA implemented.

FIG. 11. Application of the method to produce Δ_{Z11}-14:OAc by expression of a Δ_{Z11}-14-desaturase, a fatty acyl-CoA reductase, and an acetyltransferase in a background strain that has fatty acyl-CoA carbon chain shortening to 14:CoA implemented.

FIG. 12. Application of the method to produce Δ_{Z7}-16:OH by expression of a Δ_{Z9}-18-desaturase and a fatty acyl-CoA reductase in a background strain that synthesizes 16:CoA.

FIG. 13. Application of the method to produce Δ_{Z7}Δ_{Z11}-16:OAc by expression of a Δ_{Z11}-16-desaturase, a fatty acyl-CoA reductase and an acetyltransferase in a background strain that synthesizes 16:CoA.

FIG. 14. Application of the method to produce Δ_{Z9}-16:OH by expression of a Δ_{Z9}-16-desaturase and a fatty acyl-CoA reductase in a background strain that synthesizes 16:CoA.

FIG. 15. Application of the method to produce Δ_{Z11}Δ_{Z13}-16:OH by expression of a Δ_{Z11}-16-desaturase, a Δ_{Z13}-16-desaturase, and a fatty acyl-CoA reductase in a background strain that synthesizes 16:CoA.

FIG. 16. GC-MS chromatogram from cell extracts of strain ST9640 (upper panel). Production of ΔE11-14:OH and ΔZ11-14:OH could be detected. The retention times and spectra of the peaks match those of the reference standards (lower panel).

FIG. 17. GC-MS chromatogram of cell extracts of strains expressing A11-16 desaturase SlitDes5. Production of A) ΔZ5-12:Me, AZ7-12:Me, B) Z5-14:Me, Z9-14:Me and C) Z7-16:Me could be detected in extracts of strain ST9344 expressing an peroxisomal oxidase (middle panel) but not or less in the control parent strain ST9285 (upper panel). The retention times and spectra of the peaks match those of the reference standards (lower panel).

FIG. 18. A and B) GC-MS chromatogram of cell extracts of strains expressing Δ9-14 desaturase Dmd9. Production of A) ΔZ5-12:Me, AZ7-12:Me, B) Z5-14:Me and Z7-14:Me could be detected in extracts of strain ST9347 expressing an peroxisomal oxidase (middle panel) but not or less in the control parent strain ST9294 (upper panel). The retention times and spectra of the peaks match those of the reference standards (lower panel). C) GC-MS chromatogram of cell extracts of strains expressing Δ11-14 desaturase Lbo_PPTQ. Production of Z9-12:Me and most likely E9-12:Me could be detected in extracts of strain ST9350 expressing an peroxisomal oxidase (upper panel) but not in the control parent strain ST9314 (middle panel). The retention time and spectrum of the 41.8-min-peak matches the one of the reference standard (lower panel).

FIG. 19. GC-MS chromatogram of cell extracts of strains expressing Δ11-14 desaturase Lbo_PPTQ. Production of Z9-12:OH could be detected in extracts of strain ST9350 expressing an peroxisomal oxidase (middle panel) but not or less in the control parent strain ST9314 (upper panel). The retention time and spectrum of the 57.7-min-peak matches the one of the reference standard (lower panel).

FIG. 20. GC-MS chromatogram of cell extracts of strain ST9138 (control), ST9435 (expressing desaturase PGDes8) and ST9443 (expressing PGDes8 and a peroxisomal oxidase). Production of Z7-16:Me and a double unsaturated 16-2:Me could be detected in strain ST9443 but not in strain ST9435 and ST9138. The spectra of the Z7-16:Me-peak and the 16-2:Me peak from the extracts of strain ST9443 are shown in the two lower panels.

### DETAILED DESCRIPTION

### Definitions

Desaturated: the term “desaturated” will be herein used interchangeably with the term “unsaturated” and refers to a compound containing one or more double or triple carbon-carbon bonds.

Fatty acid: the term “fatty acid” refers to a carboxylic acid having a long aliphatic chain, i.e. an aliphatic chain between 4 and 28 carbon atoms, such as 4, 5, 6, 7, 8, 9, 10, 11, 12, 13, 14, 15, 16, 17, 18, 19, 20, 21, 22, 23, 24, 25, 26, 27 or 28 carbon atoms. Most naturally occurring fatty acids are unbranched. They can be saturated, or desaturated.

Fatty acyl acetate: the term will herein be used interchangeably with “fatty acetate” and refers to an acetate having a fatty carbon chain, i.e. an aliphatic chain between 4 and 28 carbon atoms, such as 4, 5, 6, 7, 8, 9, 10, 11, 12, 13, 14, 15, 16, 17, 18, 19, 20, 21, 22, 23, 24, 25, 26, 27 or 28 carbon atoms. Fatty acyl acetates can be saturated or desaturated.

Fatty acyl-CoA: the term will herein be used interchangeably with “fatty acyl-CoA ester”, and refers to compounds of general formula R-CO-SCoA, where R is a fatty carbon chain. The fatty carbon chain is joined to the -SH group of coenzyme A by a thioester bond. Fatty acyl-CoAs can be saturated or desaturated, depending on whether the fatty acid which it is derived from is saturated or desaturated.

Fatty alcohol: the term “fatty alcohol” refers herein to an alcohol derived from a fatty acyl-CoA, having a carbon chain length of 4 to 28 carbon atoms, such as 4, 5, 6, 7, 8, 9, 10, 11, 12, 13, 14, 15, 16, 17, 18, 19, 20, 21, 22, 23, 24, 25, 26, 27 or 28 carbon atoms. Fatty alcohols can be saturated or desaturated.

Fatty aldehyde: the term refers herein to an aldehyde derived from a fatty acyl-CoA, having a carbon chain length of 4 to 28 carbon atoms, such as 4, 5, 6, 7, 8, 9, 10, 11, 12, 13, 14, 15, 16, 17, 18, 19, 20, 21, 22, 23, 24, 25, 26, 27 or 28 carbon atoms. Fatty aldehydes can be saturated or desaturated.

Functional variant: the term refers herein to functional variants of an enzyme, which retain at least some of the activity of the parent enzyme. Thus, a functional variant of an acyl-CoA oxidase, a desaturase, an alcohol-forming fatty acyl-CoA reductase, an alcohol dehydrogenase, an aldehyde-forming fatty acyl-CoA reductase, or an acetyltransferase can catalyse the same conversion as the acyl-CoA oxidase, the desaturase, the alcohol-forming fatty acyl-CoA reductase, the alcohol dehydrogenase, the aldehyde-forming fatty acyl-CoA reductase, or the acetyltransferase, respectively, from which they are derived, although the efficiency of reaction may be different, e.g. the efficiency is decreased or increased compared to the parent enzyme or the substrate specificity is modified.

Heterologous: the term “heterologous” when referring to a polypeptide, such as a protein or an enzyme, or to a polynucleotide, shall herein be construed to refer to a polypeptide or a polynucleotide which is not naturally present in a wild type cell. For example, the term “heterologous Δ9 desaturase” when applied to Yarrowia lipolytica refers to a Δ9 desaturase which is not naturally present in a wild type Y. lipolytica cell, e.g. a Δ9 desaturase derived from Drosophila melanogaster.

Native: the term “native” when referring to a polypeptide, such as a protein or an enzyme, or to a polynucleotide, shall herein be construed to refer to a polypeptide or a polynucleotide which is naturally present in a wild type cell.

Pest: as used herein, the term ‘pest’ shall refer to an organism, in particular an animal, detrimental to humans or human concerns, in particular in the context of agriculture or livestock production. A pest is any living organism that is invasive or prolific, detrimental, troublesome, noxious, destructive, a nuisance to either plants or animals, human or human concerns, livestock, human structures, wild ecosystems etc. The term often overlaps with the related terms vermin, weed, plant and animal parasites and pathogens. It is possible for an organism to be a pest in one setting but beneficial, domesticated or acceptable in another.

Pheromone: pheromones are naturally occurring compounds designated by an unbranched aliphatic chain (typically between 9 and 18 carbons) ending in an alcohol, aldehyde or acetate functional group and containing up to 3 double bonds in the aliphatic backbone. Pheromone compositions may be produced chemically or biochemically, for example as described herein. Pheromones may thus comprise desaturated fatty alcohols, fatty aldehydes or fatty acyl acetates, such as can be obtained by the methods and cells described herein.

Reduced activity: the term “reduced activity” may herein refer to a total or a partial loss of activity of a given peptide, such as a protein or an enzyme. In some cases, peptides are encoded by essential genes, which cannot be deleted. In these cases, activity of the peptide can be reduced by methods known in the art, such as down-regulation of transcription or translation, or inhibition of the peptide. In other cases, the peptide is encoded by a non-essential gene, and the activity may be reduced or it may be completely lost, e.g. as a consequence of a deletion of the gene encoding the peptide.

Saturated: the term “saturated” refers to a compound which is devoid of double or triple carbon-carbon bonds.

Shortened: the term herein refers to a compound having a shorter carbon chain length than the compound it is obtained from. For example, a shortened fatty acyl-CoA has a carbon chain length shorter than the fatty acyl-CoA it is derived from.

### Yeast Cell

The present invention provides yeast cells useful as microbial factories for producing desaturated fatty alcohols and optionally derivatives thereof such as desaturated fatty acyl acetates and/or desaturated fatty aldehydes. Desaturated fatty alcohols and desaturated fatty acyl acetates are components of pheromones, in particular of moth pheromones. The yeast cell disclosed herein thus provides a platform for environment-friendly moth pheromone production.

The yeast cell may be a non-naturally occurring yeast cell, for example a yeast cell which has been engineered to produce desaturated fatty alcohols and desaturated fatty acyl acetates.

In some embodiments, the cell has been modified at the genomic level, e.g. by gene editing in the genome. The cell may also be modified by insertion of at least one nucleic acid construct such as at least one vector. The vector may be designed as is known to the skilled person to either enable integration of nucleic acid sequences in the genome, or to enable expression of a polypeptide encoded by a nucleic acid sequence comprised in the vector without genome integration or to enable deletion/inactivation of an open reading frame or promoter truncation or other genome edits.

The activity of the acyl-CoA oxidases normally present in the yeast cell, i.e. the native enzyme(s), is reduced or abolished by mutating the genes encoding said enzyme(s) in the cell. In order to direct carbon chain shortening to obtain fatty alcohols and derivatives thereof of a desired carbon chain length, one or more acyl-CoA oxidase is expressed in the yeast cell. These acyl-coA oxidases may be native to the yeast cell, or they may be derived from another organism. Genes encoding other enzymes required for oxidising a fatty acyl-CoA of a given chain length may also be introduced in the cell, if the cell does not express them already, or if increased activity or substrate specificity is desired. The acyl-CoA oxidase(s) thus expressed allow a fatty acyl-CoA to be oxidised and shortened to a fatty acyl-CoA having a shorter carbon chain length than the substrate. Thus in some embodiments, the reduced activity of the one or more native acyl-CoA oxidases is a reduced activity on acyl-CoAs having a carbon chain length smaller than X, such as smaller than X′.

The yeast cell is further modified to express one or more heterologous desaturases capable of introducing at least one double bond at least in the shortened fatty acyl-CoA thus obtained. A heterologous fatty acyl-CoA reductase, or alcohol-forming fatty acyl-CoA reductase, is also expressed, which can convert the resulting desaturated fatty acyl-CoAs to the corresponding desaturated fatty alcohols. The yeast cell may also express an acetyltransferase, which can further convert the desaturated fatty alcohols to the corresponding desaturated fatty acyl acetates. Alternatively, or in addition, the yeast cell may also express an alcohol dehydrogenase and/or an aldehyde-forming fatty acyl reductase capable of converting the desaturated fatty alcohols to the corresponding desaturated fatty aldehydes.

Accordingly is provided herein a yeast cell capable of producing a desaturated fatty alcohol and optionally a desaturated fatty acyl acetate and/or a desaturated fatty aldehyde, said yeast cell:
i) having one or more mutations resulting in reduced activity of one or more native acyl-CoA oxidases; and
ii) expressing at least one first group of enzymes comprising at least one acyl-CoA oxidase capable of oxidising a fatty acyl-CoA, wherein the first group of enzymes is capable of shortening a fatty acyl-CoA of a first carbon chain length X to a shortened fatty acyl-CoA having a second carbon chain length X′, wherein X′ ≤ X-2; and
iii) expressing at least one heterologous desaturase capable of introducing at least one double bond in said fatty acyl-CoA and/or in said shortened fatty acyl-CoA; and
iv) expressing at least one heterologous fatty acyl-CoA reductase, capable of converting at least part of said desaturated fatty acyl-CoA to a desaturated fatty alcohol; and
v) optionally expressing at least one acetyltransferase capable of converting at least part of said desaturated fatty alcohol to a desaturated fatty acyl acetate, and/or at least one alcohol dehydrogenase and/or fatty alcohol oxidase capable of converting at least part of said desaturated fatty alcohol to a desaturated fatty aldehyde.

In step ii), the first group of enzymes comprising at least one acyl-CoA oxidase capable of oxidising a fatty acyl-CoA, where the first group of enzyme is capable of shortening a fatty acyl-CoA of a first carbon chain length X can shorten said fatty acyl-CoA n number of times, and the shortened fatty acyl-CoA has a second carbon chain length X′= X-2n. accordingly, the a yeast cell expressing the first group of enzymes comprising at least one acyl-CoA oxidase capable of oxidising a fatty acyl-CoA is capable of converting a fatty acyl-CoA of carbon chain length X to a shortened fatty acyl-CoA of carbon chain length X′.

A fatty alcohol, a fatty acyl acetate or a fatty acyl aldehyde “corresponding” to another compound such as a fatty acyl-CoA, a fatty alcohol, a fatty acyl acetate or a fatty acyl aldehyde, shall be construed as having the same carbon chain length as said other compound.

In some embodiments, the genus of said yeast is selected from Saccharomyces, Pichia, Yarrowia, Kluyveromyces, Candida, Rhodotorula, Rhodosporidium, Cryptococcus, Trichosporon and Lipomyces. In some embodiments, the genus of said yeast is Saccharomyces or Yarrowia.

The yeast cell may in some embodiments be selected from the group consisting of Saccharomyces cerevisiae, Pichia pastoris, Kluyveromyces marxianus, Cryptococcus albidus, Lipomyces lipofera, Lipomyces starkeyi, Rhodosporidium toruloides, Rhodotorula glutinis, Trichosporon pullulan and Yarrowia lipolytica. In preferred embodiments, the yeast cell is a Saccharomyces cerevisiae cell or a Yarrowia lipolytica cell.

### Acyl-CoA Oxidase

The term acyl-CoA oxidase in the present disclosure refers to an enzyme such as an enzyme of EC number 1.3.3.6, capable of catalysing the following reaction:

acyl-CoA + O₂ ⇔ trans-2,3-dehydroacyl-CoA + H₂O₂

This enzyme belongs to the family of oxidoreductases, specifically those acting on the CH-CH group of donor with oxygen as acceptor. The systematic name of this enzyme class is acyl-CoA:oxygen 2-oxidoreductase. Other names use include fatty acyl-CoA oxidase, acyl coenzyme A oxidase, and fatty acyl-coenzyme A oxidase.

Acyl-CoA oxidases are often located in the cytosol of prokaryotic organisms, and in mitochondria and/or peroxisomes in eukaryotic organisms.

Labeling studies suggest that chain shortening of acyl-CoAs constitutes a step in biosynthesis of some pheromones, e.g. in Lepidoptera. The mechanism of the process is, however, poorly understood. In general, fatty acids are broken down in a process of β-oxidation, which typically occurs in the cytosol in prokaryotes and in mitochondria and peroxisomes in eukaryotes. During this process, fatty acyl-CoAs undergo repeated cycles of chain shortening, where each cycle releases an acetyl-CoA molecule, thereby shortening the acyl-CoA carbon chain by 2 carbons in each cycle. The first step of acyl-CoA degradation is oxidation of carbon in β-position by acyl-CoA oxidase. Some acyl-CoA oxidases have higher activity towards acyl-CoAs of specific chain lengths.

The yeast cell of the present disclosure may be engineered starting from a yeast cell which has one or more native acyl-CoA oxidases. The modified yeast cell disclosed herein preferably has reduced activity of said one or more native acyl-CoA oxidases; this can be achieved by using a yeast cell which has one or more mutations resulting in reduced activity of at least one of its native acyl-CoA oxidases. The native acyl-CoA oxidases may be peroxisomal, mitochondrial or cytosolic. In some embodiments, the one or more mutations results in reduced activity of all the native acyl-CoA oxidases. By reduced activity it is to be understood that the yeast cell due to said mutations has reduced ability to catalyse the above reaction, in particular to convert an acyl-CoA to the corresponding trans-2,3-dehydroacyl-CoA. In some embodiments, “reduced capability” means that the ability to catalyse said reaction is abolished completely or partially. In some embodiments, “reduced capability” means that the ability to catalyse the reaction is limited to a subgroup of the substrates which can be used for the reaction under normal circumstances, i.e. by using enzymes having normal capability.

The yeast cell of the present disclosure expresses at least one first group of enzymes comprising at least one acyl-CoA oxidase capable of oxidising a fatty acyl-CoA. The first group of enzymes comprises, besides the at least one acyl-CoA, the other enzymes required for converting a fatty acyl-CoA of a given carbon chain length to a fatty acyl-CoA of a shorter carbon chain length. These other enzymes may preferably be native to the yeast cell; in such embodiments, only the introduction of a gene encoding an acyl-CoA oxidase is required for the yeast cell to express the first group of enzymes.

The acyl-CoA oxidase of the first group of enzymes can be an acyl-CoA oxidase which is native to the yeast cell, or a heterologous acyl-CoA oxidase which is native to a different organism than the yeast cell. It may be a cytosolic acyl-CoA oxidase, a mitochondrial acyl-CoA oxidase or a peroxisomal acyl-CoA oxidase. Preferably, the acyl-CoA oxidase is peroxisomal.

The yeast cell expressing at least one acyl-CoA oxidase is thus capable of converting a fatty acyl-CoA of carbon chain length X to a shortened fatty acyl-CoA of carbon chain length X′, wherein X′ ≤ X-2.

In embodiments where the acyl-CoA oxidase is native to the yeast cell, said acyl-CoA oxidase may be modified as is known in the art, e.g. by the introduction of a promoter such as a constitutive or inducible promoter, or a promoter enabling overexpression of the acyl-CoA oxidase. The native acyl-CoA oxidase reintroduced in the first group of enzymes may be a mutated version with modified activity, such as modified substrate specificity and/or modified activity such as increased reaction efficiency.

In other embodiments, the acyl-CoA oxidase is derived from another organism.

The acyl-CoA comprised in the first group of enzymes may be an acyl-CoA oxidase derived from a yeast, a fungus, an insect, a mammalian, a bird or a plant, such as the at least one acyl-CoA oxidase of the first group of enzymes is derived from a yeast, a fungus, an insect, a mammalian, a bird or a plant. For example, the acyl-CoA oxidase is derived from an organism of a genus selected from Yarrowia, Agrotis, Arabidopsis, Aspergillus, Cucurbita, Homo, Paenarthrobacter, Lobesia and Rattus, such as the at least one acyl-CoA oxidase of the first group of enzymes is derived from an organism of a genus selected from Yarrowia, Agrotis, Arabidopsis, Aspergillus, Cucurbita, Homo, Paenarthrobacter, Lobesia and Rattus. In some embodiments, the at least one first group of enzymes comprises an acyl-CoA oxidase derived from Yarrowia lipolytica, Agrotis segetum, Arabidopsis thaliana, Aspergillus nidulans, Cucurbita maxima, Homo sapiens, Paenarthrobacter ureafaciens, Lobesia botrana or Rattus norvegicus.

The acyl-CoA oxidase thus introduced in the yeast cell may be an acyl-CoA oxidase native to Yarrowia lipolytica, Agrotis segetum, Arabidopsis thaliana, Aspergillus nidulans, Cucurbita maxima, Homo sapiens, Paenarthrobacter ureafaciens, Lobesia botrana or Rattus norvegicus. The yeast cell may be as described herein above.

The yeast cell of the present disclosure may thus express at least one first group of enzymes comprising at least one acyl-CoA oxidase capable of oxidising a fatty acyl-CoA, wherein said at least one acyl-CoA oxidase is selected from the group consisting of Yli_POX1 (SEQ ID NO: 2), Yli_POX2 (SEQ ID NO: 4), Yli_POX3 (SEQ ID NO: 6), Yli_POX4 (SEQ ID NO: 8), Yli_POX5 (SEQ ID NO: 10), Yli_POX6 (SEQ ID NO: 12), Ase_POX (SEQ ID NO: 14), Ath_POX1 (SEQ ID NO: 16), Ath_POX2 (SEQ ID NO: 18), Ani_POX (SEQ ID NO: 20), Cma_POX (SEQ ID NO: 22), Hsa_POX1-2 (SEQ ID NO: 24), Pur_POX (SEQ ID NO: 26), Lbo31670 (SEQ ID NO: 81), Lbo49554 (SEQ ID NO: 83), Lbo49602 (SEQ ID NO: 85) and Rno_POX2 (SEQ ID NO: 28), as well as functional variants thereof having at least 60% homology thereto, such as at least 65%, such as at least 70%, such as at least 75%, such as at least 80%, such as at least 81%, such as at least 82%, such as at least 83%, such as at least 84%, such as at least 85%, such as at least 86%, such as at least 87%, such as at least 88%, such as at least 89%, such as at least 90%, such as at least 91%, such as at least 92%, such as at least 93%, such as at least 94%, such as at least 95%, such as at least 96%, such as at least 97%, such as at least 98%, such as at least 99% homology thereto.

In some embodiments, the at least one acyl-CoA oxidase is selected from the group consisting of Yli_POX2 (SEQ ID NO: 4), Ani_POX (SEQ ID NO: 20), Cma_POX (SEQ ID NO: 22), Pur_POX (SEQ ID NO: 26), Lbo31670 (SEQ ID NO: 81), Lbo49554 (SEQ ID NO: 83), Lbo49602 (SEQ ID NO: 85) and Rno_POX2 (SEQ ID NO: 28), or functional variants thereof having at least 60% homology thereto, such as at least 65%, such as at least 70%, such as at least 75%, such as at least 80%, such as at least 81%, such as at least 82%, such as at least 83%, such as at least 84%, such as at least 85%, such as at least 86%, such as at least 87%, such as at least 88%, such as at least 89%, such as at least 90%, such as at least 91%, such as at least 92%, such as at least 93%, such as at least 94%, such as at least 95%, such as at least 96%, such as at least 97%, such as at least 98%, such as at least 99% homology thereto.

In some embodiments, the at least one acyl-CoA oxidase is selected from the group consisting of Ani_POX (SEQ ID NO: 20), Cma_POX (SEQ ID NO: 22), Pur_POX (SEQ ID NO: 26), Lbo31670 (SEQ ID NO: 81), Lbo49554 (SEQ ID NO: 83), Lbo49602 (SEQ ID NO: 85) and Rno_POX2 (SEQ ID NO: 28), or functional variants thereof having at least 60% homology thereto, such as at least 65%, such as at least 70%, such as at least 75%, such as at least 80%, such as at least 81%, such as at least 82%, such as at least 83%, such as at least 84%, such as at least 85%, such as at least 86%, such as at least 87%, such as at least 88%, such as at least 89%, such as at least 90%, such as at least 91%, such as at least 92%, such as at least 93%, such as at least 94%, such as at least 95%, such as at least 96%, such as at least 97%, such as at least 98%, such as at least 99% homology thereto.

In some embodiments, the at least one acyl-CoA oxidase is not a single acyl-CoA oxidase selected from Yli_POX1 (SEQ ID NO: 2), Yli_POX2 (SEQ ID NO: 4), Yli_POX3 (SEQ ID NO: 6), Yli_POX4 (SEQ ID NO: 8), Yli_POX5 (SEQ ID NO: 10), Yli_POX6 (SEQ ID NO: 12).

The choice of the acyl-CoA oxidase or acyl-CoA oxidases to be expressed in the yeast cell of the present disclosure will typically be dictated by the desired carbon chain length of the desaturated fatty alcohol, desaturated fatty acyl acetate and/or fatty aldehydes which are to be produced.

The one or more acyl-CoA oxidases introduced expressed by the yeast cell are capable of oxidising a fatty acyl-CoA having a first carbon chain length X to a shortened fatty acyl-CoA having a second carbon chain length X′, wherein X′ is smaller than X. X and X′ are integers. X may be 8, 9, 10, 11, 12, 13, 14, 15, 16, 17, 18, 19, 20, 21 or 22.

In some embodiments, the first group of enzymes has greater activity towards fatty acyl-CoAs of carbon chain length greater than X′ than towards fatty acyl-CoAs of carbon chain length equal to or smaller than X′. In some embodiments, the at least one acyl-CoA oxidase has greater activity towards fatty acyl-CoAs of carbon chain length greater than X′ than towards fatty acyl-CoAs of carbon chain length equal to or smaller than X′. In some embodiments, the first group of enzymes has greater activity towards fatty acyl-CoAs of carbon chain length equal to or greater than X than towards fatty acyl-CoAs of carbon chain length smaller than X. In some embodiments, the at least one acyl-CoA oxidase has greater activity towards fatty acyl-CoAs of carbon chain length equal to or greater than X than towards fatty acyl-CoAs of carbon chain length smaller than X.

Preferably, X′ ≤ X-2. In some embodiments, X′ = X-2. In other embodiments, X′ = X-4. In other embodiments, X′ = X-6. This means that the carbon chain is shortened by 2, 4 or 6 carbon atoms. In some embodiments, X′ ≤ X-6.

In some embodiments, multiple acyl-CoA oxidases are expressed simultaneously in the yeast cell. This may be useful to obtain multiple shortened fatty acyl-CoAs of different carbon chain length X′₁, X′₂,... X′ₙ, from a fatty acyl-CoA of carbon chain length X, where X′₁, X′₂,... X′ₙ ≤ X-2. In some embodiments, the yeast cell expresses one acyl-CoA oxidase. In other embodiments, the yeast cell expresses at least two acyl-CoA oxidases, such as at least three acyl-CoA oxidases, such as at least four, five, six, seven, eight, nine or ten acyl-CoA oxidases. These may each independently be as described herein.

In some embodiments, the yeast cell expresses one acyl-CoA oxidase, such as an acyl-CoA oxidase selected from the group consisting of Yli_POX1 (SEQ ID NO: 2), Yli_POX2 (SEQ ID NO: 4), Yli_POX3 (SEQ ID NO: 6), Yli_POX4 (SEQ ID NO: 8), Yli_POX5 (SEQ ID NO: 10), Yli_POX6 (SEQ ID NO: 12), Ase_POX (SEQ ID NO: 14), Ath_POX1 (SEQ ID NO: 16), Ath_POX2 (SEQ ID NO: 18), Ani_POX (SEQ ID NO: 20), Cma_POX (SEQ ID NO: 22), Hsa_POX1-2 (SEQ ID NO: 24), Pur_POX (SEQ ID NO: 26), Lbo31670 (SEQ ID NO: 81), Lbo49554 (SEQ ID NO: 83), Lbo49602 (SEQ ID NO: 85) and Rno_POX2 (SEQ ID NO: 28), or a functional variant thereof having at least 60% homology thereto, such as at least 65%, such as at least 70%, such as at least 75%, such as at least 80%, such as at least 81%, such as at least 82%, such as at least 83%, such as at least 84%, such as at least 85%, such as at least 86%, such as at least 87%, such as at least 88%, such as at least 89%, such as at least 90%, such as at least 91%, such as at least 92%, such as at least 93%, such as at least 94%, such as at least 95%, such as at least 96%, such as at least 97%, such as at least 98%, such as at least 99% homology thereto.

In some embodiments, the yeast cell expresses one acyl-CoA oxidase selected from the group consisting of Yli_POX2 (SEQ ID NO: 4), Ani_POX (SEQ ID NO: 20), Cma_POX (SEQ ID NO: 22), Pur_POX (SEQ ID NO: 26), Lbo31670 (SEQ ID NO: 81), Lbo49554 (SEQ ID NO: 83), Lbo49602 (SEQ ID NO: 85) and Rno_POX2 (SEQ ID NO: 28), or functional variants thereof having at least 60% homology thereto, such as at least 65%, such as at least 70%, such as at least 75%, such as at least 80%, such as at least 81%, such as at least 82%, such as at least 83%, such as at least 84%, such as at least 85%, such as at least 86%, such as at least 87%, such as at least 88%, such as at least 89%, such as at least 90%, such as at least 91%, such as at least 92%, such as at least 93%, such as at least 94%, such as at least 95%, such as at least 96%, such as at least 97%, such as at least 98%, such as at least 99% homology thereto.

In some embodiments, the yeast cell does not express Ase_POX (SEQ ID NO: 14).

In one embodiment, the acyl-CoA oxidase is Yli_POX1 (SEQ ID NO: 2) or a functional variant thereof having at least 60% homology thereto, such as at least 65%, such as at least 70%, such as at least 75%, such as at least 80%, such as at least 81%, such as at least 82%, such as at least 83%, such as at least 84%, such as at least 85%, such as at least 86%, such as at least 87%, such as at least 88%, such as at least 89%, such as at least 90%, such as at least 91%, such as at least 92%, such as at least 93%, such as at least 94%, such as at least 95%, such as at least 96%, such as at least 97%, such as at least 98%, such as at least 99% homology thereto.

In another embodiment, the acyl-CoA oxidase is Yli_POX2 (SEQ ID NO: 4) or a functional variant thereof having at least 60% homology thereto, such as at least 65%, such as at least 70%, such as at least 75%, such as at least 80%, such as at least 81%, such as at least 82%, such as at least 83%, such as at least 84%, such as at least 85%, such as at least 86%, such as at least 87%, such as at least 88%, such as at least 89%, such as at least 90%, such as at least 91%, such as at least 92%, such as at least 93%, such as at least 94%, such as at least 95%, such as at least 96%, such as at least 97%, such as at least 98%, such as at least 99% homology thereto.

In another embodiment, the acyl-CoA oxidase is Yli_POX3 (SEQ ID NO: 6) or a functional variant thereof having at least 60% homology thereto, such as at least 65%, such as at least 70%, such as at least 75%, such as at least 80%, such as at least 81%, such as at least 82%, such as at least 83%, such as at least 84%, such as at least 85%, such as at least 86%, such as at least 87%, such as at least 88%, such as at least 89%, such as at least 90%, such as at least 91%, such as at least 92%, such as at least 93%, such as at least 94%, such as at least 95%, such as at least 96%, such as at least 97%, such as at least 98%, such as at least 99% homology thereto.

In another embodiment, the acyl-CoA oxidase is Yli_POX4 (SEQ ID NO: 8) or a functional variant thereof having at least 60% homology thereto, such as at least 65%, such as at least 70%, such as at least 75%, such as at least 80%, such as at least 81%, such as at least 82%, such as at least 83%, such as at least 84%, such as at least 85%, such as at least 86%, such as at least 87%, such as at least 88%, such as at least 89%, such as at least 90%, such as at least 91%, such as at least 92%, such as at least 93%, such as at least 94%, such as at least 95%, such as at least 96%, such as at least 97%, such as at least 98%, such as at least 99% homology thereto.

In another embodiment, the acyl-CoA oxidase is Yli_POX5 (SEQ ID NO: 10) or a functional variant thereof having at least 60% homology thereto, such as at least 65%, such as at least 70%, such as at least 75%, such as at least 80%, such as at least 81%, such as at least 82%, such as at least 83%, such as at least 84%, such as at least 85%, such as at least 86%, such as at least 87%, such as at least 88%, such as at least 89%, such as at least 90%, such as at least 91%, such as at least 92%, such as at least 93%, such as at least 94%, such as at least 95%, such as at least 96%, such as at least 97%, such as at least 98%, such as at least 99% homology thereto.

In another embodiment, the acyl-CoA oxidase is Yli_POX6 (SEQ ID NO: 12) or a functional variant thereof having at least 60% homology thereto, such as at least 65%, such as at least 70%, such as at least 75%, such as at least 80%, such as at least 81%, such as at least 82%, such as at least 83%, such as at least 84%, such as at least 85%, such as at least 86%, such as at least 87%, such as at least 88%, such as at least 89%, such as at least 90%, such as at least 91%, such as at least 92%, such as at least 93%, such as at least 94%, such as at least 95%, such as at least 96%, such as at least 97%, such as at least 98%, such as at least 99% homology thereto.

In another embodiment, the acyl-CoA oxidase is Ase_POX (SEQ ID NO: 14) or a functional variant thereof having at least 60% homology thereto, such as at least 65%, such as at least 70%, such as at least 75%, such as at least 80%, such as at least 81%, such as at least 82%, such as at least 83%, such as at least 84%, such as at least 85%, such as at least 86%, such as at least 87%, such as at least 88%, such as at least 89%, such as at least 90%, such as at least 91%, such as at least 92%, such as at least 93%, such as at least 94%, such as at least 95%, such as at least 96%, such as at least 97%, such as at least 98%, such as at least 99% homology thereto.

In another embodiment, the acyl-CoA oxidase is Ath_POX1 (SEQ ID NO: 16) or a functional variant thereof having at least 60% homology thereto, such as at least 65%, such as at least 70%, such as at least 75%, such as at least 80%, such as at least 81%, such as at least 82%, such as at least 83%, such as at least 84%, such as at least 85%, such as at least 86%, such as at least 87%, such as at least 88%, such as at least 89%, such as at least 90%, such as at least 91%, such as at least 92%, such as at least 93%, such as at least 94%, such as at least 95%, such as at least 96%, such as at least 97%, such as at least 98%, such as at least 99% homology thereto.

In another embodiment, the acyl-CoA oxidase is Ath_POX2 (SEQ ID NO: 18) or a functional variant thereof having at least 60% homology thereto, such as at least 65%, such as at least 70%, such as at least 75%, such as at least 80%, such as at least 81%, such as at least 82%, such as at least 83%, such as at least 84%, such as at least 85%, such as at least 86%, such as at least 87%, such as at least 88%, such as at least 89%, such as at least 90%, such as at least 91%, such as at least 92%, such as at least 93%, such as at least 94%, such as at least 95%, such as at least 96%, such as at least 97%, such as at least 98%, such as at least 99% homology thereto.

In another embodiment, the acyl-CoA oxidase is Ani_POX (SEQ ID NO: 20) or a functional variant thereof having at least 60% homology thereto, such as at least 65%, such as at least 70%, such as at least 75%, such as at least 80%, such as at least 81%, such as at least 82%, such as at least 83%, such as at least 84%, such as at least 85%, such as at least 86%, such as at least 87%, such as at least 88%, such as at least 89%, such as at least 90%, such as at least 91%, such as at least 92%, such as at least 93%, such as at least 94%, such as at least 95%, such as at least 96%, such as at least 97%, such as at least 98%, such as at least 99% homology thereto.

In another embodiment, the acyl-CoA oxidase is Cma_POX (SEQ ID NO: 22) or a functional variant thereof having at least 60% homology thereto, such as at least 65%, such as at least 70%, such as at least 75%, such as at least 80%, such as at least 81%, such as at least 82%, such as at least 83%, such as at least 84%, such as at least 85%, such as at least 86%, such as at least 87%, such as at least 88%, such as at least 89%, such as at least 90%, such as at least 91%, such as at least 92%, such as at least 93%, such as at least 94%, such as at least 95%, such as at least 96%, such as at least 97%, such as at least 98%, such as at least 99% homology thereto.

In another embodiment, the acyl-CoA oxidase is Has_POX1-2 (SEQ ID NO: 24) or a functional variant thereof having at least 60% homology thereto, such as at least 65%, such as at least 70%, such as at least 75%, such as at least 80%, such as at least 81%, such as at least 82%, such as at least 83%, such as at least 84%, such as at least 85%, such as at least 86%, such as at least 87%, such as at least 88%, such as at least 89%, such as at least 90%, such as at least 91%, such as at least 92%, such as at least 93%, such as at least 94%, such as at least 95%, such as at least 96%, such as at least 97%, such as at least 98%, such as at least 99% homology thereto.

In another embodiment, the acyl-CoA oxidase is Pur_POX (SEQ ID NO: 26) or a functional variant thereof having at least 60% homology thereto, such as at least 65%, such as at least 70%, such as at least 75%, such as at least 80%, such as at least 81%, such as at least 82%, such as at least 83%, such as at least 84%, such as at least 85%, such as at least 86%, such as at least 87%, such as at least 88%, such as at least 89%, such as at least 90%, such as at least 91%, such as at least 92%, such as at least 93%, such as at least 94%, such as at least 95%, such as at least 96%, such as at least 97%, such as at least 98%, such as at least 99% homology thereto.

In another embodiment, the acyl-CoA oxidase is Rno_POX-2 (SEQ ID NO: 28) or a functional variant thereof having at least 60% homology thereto, such as at least 65%, such as at least 70%, such as at least 75%, such as at least 80%, such as at least 81%, such as at least 82%, such as at least 83%, such as at least 84%, such as at least 85%, such as at least 86%, such as at least 87%, such as at least 88%, such as at least 89%, such as at least 90%, such as at least 91%, such as at least 92%, such as at least 93%, such as at least 94%, such as at least 95%, such as at least 96%, such as at least 97%, such as at least 98%, such as at least 99% homology thereto.

In another embodiment, the acyl-CoA oxidase is Lbo31670 (SEQ ID NO: 81) or a functional variant thereof having at least 60% homology thereto, such as at least 65%, such as at least 70%, such as at least 75%, such as at least 80%, such as at least 81%, such as at least 82%, such as at least 83%, such as at least 84%, such as at least 85%, such as at least 86%, such as at least 87%, such as at least 88%, such as at least 89%, such as at least 90%, such as at least 91%, such as at least 92%, such as at least 93%, such as at least 94%, such as at least 95%, such as at least 96%, such as at least 97%, such as at least 98%, such as at least 99% homology thereto.

In another embodiment, the acyl-CoA oxidase is Lbo49554 (SEQ ID NO: 83) or a functional variant thereof having at least 60% homology thereto, such as at least 65%, such as at least 70%, such as at least 75%, such as at least 80%, such as at least 81%, such as at least 82%, such as at least 83%, such as at least 84%, such as at least 85%, such as at least 86%, such as at least 87%, such as at least 88%, such as at least 89%, such as at least 90%, such as at least 91%, such as at least 92%, such as at least 93%, such as at least 94%, such as at least 95%, such as at least 96%, such as at least 97%, such as at least 98%, such as at least 99% homology thereto.

In another embodiment, the acyl-CoA oxidase is Lbo49602 (SEQ ID NO: 85) or a functional variant thereof having at least 60% homology thereto, such as at least 65%, such as at least 70%, such as at least 75%, such as at least 80%, such as at least 81%, such as at least 82%, such as at least 83%, such as at least 84%, such as at least 85%, such as at least 86%, such as at least 87%, such as at least 88%, such as at least 89%, such as at least 90%, such as at least 91%, such as at least 92%, such as at least 93%, such as at least 94%, such as at least 95%, such as at least 96%, such as at least 97%, such as at least 98%, such as at least 99% homology thereto.

In some embodiments, the yeast cell expresses two acyl-CoA oxidases selected from the group consisting of Yli_POX1 (SEQ ID NO: 2), Yli_POX2 (SEQ ID NO: 4), Yli_POX3 (SEQ ID NO: 6), Yli_POX4 (SEQ ID NO: 8), Yli_POX5 (SEQ ID NO: 10), Yli_POX6 (SEQ ID NO: 12), Ase_POX (SEQ ID NO: 14), Ath_POX1 (SEQ ID NO: 16), Ath_POX2 (SEQ ID NO: 18), Ani_POX (SEQ ID NO: 20), Cma_POX (SEQ ID NO: 22), Hsa_POX1-2 (SEQ ID NO: 24), Pur_POX (SEQ ID NO: 26), Lbo31670 (SEQ ID NO: 81), Lbo49554 (SEQ ID NO: 83), Lbo49602 (SEQ ID NO: 85) and Rno_POX2 (SEQ ID NO: 28), or functional variants thereof having at least 60% homology thereto, such as at least 65%, such as at least 70%, such as at least 75%, such as at least 80%, such as at least 81%, such as at least 82%, such as at least 83%, such as at least 84%, such as at least 85%, such as at least 86%, such as at least 87%, such as at least 88%, such as at least 89%, such as at least 90%, such as at least 91%, such as at least 92%, such as at least 93%, such as at least 94%, such as at least 95%, such as at least 96%, such as at least 97%, such as at least 98%, such as at least 99% homology thereto. In some embodiments, the two acyl-CoA oxidases are selected from Yli_POX2 (SEQ ID NO: 4), Ani_POX (SEQ ID NO: 20), Cma_POX (SEQ ID NO: 22), Pur_POX (SEQ ID NO: 26), Lbo31670 (SEQ ID NO: 81), Lbo49554 (SEQ ID NO: 83), Lbo49602 (SEQ ID NO: 85) and Rno_POX2 (SEQ ID NO: 28).

In some embodiments, the yeast cell expresses two, three, four, five, six, seven, eight, nine or ten acyl-CoA oxidases independently selected from the group consisting of Yli_POX1 (SEQ ID NO: 2), Yli_POX2 (SEQ ID NO: 4), Yli_POX3 (SEQ ID NO: 6), Yli_POX4 (SEQ ID NO: 8), Yli_POX5 (SEQ ID NO: 10), Yli_POX6 (SEQ ID NO: 12), Ase_POX (SEQ ID NO: 14), Ath_POX1 (SEQ ID NO: 16), Ath_POX2 (SEQ ID NO: 18), Ani_POX (SEQ ID NO: 20), Cma_POX (SEQ ID NO: 22), Hsa_POX1-2 (SEQ ID NO: 24), Pur_POX (SEQ ID NO: 26), Lbo31670 (SEQ ID NO: 81), Lbo49554 (SEQ ID NO: 83), Lbo49602 (SEQ ID NO: 85) and Rno_POX2 (SEQ ID NO: 28), or a functional variant thereof having at least 60% homology thereto, such as at least 65%, such as at least 70%, such as at least 75%, such as at least 80%, such as at least 81%, such as at least 82%, such as at least 83%, such as at least 84%, such as at least 85%, such as at least 86%, such as at least 87%, such as at least 88%, such as at least 89%, such as at least 90%, such as at least 91%, such as at least 92%, such as at least 93%, such as at least 94%, such as at least 95%, such as at least 96%, such as at least 97%, such as at least 98%, such as at least 99% homology thereto. In some embodiments, the two, three, four, five, six, seven, eight, nine or ten acyl-CoA oxidases are independently selected from Yli_POX2 (SEQ ID NO: 4), Ani_POX (SEQ ID NO: 20), Cma_POX (SEQ ID NO: 22), Pur_POX (SEQ ID NO: 26), Lbo31670 (SEQ ID NO: 81), Lbo49554 (SEQ ID NO: 83), Lbo49602 (SEQ ID NO: 85) and Rno_POX2 (SEQ ID NO: 28).

In some embodiments, a nucleic acid encoding the one or more acyl-CoA oxidases which it is desired to express in the cell is introduced by methods known in the art or as described in the section ‘Nucleic acids’ herein.

In some embodiments, the yeast cell disclosed herein has been modified by introduction of one or more nucleic acids encoding an acyl-CoA oxidase selected from the group of Yli_POX1 (SEQ ID NO: 2), Yli_POX2 (SEQ ID NO: 4), Yli_POX3 (SEQ ID NO: 6), Yli_POX4 (SEQ ID NO: 8), Yli_POX5 (SEQ ID NO: 10), Yli_POX6 (SEQ ID NO: 12), Ase_POX (SEQ ID NO: 14), Ath_POX1 (SEQ ID NO: 16), Ath_POX2 (SEQ ID NO: 18), Ani_POX (SEQ ID NO: 20), Cma_POX (SEQ ID NO: 22), Hsa_POX1-2 (SEQ ID NO: 24), Pur_POX (SEQ ID NO: 26), Lbo31670 (SEQ ID NO: 81), Lbo49554 (SEQ ID NO: 83), Lbo49602 (SEQ ID NO: 85) and Rno_POX2 (SEQ ID NO: 28), as well as functional variants thereof having at least 60% homology thereto, such as at least 65%, such as at least 70%, such as at least 75%, such as at least 80%, such as at least 81%, such as at least 82%, such as at least 83%, such as at least 84%, such as at least 85%, such as at least 86%, such as at least 87%, such as at least 88%, such as at least 89%, such as at least 90%, such as at least 91%, such as at least 92%, such as at least 93%, such as at least 94%, such as at least 95%, such as at least 96%, such as at least 97%, such as at least 98%, such as at least 99% homology thereto. In some embodiments, the nucleic acid encoding the acyl-CoA oxidase is selected from SEQ ID NO: 1, SEQ ID NO: 3, SEQ ID NO: 5, SEQ ID NO: 7, SEQ ID NO: 9, SEQ ID NO: 11, SEQ ID NO: 13, SEQ ID NO: 15, SEQ ID NO: 17, SEQ ID NO: 19, SEQ ID NO: 21, SEQ ID NO: 23, SEQ ID NO: 25, SEQ ID NO: 27, SEQ ID NO: 80, SEQ ID NO: 82, and SEQ ID NO: 84, or homologues thereof having at least 60% homology thereto, such as at least 65%, such as at least 70%, such as at least 75%, such as at least 80%, such as at least 81%, such as at least 82%, such as at least 83%, such as at least 84%, such as at least 85%, such as at least 86%, such as at least 87%, such as at least 88%, such as at least 89%, such as at least 90%, such as at least 91%, such as at least 92%, such as at least 93%, such as at least 94%, such as at least 95%, such as at least 96%, such as at least 97%, such as at least 98%, such as at least 99% homology thereto.

In one embodiment, the yeast cell is modified by introduction of a nucleic acid encoding Yli_POX1, wherein said nucleic acid comprises or consists of SEQ ID NO: 1 or a homologue thereof having at least 60% homology thereto, such as at least 65%, such as at least 70%, such as at least 75%, such as at least 80%, such as at least 81%, such as at least 82%, such as at least 83%, such as at least 84%, such as at least 85%, such as at least 86%, such as at least 87%, such as at least 88%, such as at least 89%, such as at least 90%, such as at least 91%, such as at least 92%, such as at least 93%, such as at least 94%, such as at least 95%, such as at least 96%, such as at least 97%, such as at least 98%, such as at least 99% homology thereto.

In another embodiment, the yeast cell is modified by introduction of a nucleic acid encoding Yli_POX2, wherein said nucleic acid comprises or consists of SEQ ID NO: 3 or a homologue thereof having at least 60% homology thereto, such as at least 65%, such as at least 70%, such as at least 75%, such as at least 80%, such as at least 81%, such as at least 82%, such as at least 83%, such as at least 84%, such as at least 85%, such as at least 86%, such as at least 87%, such as at least 88%, such as at least 89%, such as at least 90%, such as at least 91%, such as at least 92%, such as at least 93%, such as at least 94%, such as at least 95%, such as at least 96%, such as at least 97%, such as at least 98%, such as at least 99% homology thereto.

In another embodiment, the yeast cell is modified by introduction of a nucleic acid encoding Yli_POX3, wherein said nucleic acid comprises or consists of SEQ ID NO: 5 or a homologue thereof having at least 60% homology thereto, such as at least 65%, such as at least 70%, such as at least 75%, such as at least 80%, such as at least 81%, such as at least 82%, such as at least 83%, such as at least 84%, such as at least 85%, such as at least 86%, such as at least 87%, such as at least 88%, such as at least 89%, such as at least 90%, such as at least 91%, such as at least 92%, such as at least 93%, such as at least 94%, such as at least 95%, such as at least 96%, such as at least 97%, such as at least 98%, such as at least 99% homology thereto.

In another embodiment, the yeast cell is modified by introduction of a nucleic acid encoding Yli_POX4, wherein said nucleic acid comprises or consists of SEQ ID NO: 7 or a homologue thereof having at least 60% homology thereto, such as at least 65%, such as at least 70%, such as at least 75%, such as at least 80%, such as at least 81%, such as at least 82%, such as at least 83%, such as at least 84%, such as at least 85%, such as at least 86%, such as at least 87%, such as at least 88%, such as at least 89%, such as at least 90%, such as at least 91%, such as at least 92%, such as at least 93%, such as at least 94%, such as at least 95%, such as at least 96%, such as at least 97%, such as at least 98%, such as at least 99% homology thereto.

In another embodiment, the yeast cell is modified by introduction of a nucleic acid encoding Yli_POX5, wherein said nucleic acid comprises or consists of SEQ ID NO: 9 or a homologue thereof having at least 60% homology thereto, such as at least 65%, such as at least 70%, such as at least 75%, such as at least 80%, such as at least 81%, such as at least 82%, such as at least 83%, such as at least 84%, such as at least 85%, such as at least 86%, such as at least 87%, such as at least 88%, such as at least 89%, such as at least 90%, such as at least 91%, such as at least 92%, such as at least 93%, such as at least 94%, such as at least 95%, such as at least 96%, such as at least 97%, such as at least 98%, such as at least 99% homology thereto.

In another embodiment, the yeast cell is modified by introduction of a nucleic acid encoding Yli_POX6, wherein said nucleic acid comprises or consists of SEQ ID NO: 11 or a homologue thereof having at least 60% homology thereto, such as at least 65%, such as at least 70%, such as at least 75%, such as at least 80%, such as at least 81%, such as at least 82%, such as at least 83%, such as at least 84%, such as at least 85%, such as at least 86%, such as at least 87%, such as at least 88%, such as at least 89%, such as at least 90%, such as at least 91%, such as at least 92%, such as at least 93%, such as at least 94%, such as at least 95%, such as at least 96%, such as at least 97%, such as at least 98%, such as at least 99% homology thereto.

In another embodiment, the yeast cell is modified by introduction of a nucleic acid encoding Ase_POX, wherein said nucleic acid comprises or consists of SEQ ID NO: 13 or a homologue thereof having at least 60% homology thereto, such as at least 65%, such as at least 70%, such as at least 75%, such as at least 80%, such as at least 81%, such as at least 82%, such as at least 83%, such as at least 84%, such as at least 85%, such as at least 86%, such as at least 87%, such as at least 88%, such as at least 89%, such as at least 90%, such as at least 91%, such as at least 92%, such as at least 93%, such as at least 94%, such as at least 95%, such as at least 96%, such as at least 97%, such as at least 98%, such as at least 99% homology thereto.

In another embodiment, the yeast cell is modified by introduction of a nucleic acid encoding Ath_POX1, wherein said nucleic acid comprises or consists of SEQ ID NO: 15 or a homologue thereof having at least 60% homology thereto, such as at least 65%, such as at least 70%, such as at least 75%, such as at least 80%, such as at least 81%, such as at least 82%, such as at least 83%, such as at least 84%, such as at least 85%, such as at least 86%, such as at least 87%, such as at least 88%, such as at least 89%, such as at least 90%, such as at least 91%, such as at least 92%, such as at least 93%, such as at least 94%, such as at least 95%, such as at least 96%, such as at least 97%, such as at least 98%, such as at least 99% homology thereto.

In another embodiment, the yeast cell is modified by introduction of a nucleic acid encoding Ath_POX2, wherein said nucleic acid comprises or consists of SEQ ID NO: 17 or a homologue thereof having at least 60% homology thereto, such as at least 65%, such as at least 70%, such as at least 75%, such as at least 80%, such as at least 81%, such as at least 82%, such as at least 83%, such as at least 84%, such as at least 85%, such as at least 86%, such as at least 87%, such as at least 88%, such as at least 89%, such as at least 90%, such as at least 91%, such as at least 92%, such as at least 93%, such as at least 94%, such as at least 95%, such as at least 96%, such as at least 97%, such as at least 98%, such as at least 99% homology thereto.

In another embodiment, the yeast cell is modified by introduction of a nucleic acid encoding Ani_POX, wherein said nucleic acid comprises or consists of SEQ ID NO: 19 or a homologue thereof having at least 60% homology thereto, such as at least 65%, such as at least 70%, such as at least 75%, such as at least 80%, such as at least 81%, such as at least 82%, such as at least 83%, such as at least 84%, such as at least 85%, such as at least 86%, such as at least 87%, such as at least 88%, such as at least 89%, such as at least 90%, such as at least 91%, such as at least 92%, such as at least 93%, such as at least 94%, such as at least 95%, such as at least 96%, such as at least 97%, such as at least 98%, such as at least 99% homology thereto.

In another embodiment, the yeast cell is modified by introduction of a nucleic acid encoding Cma_POX, wherein said nucleic acid comprises or consists of SEQ ID NO: 21 or a homologue thereof having at least 60% homology thereto, such as at least 65%, such as at least 70%, such as at least 75%, such as at least 80%, such as at least 81%, such as at least 82%, such as at least 83%, such as at least 84%, such as at least 85%, such as at least 86%, such as at least 87%, such as at least 88%, such as at least 89%, such as at least 90%, such as at least 91%, such as at least 92%, such as at least 93%, such as at least 94%, such as at least 95%, such as at least 96%, such as at least 97%, such as at least 98%, such as at least 99% homology thereto.

In another embodiment, the yeast cell is modified by introduction of a nucleic acid encoding Hsa_POX1-2, wherein said nucleic acid comprises or consists of SEQ ID NO: 23 or a homologue thereof having at least 60% homology thereto, such as at least 65%, such as at least 70%, such as at least 75%, such as at least 80%, such as at least 81%, such as at least 82%, such as at least 83%, such as at least 84%, such as at least 85%, such as at least 86%, such as at least 87%, such as at least 88%, such as at least 89%, such as at least 90%, such as at least 91%, such as at least 92%, such as at least 93%, such as at least 94%, such as at least 95%, such as at least 96%, such as at least 97%, such as at least 98%, such as at least 99% homology thereto.

In another embodiment, the yeast cell is modified by introduction of a nucleic acid encoding Pur_POX, wherein said nucleic acid comprises or consists of SEQ ID NO: 25 or a homologue thereof having at least 60% homology thereto, such as at least 65%, such as at least 70%, such as at least 75%, such as at least 80%, such as at least 81%, such as at least 82%, such as at least 83%, such as at least 84%, such as at least 85%, such as at least 86%, such as at least 87%, such as at least 88%, such as at least 89%, such as at least 90%, such as at least 91%, such as at least 92%, such as at least 93%, such as at least 94%, such as at least 95%, such as at least 96%, such as at least 97%, such as at least 98%, such as at least 99% homology thereto.

In another embodiment, the yeast cell is modified by introduction of a nucleic acid encoding Rno_POX-2, wherein said nucleic acid comprises or consists of SEQ ID NO: 27 or a homologue thereof having at least 60% homology thereto, such as at least 65%, such as at least 70%, such as at least 75%, such as at least 80%, such as at least 81%, such as at least 82%, such as at least 83%, such as at least 84%, such as at least 85%, such as at least 86%, such as at least 87%, such as at least 88%, such as at least 89%, such as at least 90%, such as at least 91%, such as at least 92%, such as at least 93%, such as at least 94%, such as at least 95%, such as at least 96%, such as at least 97%, such as at least 98%, such as at least 99% homology thereto.

In another embodiment, the yeast cell is modified by introduction of a nucleic acid encoding Lbo31670, wherein said nucleic acid comprises or consists of SEQ ID NO: 80 or a homologue thereof having at least 60% homology thereto, such as at least 65%, such as at least 70%, such as at least 75%, such as at least 80%, such as at least 81%, such as at least 82%, such as at least 83%, such as at least 84%, such as at least 85%, such as at least 86%, such as at least 87%, such as at least 88%, such as at least 89%, such as at least 90%, such as at least 91%, such as at least 92%, such as at least 93%, such as at least 94%, such as at least 95%, such as at least 96%, such as at least 97%, such as at least 98%, such as at least 99% homology thereto.

In another embodiment, the yeast cell is modified by introduction of a nucleic acid encoding Lbo49554, wherein said nucleic acid comprises or consists of SEQ ID NO: 82 or a homologue thereof having at least 60% homology thereto, such as at least 65%, such as at least 70%, such as at least 75%, such as at least 80%, such as at least 81%, such as at least 82%, such as at least 83%, such as at least 84%, such as at least 85%, such as at least 86%, such as at least 87%, such as at least 88%, such as at least 89%, such as at least 90%, such as at least 91%, such as at least 92%, such as at least 93%, such as at least 94%, such as at least 95%, such as at least 96%, such as at least 97%, such as at least 98%, such as at least 99% homology thereto.

In another embodiment, the yeast cell is modified by introduction of a nucleic acid encoding Lbo49602, wherein said nucleic acid comprises or consists of SEQ ID NO: 84 or a homologue thereof having at least 60% homology thereto, such as at least 65%, such as at least 70%, such as at least 75%, such as at least 80%, such as at least 81%, such as at least 82%, such as at least 83%, such as at least 84%, such as at least 85%, such as at least 86%, such as at least 87%, such as at least 88%, such as at least 89%, such as at least 90%, such as at least 91%, such as at least 92%, such as at least 93%, such as at least 94%, such as at least 95%, such as at least 96%, such as at least 97%, such as at least 98%, such as at least 99% homology thereto.

Once the genes encoding the one or more acyl-CoA oxidases have been introduced in the yeast cell, candidate strains are obtained, which can be tested for the desired activity, i.e. the ability to oxidise fatty acyl-CoAs of a given carbon chain length.

In order to test whether an acyl-CoA oxidase is suitably expressed in the modified yeast cell and has the desired activity, candidate strains are cultivated as is known in the art and extracts are prepared. The activity of the extracts on fatty acyl-CoAs having the desired carbon chain length is tested, as is known in the art. This can for example be done using enzymatic assays as described in [6, 7]. Another method involves testing the ability of the candidate strains to grow on fatty acid substrates of specific lengths using e.g. spot assays on solid medium.

Strains are then identified which are capable of oxidising fatty acyl-CoAs of carbon chain length equal to or greater than X to shortened fatty acyl-CoAs of carbon chain length X′, wherein X′ is smaller than X; preferably the strain has reduced or no capability of oxidising fatty acyl-CoAs of carbon chain length smaller than X or X′. In practice, for example, a candidate strain having activity on fatty acyl-CoAs having a carbon chain length X ≥ 16 but reduced activity or no activity on fatty acyl-CoAs having a carbon chain length X < 16 is considered suitable for producing desaturated fatty alcohols, acetates and aldehydes of carbon chain length X′ = 14. A candidate strain having activity on fatty acyl-CoAs having a carbon chain length X ≥ 14 but reduced activity or no activity on fatty acyl-CoAs having a carbon chain length X < 14 is considered suitable for producing desaturated fatty alcohols, acetates and aldehydes of carbon chain length X′ = 12.

### Desaturase (FAD)

In the present disclosure, the terms ‘fatty acyl-CoA desaturase’, ‘desaturase’, ‘fatty acyl desaturase’ and ‘FAD’ will be used interchangeably. The term refers to an enzyme capable of introducing at least one double bond in E/Z conformations in a fatty acyl-CoA having a carbon chain length X or X′. X or X′ may be 8, 9, 10, 11, 12, 13, 14, 15, 16, 17, 18, 19, 20, 21 or 22 carbon atoms. In some embodiments, the desaturase is capable of introducing at least one double in E/Z conformations in a fatty acyl-CoA having a chain length of X′, wherein X′ is as defined above.

In addition to the first group of enzymes comprising an acyl-CoA oxidase as described herein above, the yeast cell of the present disclosure expresses a heterologous desaturase capable of introducing at least one double bond in said fatty acyl-CoA and/or in said shortened fatty acyl-CoA of carbon chain length X and X′, respectively. Preferably, the desaturase is capable of introducing at least one double bond in at least the shortened fatty acyl-CoA of carbon chain length X′. The ability to introduce at least one double bond in a fatty acyl-CoA is equivalent to the ability of converting said fatty acyl-CoA to a desaturated fatty acyl-CoA. The yeast cell expressing such desaturases is thus capable of converting a fatty acyl-CoA, in particular a shortened fatty acyl-CoA of carbon chain length X′, to a desaturated fatty acyl-CoA of carbon chain length X′.

The double bond may be introduced in any position. For example, a desaturase introducing a double bond in position 3 is termed Δ3 desaturase. A desaturase introducing a double bond in position 5 is termed Δ5 desaturase.A desaturase introducing a double bond in position 6 is termed Δ6 desaturase. A desaturase introducing a double bond in position 7 is termed Δ7 desaturase. A desaturase introducing a double bond in position 8 is termed Δ8 desaturase. A desaturase introducing a double bond in position 9 is termed Δ9 desaturase. A desaturase introducing a double bond in position 10 is termed Δ10 desaturase. A desaturase introducing a double bond in position 11 is termed Δ11 desaturase. A desaturase introducing a double bond in position 12 is termed Δ12 desaturase. A desaturase introducing a double bond in position 13 is termed Δ13 desaturase. A desaturase introducing a double bond in position 14 is termed Δ14 desaturase. A desaturase introducing a double bond in position 15 is termed Δ15 desaturase. A desaturase introducing a double bond in position 16 is termed Δ16 desaturase. A desaturase introducing a double bond in position 17 is termed Δ17 desaturase. A desaturase introducing a double bond in position 18 is termed Δ18 desaturase. A desaturase introducing a double bond in position 19 is termed Δ19 desaturase. A desaturase introducing a double bond in position 20 is termed Δ20 desaturase.

In one embodiment, the cell is capable of expressing at least one heterologous Δ5 desaturase. In another embodiment, the cell is capable of expressing at least one heterologous Δ6 desaturase. In another embodiment, the cell is capable of expressing at least one heterologous Δ7 desaturase. In another embodiment, the cell is capable of expressing at least one heterologous Δ8 desaturase. In another embodiment, the cell is capable of expressing at least one heterologous Δ9 desaturase. In another embodiment, the cell is capable of expressing at least one heterologous Δ10 desaturase. In another embodiment, the cell is capable of expressing at least one heterologous Δ11 desaturase. In another embodiment, the cell is capable of expressing at least one heterologous Δ12 desaturase. In another embodiment, the cell is capable of expressing at least one heterologous Δ13 desaturase. In another embodiment, the cell is capable of expressing at least one heterologous Δ14 desaturase. In another embodiment, the cell is capable of expressing at least one heterologous Δ15 desaturase. In another embodiment, the cell is capable of expressing at least one heterologous Δ16 desaturase. In another embodiment, the cell is capable of expressing at least one heterologous Δ17 desaturase. In another embodiment, the cell is capable of expressing at least one heterologous Δ18 desaturase. In another embodiment, the cell is capable of expressing at least one heterologous Δ19 desaturase. In another embodiment, the cell is capable of expressing at least one heterologous Δ20 desaturase.

The heterologous desaturase may be derived from an insect, for example from the order of Lepidoptera. A desaturase is derived from a given insect if it is native to that insect. In one embodiment, the heterologous desaturase is derived from Drosophila melanogaster. In another embodiment, the heterologous desaturase is derived from Amyelois transitella. In another embodiment, the heterologous desaturase is derived from Helicoverpa assulta. In another embodiment, the heterologous desaturase is derived from Helicoverpa armigera. In another embodiment, the heterologous desaturase is derived from Choristoneura rosaceana. In another embodiment, the heterologous desaturase is derived from Choristoneura parallela. In another embodiment, the heterologous desaturase is derived from Ostrinia nubilalis. In another embodiment, the heterologous desaturase is derived from Thaumetopoea pityocampa. In another embodiment, the heterologous desaturase is derived from Dendrophilus punctatus. In another embodiment, the heterologous desaturase is derived from Grapholita molesta. In another embodiment, the heterologous desaturase is derived from Cydia pomonella. In another embodiment, the heterologous desaturase is derived from Epiphyas postvittana. In another embodiment, the heterologous desaturase is derived from Spodoptera littoralis. In another embodiment, the heterologous desaturase is derived from Spodoptera litura. In another embodiment, the heterologous desaturase is derived from Lobesia botrana. In another embodiment, the heterologous desaturase is derived from Chilo suppressalis. In another embodiment, the heterologous desaturase is derived from Pectinophora gossypiella.

The heterologous desaturase may be derived from a yeast cell, for example a Saccharomyces cell or a Yarrowia cell. In one embodiment, the desaturase is derived from Saccharomyces cerevisiae or Yarrowia lipolytica.

A heterologous desaturase may be expressed from a nucleic acid introduced in the cell, e.g. on a vector such as a plasmid, or by genomic integration. The nucleic acid may be codon-optimised for any purpose as is known in the art for the specific yeast cell used.

The yeast cell to be modified may express a native desaturase, which may have a negative impact on the production of desaturated fatty alcohol and/or desaturated fatty acyl acetate. Accordingly, if the yeast cell to be modified expresses such a native desaturase, the cell may be further modified so that activity of the native desaturase is reduced or absent.

To ensure lack of activity of a native desaturase, methods known in the art can be employed. The gene encoding the native desaturase may be deleted or partly deleted in order to ensure that the native desaturase is not expressed. Alternatively, the gene may be mutated so that the native desaturase is expressed but lacks activity, e.g. by mutation of the catalytical site of the enzyme. Alternatively, translation of mRNA to an active protein may be prevented by methods such as silencing RNA or siRNA. Alternatively, the yeast cell may be incubated in a medium comprising an inhibitor which inhibits activity of the native desaturase. A compound inhibiting transcription of the gene encoding the native desaturase may also be provided so that transcription is inactivated when said compound is present.

Inactivation of the native desaturase may thus be permanent or long-term, i.e. the modified yeast cell does not exhibit activity of the native desaturase in stable conditions, or it may be transient, i.e. the modified yeast cell may exhibit activity of the native desaturase for periods of time, but this activity can be suppressed for other periods of time.

The skilled person will know, depending on which desaturated fatty alcohol is desired, which kind of desaturase to use. For example, for the production of a fatty alcohol desaturated in position 11, a Δ11 desaturase having at least 60% homology to the Δ11 desaturase from Amyelois transitella as set forth in SEQ ID NO: 38, a Δ11 desaturase having at least 60% homology to the Δ11 desaturase from Choristoneura rosaceana as set forth in SEQ ID NO: 40, a Δ11 desaturase having at least 60% homology to the Δ11 desaturase from Choristoneura parallela as set forth in SEQ ID NO: 56, a Δ11 desaturase having at least 60% homology to the Δ11 desaturase from Ostinia nubilalis as set forth in SEQ ID NO: 42, a Δ11 desaturase having at least 60% homology to the Δ11 desaturase from Thaumetopoea pityocampa as set forth in SEQ ID NO: 44, a desaturase having at least 60% homology to the SltDes5 from Spodoptera litura as set forth in SEQ ID NO: 87 or a desaturase having at least 60% homology to the Lbo_PPTQ desaturase from Lobesia botrana as set forth in SEQ ID NO: 79 may be used.

If a fatty alcohol desaturated in position 9 is desired, a Δ9 desaturase having at least 60% homology to the Δ9 desaturase from Drosophila melanogaster as set forth in SEQ ID NO: 35, a Δ9 desaturase having at least 60% homology to the Δ9 desaturase from Saccharomyces cerevisiae as set forth in SEQ ID NO: 30, a Δ9 desaturase having at least 60% homology to the Δ9 desaturase from Yarrowia lipolytica as set forth in SEQ ID NO: 32, a Δ9 desaturase having at least 60% homology to the Δ9 desaturase from Dendrophilus punctatus as set forth in SEQ ID NO: 46 may be used, a desaturase having at least 60% homology to the SltDes5 from Spodoptera litura as set forth in SEQ ID NO: 87 or a desaturase having at least 60% homology to the Lbo_PPTQ desaturase from Lobesia botrana as set forth in SEQ ID NO: 79 may be used.

If a fatty alcohol desaturated in position 10 is desired, a Δ10 desaturase having at least 60% homology to the Δ1 0 desaturase from Grapholita molesta as set forth in SEQ ID NO: 48 or SEQ ID NO: 96 may be used.

Other useful desaturases are desaturases having at least 60% homology to the desaturase from Cydia pomonella as set forth in SEQ ID NO: 50, SEQ ID NO: 129, SEQ ID NO: 131, SEQ ID NO: 133, SEQ ID NO: 135, SEQ ID NO: 137, SEQ ID NO: 139, SEQ ID NO: 141, SEQ ID NO: 143 or SEQ ID NO: 145, at least 60% homology to the desaturase from Epiphyas postvittana as set forth in SEQ ID NO: 52, at least 60% homology to the desaturase from Spodoptera littoralis as set forth in SEQ ID NO: 54, at least 60% homology to the desaturase from Choristoneura parallela as set forth in SEQ ID NO: 56, at least 60% homology to the desaturase from Saccharomyces cerevisiae as set forth in SEQ ID NO: 30, at least 60% homology to the desaturase from Yarrowia lipolytica as set forth in SEQ ID NO: 32, at least 60% homology to the desaturase from Drosophila melanogaster as set forth in SEQ ID NO: 34, at least 60% homology to a desaturase from Amyelois transitella as set forth in SEQ ID NO: 38, at least 60% homology to the desaturase from Choristoneura rosaceana as set forth in SEQ ID NO: 40, at least 60% homology to the desaturase from Ostrinia nubilalis as set forth in SEQ ID NO: 42, at least 60% homology to the desaturase from Thaumetopoea pityocampa as set forth in SEQ ID NO: 44, at least 60% homology to the desaturase from Dendrophilus punctatus as set forth in SEQ ID NO: 46, at least 60% homology to the desaturase from Grapholita molesta as set forth in SEQ ID NO: 48 or SEQ ID NO: 96, at least 60% homology to the desaturase from Epiphyas postvittana as set forth in SEQ ID NO: 52, at least 60% homology to the desaturase from Spodoptera littoralis as set forth in SEQ ID NO: 54, at least 60% homology to a desaturase from Cydia pomonella as set forth in SEQ ID NO: 56 or SEQ ID NO: 50, SEQ ID NO: 129, SEQ ID NO: 131, SEQ ID NO: 133, SEQ ID NO: 135, SEQ ID NO: 137, SEQ ID NO: 139, SEQ ID NO: 141, SEQ ID NO: 143 or SEQ ID NO: 145, at least 60% homology to a desaturase from Lobesia botrana as set forth in SEQ ID NO: 79 or SEQ ID NO: 89, at least 60% homology to the desaturase from Spodoptera litura as set forth in SEQ ID NO: 87, at least 60% homology to the desaturase from Chilo suppressalis as set forth in SEQ ID NO: 91, or at least 60% homology to the desaturase of Pectinophora gossypiella as set forth in SEQ ID NO: 101, such as at least 65%, such as at least 70%, such as at least 75%, such as at least 80%, such as at least 81%, such as at least 82%, such as at least 83%, such as at least 84%, such as at least 85%, such as at least 86%, such as at least 87%, such as at least 88%, such as at least 89%, such as at least 90%, such as at least 91%, such as at least 92%, such as at least 93%, such as at least 94%, such as at least 95%, such as at least 96%, such as at least 97%, such as at least 98%, such as at least 99% homology to any of said desaturases.

The desaturase SltDes5 from Spodoptera litura as set forth in SEQ ID NO: 87, the desaturase Dmd9 from Drosophila melanogaster as set forth in SEQ ID NO: 35, or the Lbo_PPTQ desaturase from Lobesia botrana as set forth in SEQ ID NO: 79 may be used to obtain fatty alcohols desaturated in position 5 or 7.

The yeast cell of the present disclosure may thus express a desaturase selected from Sce_OLE1 (SEQ ID NO: 30), Yli_OLE1 (SEQ ID NO: 32), Dme_D9 (SEQ ID NO: 35), Atr_D11 (SEQ ID NO: 38), Cro_Z11 (SEQ ID NO: 40), Onu_11 (SEQ ID NO: 42), Tpi_D13 (SEQ ID NO: 44), Dpu_E9-14 (SEQ ID NO: 46), Gmo_CPRQ (SEQ ID NO: 48), Gmo_KPSQ (SEQ ID NO: 96), Cpo_CPRQ (SEQ ID NO: 50, SEQ ID NO: 129, SEQ ID NO: 131, SEQ ID NO: 133, SEQ ID NO: 135, SEQ ID NO: 137, SEQ ID NO: 139, SEQ ID NO: 141, SEQ ID NO: 143 or SEQ ID NO: 145), Epo_E11 (SEQ ID NO: 52), Sls_ZE11 (SEQ ID NO: 54), Cpa_E11 (SEQ ID NO: 56), Lbo_PTTQ (SEQ ID NO: 79), Slitdes5 (SEQ ID NO: 87), Lbo_KPSE (SEQ ID NO: 89), Csup_KPSE (SEQ ID NO: 91), Cpo_CPRQ (SEQ ID NO: 50, SEQ ID NO: 129, SEQ ID NO: 131, SEQ ID NO: 133, SEQ ID NO: 135, SEQ ID NO: 137, SEQ ID NO: 139, SEQ ID NO: 141, SEQ ID NO: 143 or SEQ ID NO: 145) or PGDes8 (SEQ ID NO: 101) or a functional variant thereof having at least 60% homology thereto, such as at least 65%, such as at least 70%, such as at least 75%, such as at least 80%, such as at least 81%, such as at least 82%, such as at least 83%, such as at least 84%, such as at least 85%, such as at least 86%, such as at least 87%, such as at least 88%, such as at least 89%, such as at least 90%, such as at least 91%, such as at least 92%, such as at least 93%, such as at least 94%, such as at least 95%, such as at least 96%, such as at least 97%, such as at least 98%, such as at least 99% homology thereto. In some embodiments, the desaturase is selected from the group consisting of Onu_11 (SEQ ID NO: 42), Tpi_D13 (SEQ ID NO: 44), Dpu_E9-14 (SEQ ID NO: 46), Gmo_CPRQ (SEQ ID NO: 48), Gmo_KPSQ (SEQ ID NO: 96), Cpo_CPRQ (SEQ ID NO: 50, SEQ ID NO: 129, SEQ ID NO: 131, SEQ ID NO: 133, SEQ ID NO: 135, SEQ ID NO: 137, SEQ ID NO: 139, SEQ ID NO: 141, SEQ ID NO: 143 or SEQ ID NO: 145), Epo_E11 (SEQ ID NO: 52), Sls_ZE11 (SEQ ID NO: 54), Cpa_E11 (SEQ ID NO: 56), Lbo_PTTQ (SEQ ID NO: 79), Slitdes5 (SEQ ID NO: 87), Lbo_KPSE (SEQ ID NO: 89), Csup_KPSE (SEQ ID NO: 91), Cpo_CPRQ (SEQ ID NO: 50, SEQ ID NO: 129, SEQ ID NO: 131, SEQ ID NO: 133, SEQ ID NO: 135, SEQ ID NO: 137, SEQ ID NO: 139, SEQ ID NO: 141, SEQ ID NO: 143 or SEQ ID NO: 145) and PGDes8 (SEQ ID NO: 101) or a functional variant thereof having at least 60% homology thereto

In one embodiment, the desaturase is Sce_OLE1 (SEQ ID NO: 30) or a functional variant thereof having at least 60% homology thereto, such as at least 65%, such as at least 70%, such as at least 75%, such as at least 80%, such as at least 81%, such as at least 82%, such as at least 83%, such as at least 84%, such as at least 85%, such as at least 86%, such as at least 87%, such as at least 88%, such as at least 89%, such as at least 90%, such as at least 91%, such as at least 92%, such as at least 93%, such as at least 94%, such as at least 95%, such as at least 96%, such as at least 97%, such as at least 98%, such as at least 99% homology thereto.

In another embodiment, the desaturase is Yli_OLE1 (SEQ ID NO: 32) or a functional variant thereof having at least 60% homology thereto, such as at least 65%, such as at least 70%, such as at least 75%, such as at least 80%, such as at least 81%, such as at least 82%, such as at least 83%, such as at least 84%, such as at least 85%, such as at least 86%, such as at least 87%, such as at least 88%, such as at least 89%, such as at least 90%, such as at least 91%, such as at least 92%, such as at least 93%, such as at least 94%, such as at least 95%, such as at least 96%, such as at least 97%, such as at least 98%, such as at least 99% homology thereto.

In another embodiment, the desaturase is Dme_D9 (SEQ ID NO: 35) or a functional variant thereof having at least 60% homology thereto, such as at least 65%, such as at least 70%, such as at least 75%, such as at least 80%, such as at least 81%, such as at least 82%, such as at least 83%, such as at least 84%, such as at least 85%, such as at least 86%, such as at least 87%, such as at least 88%, such as at least 89%, such as at least 90%, such as at least 91%, such as at least 92%, such as at least 93%, such as at least 94%, such as at least 95%, such as at least 96%, such as at least 97%, such as at least 98%, such as at least 99% homology thereto.

In another embodiment, the desaturase is Atr_D11 (SEQ ID NO: 38) or a functional variant thereof having at least 60% homology thereto, such as at least 65%, such as at least 70%, such as at least 75%, such as at least 80%, such as at least 81%, such as at least 82%, such as at least 83%, such as at least 84%, such as at least 85%, such as at least 86%, such as at least 87%, such as at least 88%, such as at least 89%, such as at least 90%, such as at least 91%, such as at least 92%, such as at least 93%, such as at least 94%, such as at least 95%, such as at least 96%, such as at least 97%, such as at least 98%, such as at least 99% homology thereto.

In another embodiment, the desaturase is Cro_Z11 (SEQ ID NO: 40) or a functional variant thereof having at least 60% homology thereto, such as at least 65%, such as at least 70%, such as at least 75%, such as at least 80%, such as at least 81%, such as at least 82%, such as at least 83%, such as at least 84%, such as at least 85%, such as at least 86%, such as at least 87%, such as at least 88%, such as at least 89%, such as at least 90%, such as at least 91%, such as at least 92%, such as at least 93%, such as at least 94%, such as at least 95%, such as at least 96%, such as at least 97%, such as at least 98%, such as at least 99% homology thereto.

In another embodiment, the desaturase is Onu_11 (SEQ ID NO: 42) or a functional variant thereof having at least 60% homology thereto, such as at least 65%, such as at least 70%, such as at least 75%, such as at least 80%, such as at least 81%, such as at least 82%, such as at least 83%, such as at least 84%, such as at least 85%, such as at least 86%, such as at least 87%, such as at least 88%, such as at least 89%, such as at least 90%, such as at least 91%, such as at least 92%, such as at least 93%, such as at least 94%, such as at least 95%, such as at least 96%, such as at least 97%, such as at least 98%, such as at least 99% homology thereto.

In another embodiment, the desaturase is Tpi_D13 (SEQ ID NO: 44) or a functional variant thereof having at least 60% homology thereto, such as at least 65%, such as at least 70%, such as at least 75%, such as at least 80%, such as at least 81%, such as at least 82%, such as at least 83%, such as at least 84%, such as at least 85%, such as at least 86%, such as at least 87%, such as at least 88%, such as at least 89%, such as at least 90%, such as at least 91%, such as at least 92%, such as at least 93%, such as at least 94%, such as at least 95%, such as at least 96%, such as at least 97%, such as at least 98%, such as at least 99% homology thereto.

In another embodiment, the desaturase is Dpu_E9-14 (SEQ ID NO: 46) or a functional variant thereof having at least 60% homology thereto, such as at least 65%, such as at least 70%, such as at least 75%, such as at least 80%, such as at least 81%, such as at least 82%, such as at least 83%, such as at least 84%, such as at least 85%, such as at least 86%, such as at least 87%, such as at least 88%, such as at least 89%, such as at least 90%, such as at least 91%, such as at least 92%, such as at least 93%, such as at least 94%, such as at least 95%, such as at least 96%, such as at least 97%, such as at least 98%, such as at least 99% homology thereto.

In another embodiment, the desaturase is Gmo_CPRQ (SEQ ID NO: 48) or a functional variant thereof having at least 60% homology thereto, such as at least 65%, such as at least 70%, such as at least 75%, such as at least 80%, such as at least 81%, such as at least 82%, such as at least 83%, such as at least 84%, such as at least 85%, such as at least 86%, such as at least 87%, such as at least 88%, such as at least 89%, such as at least 90%, such as at least 91%, such as at least 92%, such as at least 93%, such as at least 94%, such as at least 95%, such as at least 96%, such as at least 97%, such as at least 98%, such as at least 99% homology thereto.

In another embodiment, the desaturase is Gmo_KPSQ (SEQ ID NO: 96) or a functional variant thereof having at least 60% homology thereto, such as at least 65%, such as at least 70%, such as at least 75%, such as at least 80%, such as at least 81%, such as at least 82%, such as at least 83%, such as at least 84%, such as at least 85%, such as at least 86%, such as at least 87%, such as at least 88%, such as at least 89%, such as at least 90%, such as at least 91%, such as at least 92%, such as at least 93%, such as at least 94%, such as at least 95%, such as at least 96%, such as at least 97%, such as at least 98%, such as at least 99% homology thereto.

In another embodiment, the desaturase is Cpo_CPRQ (SEQ ID NO: 50, SEQ ID NO: 129, SEQ ID NO: 131, SEQ ID NO: 133, SEQ ID NO: 135, SEQ ID NO: 137, SEQ ID NO: 139, SEQ ID NO: 141, SEQ ID NO: 143 or SEQ ID NO: 145) or a functional variant thereof having at least 60% homology thereto, such as at least 65%, such as at least 70%, such as at least 75%, such as at least 80%, such as at least 81%, such as at least 82%, such as at least 83%, such as at least 84%, such as at least 85%, such as at least 86%, such as at least 87%, such as at least 88%, such as at least 89%, such as at least 90%, such as at least 91%, such as at least 92%, such as at least 93%, such as at least 94%, such as at least 95%, such as at least 96%, such as at least 97%, such as at least 98%, such as at least 99% homology thereto.

In another embodiment, the desaturase is Epo_E11 (SEQ ID NO: 52) or a functional variant thereof having at least 60% homology thereto, such as at least 65%, such as at least 70%, such as at least 75%, such as at least 80%, such as at least 81%, such as at least 82%, such as at least 83%, such as at least 84%, such as at least 85%, such as at least 86%, such as at least 87%, such as at least 88%, such as at least 89%, such as at least 90%, such as at least 91%, such as at least 92%, such as at least 93%, such as at least 94%, such as at least 95%, such as at least 96%, such as at least 97%, such as at least 98%, such as at least 99% homology thereto.

In another embodiment, the desaturase is Sls_ZE11 (SEQ ID NO: 54) or a functional variant thereof having at least 60% homology thereto, such as at least 65%, such as at least 70%, such as at least 75%, such as at least 80%, such as at least 81%, such as at least 82%, such as at least 83%, such as at least 84%, such as at least 85%, such as at least 86%, such as at least 87%, such as at least 88%, such as at least 89%, such as at least 90%, such as at least 91%, such as at least 92%, such as at least 93%, such as at least 94%, such as at least 95%, such as at least 96%, such as at least 97%, such as at least 98%, such as at least 99% homology thereto.

In another embodiment, the desaturase is Cpa_E11 (SEQ ID NO: 56) or a functional variant thereof having at least 60% homology thereto, such as at least 65%, such as at least 70%, such as at least 75%, such as at least 80%, such as at least 81%, such as at least 82%, such as at least 83%, such as at least 84%, such as at least 85%, such as at least 86%, such as at least 87%, such as at least 88%, such as at least 89%, such as at least 90%, such as at least 91%, such as at least 92%, such as at least 93%, such as at least 94%, such as at least 95%, such as at least 96%, such as at least 97%, such as at least 98%, such as at least 99% homology thereto.

In another embodiment, the desaturase is Lbo_PTTQ (SEQ ID NO: 79), or a functional variant thereof having at least 60% homology thereto, such as at least 65%, such as at least 70%, such as at least 75%, such as at least 80%, such as at least 81%, such as at least 82%, such as at least 83%, such as at least 84%, such as at least 85%, such as at least 86%, such as at least 87%, such as at least 88%, such as at least 89%, such as at least 90%, such as at least 91%, such as at least 92%, such as at least 93%, such as at least 94%, such as at least 95%, such as at least 96%, such as at least 97%, such as at least 98%, such as at least 99% homology thereto.

In another embodiment, the desaturase is Slitdes5 (SEQ ID NO: 87), or a functional variant thereof having at least 60% homology thereto, such as at least 65%, such as at least 70%, such as at least 75%, such as at least 80%, such as at least 81%, such as at least 82%, such as at least 83%, such as at least 84%, such as at least 85%, such as at least 86%, such as at least 87%, such as at least 88%, such as at least 89%, such as at least 90%, such as at least 91%, such as at least 92%, such as at least 93%, such as at least 94%, such as at least 95%, such as at least 96%, such as at least 97%, such as at least 98%, such as at least 99% homology thereto.

In another embodiment, the desaturase is Lbo_KPSE (SEQ ID NO: 89), or a functional variant thereof having at least 60% homology thereto, such as at least 65%, such as at least 70%, such as at least 75%, such as at least 80%, such as at least 81%, such as at least 82%, such as at least 83%, such as at least 84%, such as at least 85%, such as at least 86%, such as at least 87%, such as at least 88%, such as at least 89%, such as at least 90%, such as at least 91%, such as at least 92%, such as at least 93%, such as at least 94%, such as at least 95%, such as at least 96%, such as at least 97%, such as at least 98%, such as at least 99% homology thereto.

In another embodiment, the desaturase is Csup_KPSE (SEQ ID NO: 91), or a functional variant thereof having at least 60% homology thereto, such as at least 65%, such as at least 70%, such as at least 75%, such as at least 80%, such as at least 81%, such as at least 82%, such as at least 83%, such as at least 84%, such as at least 85%, such as at least 86%, such as at least 87%, such as at least 88%, such as at least 89%, such as at least 90%, such as at least 91%, such as at least 92%, such as at least 93%, such as at least 94%, such as at least 95%, such as at least 96%, such as at least 97%, such as at least 98%, such as at least 99% homology thereto.

In another embodiment, the desaturase is Cpo_CPRQ (SEQ ID NO: 50, SEQ ID NO: 129, SEQ ID NO: 131, SEQ ID NO: 133, SEQ ID NO: 135, SEQ ID NO: 137, SEQ ID NO: 139, SEQ ID NO: 141, SEQ ID NO: 143 or SEQ ID NO: 145), or a functional variant thereof having at least 60% homology thereto, such as at least 65%, such as at least 70%, such as at least 75%, such as at least 80%, such as at least 81%, such as at least 82%, such as at least 83%, such as at least 84%, such as at least 85%, such as at least 86%, such as at least 87%, such as at least 88%, such as at least 89%, such as at least 90%, such as at least 91%, such as at least 92%, such as at least 93%, such as at least 94%, such as at least 95%, such as at least 96%, such as at least 97%, such as at least 98%, such as at least 99% homology thereto.

In another embodiment, the desaturase is PGDes8 (SEQ ID NO: 101), or a functional variant thereof having at least 60% homology thereto, such as at least 65%, such as at least 70%, such as at least 75%, such as at least 80%, such as at least 81%, such as at least 82%, such as at least 83%, such as at least 84%, such as at least 85%, such as at least 86%, such as at least 87%, such as at least 88%, such as at least 89%, such as at least 90%, such as at least 91%, such as at least 92%, such as at least 93%, such as at least 94%, such as at least 95%, such as at least 96%, such as at least 97%, such as at least 98%, such as at least 99% homology thereto.

In some embodiments, the yeast cell expresses more than one desaturase, such as at least two desaturases, such as at least three desaturases, such as at least four desaturases, such as at least five desaturases, or more. In some embodiments, the more than one desaturase is two, three, four or five desaturases selected from Sce_OLE1 (SEQ ID NO: 30), Yli_OLE1 (SEQ ID NO: 32), Dme_D9 (SEQ ID NO: 35), Atr_D11 (SEQ ID NO: 38), Cro_Z11 (SEQ ID NO: 40), Onu_11 (SEQ ID NO: 42), Tpi_D13 (SEQ ID NO: 44), Dpu_E9-14 (SEQ ID NO: 46), Gmo_CPRQ (SEQ ID NO: 48), Gmo_KPSQ (SEQ ID NO: 96), Cpo_CPRQ (SEQ ID NO: 50, SEQ ID NO: 129, SEQ ID NO: 131, SEQ ID NO: 133, SEQ ID NO: 135, SEQ ID NO: 137, SEQ ID NO: 139, SEQ ID NO: 141, SEQ ID NO: 143 or SEQ ID NO: 145), Epo_E11 (SEQ ID NO: 52), Sls_ZE11 (SEQ ID NO: 54), Cpa_E11 (SEQ ID NO: 56), Lbo_PTTQ (SEQ ID NO: 79), Slitdes5 (SEQ ID NO: 87), Lbo_KPSE (SEQ ID NO: 89), Csup_KPSE (SEQ ID NO: 91), Cpo_CPRQ (SEQ ID NO: 50, SEQ ID NO: 129, SEQ ID NO: 131, SEQ ID NO: 133, SEQ ID NO: 135, SEQ ID NO: 137, SEQ ID NO: 139, SEQ ID NO: 141, SEQ ID NO: 143 or SEQ ID NO: 145) or PGDes8 (SEQ ID NO: 101), or a functional variant thereof having at least 60% homology thereto, such as at least 65%, such as at least 70%, such as at least 75%, such as at least 80%, such as at least 81%, such as at least 82%, such as at least 83%, such as at least 84%, such as at least 85%, such as at least 86%, such as at least 87%, such as at least 88%, such as at least 89%, such as at least 90%, such as at least 91%, such as at least 92%, such as at least 93%, such as at least 94%, such as at least 95%, such as at least 96%, such as at least 97%, such as at least 98%, such as at least 99% homology thereto.

The gene encoding the heterologous desaturase may be codon-optimised for any purpose for the given host cell, e.g. Yarrowia lipolytica, as is known in the art.

In one embodiment, the at least one heterologous desaturase is encoded by a nucleic acid having at least 60% homology to the nucleic acid as set forth in SEQ ID NO: 36 or SEQ ID NO: 37, such as at least 61% homology, such as at least 62% homology, such as at least 63% homology, such as at least 64% homology, such as at least 65% homology, such as at least 66% homology, such as at least 67% homology, such as at least 68% homology, such as at least 69% homology, such as at least 70% homology, such as at least 71% homology, such as at least 72%, such as at least 73%, such as at least 74%, such as at least 75%, such as at least 76%, such as at least 77%, such as at least 78%, such as at least 79%, such as at least 80%, such as at least 81%, such as at least 82%, such as at least 83%, such as at least 84%, such as at least 85%, such as at least 86%, such as at least 87%, such as at least 88%, such as at least 89%, such as at least 90%, such as at least 91%, such as at least 92%, such as at least 93%, such as at least 94%, such as at least 95%, such as at least 96%, such as at least 97%, such as at least 98%, such as at least 99% homology to SEQ ID NO: 36 or SEQ ID NO: 37.

In another embodiment, the at least one heterologous desaturase is encoded by a nucleic acid having at least 60% homology to the nucleic acid as set forth in SEQ ID NO: 39, such as at least 61% homology, such as at least 62% homology, such as at least 63% homology, such as at least 64% homology, such as at least 65% homology, such as at least 66% homology, such as at least 67% homology, such as at least 68% homology, such as at least 69% homology, such as at least 70% homology, such as at least 71% homology, such as at least 72%, such as at least 73%, such as at least 74%, such as at least 75%, such as at least 76%, such as at least 77%, such as at least 78%, such as at least 79%, such as at least 80%, such as at least 81%, such as at least 82%, such as at least 83%, such as at least 84%, such as at least 85%, such as at least 86%, such as at least 87%, such as at least 88%, such as at least 89%, such as at least 90%, such as at least 91%, such as at least 92%, such as at least 93%, such as at least 94%, such as at least 95%, such as at least 96%, such as at least 97%, such as at least 98%, such as at least 99%, such as 100% homology to the nucleic acid as set forth in SEQ ID NO: 39.

In another embodiment, the at least one heterologous desaturase is encoded by a nucleic acid having at least 60% homology to the nucleic acid encoding as set forth in SEQ ID NO: 55, such as at least 61% homology, such as at least 62% homology, such as at least 63% homology, such as at least 64% homology, such as at least 65% homology, such as at least 66% homology, such as at least 67% homology, such as at least 68% homology, such as at least 69% homology, such as at least 70% homology, such as at least 71% homology, such as at least 72%, such as at least 73%, such as at least 74%, such as at least 75%, such as at least 76%, such as at least 77%, such as at least 78%, such as at least 79%, such as at least 80%, such as at least 81%, such as at least 82%, such as at least 83%, such as at least 84%, such as at least 85%, such as at least 86%, such as at least 87%, such as at least 88%, such as at least 89%, such as at least 90%, such as at least 91%, such as at least 92%, such as at least 93%, such as at least 94%, such as at least 95%, such as at least 96%, such as at least 97%, such as at least 98%, such as at least 99%, such as 100% homology to the nucleic acid as set forth in SEQ ID NO: 55.

In another embodiment, the at least one heterologous desaturase is encoded by a nucleic acid having at least 60% homology to the nucleic acid encoding as set forth in SEQ ID NO: 41, such as at least 61% homology, such as at least 62% homology, such as at least 63% homology, such as at least 64% homology, such as at least 65% homology, such as at least 66% homology, such as at least 67% homology, such as at least 68% homology, such as at least 69% homology, such as at least 70% homology, such as at least 71% homology, such as at least 72%, such as at least 73%, such as at least 74%, such as at least 75%, such as at least 76%, such as at least 77%, such as at least 78%, such as at least 79%, such as at least 80%, such as at least 81%, such as at least 82%, such as at least 83%, such as at least 84%, such as at least 85%, such as at least 86%, such as at least 87%, such as at least 88%, such as at least 89%, such as at least 90%, such as at least 91%, such as at least 92%, such as at least 93%, such as at least 94%, such as at least 95%, such as at least 96%, such as at least 97%, such as at least 98%, such as at least 99%, such as 100% homology to the nucleic acid as set forth in SEQ ID NO: 41.

In another embodiment, the at least one heterologous desaturase is encoded by a nucleic acid having at least 60% homology to the nucleic acid encoding as set forth in SEQ ID NO: 43, such as at least 61% homology, such as at least 62% homology, such as at least 63% homology, such as at least 64% homology, such as at least 65% homology, such as at least 66% homology, such as at least 67% homology, such as at least 68% homology, such as at least 69% homology, such as at least 70% homology, such as at least 71% homology, such as at least 72%, such as at least 73%, such as at least 74%, such as at least 75%, such as at least 76%, such as at least 77%, such as at least 78%, such as at least 79%, such as at least 80%, such as at least 81%, such as at least 82%, such as at least 83%, such as at least 84%, such as at least 85%, such as at least 86%, such as at least 87%, such as at least 88%, such as at least 89%, such as at least 90%, such as at least 91%, such as at least 92%, such as at least 93%, such as at least 94%, such as at least 95%, such as at least 96%, such as at least 97%, such as at least 98%, such as at least 99%, such as 100% homology to the nucleic acid as set forth in SEQ ID NO: 43.

In another embodiment, the at least one heterologous desaturase is encoded by a nucleic acid having at least 60% homology to the nucleic acid encoding as set forth in SEQ ID NO: 34 or SEQ ID NO: 33, such as at least 61% homology, such as at least 62% homology, such as at least 63% homology, such as at least 64% homology, such as at least 65% homology, such as at least 66% homology, such as at least 67% homology, such as at least 68% homology, such as at least 69% homology, such as at least 70% homology, such as at least 71% homology, such as at least 72%, such as at least 73%, such as at least 74%, such as at least 75%, such as at least 76%, such as at least 77%, such as at least 78%, such as at least 79%, such as at least 80%, such as at least 81%, such as at least 82%, such as at least 83%, such as at least 84%, such as at least 85%, such as at least 86%, such as at least 87%, such as at least 88%, such as at least 89%, such as at least 90%, such as at least 91%, such as at least 92%, such as at least 93%, such as at least 94%, such as at least 95%, such as at least 96%, such as at least 97%, such as at least 98%, such as at least 99%, such as 100% homology to the nucleic acid as set forth in SEQ ID NO: 34 or SEQ ID NO: 33.

In another embodiment, the at least one heterologous desaturase is encoded by a nucleic acid having at least 60% homology to the nucleic acid encoding as set forth in SEQ ID NO: 29, such as at least 61% homology, such as at least 62% homology, such as at least 63% homology, such as at least 64% homology, such as at least 65% homology, such as at least 66% homology, such as at least 67% homology, such as at least 68% homology, such as at least 69% homology, such as at least 70% homology, such as at least 71% homology, such as at least 72%, such as at least 73%, such as at least 74%, such as at least 75%, such as at least 76%, such as at least 77%, such as at least 78%, such as at least 79%, such as at least 80%, such as at least 81%, such as at least 82%, such as at least 83%, such as at least 84%, such as at least 85%, such as at least 86%, such as at least 87%, such as at least 88%, such as at least 89%, such as at least 90%, such as at least 91%, such as at least 92%, such as at least 93%, such as at least 94%, such as at least 95%, such as at least 96%, such as at least 97%, such as at least 98%, such as at least 99%, such as 100% homology to the nucleic acid as set forth in SEQ ID NO: 29.

In another embodiment, the at least one heterologous desaturase is encoded by a nucleic acid having at least 60% homology to the nucleic acid encoding as set forth in SEQ ID NO: 31, such as at least 61% homology, such as at least 62% homology, such as at least 63% homology, such as at least 64% homology, such as at least 65% homology, such as at least 66% homology, such as at least 67% homology, such as at least 68% homology, such as at least 69% homology, such as at least 70% homology, such as at least 71% homology, such as at least 72%, such as at least 73%, such as at least 74%, such as at least 75%, such as at least 76%, such as at least 77%, such as at least 78%, such as at least 79%, such as at least 80%, such as at least 81%, such as at least 82%, such as at least 83%, such as at least 84%, such as at least 85%, such as at least 86%, such as at least 87%, such as at least 88%, such as at least 89%, such as at least 90%, such as at least 91%, such as at least 92%, such as at least 93%, such as at least 94%, such as at least 95%, such as at least 96%, such as at least 97%, such as at least 98%, such as at least 99%, such as 100% homology to the nucleic acid as set forth in SEQ ID NO: 31.

In another embodiment, the at least one heterologous desaturase is encoded by a nucleic acid having at least 60% homology to the nucleic acid encoding as set forth in SEQ ID NO: 45, such as at least 61% homology, such as at least 62% homology, such as at least 63% homology, such as at least 64% homology, such as at least 65% homology, such as at least 66% homology, such as at least 67% homology, such as at least 68% homology, such as at least 69% homology, such as at least 70% homology, such as at least 71% homology, such as at least 72%, such as at least 73%, such as at least 74%, such as at least 75%, such as at least 76%, such as at least 77%, such as at least 78%, such as at least 79%, such as at least 80%, such as at least 81%, such as at least 82%, such as at least 83%, such as at least 84%, such as at least 85%, such as at least 86%, such as at least 87%, such as at least 88%, such as at least 89%, such as at least 90%, such as at least 91%, such as at least 92%, such as at least 93%, such as at least 94%, such as at least 95%, such as at least 96%, such as at least 97%, such as at least 98%, such as at least 99%, such as 100% homology to the nucleic acid as set forth in SEQ ID NO: 45.

In another embodiment, the at least one heterologous desaturase is encoded by a nucleic acid having at least 60% homology to the nucleic acid encoding as set forth in SEQ ID NO: 47, such as at least 61% homology, such as at least 62% homology, such as at least 63% homology, such as at least 64% homology, such as at least 65% homology, such as at least 66% homology, such as at least 67% homology, such as at least 68% homology, such as at least 69% homology, such as at least 70% homology, such as at least 71% homology, such as at least 72%, such as at least 73%, such as at least 74%, such as at least 75%, such as at least 76%, such as at least 77%, such as at least 78%, such as at least 79%, such as at least 80%, such as at least 81%, such as at least 82%, such as at least 83%, such as at least 84%, such as at least 85%, such as at least 86%, such as at least 87%, such as at least 88%, such as at least 89%, such as at least 90%, such as at least 91%, such as at least 92%, such as at least 93%, such as at least 94%, such as at least 95%, such as at least 96%, such as at least 97%, such as at least 98%, such as at least 99%, such as 100% homology to the nucleic acid as set forth in SEQ ID NO: 47.

In another embodiment, the at least one heterologous desaturase is encoded by a nucleic acid having at least 60% homology to the nucleic acid encoding as set forth in SEQ ID NO: 49, such as at least 61% homology, such as at least 62% homology, such as at least 63% homology, such as at least 64% homology, such as at least 65% homology, such as at least 66% homology, such as at least 67% homology, such as at least 68% homology, such as at least 69% homology, such as at least 70% homology, such as at least 71% homology, such as at least 72%, such as at least 73%, such as at least 74%, such as at least 75%, such as at least 76%, such as at least 77%, such as at least 78%, such as at least 79%, such as at least 80%, such as at least 81%, such as at least 82%, such as at least 83%, such as at least 84%, such as at least 85%, such as at least 86%, such as at least 87%, such as at least 88%, such as at least 89%, such as at least 90%, such as at least 91%, such as at least 92%, such as at least 93%, such as at least 94%, such as at least 95%, such as at least 96%, such as at least 97%, such as at least 98%, such as at least 99%, such as 100% homology to the nucleic acid as set forth in SEQ ID NO: 49.

In another embodiment, the at least one heterologous desaturase is encoded by a nucleic acid having at least 60% homology to the nucleic acid encoding as set forth in SEQ ID NO: 51, such as at least 61% homology, such as at least 62% homology, such as at least 63% homology, such as at least 64% homology, such as at least 65% homology, such as at least 66% homology, such as at least 67% homology, such as at least 68% homology, such as at least 69% homology, such as at least 70% homology, such as at least 71% homology, such as at least 72%, such as at least 73%, such as at least 74%, such as at least 75%, such as at least 76%, such as at least 77%, such as at least 78%, such as at least 79%, such as at least 80%, such as at least 81%, such as at least 82%, such as at least 83%, such as at least 84%, such as at least 85%, such as at least 86%, such as at least 87%, such as at least 88%, such as at least 89%, such as at least 90%, such as at least 91%, such as at least 92%, such as at least 93%, such as at least 94%, such as at least 95%, such as at least 96%, such as at least 97%, such as at least 98%, such as at least 99%, such as 100% homology to the nucleic acid as set forth in SEQ ID NO: 51.

In another embodiment, the at least one heterologous desaturase is encoded by a nucleic acid having at least 60% homology to the nucleic acid encoding as set forth in SEQ ID NO: 53, such as at least 61% homology, such as at least 62% homology, such as at least 63% homology, such as at least 64% homology, such as at least 65% homology, such as at least 66% homology, such as at least 67% homology, such as at least 68% homology, such as at least 69% homology, such as at least 70% homology, such as at least 71% homology, such as at least 72%, such as at least 73%, such as at least 74%, such as at least 75%, such as at least 76%, such as at least 77%, such as at least 78%, such as at least 79%, such as at least 80%, such as at least 81%, such as at least 82%, such as at least 83%, such as at least 84%, such as at least 85%, such as at least 86%, such as at least 87%, such as at least 88%, such as at least 89%, such as at least 90%, such as at least 91%, such as at least 92%, such as at least 93%, such as at least 94%, such as at least 95%, such as at least 96%, such as at least 97%, such as at least 98%, such as at least 99%, such as 100% homology to the nucleic acid as set forth in SEQ ID NO: 53.

In another embodiment, the at least one heterologous desaturase is encoded by a nucleic acid having at least 60% homology to the nucleic acid encoding as set forth in SEQ ID NO: 78, such as at least 61% homology, such as at least 62% homology, such as at least 63% homology, such as at least 64% homology, such as at least 65% homology, such as at least 66% homology, such as at least 67% homology, such as at least 68% homology, such as at least 69% homology, such as at least 70% homology, such as at least 71% homology, such as at least 72%, such as at least 73%, such as at least 74%, such as at least 75%, such as at least 76%, such as at least 77%, such as at least 78%, such as at least 79%, such as at least 80%, such as at least 81%, such as at least 82%, such as at least 83%, such as at least 84%, such as at least 85%, such as at least 86%, such as at least 87%, such as at least 88%, such as at least 89%, such as at least 90%, such as at least 91%, such as at least 92%, such as at least 93%, such as at least 94%, such as at least 95%, such as at least 96%, such as at least 97%, such as at least 98%, such as at least 99%, such as 100% homology to the nucleic acid as set forth in SEQ ID NO: 78.

In another embodiment, the at least one heterologous desaturase is encoded by a nucleic acid having at least 60% homology to the nucleic acid encoding as set forth in SEQ ID NO: 86, such as at least 61% homology, such as at least 62% homology, such as at least 63% homology, such as at least 64% homology, such as at least 65% homology, such as at least 66% homology, such as at least 67% homology, such as at least 68% homology, such as at least 69% homology, such as at least 70% homology, such as at least 71% homology, such as at least 72%, such as at least 73%, such as at least 74%, such as at least 75%, such as at least 76%, such as at least 77%, such as at least 78%, such as at least 79%, such as at least 80%, such as at least 81%, such as at least 82%, such as at least 83%, such as at least 84%, such as at least 85%, such as at least 86%, such as at least 87%, such as at least 88%, such as at least 89%, such as at least 90%, such as at least 91%, such as at least 92%, such as at least 93%, such as at least 94%, such as at least 95%, such as at least 96%, such as at least 97%, such as at least 98%, such as at least 99%, such as 100% homology to the nucleic acid as set forth in SEQ ID NO: 86.

In another embodiment, the at least one heterologous desaturase is encoded by a nucleic acid having at least 60% homology to the nucleic acid encoding as set forth in SEQ ID NO: 88, such as at least 61% homology, such as at least 62% homology, such as at least 63% homology, such as at least 64% homology, such as at least 65% homology, such as at least 66% homology, such as at least 67% homology, such as at least 68% homology, such as at least 69% homology, such as at least 70% homology, such as at least 71% homology, such as at least 72%, such as at least 73%, such as at least 74%, such as at least 75%, such as at least 76%, such as at least 77%, such as at least 78%, such as at least 79%, such as at least 80%, such as at least 81%, such as at least 82%, such as at least 83%, such as at least 84%, such as at least 85%, such as at least 86%, such as at least 87%, such as at least 88%, such as at least 89%, such as at least 90%, such as at least 91%, such as at least 92%, such as at least 93%, such as at least 94%, such as at least 95%, such as at least 96%, such as at least 97%, such as at least 98%, such as at least 99%, such as 100% homology to the nucleic acid as set forth in SEQ ID NO: 88.

In another embodiment, the at least one heterologous desaturase is encoded by a nucleic acid having at least 60% homology to the nucleic acid encoding as set forth in SEQ ID NO: 90, such as at least 61% homology, such as at least 62% homology, such as at least 63% homology, such as at least 64% homology, such as at least 65% homology, such as at least 66% homology, such as at least 67% homology, such as at least 68% homology, such as at least 69% homology, such as at least 70% homology, such as at least 71% homology, such as at least 72%, such as at least 73%, such as at least 74%, such as at least 75%, such as at least 76%, such as at least 77%, such as at least 78%, such as at least 79%, such as at least 80%, such as at least 81%, such as at least 82%, such as at least 83%, such as at least 84%, such as at least 85%, such as at least 86%, such as at least 87%, such as at least 88%, such as at least 89%, such as at least 90%, such as at least 91%, such as at least 92%, such as at least 93%, such as at least 94%, such as at least 95%, such as at least 96%, such as at least 97%, such as at least 98%, such as at least 99%, such as 100% homology to the nucleic acid as set forth in SEQ ID NO: 90.

In another embodiment, the at least one heterologous desaturase is encoded by a nucleic acid having at least 60% homology to the nucleic acid encoding as set forth in SEQ ID NO: 98, such as at least 61% homology, such as at least 62% homology, such as at least 63% homology, such as at least 64% homology, such as at least 65% homology, such as at least 66% homology, such as at least 67% homology, such as at least 68% homology, such as at least 69% homology, such as at least 70% homology, such as at least 71% homology, such as at least 72%, such as at least 73%, such as at least 74%, such as at least 75%, such as at least 76%, such as at least 77%, such as at least 78%, such as at least 79%, such as at least 80%, such as at least 81%, such as at least 82%, such as at least 83%, such as at least 84%, such as at least 85%, such as at least 86%, such as at least 87%, such as at least 88%, such as at least 89%, such as at least 90%, such as at least 91%, such as at least 92%, such as at least 93%, such as at least 94%, such as at least 95%, such as at least 96%, such as at least 97%, such as at least 98%, such as at least 99%, such as 100% homology to the nucleic acid as set forth in SEQ ID NO: 98.

In another embodiment, the at least one heterologous desaturase is encoded by a nucleic acid having at least 60% homology to the nucleic acid encoding as set forth in SEQ ID NO: 100, such as at least 61% homology, such as at least 62% homology, such as at least 63% homology, such as at least 64% homology, such as at least 65% homology, such as at least 66% homology, such as at least 67% homology, such as at least 68% homology, such as at least 69% homology, such as at least 70% homology, such as at least 71% homology, such as at least 72%, such as at least 73%, such as at least 74%, such as at least 75%, such as at least 76%, such as at least 77%, such as at least 78%, such as at least 79%, such as at least 80%, such as at least 81%, such as at least 82%, such as at least 83%, such as at least 84%, such as at least 85%, such as at least 86%, such as at least 87%, such as at least 88%, such as at least 89%, such as at least 90%, such as at least 91%, such as at least 92%, such as at least 93%, such as at least 94%, such as at least 95%, such as at least 96%, such as at least 97%, such as at least 98%, such as at least 99%, such as 100% homology to the nucleic acid as set forth in SEQ ID NO: 100.

In another embodiment, the at least one heterologous desaturase is encoded by a nucleic acid having at least 60% homology to the nucleic acid encoding as set forth in SEQ ID NO: 55, such as at least 61% homology, such as at least 62% homology, such as at least 63% homology, such as at least 64% homology, such as at least 65% homology, such as at least 66% homology, such as at least 67% homology, such as at least 68% homology, such as at least 69% homology, such as at least 70% homology, such as at least 71% homology, such as at least 72%, such as at least 73%, such as at least 74%, such as at least 75%, such as at least 76%, such as at least 77%, such as at least 78%, such as at least 79%, such as at least 80%, such as at least 81%, such as at least 82%, such as at least 83%, such as at least 84%, such as at least 85%, such as at least 86%, such as at least 87%, such as at least 88%, such as at least 89%, such as at least 90%, such as at least 91%, such as at least 92%, such as at least 93%, such as at least 94%, such as at least 95%, such as at least 96%, such as at least 97%, such as at least 98%, such as at least 99%, such as 100% homology to the nucleic acid as set forth in SEQ ID NO: 55.

### Alcohol-Forming Fatty acyl-CoA Reductase (EC 1.2.1.84)

The terms ‘alcohol-forming fatty acyl-CoA reductase’, ‘fatty acyl-CoA reductase’ and ‘FAR’ will be used herein interchangeably. The term “heterologous FAR” refers to a FAR which is not naturally expressed by the yeast cell.

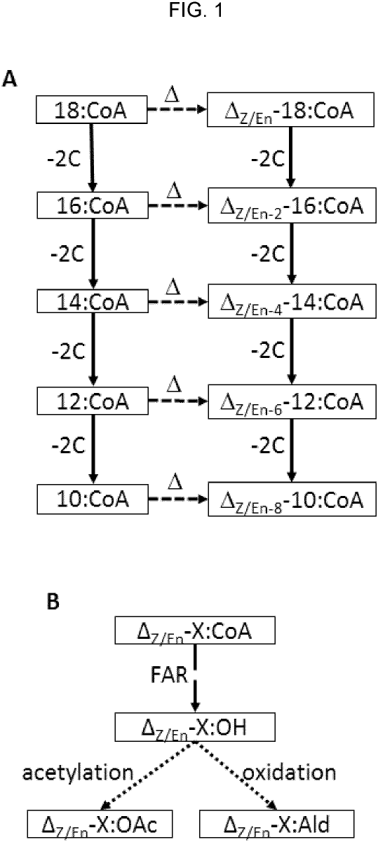

FARs catalyse the two-step reaction (FIG. 1): acyl-CoA + 2 NADPH <=> CoA + alcohol + 2 NADP(+) wherein in a first step, the fatty acyl-CoA is reduced to a fatty aldehyde, before the fatty aldehyde is further reduced into a fatty alcohol in a second step. The fatty acyl-CoA may be a desaturated fatty acyl-CoA.

Reductases reduce fatty acyl-CoAs into alcohols of the corresponding chain length. Thus, using the present yeast cell, a fatty acyl-CoA of carbon chain length X′ can be reduced to a desaturated fatty alcohol of carbon chain length X′.

The FARs capable of catalyzing such reaction are alcohol-forming fatty acyl-CoA reductases with an EC number 1.2.1.84. The yeast cell of the present disclosure has one or more mutations resulting in reduced activity of one or more native acyl-CoA oxidases, and expresses at least one heterologous desaturase capable of introducing at least one double bond in a fatty acyl-CoA as described above, and at least one heterologous fatty acyl-CoA reductase capable of converting at least part of said desaturated fatty acyl-CoA to a desaturated fatty alcohol.

The yeast cell of the present disclosure expressing a FAR is thus capable of converting at least part of the desaturated fatty acyl-CoA to a desaturated fatty alcohol.

In some embodiments, the at least one heterologous FAR is derived from an organism belonging to the Lepidoptera order. For example, the fatty acyl-CoA reductase is derived from an insect of the genus Helicoverpa, Agrotis, Heliothis or Bicyclus. In specific embodiments, the fatty acyl-CoA reductase is a fatty acyl-CoA reductase native to Helicoverpa armigera, Helicoverpa assulta, Agrotis segetum, Heliothis subflexa, Bicyclus anynana, or a functional variant thereof.

A heterologous fatty acyl-CoA reductase may be expressed from a nucleic acid introduced in the cell, e.g. on a vector such as a plasmid, or by genomic integration. The nucleic acid may be codon-optimised for any purpose as is known in the art for the specific yeast cell used.

In one embodiment, the at least one heterologous FAR is capable of converting a Δ3 fatty acyl-CoA into a Δ3 fatty alcohol. In another embodiment, the at least one heterologous FAR is capable of converting a Δ5 fatty acyl-CoA into a Δ5 fatty alcohol. In another embodiment, the at least one heterologous FAR is capable of converting a Δ6 fatty acyl-CoA into a Δ6 fatty alcohol. In another embodiment, the at least one heterologous FAR is capable of converting a Δ7 fatty acyl-CoA into a Δ7 fatty alcohol. In another embodiment, the at least one heterologous FAR is capable of converting a Δ8 fatty acyl-CoA into a Δ8 fatty alcohol. In another embodiment, the at least one heterologous FAR is capable of converting a Δ9 fatty acyl-CoA into a Δ9 fatty alcohol. In another embodiment, the at least one heterologous FAR is capable of converting a Δ10 fatty acyl-CoA into a Δ10 fatty alcohol. In another embodiment, the at least one heterologous FAR is capable of converting a Δ11 fatty acyl-CoA into a Δ11 fatty alcohol. In another embodiment, the at least one heterologous FAR is capable of converting a Δ12 fatty acyl-CoA into a Δ12 fatty alcohol. In another embodiment, the at least one heterologous FAR is capable of converting a Δ13 fatty acyl-CoA into a Δ13 fatty alcohol. In another embodiment, the at least one heterologous FAR is capable of converting a Δ14 fatty acyl-CoA into a Δ14 fatty alcohol. In another embodiment, the at least one heterologous FAR is capable of converting a Δ15 fatty acyl-CoA into a Δ15 fatty alcohol. In another embodiment, the at least one heterologous FAR is capable of converting a Δ16 fatty acyl-CoA into a Δ16 fatty alcohol. In another embodiment, the at least one heterologous FAR is capable of converting a Δ17 fatty acyl-CoA into a Δ17 fatty alcohol. In another embodiment, the at least one heterologous FAR is capable of converting a Δ18 fatty acyl-CoA into a Δ18 fatty alcohol. In another embodiment, the at least one heterologous FAR is capable of converting a Δ19 fatty acyl-CoA into a Δ19 fatty alcohol. In another embodiment, the at least one heterologous FAR is capable of converting a Δ20 fatty acyl-CoA into a Δ20 fatty alcohol. In another embodiment, the at least one heterologous FAR is capable of converting a Δ21 fatty acyl-CoA into a Δ21 fatty alcohol. In another embodiment, the at least one heterologous FAR is capable of converting a Δ22 fatty acyl-CoA into a Δ22 fatty alcohol.

In some embodiments, the FAR is selected from a FAR having at least 80% homology to the FAR derived from Helicoverpa armigera, as set forth in SEQ ID NO: 59, a FAR having at least 80% homology to the FAR from Helicoverpa assulta as set forth in SEQ ID NO: 75, a FAR having at least 80% homology to the FAR from Agrotis segetum as set forth in SEQ ID NO: 93, a FAR having at least 80% homology to the FAR from Heliothis subflexa as set forth in SEQ ID NO: 73 and a FAR having at least 80% homology to the FAR from Bicyclus anynana as set forth in SEQ ID NO: 77. Preferably, the FAR is selected from a FAR having at least 80% homology to the FAR derived from Helicoverpa armigera as set forth in SEQ ID NO: 59, a FAR having at least 80% homology to the FAR from Agrotis segetum as set forth in SEQ ID NO: 93, and a FAR having at least 80% homology to the FAR from Heliothis subflexa as set forth in SEQ ID NO: 73.

In one embodiment, the FAR is Har_FAR (SEQ ID NO: 59, FAR from Helicoverpa armigera) or a variant thereof having at least 75% homology to Har_FAR, such as at least 76%, such as at least 77%, such as at least 78%, such as at least 79%, such as at least 80%, such as at least 81%, such as at least 82%, such as at least 83%, such as at least 84%, such as at least 85%, such as at least 86%, such as at least 87%, such as at least 88%, such as at least 89%, such as at least 90%, such as at least 91%, such as at least 92%, such as at least 93%, such as at least 94%, such as at least 95%, such as at least 96%, such as at least 97%, such as at least 98%, such as at least 99%, such as 100% homology to Har_FAR (SEQ ID NO: 59).

In another embodiment, the FAR is Has_FAR (SEQ ID NO: 75, FAR from Helicoverpa assulta) or a variant thereof having at least 75% homology to Has_FAR, such as at least 76%, such as at least 77%, such as at least 78%, such as at least 79%, such as at least 80%, such as at least 81%, such as at least 82%, such as at least 83%, such as at least 84%, such as at least 85%, such as at least 86%, such as at least 87%, such as at least 88%, such as at least 89%, such as at least 90%, such as at least 91%, such as at least 92%, such as at least 93%, such as at least 94%, such as at least 95%, such as at least 96%, such as at least 97%, such as at least 98%, such as at least 99%, such as 100% homology to Has_FAR (SEQ ID NO: 75).

In another embodiment, the FAR is Hs_FAR (SEQ ID NO: 73, FAR from Heliothis subflexa) or a variant thereof having at least 75% homology to Hs_FAR, such as at least 76%, such as at least 77%, such as at least 78%, such as at least 79%, such as at least 80%, such as at least 81%, such as at least 82%, such as at least 83%, such as at least 84%, such as at least 85%, such as at least 86%, such as at least 87%, such as at least 88%, such as at least 89%, such as at least 90%, such as at least 91%, such as at least 92%, such as at least 93%, such as at least 94%, such as at least 95%, such as at least 96%, such as at least 97%, such as at least 98%, such as at least 99%, such as 100% homology to Hs_FAR (SEQ ID NO: 73).

In another embodiment, the FAR is Ban_FAR (SEQ ID NO: 77, FAR from Bicyclus anynana) or a variant thereof having at least 75% homology to Ban_FAR, such as at least 76%, such as at least 77%, such as at least 78%, such as at least 79%, such as at least 80%, such as at least 81%, such as at least 82%, such as at least 83%, such as at least 84%, such as at least 85%, such as at least 86%, such as at least 87%, such as at least 88%, such as at least 89%, such as at least 90%, such as at least 91%, such as at least 92%, such as at least 93%, such as at least 94%, such as at least 95%, such as at least 96%, such as at least 97%, such as at least 98%, such as at least 99%, such as 100% homology to Ban_FAR (SEQ ID NO: 77).

In another embodiment, the FAR is AseFAR (SEQ ID NO: 93) or a variant thereof having at least 75% homology to Ase_FAR, such as at least 76%, such as at least 77%, such as at least 78%, such as at least 79%, such as at least 80%, such as at least 81%, such as at least 82%, such as at least 83%, such as at least 84%, such as at least 85%, such as at least 86%, such as at least 87%, such as at least 88%, such as at least 89%, such as at least 90%, such as at least 91%, such as at least 92%, such as at least 93%, such as at least 94%, such as at least 95%, such as at least 96%, such as at least 97%, such as at least 98%, such as at least 99%, such as 100% homology to Ase_FAR (SEQ ID NO: 93).

In some embodiments, the heterologous FAR is encoded by a nucleic acid having at least 60% homology to SEQ ID NO: 57, SEQ ID NO: 58, SEQ ID NO: 74, SEQ ID NO: 72, SEQ ID NO: 92 or SEQ ID NO: 76, such as at least 61% homology, such as at least 62% homology, such as at least 63% homology, such as at least 64% homology, such as at least 65% homology, such as at least 66% homology, such as at least 67% homology, such as at least 68% homology, such as at least 69% homology, such as at least 70% homology, such as at least 71% homology, such as at least 72%, such as at least 73%, such as at least 74%, such as at least 75%, such as at least 76%, such as at least 77%, such as at least 78%, such as at least 79%, such as at least 80%, such as at least 81%, such as at least 82%, such as at least 83%, such as at least 84%, such as at least 85%, such as at least 86%, such as at least 87%, such as at least 88%, such as at least 89%, such as at least 90%, such as at least 91%, such as at least 92%, such as at least 93%, such as at least 94%, such as at least 95%, such as at least 96%, such as at least 97%, such as at least 98%, such as at least 99%, such as 100% homology thereto.

In one embodiment, the heterologous FAR is encoded by a nucleic acid having at least 60% homology to the nucleic acid encoding as set forth in SEQ ID NO: 58 or SEQ ID NO: 57, such as at least 61% homology, such as at least 62% homology, such as at least 63% homology, such as at least 64% homology, such as at least 65% homology, such as at least 66% homology, such as at least 67% homology, such as at least 68% homology, such as at least 69% homology, such as at least 70% homology, such as at least 71% homology, such as at least 72%, such as at least 73%, such as at least 74%, such as at least 75%, such as at least 76%, such as at least 77%, such as at least 78%, such as at least 79%, such as at least 80%, such as at least 81%, such as at least 82%, such as at least 83%, such as at least 84%, such as at least 85%, such as at least 86%, such as at least 87%, such as at least 88%, such as at least 89%, such as at least 90%, such as at least 91%, such as at least 92%, such as at least 93%, such as at least 94%, such as at least 95%, such as at least 96%, such as at least 97%, such as at least 98%, such as at least 99%, such as 100% homology to the nucleic acid as set forth in SEQ ID NO: 58 or SEQ ID NO: 57.

In another embodiment, the heterologous FAR is encoded by a nucleic acid having at least 60% homology to the nucleic acid encoding as set forth in SEQ ID NO: 74, such as at least 61% homology, such as at least 62% homology, such as at least 63% homology, such as at least 64% homology, such as at least 65% homology, such as at least 66% homology, such as at least 67% homology, such as at least 68% homology, such as at least 69% homology, such as at least 70% homology, such as at least 71% homology, such as at least 72%, such as at least 73%, such as at least 74%, such as at least 75%, such as at least 76%, such as at least 77%, such as at least 78%, such as at least 79%, such as at least 80%, such as at least 81%, such as at least 82%, such as at least 83%, such as at least 84%, such as at least 85%, such as at least 86%, such as at least 87%, such as at least 88%, such as at least 89%, such as at least 90%, such as at least 91%, such as at least 92%, such as at least 93%, such as at least 94%, such as at least 95%, such as at least 96%, such as at least 97%, such as at least 98%, such as at least 99%, such as 100% homology to the nucleic acid as set forth in SEQ ID NO: 74.

In another embodiment, the heterologous FAR is encoded by a nucleic acid having at least 60% homology to the nucleic acid encoding as set forth in SEQ ID NO: 72, such as at least 61% homology, such as at least 62% homology, such as at least 63% homology, such as at least 64% homology, such as at least 65% homology, such as at least 66% homology, such as at least 67% homology, such as at least 68% homology, such as at least 69% homology, such as at least 70% homology, such as at least 71% homology, such as at least 72%, such as at least 73%, such as at least 74%, such as at least 75%, such as at least 76%, such as at least 77%, such as at least 78%, such as at least 79%, such as at least 80%, such as at least 81%, such as at least 82%, such as at least 83%, such as at least 84%, such as at least 85%, such as at least 86%, such as at least 87%, such as at least 88%, such as at least 89%, such as at least 90%, such as at least 91%, such as at least 92%, such as at least 93%, such as at least 94%, such as at least 95%, such as at least 96%, such as at least 97%, such as at least 98%, such as at least 99%, such as 100% homology to the nucleic acid as set forth in SEQ ID NO: 72.

In another embodiment, the heterologous FAR is encoded by a nucleic acid having at least 60% homology to the nucleic acid encoding as set forth in SEQ ID NO: 76, such as at least 61% homology, such as at least 62% homology, such as at least 63% homology, such as at least 64% homology, such as at least 65% homology, such as at least 66% homology, such as at least 67% homology, such as at least 68% homology, such as at least 69% homology, such as at least 70% homology, such as at least 71% homology, such as at least 72%, such as at least 73%, such as at least 74%, such as at least 75%, such as at least 76%, such as at least 77%, such as at least 78%, such as at least 79%, such as at least 80%, such as at least 81%, such as at least 82%, such as at least 83%, such as at least 84%, such as at least 85%, such as at least 86%, such as at least 87%, such as at least 88%, such as at least 89%, such as at least 90%, such as at least 91%, such as at least 92%, such as at least 93%, such as at least 94%, such as at least 95%, such as at least 96%, such as at least 97%, such as at least 98%, such as at least 99%, such as 100% homology to the nucleic acid as set forth in SEQ ID NO: 76.

In another embodiment, the heterologous FAR is encoded by a nucleic acid having at least 60% homology to the nucleic acid encoding as set forth in SEQ ID NO: 91, such as at least 61% homology, such as at least 62% homology, such as at least 63% homology, such as at least 64% homology, such as at least 65% homology, such as at least 66% homology, such as at least 67% homology, such as at least 68% homology, such as at least 69% homology, such as at least 70% homology, such as at least 71% homology, such as at least 72%, such as at least 73%, such as at least 74%, such as at least 75%, such as at least 76%, such as at least 77%, such as at least 78%, such as at least 79%, such as at least 80%, such as at least 81%, such as at least 82%, such as at least 83%, such as at least 84%, such as at least 85%, such as at least 86%, such as at least 87%, such as at least 88%, such as at least 89%, such as at least 90%, such as at least 91%, such as at least 92%, such as at least 93%, such as at least 94%, such as at least 95%, such as at least 96%, such as at least 97%, such as at least 98%, such as at least 99%, such as 100% homology to the nucleic acid as set forth in SEQ ID NO: 91.

In some embodiments, expression of the desaturase and/or of the FAR can be induced, for example if the genes encoding these enzymes are under the control of inducible promoters, as is known in the art. The yeast cell is incubated under suitable conditions, such as in an appropriate medium and at an appropriate temperature as is known to a person of skill in the art. Suitable media supporting yeast growth are known in the art and include, but are not limited to: undefined, complete media such as YEPD (or YPD, Yeast Extract Peptone Dextrose); defined, complete medium such as SC (Synthetic Complete); defined, drop-out medium such as SD (Synthetic Dextrose) lacking one or more elements such as an amino acid or an inducer; or mineral medium, consisting of salts, vitamins and a carbon source, and others.

In addition to the one or more acyl-CoA oxidases as described herein, in one embodiment, the yeast cell of the disclosure expresses Sce_OLE1 (SEQ ID NO: 30) and Har_FAR (SEQ ID NO: 59). In another embodiment, the yeast cell expresses Yli_OLE1 (SEQ ID NO: 32) and Har_FAR (SEQ ID NO: 59). In another embodiment, the yeast cell expresses Sce_OLE1 (SEQ ID NO: 30) and Has_FAR (SEQ ID NO: 75). In another embodiment, the yeast cell expresses Yli_OLE1 (SEQ ID NO: 32) and Has_FAR (SEQ ID NO: 75). In another embodiment, the yeast cell expresses Sce_OLE1 (SEQ ID NO: 30) and Hs_FAR (SEQ ID NO: 73). In another embodiment, the yeast cell expresses Yli_OLE1 (SEQ ID NO: 32) and Hs_FAR (SEQ ID NO: 75). In another embodiment, the yeast cell expresses Sce_OLE1 (SEQ ID NO: 30) and Ban_FAR (SEQ ID NO: 77). In another embodiment, the yeast cell expresses Yli_OLE1 (SEQ ID NO: 32) and Ban_FAR (SEQ ID NO: 77). In another embodiment, the yeast cell expresses Sce_OLE1 (SEQ ID NO: 30) and Ase_FAR (SEQ ID NO: 93). In another embodiment, the yeast cell expresses Yli_OLE1 (SEQ ID NO: 32) and Ase_FAR (SEQ ID NO: 93). In addition, the yeast cell may express an acetyltransferase such as Sce_ATF1 as set forth in SEQ ID NO: 61 or a functional variant thereof having at least 60% homology thereto. Such yeast cells may be particularly useful for the production of Δ_{Z5}-C12 fatty alcohols, acetates or aldehydes, Δ_{Z7}-C14 fatty alcohols, acetates or aldehydes, Δ_{Z7}-C16 fatty alcohols acetates or aldehydes, or Δ_{Z9}-C16 fatty alcohols acetates or aldehydes, and derivatives thereof.

In addition to the one or more acyl-CoA oxidases as described herein, in one embodiment, the yeast cell of the disclosure expresses Dme_D9 (SEQ ID NO: 35) and Har_FAR (SEQ ID NO: 59). In another embodiment, the yeast cell expresses Dme_D9 (SEQ ID NO: 35) and Has_FAR (SEQ ID NO: 75). In another embodiment, the yeast cell expresses Dme_D9 (SEQ ID NO: 35) and Ban_FAR (SEQ ID NO: 77). In another embodiment, the yeast cell expresses Dme_D9 (SEQ ID NO: 35) and Hs_FAR (SEQ ID NO: 73). In another embodiment, the yeast cell expresses Dme_D9 (SEQ ID NO: 35) and Ase_FAR (SEQ ID NO: 93). In addition, the yeast cell may express an acetyltransferase such as Sce_ATF1 as set forth in SEQ ID NO: 61 or a functional variant thereof having at least 60% homology thereto. Such yeast cells may be particularly useful for the production of Δ_{Z7}-C12 fatty alcohols, acetates or aldehydes, and derivatives thereof.

In addition to the one or more acyl-CoA oxidases as described herein, in one embodiment, the yeast cell of the disclosure expresses Atr_D11 (SEQ ID NO: 38) and Har_FAR (SEQ ID NO: 59). In another embodiment, the yeast cell expresses Atr_D11 (SEQ ID NO: 38) and Has_FAR (SEQ ID NO: 75). In another embodiment, the yeast cell expresses Atr_D11 (SEQ ID NO: 38) and Ban_FAR (SEQ ID NO: 77). In another embodiment, the yeast cell expresses Atr_D11 (SEQ ID NO: 38) and Hs_FAR (SEQ ID NO: 73). In another embodiment, the yeast cell expresses Atr_D11 (SEQ ID NO: 38) and Ase_FAR (SEQ ID NO: 93). In addition, the yeast cell may express an acetyltransferase such as Sce_ATF1 as set forth in SEQ ID NO: 61 or a functional variant thereof having at least 60% homology thereto. Such yeast cells may be particularly useful for the production of Δ_{Z7}-C12 fatty alcohols, Δ_{Z9}-C14 fatty alcohols, acetates or aldehydes, or Δ_{Z7,Z11}-C16 fatty alcohols, acetates or aldehydes, and derivatives thereof.

In addition to the one or more acyl-CoA oxidases as described herein, in one embodiment, the yeast cell of the disclosure expresses Cro_Z11 (SEQ ID NO: 40) and Har_FAR (SEQ ID NO: 59). In another embodiment, the yeast cell expresses Onu_11 (SEQ ID NO: 42) and Har_FAR (SEQ ID NO: 59). In another embodiment, the yeast cell expresses Tpi_D13 (SEQ ID NO: 44) and Har_FAR (SEQ ID NO: 59). In another embodiment, the yeast cell expresses Dpu_E9-14 (SEQ ID NO: 46) and Har_FAR (SEQ ID NO: 59). In another embodiment, the yeast cell expresses Cro_Z11 (SEQ ID NO: 40) and Has_FAR (SEQ ID NO: 75). In another embodiment, the yeast cell expresses Onu_11 (SEQ ID NO: 42) and Has_FAR (SEQ ID NO: 75). In another embodiment, the yeast cell expresses Tpi_D13 (SEQ ID NO: 44) and Has_FAR (SEQ ID NO: 75). In another embodiment, the yeast cell expresses Dpu_E9-14 (SEQ ID NO: 46) and Has_FAR (SEQ ID NO: 75). In another embodiment, the yeast cell expresses Cro_Z11 (SEQ ID NO: 40) and Ban_FAR (SEQ ID NO: 77). In another embodiment, the yeast cell expresses Onu_11 (SEQ ID NO: 42) and Ban_FAR (SEQ ID NO: 77). In another embodiment, the yeast cell expresses Tpi_D13 (SEQ ID NO: 44) and Ban_FAR (SEQ ID NO: 77). In another embodiment, the yeast cell expresses Dpu_E9-14 (SEQ ID NO: 46) and Ban_FAR (SEQ ID NO: 77). In another embodiment, the yeast cell expresses Cro_Z11 (SEQ ID NO: 40) and Hs_FAR (SEQ ID NO: 73). In another embodiment, the yeast cell expresses Onu_11 (SEQ ID NO: 42) and Hs_FAR (SEQ ID NO: 73). In another embodiment, the yeast cell expresses Tpi_D13 (SEQ ID NO: 44) and Hs_FAR (SEQ ID NO: 73). In another embodiment, the yeast cell expresses Dpu_E9-14 (SEQ ID NO: 46) and Hs_FAR (SEQ ID NO: 73). In another embodiment, the yeast cell expresses Cro_Z11 (SEQ ID NO: 40) and Ase_FAR (SEQ ID NO: 93). In another embodiment, the yeast cell expresses Onu_11 (SEQ ID NO: 42) and Ase_FAR (SEQ ID NO: 93). In another embodiment, the yeast cell expresses Tpi_D13 (SEQ ID NO: 44) and Ase_FAR (SEQ ID NO: 93). In another embodiment, the yeast cell expresses Dpu_E9-14 (SEQ ID NO: 46) and Ase_FAR (SEQ ID NO: 93). In addition, the yeast cell may express an acetyltransferase such as Sce_ATF1 as set forth in SEQ ID NO: 61 or a functional variant thereof having at least 60% homology thereto. Such yeast cells may be particularly useful for the production of Δ_{E7,E9}-C12 fatty alcohols, acetates or aldehydes, and derivatives thereof.

In addition to the one or more acyl-CoA oxidases as described herein, in one embodiment, the yeast cell of the disclosure expresses Gmo_CPRQ (SEQ ID NO: 48) and Har_FAR (SEQ ID NO: 59). In another embodiment, the yeast cell expresses Gmo_CPRQ (SEQ ID NO: 48) and Has_FAR (SEQ ID NO: 75). In another embodiment, the yeast cell expresses Gmo_CPRQ (SEQ ID NO: 48) and Ban_FAR (SEQ ID NO: 77). In another embodiment, the yeast cell expresses Gmo_CPRQ (SEQ ID NO: 48) and Hs_FAR (SEQ ID NO: 73). In another embodiment, the yeast cell expresses Gmo_CPRQ (SEQ ID NO: 48) and Ase_FAR (SEQ ID NO: 93). In addition, the yeast cell may express an acetyltransferase such as Sce_ATF1 as set forth in SEQ ID NO: 61 or a functional variant thereof having at least 60% homology thereto. Such yeast cells may be particularly useful for the production of Δ_{Z8}-C12 and/or Δ_{E8}-C12 fatty alcohols, acetates or aldehydes, and derivatives thereof.

In addition to the one or more acyl-CoA oxidases as described herein, in one embodiment, the yeast cell of the disclosure expresses Gmo_KPSQ (SEQ ID NO: 96) and Har_FAR (SEQ ID NO: 59). In another embodiment, the yeast cell expresses Gmo_KPSQ (SEQ ID NO: 96) and Has_FAR (SEQ ID NO: 75). In another embodiment, the yeast cell expresses Gmo_KPSQ (SEQ ID NO: 96) and Ban_FAR (SEQ ID NO: 77). In another embodiment, the yeast cell expresses Gmo_KPSQ (SEQ ID NO: 96) and Hs_FAR (SEQ ID NO: 73). In another embodiment, the yeast cell expresses Gmo_KPSQ (SEQ ID NO: 96) and Ase_FAR (SEQ ID NO: 93). In addition, the yeast cell may express an acetyltransferase such as Sce_ATF1 as set forth in SEQ ID NO: 61 or a functional variant thereof having at least 60% homology thereto. Such yeast cells may be particularly useful for the production of Δ_{Z8}-C12 and/or Δ_{E8}-C12 fatty alcohols, acetates or aldehydes, and derivatives thereof.

In addition to the one or more acyl-CoA oxidases as described herein, in one embodiment, the yeast cell of the disclosure expresses Cpo_CPRQ (SEQ ID NO: 50, SEQ ID NO: 129, SEQ ID NO: 131, SEQ ID NO: 133, SEQ ID NO: 135, SEQ ID NO: 137, SEQ ID NO: 139, SEQ ID NO: 141, SEQ ID NO: 143 or SEQ ID NO: 145) and Har_FAR (SEQ ID NO: 59). In another embodiment, the yeast cell expresses Cpo_CPRQ (SEQ ID NO: 50, SEQ ID NO: 129, SEQ ID NO: 131, SEQ ID NO: 133, SEQ ID NO: 135, SEQ ID NO: 137, SEQ ID NO: 139, SEQ ID NO: 141, SEQ ID NO: 143 or SEQ ID NO: 145) and Has_FAR (SEQ ID NO: 75). In another embodiment, the yeast cell expresses Cpo_CPRQ (SEQ ID NO: 50, SEQ ID NO: 129, SEQ ID NO: 131, SEQ ID NO: 133, SEQ ID NO: 135, SEQ ID NO: 137, SEQ ID NO: 139, SEQ ID NO: 141, SEQ ID NO: 143 or SEQ ID NO: 145) and Ban_FAR (SEQ ID NO: 77). In another embodiment, the yeast cell expresses Cpo_CPRQ (SEQ ID NO: 50, SEQ ID NO: 129, SEQ ID NO: 131, SEQ ID NO: 133, SEQ ID NO: 135, SEQ ID NO: 137, SEQ ID NO: 139, SEQ ID NO: 141, SEQ ID NO: 143 or SEQ ID NO: 145) and Hs_FAR (SEQ ID NO: 73). In another embodiment, the yeast cell expresses Cpo_CPRQ (SEQ ID NO: 50, SEQ ID NO: 129, SEQ ID NO: 131, SEQ ID NO: 133, SEQ ID NO: 135, SEQ ID NO: 137, SEQ ID NO: 139, SEQ ID NO: 141, SEQ ID NO: 143 or SEQ ID NO: 145) and Ase_FAR (SEQ ID NO: 93). In addition, the yeast cell may express an acetyltransferase such as Sce_ATF1 as set forth in SEQ ID NO: 61 or a functional variant thereof having at least 60% homology thereto. Such yeast cells may be particularly useful for the production of Δ_{E8,E10}-C12 fatty alcohols, acetates or aldehydes, and derivatives thereof.

In addition to the one or more acyl-CoA oxidases as described herein, in one embodiment, the yeast cell of the disclosure expresses Cro_Z11 (SEQ ID NO: 40) and Har_FAR (SEQ ID NO: 59). In another embodiment, the yeast cell expresses Onu_11 (SEQ ID NO: 42) and Har_FAR (SEQ ID NO: 59). In another embodiment, the yeast cell expresses Tpi_D13 (SEQ ID NO: 44) and Har_FAR (SEQ ID NO: 59). In another embodiment, the yeast cell expresses Cro_Z11 (SEQ ID NO: 40) and Has_FAR (SEQ ID NO: 75). In another embodiment, the yeast cell expresses Onu_11 (SEQ ID NO: 42) and Has_FAR (SEQ ID NO: 75). In another embodiment, the yeast cell expresses Tpi_D13 (SEQ ID NO: 44) and Has_FAR (SEQ ID NO: 75). In another embodiment, the yeast cell expresses Cro_Z11 (SEQ ID NO: 40) and Ban_FAR (SEQ ID NO: 77). In another embodiment, the yeast cell expresses Onu_11 (SEQ ID NO: 42) and Ban_FAR (SEQ ID NO: 77). In another embodiment, the yeast cell expresses Tpi_D13 (SEQ ID NO: 44) and Ban_FAR (SEQ ID NO: 77). In another embodiment, the yeast cell expresses Cro_Z11 (SEQ ID NO: 40) and Hs_FAR (SEQ ID NO: 73). In another embodiment, the yeast cell expresses Onu_11 (SEQ ID NO: 42) and Hs_FAR (SEQ ID NO: 73). In another embodiment, the yeast cell expresses Tpi_D13 (SEQ ID NO: 44) and Hs_FAR (SEQ ID NO: 73). In another embodiment, the yeast cell expresses Cro_Z11 (SEQ ID NO: 40) and Ase_FAR (SEQ ID NO: 93). In another embodiment, the yeast cell expresses Onu_11 (SEQ ID NO: 42) and Ase_FAR (SEQ ID NO: 93). In another embodiment, the yeast cell expresses Tpi_D13 (SEQ ID NO: 44) and Ase_FAR (SEQ ID NO: 93). In addition, the yeast cell may express an acetyltransferase such as Sce_ATF1 as set forth in SEQ ID NO: 61 or a functional variant thereof having at least 60% homology thereto. Such yeast cells may be particularly useful for the production of Δ_{z9}-C12 or Δ_{Z11}-C14 fatty alcohols, acetates or aldehydes, and derivatives thereof.

In addition to the one or more acyl-CoA oxidases as described herein, in one embodiment, the yeast cell of the disclosure expresses Cro_Z11 (SEQ ID NO: 40) and Har_FAR (SEQ ID NO: 59). In another embodiment, the yeast cell expresses Onu_11 (SEQ ID NO: 42) and Har_FAR (SEQ ID NO: 59). In another embodiment, the yeast cell expresses Tpi_D13 (SEQ ID NO: 44) and Har_FAR (SEQ ID NO: 59). In another embodiment, the yeast cell expresses Epo_E11 (SEQ ID NO: 52) and Har_FAR (SEQ ID NO: 59). In another embodiment, the yeast cell expresses Sls_ZE11 (SEQ ID NO: 54) and Har_FAR (SEQ ID NO: 59). In another embodiment, the yeast cell expresses Cro_Z11 (SEQ ID NO: 40) and Has_FAR (SEQ ID NO: 75). In another embodiment, the yeast cell expresses Onu_11 (SEQ ID NO: 42) and Has_FAR (SEQ ID NO: 75). In another embodiment, the yeast cell expresses Tpi_D13 (SEQ ID NO: 44) and Has_FAR (SEQ ID NO: 75). In another embodiment, the yeast cell expresses Epo_E11 (SEQ ID NO: 52) and Has_FAR (SEQ ID NO: 75). In another embodiment, the yeast cell expresses Sls_ZE11 (SEQ ID NO: 54) and Has_FAR (SEQ ID NO: 75). In another embodiment, the yeast cell expresses Cro_Z11 (SEQ ID NO: 40) and Ban_FAR (SEQ ID NO: 77). In another embodiment, the yeast cell expresses Onu_11 (SEQ ID NO: 42) and Ban_FAR (SEQ ID NO: 77). In another embodiment, the yeast cell expresses Tpi_D13 (SEQ ID NO: 44) and Ban_FAR (SEQ ID NO: 77). In another embodiment, the yeast cell expresses Epo_E11 (SEQ ID NO: 52) and Ban_FAR (SEQ ID NO: 77). In another embodiment, the yeast cell expresses Sls_ZE11 (SEQ ID NO: 54) and Ban_FAR (SEQ ID NO: 77). In another embodiment, the yeast cell expresses Cro_Z11 (SEQ ID NO: 40) and Hs_FAR (SEQ ID NO: 73). In another embodiment, the yeast cell expresses Onu_11 (SEQ ID NO: 42) and Hs_FAR (SEQ ID NO: 73). In another embodiment, the yeast cell expresses Tpi_D13 (SEQ ID NO: 44) and Hs_FAR (SEQ ID NO: 73). In another embodiment, the yeast cell expresses Epo_E11 (SEQ ID NO: 52) and Hs_FAR (SEQ ID NO: 73). In another embodiment, the yeast cell expresses Sls_ZE11 (SEQ ID NO: 54) and Hs_FAR (SEQ ID NO: 73). In another embodiment, the yeast cell expresses Cro_Z11 (SEQ ID NO: 40) and Ase_FAR (SEQ ID NO: 93). In another embodiment, the yeast cell expresses Onu_11 (SEQ ID NO: 42) and Ase_FAR (SEQ ID NO: 93). In another embodiment, the yeast cell expresses Tpi_D13 (SEQ ID NO: 44) and Ase_FAR (SEQ ID NO: 93). In another embodiment, the yeast cell expresses Epo_E11 (SEQ ID NO: 52) and Ase_FAR (SEQ ID NO: 93). In another embodiment, the yeast cell expresses Sls_ZE11 (SEQ ID NO: 54) and Ase_FAR (SEQ ID NO: 93). In addition, the yeast cell may express an acetyltransferase such as Sce_ATF1 as set forth in SEQ ID NO: 61 or a functional variant thereof having at least 60% homology thereto. Such yeast cells may be particularly useful for the production of Δ_{E11}-C14 fatty alcohols, acetates or aldehydes, and derivatives thereof.

In addition to the one or more acyl-CoA oxidases as described herein, in one embodiment, the yeast cell of the disclosure expresses Atr_D11 (SEQ ID NO: 38) and/or Tpi_D13 (SEQ ID NO: 44), and Har_FAR (SEQ ID NO: 59). In another embodiment, the yeast cell expresses Atr_D11 (SEQ ID NO: 38) and/or Tpi_D13 (SEQ ID NO: 44), and Has_FAR (SEQ ID NO: 75). In another embodiment, the yeast cell expresses Atr_D11 (SEQ ID NO: 38) and/or Tpi_D13 (SEQ ID NO: 44), and Ban_FAR (SEQ ID NO: 77). In another embodiment, the yeast cell expresses Atr_D11 (SEQ ID NO: 38) and Hs_FAR (SEQ ID NO: 73). In another embodiment, the yeast cell expresses Atr_D11 (SEQ ID NO: 38) and Ase_FAR (SEQ ID NO: 93). In addition, the yeast cell may express an acetyltransferase such as Sce_ATF1 as set forth in SEQ ID NO: 61 or a functional variant thereof having at least 60% homology thereto. Such yeast cells may be particularly useful for the production of Δ_{Z11,Z13}-C16 fatty alcohols, acetates or aldehydes, and derivatives thereof.

In addition to the one or more acyl-CoA oxidases as described herein, in one embodiment, the yeast cell of the disclosure expresses Cpa_E11 (SEQ ID NO: 56), Lbo_PTTQ (SEQ ID NO: 79), Slitdes5 (SEQ ID NO: 87), Lbo_KPSE (SEQ ID NO: 89), Csup_KPSE (SEQ ID NO: 91), Cpo_CPRQ (SEQ ID NO: 50, SEQ ID NO: 129, SEQ ID NO: 131, SEQ ID NO: 133, SEQ ID NO: 135, SEQ ID NO: 137, SEQ ID NO: 139, SEQ ID NO: 141, SEQ ID NO: 143 or SEQ ID NO: 145) or PGDes8 (SEQ ID NO: 101), and Har_FAR (SEQ ID NO: 59). In another embodiment, the yeast cell expresses Cpa_E11 (SEQ ID NO: 56), Lbo_PTTQ (SEQ ID NO: 79), Slitdes5 (SEQ ID NO: 87), Lbo_KPSE (SEQ ID NO: 89), Csup_KPSE (SEQ ID NO: 91), Cpo_CPRQ (SEQ ID NO: 50, SEQ ID NO: 129, SEQ ID NO: 131, SEQ ID NO: 133, SEQ ID NO: 135, SEQ ID NO: 137, SEQ ID NO: 139, SEQ ID NO: 141, SEQ ID NO: 143 or SEQ ID NO: 145) or PGDes8 (SEQ ID NO: 101), and Has_FAR (SEQ ID NO: 75). In another embodiment, the yeast cell expresses Cpa_E11 (SEQ ID NO: 56), Lbo_PTTQ (SEQ ID NO: 79), Slitdes5 (SEQ ID NO: 87), Lbo_KPSE (SEQ ID NO: 89), Csup_KPSE (SEQ ID NO: 91), Cpo_CPRQ (SEQ ID NO: 50, SEQ ID NO: 129, SEQ ID NO: 131, SEQ ID NO: 133, SEQ ID NO: 135, SEQ ID NO: 137, SEQ ID NO: 139, SEQ ID NO: 141, SEQ ID NO: 143 or SEQ ID NO: 145) or PGDes8 (SEQ ID NO: 101), and Ban_FAR (SEQ ID NO: 77). In another embodiment, the yeast cell expresses Cpa_E11 (SEQ ID NO: 56), Lbo_PTTQ (SEQ ID NO: 79), Slitdes5 (SEQ ID NO: 87), Lbo_KPSE (SEQ ID NO: 89), Csup_KPSE (SEQ ID NO: 91), Cpo_CPRQ (SEQ ID NO: 50, SEQ ID NO: 129, SEQ ID NO: 131, SEQ ID NO: 133, SEQ ID NO: 135, SEQ ID NO: 137, SEQ ID NO: 139, SEQ ID NO: 141, SEQ ID NO: 143 or SEQ ID NO: 145) or PGDes8 (SEQ ID NO: 101) and Hs_FAR (SEQ ID NO: 73). In another embodiment, the yeast cell expresses Cpa_E11 (SEQ ID NO: 56), Lbo_PTTQ (SEQ ID NO: 79), Slitdes5 (SEQ ID NO: 87), Lbo_KPSE (SEQ ID NO: 89), Csup_KPSE (SEQ ID NO: 91), Cpo_CPRQ (SEQ ID NO: 50, SEQ ID NO: 129, SEQ ID NO: 131, SEQ ID NO: 133, SEQ ID NO: 135, SEQ ID NO: 137, SEQ ID NO: 139, SEQ ID NO: 141, SEQ ID NO: 143 or SEQ ID NO: 145) or PGDes8 (SEQ ID NO: 101) and Ase_FAR (SEQ ID NO: 93). In addition, the yeast cell may express an acetyltransferase such as Sce_ATF1 as set forth in SEQ ID NO: 61 or a functional variant thereof having at least 60% homology thereto.

The yeast cell may express several of the above listed desaturases, such as two, three, four, five or six desaturases as described herein above, in combination with any of Har_FAR, Hs_FAR, Har_FAR, Ban_FAR and Ase_FAR.

### Acetyltransferase (EC 2.3.1.84)

The term “acetyltransferase” refers to enzymes of EC number 2.3.1.84 and can also be termed “alcohol-O-acetyltransferase” or “AcT”. It acts on aliphatic alcohols, and catalyses the reaction:

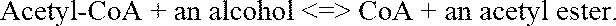

In addition to the modifications described herein above, the yeast cell of the present disclosure may also express or overexpress an acetyltransferase. The acetyltransferase may be a native acetyltransferase which the cell to be modified is already capable of expressing, or it may be a heterologous acetyltransferase. If the yeast cell expresses a native acetyltransferase, the yeast cell is preferably modified so that expression of the native acetyltransferase is increased. This can be done by methods known in the art, such as but not limited to introduction of additional copies of the nucleic acid encoding the acetyltransferase in the genome or on a vector, modification of the promoter to a constitutive promoter with a high expression level, or to an inducible promoter which upon induction leads to high expression levels. A heterologous acetyltransferase may be expressed from a nucleic acid introduced in the cell, e.g. on a vector such as a plasmid, or by genomic integration. The nucleic acid may be codon-optimised for any purpose as is known in the art for the specific yeast cell used.

If the yeast cell does not express a native acetyltransferase, a nucleic acid encoding a heterologous acetyltransferase may be introduced in the cell, either in a genomic location or on a vector, to enable expression of the acetyltransferase. Preferably, the acetyltransferase is expressed at a high level, e.g. by introducing multiple copies of the nucleic acid encoding the acetyltransferase, or by taking advantage of a constitutive promoter with a high expression level, or of an inducible promoter which upon induction leads to high expression levels.

The term “overexpress” thus refers to the overexpression of an acetyltransferase in a yeast cell when compared to a yeast cell which has not been modified to overexpress the acetyltransferase, i.e. the parent strain.

In some embodiments, the acetyltransferase is the AcT of SEQ ID NO: 61 (Atf1, the S. cerevisiae AcT) or a variant thereof having at least 75% homology to Sc_Atf1, such as at least 76%, such as at least 77%, such as at least 78%, such as at least 79%, such as at least 80%, such as at least 81%, such as at least 82%, such as at least 83%, such as at least 84%, such as at least 85%, such as at least 86%, such as at least 87%, such as at least 88%, such as at least 89%, such as at least 90%, such as at least 91%, such as at least 92%, such as at least 93%, such as at least 94%, such as at least 95%, such as at least 96%, such as at least 97%, such as at least 98%, such as at least 99%, such as 100% homology to SEQ ID NO: 61.

The acetyltransferase may be encoded by a nucleic acid having at least 60% homology to the nucleic acid as set forth in SEQ ID NO: 60, such as at least 61% homology, such as at least 62% homology, such as at least 63% homology, such as at least 64% homology, such as at least 65% homology, such as at least 66% homology, such as at least 67% homology, such as at least 68% homology, such as at least 69% homology, such as at least 70% homology, such as at least 71% homology, such as at least 72%, such as at least 73%, such as at least 74%, such as at least 75%, such as at least 76%, such as at least 77%, such as at least 78%, such as at least 79%, such as at least 80%, such as at least 81%, such as at least 82%, such as at least 83%, such as at least 84%, such as at least 85%, such as at least 86%, such as at least 87%, such as at least 88%, such as at least 89%, such as at least 90%, such as at least 91%, such as at least 92%, such as at least 93%, such as at least 94%, such as at least 95%, such as at least 96%, such as at least 97%, such as at least 98%, such as at least 99%, such as 100% homology to the nucleic acid as set forth in SEQ ID NO: 60.

In other embodiments, the conversion of at least part of the desaturated fatty alcohols to desaturated fatty acyl acetates is done chemically, as is known to the skilled person. For example, acetyl chloride can be added to the fatty alcohol and the mixture incubated at room temperature after mixing.

Yeast cells of the present disclosure expressing an acetyltransferase are thus capable of converting at least part of the desaturated fatty alcohols, in particular of carbon chain length X′, to desaturated fatty acyl acetates, in particular of carbon chain length X′.

### Dehydrogenase and Fatty Alcohol Oxidases

In some embodiments, it may be desirable to convert a desaturated fatty alcohol to the corresponding fatty aldehyde. This can be performed enzymatically by alcohol dehydrogenases (also termed ‘dehydrogenases’ herein) or fatty alcohol oxidases. The skilled person will know how to carry out enzymatic oxidation. For example, enzymatic oxidation can be carried out by contacting purified enzymes, cell extracts or whole cells, with the fatty alcohol.

Accordingly, the yeast cell may, in addition to the modifications described herein above, also express a fatty alcohol oxidase or a dehydrogenase capable of converting at least part of the produced desaturated fatty alcohols to the corresponding desaturated fatty aldehydes.

Fatty alcohol oxidases (EC 1.1.3.20) catalyse the reaction:

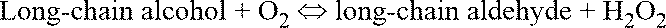

A suitable fatty alcohol oxidase is Fao1p from Yarrowia lipolytica (GenBank accession nr: XP_500864). Thus in some embodiments, the yeast cell, in addition to the modifications described herein above, also expresses a fatty alcohol oxidase such as Fao1p.

Alcohol dehydrogenases (EC 1.1.1.2) catalyse the reaction:

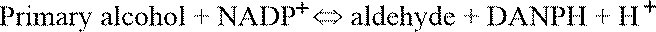

Examples of suitable alcohol dehydrogenases are Aah1p (GenBank accession nr: XM_503282) and Adh3p (GenBank accession nr: XM_500127) from Y. lipolytica. Thus in some embodiments, the yeast cell, in addition to the modifications described herein above, also expresses an alcohol dehydrogenase such as Aah1p or Adh3p.

The fatty alcohol oxidase or the dehydrogenase can be expressed directly in the cell, as is known in the art. Alternatively, the enzymes can be expressed separately, e.g. in another host cell, and used for oxidizing fatty alcohol into an aldehyde form in vitro.

Yeast cells of the present disclosure expressing a fatty alcohol oxidase and/or an alcohol dehydrogenase are thus capable of converting at least part of the desaturated fatty alcohols to the corresponding desaturated fatty aldehydes.

### Aldehyde-Forming Fatty acyl-CoA Reductase (FAR′) (EC 1.2.1.50)

The term “aldehyde-forming fatty acyl-CoA reductase” is herein interchangeably used with “aldehyde-forming FAR” or “FAR’”. Such enzymes catalyse conversion of a desaturated fatty acyl-CoA to the corresponding fatty aldehyde can catalyse a reduction reaction, where the fatty acyl-CoA is reduced to a fatty aldehyde. Such enzymes are aldehyde-forming fatty acyl-CoA reductases, herein also referred to as FAR’ or “aldehyde-forming FAR’”, with an EC number 1.2.1.50. They catalyse the following reaction:

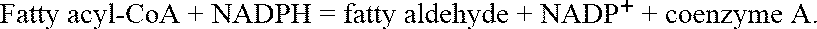

The yeast cells disclosed therein may, in addition to the other modifications described herein, also express an aldehyde-forming fatty acyl-CoA reductase, which is capable of catalysing conversion of a fatty acyl-CoA having a carbon chain length X or of a shortened fatty acyl-CoA having a carbon chain length X′ directly to the corresponding fatty aldehyde of carbon chain length X or X′, respectively.

In some embodiments, expression of the aldehyde-forming FAR′ can be induced, for example if the gene encoding this enzyme is under the control of inducible promoters, as is known in the art. The yeast cell is incubated under suitable conditions, such as in an appropriate medium and at an appropriate temperature as is known to a person of skill in the art. Suitable media supporting yeast growth are known in the art and include, but are not limited to: undefined, complete media such as YEPD (or YPD, Yeast Extract Peptone Dextrose), defined, complete medium such as SC (Synthetic Complete), or defined, drop-out medium such as SD (Synthetic Dextrose) lacking one or more elements such as an amino acid or an inducer.

Thus, the following aldehydes can be obtained from a desaturated fatty acyl-CoA of carbon chain length X after the carbon chain has been shortened to obtain a fatty acyl-CoA of carbon chain length X′:
(Z)-Δ5 desaturated fatty aldehydes having a carbon chain length of X′;
(E)-Δ5 desaturated fatty aldehydes having a carbon chain length of X′;
(Z)-Δ6 desaturated fatty aldehydes having a carbon chain length of X′;
(E)-Δ6 desaturated fatty aldehydes having a carbon chain length of X′;
(Z)-Δ7 desaturated fatty aldehydes having a carbon chain length of X′;
(E)-Δ7 desaturated fatty aldehydes having a carbon chain length of X′;
(Z)-Δ8 desaturated fatty aldehydes having a carbon chain length of X′;
(E)-Δ8 desaturated fatty aldehydes having a carbon chain length of X′;
(Z)-Δ9 desaturated fatty aldehydes having a carbon chain length of X′;
(E)-Δ9 desaturated fatty aldehydes having a carbon chain length of X′;
(Z)-Δ10 desaturated fatty aldehydes having a carbon chain length of X′;
(E)-Δ10 desaturated fatty aldehydes having a carbon chain length of X′;
(Z)-Δ11 desaturated fatty aldehydes having a carbon chain length of X′;
(E)-Δ11 desaturated fatty aldehydes having a carbon chain length of X′;
(Z)-Δ12 desaturated fatty aldehydes having a carbon chain length of X′;
(E)-Δ12 desaturated fatty aldehydes having a carbon chain length of X′;
(Z)-Δ13 desaturated fatty aldehydes having a carbon chain length of X′; and
(E)-Δ13 desaturated fatty aldehydes having a carbon chain length of X′.

The desaturated fatty aldehydes produced by the present yeast cell may be desaturated in more than one position. The desaturated fatty aldehydes may be desaturated in at least two positions, such as at least three positions, such as four positions.

### Other Useful Modifications

In order to further increase production of desaturated fatty alcohols, it may be beneficial to mutate one or more genes encoding a lipase so that the corresponding lipase has partial or total loss of activity. Accordingly, in some embodiments, the yeast cell may be as described herein and additionally carry one or more mutations resulting in total or partial loss of activity of one or more lipases.

It is known in the art that there are numerous genes encoding lipases. Their expression and/or activity may be a function of the medium in which the yeast cell is cultivated. Accordingly, the choice of medium may help choosing which lipase gene should be deleted or mutated in order for the corresponding lipase to have reduced or total loss of activity in said medium.

Several lipases may be active in one medium at the same time. Thus, in some embodiments, the yeast cell has several mutations, resulting in total or partial loss of activity of several lipases. In order to limit degradation of fatty acyl acetate, in some embodiments the yeast cell has several mutations resulting in total or partial loss of activity of all the lipases known to be or suspected of being active in a given medium.

By way of example Y. lipolytica contains at least two intracellular lipases Tgl3p (CAG81136) and Tgl4p (CAG78037). Accordingly, total or partial loss of activity of one or both lipases may be considered in embodiments where the yeast cell is a Yarrowia lipolytica cell.

In order for the yeast cell to produce desaturated fatty alcohols and desaturated fatty acyl acetates as described herein, the yeast cell needs to be provided with fatty acyl-CoAs as a substrate. The substrate has a carbon chain length of X, and can be shortened to a fatty acyl-CoA of carbon chain length X′.

Such fatty acyl-CoA can either be provided in the medium in which the yeast cell is incubated, or the yeast cell may be naturally able to produce such fatty acyl-CoA, or the yeast cell may be engineered in order to produce or to increase production of such fatty acyl-CoA. Preferaby, the yeast cell is provided with or is capable of producing myristoyl-CoA.

In some embodiments, the yeast cell is not naturally capable of producing a fatty acyl-CoA having the desired carbon chain length (X). The yeast cell may in this case be engineered as is known in the art, for example by the introduction of a heterologous thioesterase. Thus in some embodiments, a nucleic acid encoding a thioesterase is introduced in the yeast cell, on a vector or by genomic integration. The thioesterase gene may be under the control of an inducible promoter, or under the control of a constitutive promoter. The nucleic acid encoding a thioesterase may be codon-optimised for the yeast cell, as is known in the art. In particular, the nucleic acid may be codon-optimised for a Yarrowia cell, such as a Yarrowia lipolytica cell.

In some embodiments, the thioesterase is derived from an organism selected from Cuphea palustris, Cuphea hookeriana, Cinnamomum camphora, or from Escherichia coli. In preferred embodiments, the thioesterase is derived from Escherichia coli or Cinnamomum camphora. Examples of suitable thioesterases are a thioesterase derived from Cuphea palustris (GenBank accession number: AAC49180), a thioesterase derived from Cuphea hookerian (GenBank accession number: AAC72881), a thioesterase derived from Cinnamomum camphora (GenBank accession number: Q39473), and the thioesterase derived from Escherichia coli (GenBank accession number: NP_415027).

In some embodiments, availability of fatty acids having a desired carbon chain length may be increased or further increased. For instance, the yeast cell may be further modified by one or more mutations yielding a modified enzyme having a changed product profile such as reduced capability to degrade fatty acyl-CoAs or increased capability to synthesise fatty acyl-CoAs compared to an unmodified enzyme. For example, the fatty acid synthase complex may be engineered so that formation of C14-fatty acyl-CoA is increased. The fatty acid synthase complex (EC 2.3.1.86) consists of two subunits, Fas1 (beta subunit) and Fas2 (alpha subunit). The alpha subunit comprises a ketoacyl synthase domain (a “binding pocket”) which is hypothesized to be involved in determining the length of the synthesized fatty acids. In Yarrowia lipolityca, the native (wild-type) FAS2 is as set forth in SEQ ID NO: 71; the native FAS1 is as set forth in SEQ ID NO: 69.

Accordingly, in order to direct the metabolic flux towards production of desaturated fatty alcohols, acetates or aldehydes having a chain length of 14 C, the yeast cell may further express a fatty acyl synthase variant having a modified ketone synthase domain. Without being bound by theory, it is hypothesized that the modified ketone synthase domain results in a modified binding pocket, which thus more readily accommodates medium length substrates such as C14 substrates, thereby producing a higher proportion of C14 products.

In one embodiment, the yeast cell is a Yarrowia lipolytica cell as described herein, wherein the cell further expresses a modified fatty acid synthase complex. In one embodiment, the fatty acid synthase complex is modified by mutating the gene encoding the alpha subunit of the complex. In some embodiments, the mutation is in the gene encoding FAS2. The mutation may result in modification of one or more of residue 1220 (11220), residue 1217 (M1217) or residue 1226 (M1226) of SEQ ID NO: 71, resulting in a variant FAS2. The skilled person will know how to design such mutations.

Preferably, the mutation results in an I1220F variant, an I1220W variant, an I1220Y variant or an I1220H variant. In a specific embodiment, the mutation results in an I1220F variant. In some embodiments, the mutation results in an M1217F variant, an M1217W variant, an M1217Y variant or an M1217H variant. In other embodiments, the mutation results in an M1226F variant, an M1226W variant, an M1226Y variant or an M1226H variant. Yeast cells with more than one of the above mutations are also contemplated, such as two mutations or three mutations at residue I1220, M1217 or M1226.

Other modifications of yeast cells which may be useful in the cells and methods disclosed herein are described in WO 2018/109163. Such modifications include, but are not limited to, mutations resulting in partial or total loss of activity of one or more of: HFD1, HFD2, HFD3, HFD4, FAO1, POX1, POX2, POX3, POX4, POX5, POX6, GPAT and optionally PEX10.

In some embodiments, the yeast cell is thus further engineered by inactivation, e.g. deletion, of:
HFD1, HFD2, HFD3, HFD4, FAO1
HFD1, HFD2, HFD3, HFD4, FAO1, POX5, POX6
HFD1, HFD2, HFD3, HFD4, FAO1, POX1, POX4, POX5, POX6
HFD1, HFD2, HFD3, HFD4, FAO1, POX1, POX2, POX3, POX4, POX5, POX6

The yeast cell may further comprise a mutation such as a deletion of PEX10 resulting in loss of activity of Pex10, and/or of GPAT resulting in loss of activity of GPAT. Preferably, the yeast cell is a Yarrowia lipolytica cell.

### Specific Yeast Cells

Some useful yeast cells for use in the methods disclosed herein are as follows.

In some embodiments, the yeast cell is a Yarrowia lipolytica cell or a Saccharomyces cerevisiae cell.

Yeast cells useful for the production of a desaturated fatty alcohol or derivative thereof via the shortening of a fatty acyl-CoA to a shortened fatty acyl-CoA having a carbon chain length X′ = 16, such as a Z7-16 fatty acyl-CoA, include cells expressing:
At least one FAR, preferably Har_FAR (SEQ ID NO: 59) and/or AseFAR (SEQ ID NO: 93); and
At least one desaturase, preferably Lbo_PPTQ (SEQ ID NO: 79); and
one or more of: Ani_POX (SEQ ID NO: 20), Cma_POX (SEQ ID NO: 22), Pur_POX (SEQ ID NO: 26), Rno_POX2 (SEQ ID NO: 28), Yli_POX2 (SEQ ID NO: 4), Lbo31670 (SEQ ID NO: 81), Lbo49554 (SEQ ID NO: 83) and/or Lbo49602 (SEQ ID NO: 85),
or functional variants of any of the above having at least 60% homology thereto, such as at least 65%, such as at least 70%, such as at least 75%, such as at least 80%, such as at least 81%, such as at least 82%, such as at least 83%, such as at least 84%, such as at least 85%, such as at least 86%, such as at least 87%, such as at least 88%, such as at least 89%, such as at least 90%, such as at least 91%, such as at least 92%, such as at least 93%, such as at least 94%, such as at least 95%, such as at least 96%, such as at least 97%, such as at least 98%, such as at least 99% homology thereto. Such yeast cells may in addition have reduced activity of said one ormore native acyl-CoA oxidases, such as one or more of Yli_POX1, Yli_POX2, Yli_POX3, Yli_POX4, Yli_POX5 or Yli_POX6, for example a deletion in any one of the genes encoding said native acyl-CoA oxidases. In some embodiments, the yeast cell has reduced activity of Yli_POX1, Yli_POX2, Yli_POX3, Yli_POX4 and Yli_POX5, for example via deletion of these genes.

Yeast cells useful for the production of a desaturated fatty alcohol or derivative thereof via the shortening of a fatty acyl-CoA to a shortened fatty acyl-CoA having a carbon chain length X′ = 12, such as a Z7-12 fatty acyl-CoA, include cells expressing:
At least one FAR, preferably Har_FAR (SEQ ID NO: 59); and
At least one desaturase, preferably Slitdes5 (SEQ ID NO: 87); and one or more of: Yli_POX3 (SEQ ID NO: 6), YliPOX5 (SEQ ID NO: 10), Cma_POX (SEQ ID NO: 22), Hsa_POX1-2 (SEQ ID NO: 24), Pur_POX (SEQ ID NO: 26), Rno_POX-2 (SEQ ID NO: 28), Lbo31670 (SEQ ID NO: 81), Lbo49554 (SEQ ID NO: 83) and/or Lbo49602 (SEQ ID NO: 85); or
At least one desaturase, preferably Dmd9 (SEQ ID NO: 35), and one or more of Cma_POX (SEQ ID NO: 22), Ani_POX (SEQ ID NO: 20), Hsa_POX1-2 (SEQ ID NO: 24), PurPOX (SEQ ID NO: 26), Rno_POX-2 (SEQ ID NO: 28), Lbo31670 (SEQ ID NO: 81), and/or Lbo49554 (SEQ ID NO: 83);
At least one desaturase, preferably Lbo_PPTQ (SEQ ID NO: 79), and one or more of Cma_POX (SEQ ID NO: 22), Hsa_POX1-2 (SEQ ID NO: 24), Pur_POX (SEQ ID NO: 26), Rno_POX-2 (SEQ ID NO: 28), Lbo31670 (SEQ ID NO: 81) or Lbo49554 (SEQ ID NO: 83),
or functional variants of any of the above having at least 60% homology thereto, such as at least 65%, such as at least 70%, such as at least 75%, such as at least 80%, such as at least 81%, such as at least 82%, such as at least 83%, such as at least 84%, such as at least 85%, such as at least 86%, such as at least 87%, such as at least 88%, such as at least 89%, such as at least 90%, such as at least 91%, such as at least 92%, such as at least 93%, such as at least 94%, such as at least 95%, such as at least 96%, such as at least 97%, such as at least 98%, such as at least 99% homology thereto. Such yeast cells may in addition have reduced activity of said one or more native acyl-CoA oxidases, such as one or more of Yli_POX1, Yli_POX2, Yli_POX3, Yli_POX4, Yli_POX5 or Yli_POX6, for example a deletion in any one of the genes encoding said native acyl-CoA oxidases. In some embodiments, the yeast cellhas reduced activity of Yli_POX1, Yli_POX2, Yli_POX3, Yli_POX4 and Yli_POX5, for example via deletion of these genes.

Yeast cells useful for the production of a desaturated fatty alcohol or derivative thereof via the shortening of a fatty acyl-CoA to a shortened fatty acyl-CoA having a carbon chain length X′ = 12, such as a Z5-12 fatty acyl-CoA, include cells expressing:
At least one FAR, preferably Har_FAR (SEQ ID NO: 59); and
At least one desaturase, preferably Slitdes5 (SEQ ID NO: 87); and Lbo49554 (SEQ ID NO: 83); or
At least one desaturase, preferably Dmd9 (SEQ ID NO: 35), and one or more of Cma_POX (SEQ ID NO: 22), Hsa_POX1-2 (SEQ ID NO: 24), Pur_POX (SEQ ID NO: 26), Rno_POX-2 (SEQ ID NO: 28), Lbo31670 (SEQ ID NO: 81), and/or Lbo49554 (SEQ ID NO: 83);
At least one desaturase, preferably Lbo_PPTQ (SEQ ID NO: 79), and one or more of Cma_POX (SEQ ID NO: 22), Hsa_POX1-2 (SEQ ID NO: 24), Pur_POX (SEQ ID NO: 26), Rno_POX-2 (SEQ ID NO: 28), Lbo31670 (SEQ ID NO: 81) or Lbo49554 (SEQ ID NO: 83);
or functional variants of any of the above having at least 60% homology thereto, such as at least 65%, such as at least 70%, such as at least 75%, such as at least 80%, such as at least 81%, such as at least 82%, such as at least 83%, such as at least 84%, such as at least 85%, such as at least 86%, such as at least 87%, such as at least 88%, such as at least 89%, such as at least 90%, such as at least 91%, such as at least 92%, such as at least 93%, such as at least 94%, such as at least 95%, such as at least 96%, such as at least 97%, such as at least 98%, such as at least 99% homology thereto. Such yeast cells may in addition have reduced activity of said one or more native acyl-CoA oxidases, such as one or more of Yli_POX1, Yli_POX2, Yli_POX3, Yli_POX4, Yli_POX5 or Yli_POX6, for example a deletion in any one of the genes encoding said native acyl-CoA oxidases. In some embodiments, the yeast cell has reduced activity of Yli_POX1, Yli_POX2, Yli_POX3, Yli_POX4 and Yli_POX5, for example via deletion of these genes.

Yeast cells useful for the production of a desaturated fatty alcohol or derivative thereof via the shortening of a fatty acyl-CoA to a shortened fatty acyl-CoA having a carbon chain length X′ = 12, such as a Z9-12 fatty acyl-CoA, include cells expressing:
At least one FAR, preferably Har_FAR (SEQ ID NO: 59); and
At least one desaturase, preferably Lbo_PPTQ (SEQ ID NO: 79), and one or more of Cma_POX (SEQ ID NO: 22), Hsa_POX1-2 (SEQ ID NO: 24), Pur_POX (SEQ ID NO: 26), Rno_POX-2 (SEQ ID NO: 28), Lbo31670 (SEQ ID NO: 81) or Lbo49554 (SEQ ID NO: 83);
or functional variants of any of the above having at least 60% homology thereto, such as at least 65%, such as at least 70%, such as at least 75%, such as at least 80%, such as at least 81%, such as at least 82%, such as at least 83%, such as at least 84%, such as at least 85%, such as at least 86%, such as at least 87%, such as at least 88%, such as at least 89%, such as at least 90%, such as at least 91%, such as at least 92%, such as at least 93%, such as at least 94%, such as at least 95%, such as at least 96%, such as at least 97%, such as at least 98%, such as at least 99% homology thereto. Such yeast cells may in addition have reduced activity of said one or more native acyl-CoA oxidases, such as one or more of Yli_POX1, Yli_POX2, Yli_POX3, Yli_POX4, Yli_POX5 or Yli_POX6, for example a deletion in any one of the genes encoding said native acyl-CoA oxidases. In some embodiments, the yeast cell has reduced activity of Yli_POX1, Yli_POX2, Yli_POX3, Yli_POX4 and Yli_POX5, for example via deletion of these genes.

Yeast cells useful for the production of a desaturated fatty alcohol or derivative thereof via the shortening of a fatty acyl-CoA to a shortened fatty acyl-CoA having a carbon chain length X′ = 14, such as a Z5-14 fatty acyl-CoA, include cells expressing:
At least one FAR, preferably Har_FAR (SEQ ID NO: 59); and
At least one desaturase, preferably Dmd9 (SEQ ID NO: 35), and one or more of Ani_POX (SEQ ID NO: 20), Cma_POX (SEQ ID NO: 22), Hsa_POX1-2 (SEQ ID NO: 24), Pur_POX (SEQ ID NO: 26), Rno_POX-2 (SEQ ID NO: 28), Lbo31670 (SEQ ID NO: 81) and Lbo49554 (SEQ ID NO: 83); preferably, the acyl-CoA oxidase is not Ase_POX (SEQ ID NO: 14);
At least one desaturase, preferably Lbo_PPTQ (SEQ ID NO: 79), and one or more of Yli_POX2 (SEQ ID NO: 4), Yli_POX5 (SEQ ID NO: 10), Ani_POX (SEQ ID NO: 20), Cma_POX (SEQ ID NO: 22), Hsa_POX1-2 (SEQ ID NO: 24), Pur_POX (SEQ ID NO: 26), Rno_POX-2 (SEQ ID NO: 28), Lbo31670 (SEQ ID NO: 81) or Lbo49554 (SEQ ID NO: 83);
or functional variants of any of the above having at least 60% homology thereto, such as at least 65%, such as at least 70%, such as at least 75%, such as at least 80%, such as at least 81%, such as at least 82%, such as at least 83%, such as at least84%, such as at least 85%, such as at least 86%, such as at least 87%, such as at least 88%, such as at least 89%, such as at least 90%, such as at least 91%, such as at least 92%, such as at least 93%, such as at least 94%, such as at least 95%, such as at least 96%, such as at least 97%, such as at least 98%, such as at least 99% homology thereto. Preferably the desaturase is not Slitdes5 (SEQ ID NO: 87). Such yeast cells may in addition have reduced activity of said one or more native acyl-CoA oxidases, such as one or more of Yli_POX1, Yli_POX2, Yli_POX3, Yli_POX4, Yli_POX5 or Yli_POX6, for example a deletion in any one of the genes encoding said native acyl-CoA oxidases. In some embodiments, the yeast cell has reduced activity of Yli_POX1, Yli_POX2, Yli_POX3, Yli_POX4 and Yli_POX5, for example via deletion of these genes.

Yeast cells useful for the production of a desaturated fatty alcohol or derivative thereof via the shortening of a fatty acyl-CoA to a shortened fatty acyl-CoA having a carbon chain length X′ = 14, such as a Z7-14 fatty acyl-CoA, include cells expressing:
At least one FAR, preferably Har_FAR (SEQ ID NO: 59); and
At least one desaturase, preferably Dmd9 (SEQ ID NO: 35), and one or more of Ani_POX (SEQ ID NO: 20), Cma_POX (SEQ ID NO: 22), Hsa_POX1-2 (SEQ ID NO: 24), Pur_POX (SEQ ID NO: 26), Rno_POX-2 (SEQ ID NO: 28), Lbo31670 (SEQ ID NO: 81) and Lbo49554 (SEQ ID NO: 83); preferably, the acyl-CoA oxidase is not Ase_POX (SEQ ID NO: 14);
At least one desaturase, preferably Lbo_PPTQ (SEQ ID NO: 79), and one or more of Yli_POX2 (SEQ ID NO: 4), Yli_POX5, Ani_POX (SEQ ID NO: 20), Cma_POX (SEQ ID NO: 22), Hsa_POX1-2 (SEQ ID NO: 24), Pur_POX (SEQ ID NO: 26), Rno_POX-2 (SEQ ID NO: 28), Lbo31670 (SEQ ID NO: 81) or Lbo49554 (SEQ ID NO: 83);
or functional variants of any of the above having at least 60% homology thereto, such as at least 65%, such as at least 70%, such as at least 75%, such as at least 80%, such as at least 81%, such as at least 82%, such as at least 83%, such as at least 84%, such as at least 85%, such as at least 86%, such as at least 87%, such as at least 88%, such as at least 89%, such as at least 90%, such as at least 91%, such as at least 92%, such as at least 93%, such as at least 94%, such as at least 95%, such as at least 96%, such as at least 97%, such as at least 98%, such as at least 99% homology thereto. Preferably the desaturase is not Slitdes5. Such yeast cells may in addition have reduced activity of said one or more native acyl-CoA oxidases, such asone or more of Yli_POX1, Yli_POX2, Yli_POX3, Yli_POX4, Yli_POX5 or Yli_POX6, for example a deletion in any one of the genes encoding said native acyl-CoA oxidases. In some embodiments, the yeast cell has reduced activity of Yli_POX1, Yli_POX2, Yli_POX3, Yli_POX4 and Yli_POX5, for example via deletion of these genes.

Yeast cells useful for the production of a desaturated fatty alcohol or derivative thereof via the shortening of a fatty acyl-CoA to a shortened fatty acyl-CoA having a carbon chain length X′ = 14, such as a Z9-14 fatty acyl-CoA, include cells expressing:
At least one FAR, preferably Har_FAR (SEQ ID NO: 59); and
At least one desaturase, preferably Sltdes5 (SEQ ID NO: 87), and one or more of Cma_POX (SEQ ID NO: 22), Hsa_POX1-2 (SEQ ID NO: 24) and Rno_POX-2 (SEQ ID NO: 28);
or functional variants of any of the above having at least 60% homology thereto, such as at least 65%, such as at least 70%, such as at least 75%, such as at least 80%, such as at least 81%, such as at least 82%, such as at least 83%, such as at least 84%, such as at least 85%, such as at least 86%, such as at least 87%, such as at least 88%, such as at least 89%, such as at least 90%, such as at least 91%, such as at least 92%, such as at least 93%, such as at least 94%, such as at least 95%, such as at least 96%, such as at least 97%, such as at least 98%, such as at least 99% homology thereto. Preferably the desaturase is not Dmd9 (SEQ ID NO: 35) or Lbo_PPTQ (SEQ ID NO: 79). Such yeast cells may in addition have reduced activity of said one or more native acyl-CoA oxidases, such as one or more of Yli_POX1, Yli_POX2, Yli_POX3, Yli_POX4, Yli_POX5 or Yli_POX6, for example a deletion in any one of the genes encoding said native acyl-CoA oxidases. In some embodiments, the yeast cell has reduced activity of Yli_POX1, Yli_POX2, Yli_POX3, Yli_POX4 and Yli_POX5, for example via deletion of these genes.

Yeast cells useful for the production of a desaturated fatty alcohol or derivative thereof via the shortening of a fatty acyl-CoA to a shortened fatty acyl-CoA having a carbon chain length X′ = 14, such as a Z11-14 fatty acyl-CoA, include cells expressing:
At least one FAR, preferably Har_FAR (SEQ ID NO: 59); and
At least one desaturase, preferably Lbo_PPTQ (SEQ ID NO: 79), and one or more of Cma_POX (SEQ ID NO: 22), Hsa_POX1-2 (SEQ ID NO: 24), Pur_POX (SEQ ID NO: 26), Rno_POX-2 (SEQ ID NO: 28), Lbo31670 (SEQ ID NO: 81) or Lbo49554 (SEQ ID NO: 83);
or functional variants of any of the above having at least 60% homology thereto, such as at least 65%, such as at least 70%, such as at least 75%, such as at least 80%, such as at least 81%, such as at least 82%, such as at least 83%, such as at least 84%, such as at least 85%, such as at least 86%, such as at least 87%, such as at least 88%, such as at least 89%, such as at least 90%, such as at least 91%, such as at least 92%, such as at least 93%, such as at least 94%, such as at least 95%, such as at least 96%, such as at least 97%, such as at least 98%, such as at least 99% homology thereto. Preferably the desaturase is not Dmd9 (SEQ ID NO: 35) or Sltdes5 (SEQ ID NO: 87). Such yeast cells may in addition have reduced activity of said one or more native acyl-CoA oxidases, such as one or more of Yli_POX1, Yli_POX2, Yli_POX3, Yli_POX4, Yli_POX5 or Yli_POX6, for example a deletion in any one of the genes encoding said native acyl-CoA oxidases. In some embodiments, the yeast cell has reduced activity of Yli_POX1, Yli_POX2, Yli_POX3, Yli_POX4 and Yli_POX5, for example via deletion of these genes.

Yeast cells useful for the production of a desaturated fatty alcohol or derivative thereof via the shortening of a fatty acyl-CoA to a shortened fatty acyl-CoA having a carbon chain length X′ = 16, such as a Z7-16 fatty acyl-CoA, include cells expressing:
At least one FAR, preferably Har_FAR (SEQ ID NO: 59); and
At least one desaturase, preferably Sltdes5 (SEQ ID NO: 87), and one or more of Yli_POX2 (SEQ ID NO: 4), Yli_POX3 (SEQ ID NO: 6), Yli_POX5 (SEQ ID NO: 10), Ani_POX (SEQ ID NO: 20), Cma_POX (SEQ ID NO: 22), Hsa_POX1-2 (SEQ ID NO: 24), Pur_POX (SEQ ID NO: 26), Rno_POX-2 (SEQ ID NO: 28), Lbo31670 (SEQ ID NO: 81) or Lbo49554 (SEQ ID NO: 83); or
At least one desaturase, preferably Dmd9 (SEQ ID NO: 35), and one or more of Yli_POX3 (SEQ ID NO: 6), Yli_POX5 (SEQ ID NO: 10), Ani_POX (SEQ ID NO: 20), Cma_POX (SEQ ID NO: 22), Hsa_POX1-2 (SEQ ID NO: 24), Pur_POX (SEQ ID NO: 26), Rno_POX-2 (SEQ ID NO: 28), Lbo31670 (SEQ ID NO: 81) or Lbo49554 (SEQ ID NO: 83); or
At least one desaturase, preferably Lbo_PPTQ (SEQ ID NO: 79), and one or more of Yli_POX3 (SEQ ID NO: 6), Yli_POX5 (SEQ ID NO: 10), Ani_POX (SEQ ID NO: 20), Cma_POX (SEQ ID NO: 22), Hsa_POX1-2 (SEQ ID NO: 24), Pur_POX (SEQ ID NO: 26), Rno_POX-2 (SEQ ID NO: 28), Lbo31670 (SEQ ID NO: 81) or Lbo49554 (SEQ ID NO: 83);
or functional variants of any of the above having at least 60% homology thereto, such as at least 65%, such as at least 70%, such as at least 75%, such as at least 80%, such as at least 81%, such as at least 82%, such as at least 83%, such as at least 84%, such as at least 85%, such as at least 86%, such as at least 87%, such as at least 88%, such as at least 89%, such as at least 90%, such as at least 91%, such as at least 92%, such as at least 93%, such as at least 94%, such as at least 95%, such as at least 96%, such as at least 97%, such as at least 98%, such as at least 99% homology thereto. Such yeast cells may in addition have reduced activity of said one or more native acyl-CoA oxidases, such as one or more of Yli_POX1, Yli_POX2, Yli_POX3, Yli_POX4, Yli_POX5 or Yli_POX6, for example a deletion in any one of the genes encoding said native acyl-CoA oxidases. In some embodiments, the yeast cell has reduced activity of Yli_POX1, Yli_POX2, Yli_POX3, Yli_POX4 and Yli_POX5, for example via deletion of these genes.

Yeast cells useful for the production of a desaturated fatty alcohol or derivative thereof via the shortening of a fatty acyl-CoA to a shortened fatty acyl-CoA having a carbon chain length X′ = 16, such as a Z9-16 fatty acyl-CoA, include cells expressing:
At least one FAR, preferably Har_FAR (SEQ ID NO: 59); and
At least one desaturase, preferably Sltdes5 (SEQ ID NO: 87), and one or more of Ani_POX (SEQ ID NO: 20), Cma_POX (SEQ ID NO: 22), Hsa_POX1-2 (SEQ ID NO: 24), or Rno_POX-2 (SEQ ID NO: 28); or
At least one desaturase, preferably Dmd9 (SEQ ID NO: 35), and one or more of Ani_POX (SEQ ID NO: 20), Cma_POX (SEQ ID NO: 22), Ase_POX (SEQ ID NO: 14), or Lbo31670 (SEQ ID NO: 81); or
At least one desaturase, preferably Lbo_PPTQ (SEQ ID NO: 79), and one or more of Yli_POX3 (SEQ ID NO: 6), Yli_POX5 (SEQ ID NO: 10), Ani_POX (SEQ ID NO: 20), Cma_POX (SEQ ID NO: 22), Hsa_POX1-2 (SEQ ID NO: 24), Pur_POX (SEQ ID NO: 26), Rno_POX-2 (SEQ ID NO: 28), Lbo31670 (SEQ ID NO: 81) or Lbo49554 (SEQ ID NO: 83);
or functional variants of any of the above having at least 60% homology thereto, such as at least 65%, such as at least 70%, such as at least 75%, such as at least 80%, such as at least 81%, such as at least 82%, such as at least 83%, such as at least 84%, such as at least 85%, such as at least 86%, such as at least 87%, such as at least 88%, such as at least 89%, such as at least 90%, such as at least 91%, such as at least 92%, such as at least 93%, such as at least 94%, such as at least 95%, suchas at least 96%, such as at least 97%, such as at least 98%, such as at least 99% homology thereto. Such yeast cells may in addition have reduced activity of said one or more native acyl-CoA oxidases, such as one or more of Yli_POX1, Yli_POX2, Yli_POX3, Yli_POX4, Yli_POX5 or Yli_POX6, for example a deletion in any one of the genes encoding said native acyl-CoA oxidases. In some embodiments, the yeast cell has reduced activity of Yli_POX1, Yli_POX2, Yli_POX3, Yli_POX4 and Yli_POX5, for example via deletion of these genes.

Yeast cells useful for the production of a desaturated fatty alcohol or derivative thereof via the shortening of a fatty acyl-CoA to a shortened fatty acyl-CoA having a carbon chain length X′ = 16, such as a Z11-16 fatty acyl-CoA, include cells expressing:
At least one FAR, preferably Har_FAR (SEQ ID NO: 59); and
At least one desaturase, preferably Sltdes5 (SEQ ID NO: 87), and one or more of Hsa_POX1-2 (SEQ ID NO: 24), Pur_POX (SEQ ID NO: 26), or Rno_POX-2 (SEQ ID NO: 28);
or functional variants of any of the above having at least 60% homology thereto, such as at least 65%, such as at least 70%, such as at least 75%, such as at least 80%, such as at least 81%, such as at least 82%, such as at least 83%, such as at least 84%, such as at least 85%, such as at least 86%, such as at least 87%, such as at least 88%, such as at least 89%, such as at least 90%, such as at least 91%, such as at least 92%, such as at least 93%, such as at least 94%, such as at least 95%, such as at least 96%, such as at least 97%, such as at least 98%, such as at least 99% homology thereto. Preferably the desaturase is not Dmd9 (SEQ ID NO: 35) or Lbo_PPTQ (SEQ ID NO: 79). Such yeast cells may in addition have reduced activity of said one or more native acyl-CoA oxidases, such as one or more of Yli_POX1, Yli_POX2, Yli_POX3, Yli_POX4, Yli_POX5 or Yli_POX6, for example a deletion in any one of the genes encoding said native acyl-CoA oxidases. In some embodiments, the yeast cell has reduced activity of Yli_POX1, Yli_POX2, Yli_POX3, Yli_POX4 and Yli_POX5, for example via deletion of these genes.

For any of the above specific yeast cells, further modifications may be included such as described herein under “Other useful modifications”.

### Nucleic Acids

It will be understood that throughout the present disclosure, the term ‘nucleic acid encoding an activity’ shall refer to a nucleic acid molecule capable of encoding a peptide, a protein or a fragment thereof having said activity. Such nucleic acid molecules may be open reading frames or genes or fragments thereof. The nucleic acid construct may also be a group of nucleic acid molecules, which together may encode several peptides, proteins or fragments thereof having an activity of interest. The term ‘activity of interest’ refers to one of the following activities: an acyl-CoA oxidase, a desaturase, an alcohol-forming fatty acyl-CoA reductase, an aldehyde-forming fatty acyl-CoA reductase, a dehydrogenase and/or an acetyltransferase activity, as described herein. The nature of the one or more activity of interest will depend on the nature of the desired product one wishes to obtain with the present methods.

The nucleic acids employed for the purpose of the present disclosure may be codon-optimised as is known in the art to improve expression of the proteins they encode in the yeast cell to be modified.

In some embodiments of the present methods, each of the nucleic acids encoding each of the present activities, an acyl-CoA oxidase, a desaturase, an alcohol-forming fatty acyl-CoA reductase, an aldehyde-forming fatty acyl-CoA reductase, a dehydrogenase and/or an acetyltransferase, may be comprised within the genome of the yeast cell or within a vector comprised within yeast cell.

Thus is provided herein a nucleic acid construct for modifying a yeast cell, said construct comprising at least one first group of polynucleotides encoding at least one first group of enzymes comprising at least one acyl-CoA oxidase capable of oxidising a fatty acyl-CoA, wherein the first group of enzymes is capable of shortening a fatty acyl-CoA of a first carbon chain length X to a shortened fatty acyl-CoA having a second carbon chain length X′, wherein X′ ≤ X-2, preferably wherein said acyl-CoA oxidase is as defined herein.

In some embodiments, the first group of polynucleotides comprises or consists of a nucleic acid encoding the acyl-CoA oxidase which is selected from SEQ ID NO: 1, SEQ ID NO: 3, SEQ ID NO: 5, SEQ ID NO: 7, SEQ ID NO: 9, SEQ ID NO: 11, SEQ ID NO: 13, SEQ ID NO: 15, SEQ ID NO: 17, SEQ ID NO: 19, SEQ ID NO: 21, SEQ ID NO: 23, SEQ ID NO: 25, SEQ ID NO: 27, SEQ ID NO: 80, SEQ ID NO: 82, SEQ ID NO: 84, or homologues thereof having at least 60% homology thereto, such as at least 65%, such as at least 70%, such as at least 75%, such as at least 80%, such as at least 81%, such as at least 82%, such as at least 83%, such as at least 84%, such as at least 85%, such as at least 86%, such as at least 87%, such as at least 88%, such as at least 89%, such as at least 90%, such as at least 91%, such as at least 92%, such as at least 93%, such as at least 94%, such as at least 95%, such as at least 96%, such as at least 97%, such as at least 98%, such as at least 99% homology thereto.

In some embodiments, the nucleic acid encoding the acyl-CoA oxidase is selected from SEQ ID NO: 3, SEQ ID NO: 19, SEQ ID NO: 21, SEQ ID NO: 25, SEQ ID NO: 80, SEQ ID NO: 82, SEQ ID NO: 84 and SEQ ID NO: 27.

The nucleic acid construct may further comprise one or more of a second polynucleotide, a third polynucleotide, a fourth polynucleotide and a fifth polynucleotide as detailed below.

The nucleic acid construct may further comprise a second polynucleotide encoding at least one heterologous desaturase capable of introducing at least one double bond in said shortened fatty acyl-CoA, preferably wherein said heterologous desaturase is as defined herein.

In some embodiments, the second polynucleotide comprises or consists of a nucleic acid having at least 60% homology to SEQ ID NO: 36, SEQ ID NO: 37, SEQ ID NO: 39, SEQ ID NO: 55, SEQ ID NO: 41, SEQ ID NO: 43, SEQ ID NO: 34, SEQ ID NO: 33, SEQ ID NO: 29, SEQ ID NO: 31, SEQ ID NO: 45, SEQ ID NO: 47, SEQ ID NO: 49, SEQ ID NO: 51, SEQ ID NO: 53, SEQ ID NO: 55, SEQ ID NO: 78, SEQ ID NO: 86, SEQ ID NO: 88, SEQ ID NO: 90, SEQ ID NO: 98 or SEQ ID NO: 100, such as at least 61% homology, such as at least 62% homology, such as at least 63% homology, such as at least 64% homology, such as at least 65% homology, such as at least 66% homology, such as at least 67% homology, such as at least 68% homology, such as at least 69% homology, such as at least 70% homology, such as at least 71% homology, such as at least 72%, such as at least 73%, such as at least 74%, such as at least 75%, such as at least 76%, such as at least 77%, such as at least 78%, such as at least 79%, such as at least 80%, such as at least 81%, such as at least 82%, such as at least 83%, such as at least 84%, such as at least 85%, such as at least 86%, such as at least 87%, such as at least 88%, such as at least 89%, such as at least 90%, such as at least 91%, such as at least 92%, such as at least 93%, such as at least 94%, such as at least 95%, such as at least 96%, such as at least 97%, such as at least 98%, such as at least 99% homology to SEQ ID NO: 36, SEQ ID NO: 37, SEQ ID NO: 39, SEQ ID NO: 55, SEQ ID NO: 41, SEQ ID NO: 43, SEQ ID NO: 34, SEQ ID NO: 33, SEQ ID NO: 29, SEQ ID NO: 31, SEQ ID NO: 45, SEQ ID NO: 47, SEQ ID NO: 49, SEQ ID NO: 51, SEQ ID NO: 53, SEQ ID NO: 55, SEQ ID NO: 78, SEQ ID NO: 86, SEQ ID NO: 88, SEQ ID NO: 90, SEQ ID NO: 98 or SEQ ID NO: 100.

In some embodiments, the second polynucleotide comprises or consists of a nucleic acid having at least 60% homology to SEQ ID NO: 29, SEQ ID NO: 90, SEQ ID NO: 78, SEQ ID NO: 53, SEQ ID NO: 39, SEQ ID NO: 86 or SEQ ID NO: 33.

The nucleic acid construct may comprise a third polynucleotide encoding at least one heterologous fatty acyl-CoA reductase (FAR), capable of converting at least part of a desaturated fatty acyl-CoA to a desaturated fatty alcohol, preferably wherein said heterologous FAR is as defined herein.

In some embodiments, the third polynucleotide comprises or consists of a nucleic acid having at least 60% homology to SEQ ID NO: 56, SEQ ID NO: 74, SEQ ID NO: 72, SEQ ID NO: 76, or SEQ ID NO: 92, such as at least 61% homology, such as at least 62% homology, such as at least 63% homology, such as at least 64% homology, such as at least 65% homology, such as at least 66% homology, such as at least 67% homology, such as at least 68% homology, such as at least 69% homology, such as at least 70% homology, such as at least 71% homology, such as at least 72%, such as at least 73%, such as at least 74%, such as at least 75%, such as at least 76%, such as at least 77%, such as at least 78%, such as at least 79%, such as at least 80%, such as at least 81%, such as at least 82%, such as at least 83%, such as at least 84%, such as at least 85%, such as at least 86%, such as at least 87%, such as at least 88%, such as at least 89%, such as at least 90%, such as at least 91%, such as at least 92%, such as at least 93%, such as at least 94%, such as at least 95%, such as at least 96%, such as at least 97%, such as at least 98%, such as at least 99%, such as 100% homology to SEQ ID NO: 56, SEQ ID NO: 74, SEQ ID NO: 72, SEQ ID NO: 76, or SEQ ID NO: 92.

The nucleic acid construct may comprise a fourth polynucleotide encoding an acetyltransferase capable of converting at least part of a desaturated fatty alcohol to a desaturated fatty acyl acetate, preferably wherein said acetyltransferase is as defined herein.

In some embodiments, the fourth polynucleotide comprises or consists of a nucleic acid having at least 60% homology to SEQ ID NO: 60, such as at least 61% homology, such as at least 62% homology, such as at least 63% homology, such as at least 64% homology, such as at least 65% homology, such as at least 66% homology, such as at least 67% homology, such as at least 68% homology, such as at least 69% homology, such as at least 70% homology, such as at least 71% homology, such as at least 72%, such as at least 73%, such as at least 74%, such as at least 75%, such as at least 76%, such as at least 77%, such as at least 78%, such as at least 79%, such as at least 80%, such as at least 81%, such as at least 82%, such as at least 83%, such as at least 84%, such as at least 85%, such as at least 86%, such as at least 87%, such as at least 88%, such as at least 89%, such as at least 90%, such as at least 91%, such as at least 92%, such as at least 93%, such as at least 94%, such as at least 95%, such as at least 96%, such as at least 97%, such as at least 98%, such as at least 99%, such as 100% homology to SEQ ID NO: 60.

The nucleic acid construct may comprise a fifth polynucleotide encoding at least one alcohol dehydrogenase and/or fatty alcohol oxidase capable of converting at least part of a desaturated fatty alcohol to a desaturated fatty aldehyde, preferably wherein said alcohol dehydrogenase and/or fatty alcohol oxidase is as defined herein.

In some embodiments, each of the nucleic acids encoding each of the present activities may be present in the genome of said yeast cell, either because the nucleic acid is already present in the yeast cell, or because it has been integrated therein by genome engineering or genome editing or by crossing yeast cells of different mating types. Alternatively, the nucleic acids may be expressed in the cell from a vector.

Methods for integrating a nucleic acid are well known in the art. Thus in some embodiments the activity of interest is encoded by introduction of a heterologous nucleic acid in the yeast cell. The heterologous nucleic acid encoding said activity may be codon-optimised for any purpose, or may comprise features that can help improve the activity. For example, the heterologous nucleic acid may be modified so as to encode a modified protein. Such modifications include, but are not limited to, the introduction of localisation signals, gain-of-function or loss-of-function mutations, fusion of the protein to a marker or a tag such as fluorescent tag, insertion of an inducible promoter, introduction of modifications conferring increased stability and/or half-life.

The introduction of the heterologous nucleic acid encoding the activity of interest can be performed by methods known in the art. The skilled person will recognise that such methods include, but are not limited to: cloning and homologous recombination-based methods. Cloning methods may involve the design and construction of a plasmid in an organism such as Escherichia coli. The plasmid may be an integrative or a non-integrative vector. Cloning-free methods comprise homologous recombination-based methods such as adaptamer-mediated PCR or gap repair. Such methods often result in integration of the heterologous nucleic acid in the genome of the yeast cell.

The nucleic acids encoding the activities of interest may be present in high copy number.

### Production of a Desaturated Fatty Alcohol, a Desaturated Fatty Acyl Acetate and/or a Desaturated Fatty Aldehyde

The yeast cells of the present disclosure can be used for the production of a desaturated fatty alcohol of a desired carbon chain length X′ from fatty acyl-CoAs having a carbon chain length X greater than X′.

The yeast cells provided herein are thus useful for methods of producing desaturated fatty alcohols and derivatives thereof.

Accordingly, herein is provided a method for producing a desaturated fatty alcohol and optionally a desaturated fatty acyl acetate and/or a fatty aldehyde of carbon chain length X′, comprising the steps of providing a yeast cell capable of converting a fatty acyl-CoA of a first carbon chain length X to a desaturated fatty alcohol and optionally a desaturated fatty acyl acetate and/or a fatty aldehyde of carbon chain length X′ and incubating said yeast cell in a medium, wherein the yeast cell is as defined herein and wherein X′ ≤ X-2.

X and X′ may be as defined herein. The yeast cell may be as described herein above, i.e. the yeast cell is capable of producing a desaturated fatty alcohol and optionally a desaturated fatty acyl acetate and/or a desaturated fatty aldehyde, and said yeast cell:
i) has one or more mutations resulting in reduced activity of one or more native acyl-CoA oxidases; and
ii) expresses at least one first group of enzymes comprising at least one acyl-CoA oxidase capable of oxidising a fatty acyl-CoA, wherein the first group of enzymes is capable of shortening a fatty acyl-CoA of a first carbon chain length X to a shortened fatty acyl-CoA having a second carbon chain length X′, wherein X′ ≤ X-2; and
iii) expresses at least one heterologous desaturase capable of introducing at least one double bond in said fatty acyl-CoA and/or in said shortened fatty acyl-CoA; and
iv) expresses at least one heterologous fatty acyl-CoA reductase, capable of converting at least part of said desaturated fatty acyl-CoA to a desaturated fatty alcohol; and
v) optionally expresses at least one acetyltransferase capable of converting at least part of said desaturated fatty alcohol to a desaturated fatty acyl acetate, and/or at least one alcohol dehydrogenase and/or fatty alcohol oxidase capable of converting at least part of said desaturated fatty alcohol to a desaturated fatty aldehyde.

The acyl-CoA oxidases may be as described herein above in the section ‘Acyl-CoA oxidase”. The desaturase may be as described herein above in the section ‘Desaturase (FAD)’. The fatty acyl-CoA reductase may be as described herein above in the section ‘Alcohol-forming fatty acyl-CoA reductase (EC 1.2.1.84)’. The dehydrogenase may be as described herein above in the section ‘Dehydrogenase’. The acetyltransferase may be as described herein above in the section ‘Acetyltransferase (EC 2.3.1.84)’. In addition, the yeast cell may be further modified as described in the section ‘Other useful modifications’. In some embodiments, the yeast cell may further express an aldehyde-forming fatty acyl-CoA reductase EC 1.2.1.50 (FAR′) as described herein above in the section ‘Aldehyde-forming fatty acyl-CoA reductase (FAR’) (EC 1.2.1.50)′.

The yeast cell may be able to provide fatty acyl-CoAs naturally, or may be engineered to synthesis fatty acyl-CoAs to be used as a substrate, or fatty acyl-CoAs may be provided in the medium in which the cell is incubated, as described in the section ‘Fatty acyl-CoA’.

While the present disclosure provides methods for producing desaturated fatty alcohols and/or desaturated fatty acyl acetates and/or desaturated fatty aldehydes by engineering a yeast cell, it may be of interest to convert the produced desaturated fatty alcohols produced by the present yeast cells and methods to the corresponding desaturated fatty aldehydes by other methods. Thus in some embodiments, the method may further comprise the step of converting at least part of the fatty alcohols to fatty aldehydes, thereby producing fatty aldehydes, e.g. by chemical methods or enzymatic methods.

In some embodiments, the step of converting at least part of the desaturated fatty alcohols to the corresponding desaturated fatty aldehydes is a step of chemical conversion. The chemical conversion is based on the oxidation of fatty alcohols to the corresponding aldehydes. Methods for performing this conversion are known in the art. Preferred methods are environmentally friendly and minimize the amount of hazardous waste.

Thus in some embodiments, the chemical conversion may be metal free, avoiding toxic heavy metal based reagents such as manganese oxides, chromium oxides (Jones ox. PDC, PCC) or ruthenium compounds (TPAP, Ley-Griffith ox.). In some embodiments, the conversion does not involve reactions with activated dimethyl sulfoxide such as the Swern oxidation or the Pfitzner-Moffat type. Such reactions may involve the stereotypic formation of traces of intensively smelling organic sulfur compounds such as dimethyl sulfide which can be difficult to remove from the target product.

In some embodiments, the method comprises a Dess-Martin reaction [20, 21]. In some embodiments, the method comprises a Copper(l)/ABNO-catalysed aerobic alcohol oxidation reaction [22].

In other embodiments, the chemical conversion comprises the oxidation with sodium hypochlorite under aqueous/organic two phase conditions [23-25]. In some embodiments, the chemical oxidation can be performed with 1-chlorobenzotriazole in a medium of methylene chloride containing 25% pyridine [26].

Alternatively, the oxidation of a fatty alcohol to the corresponding fatty aldehyde can be performed enzymatically by alcohol dehydrogenases or fatty alcohol oxidases. The skilled person will know how to carry out enzymatic oxidation. For example, enzymatic oxidation can be carried out by contacting purified enzymes, cell extracts or whole cells, with the fatty alcohol.

Fatty aldehydes can also be obtained directly by introducing a gene encoding an aldehyde-forming fatty acyl-CoA reductase EC 1.2.1.50 (FAR′), as described herein above. In some embodiments, in addition to the other modifications described herein, the yeast cell thus also expresses a FAR′, which can convert at least part of the acyl-CoA of chain length X′ to the corresponding aldehyde.

In some embodiments, the method yields a desaturated fatty alcohol, and optionally a desaturated fatty acyl acetate and/or a desaturated fatty aldehyde, with a titre of at least 1 mg/L, such as at least 1.5 mg/L, such as at least 5 mg/L, such as at least 10 mg/L, such as at least 25 mg/L, such as at least 50 mg/L, such as at least 100 mg/L, such as at least 250 mg/L, such as at least 500 mg/L, such as at least 750 mg/L, such as at least 1 g/L, such as at least 2 g/L, such as at least 3 g/L, such as at least 4 g/L, such as at least 5 g/L, such as at least 6 g/L, such as at least 7 g/L, such as at least 8 g/L, such as at least 9 g/L, such as at least 10 g/L, such as at least 11 g/L, such as at least 12 g/L, such as at least 13 g/L, such as at least 14 g/L, such as at least 15 g/L, such as at least 16 g/L, such as at least 17 g/L, such as at least 18 g/L, such as at least 19 g/L, such as at least 20 g/L, such as at least 25 g/L, such as at least 30 g/L, such as at least 35 g/L, such as at least 40 g/L, such as at least 45 g/L, such as at least 50 g/L, or more.

In some embodiments, the method yields fatty alcohols with a total titre of at least 1 mg/L, such as at least 5 mg/L, such as at least 10 mg/L, such as at least 25 mg/L, such as at least 50 mg/L, such as at least 100 mg/L, such as at least 250 mg/L, such as at least 500 mg/L, such as at least 1 g/L, such as at least 2 g/L, such as at least 3 g/L, such as at least 4 g/L, such as at least 5 g/L, such as at least 6 g/L, such as at least 7 g/L, such as at least 8 g/L, such as at least 9 g/L, such as at least 10 g/L, wherein the total titre is the sum of the titre of desaturated fatty alcohols and the titre of saturated fatty alcohols.

In some embodiments, the total desaturated fatty alcohols produced represent at least 5% of the total fatty alcohols produced by the cell, such as at least 10%, such as at least 20%, such as at least 30%, such as at least 40%, such as at least 50%, such as at least 60%, such as at least 70%, such as at least 80%, such as at least 90% of the total fatty alcohols produced by the cell, wherein the total fatty alcohols produced by the cell are the sum of the saturated fatty alcohols produced by the cell and the desaturated fatty alcohols produced by the cell. The total desaturated fatty alcohols produced represent the sum of all the desaturated fatty alcohols produced by the yeast cell, of all carbon chain lengths.

In some embodiments, the desaturated fatty alcohol of carbon chain length X′ produced by the cell represents at least 20% of the total desaturated fatty alcohols produced by the cell, such as at least 30%, such as at least 40%, such as at least 50%, such as at least 60%, such as at least 70%, such as at least 80%, such as at least 90% of the total desaturated fatty alcohols produced by the cell.

In some embodiments, the desaturated fatty alcohol of carbon chain length X′ produced by the cell represents at least 2% of the total desaturated fatty alcohols produced by the cell, such as at least 5%, such as at least 6%, such as at least 7%, such as at least 8%, such as at least 9%, such as at least 10%, such as at least 15%, such as at least 20%, such as at least 30%, such as at least 40%, such as at least 50%, such as at least 60%, such as at least 70%, such as at least 80%, such as at least 90% of the total desaturated fatty alcohols produced by the cell.

In some embodiments, the desaturated fatty alcohol of carbon chain length X′ produced by the cell represents at least 5% of the total fatty alcohols of chain length X′ produced by the cell, such as at least 10%, such as at least 15%, such as at least 20%, such as at least 30%, such as at least 40%, such as at least 50%, such as at least 60%, such as at least 70%, such as at least 80%, such as at least 90% of the total fatty alcohols of carbon chain length X′ produced by the cell, wherein the total fatty alcohols of carbon chain length X′ is the sum of desaturated and saturated fatty alcohol of carbon chain length X′.

In some embodiments, the method provides a desaturated fatty alcohol, and/or a desaturated fatty acyl acetate and/or a desaturated fatty aldehyde, such as a desaturated fatty alcohol, and/or a desaturated fatty acyl acetate and/or a desaturated fatty aldehyde of carbon chain length X′, with an increased titer compared to the titer obtained with the same method but using a wild-type or unmodified yeast cell. In some embodiments, the increase in titer is at least 1.5-fold, such as at least 2-fold, such as at least 3-fold, such as at least 4-fold, such as at least 5-fold, such as at least 6-fold, such as at least 7-fold, such as at least 8-fold, such as at least 9-fold, such as at least 10-fold, such as at least 15-fold, such as at least 20-fold, or more. By wild-type or unmodified yeast cell is to be understood a yeast cell which is devoid of mutations resulting in reduced activity of the native acyl-CoA oxidases, does not express a heterologous acyl-CoA oxidase, does not express a heterologous desaturase and does not express a heterologous fatty acyl-CoA reductase.

The desaturated fatty alcohols, acetates or aldehydes produced by the present method may be important pheromone components of various insects, in particular of the Lepidoptera order.

For example, the following compounds can be produced using the present methods:
Δ_{Z5}-12:O-acetate, which is a major pheromone component of the Western bean cutworm Striacosta albicosta;
Δ_{Z7}-12:O-acetate, which is a major pheromone component of the soybean looper Chrysodeixis includens;
Δ_{Z7}-12:O-acetate, which is a major pheromone component of the soybean looper C. includens;
Δ_{E7,Z9}-12:O-acetate, which is a major pheromone component of the grapevine moth Lobesia botrana;
Δ_{Z8}-12:O-acetate, and Δ_{E8}-12: -acetate, which are respectively a major pheromone component and a minor pheromone component of the oriental fruit moth G. molesta;
Δ_{E8,E10}-C12:OH, which is a major pheromone component of the codling moth C. pomonella;
Δ_{Z9}-12:O-acetate, which is a major pheromone component of the grape berry moth Paralobesia viteana;
Δ_{Z7}-14-aldehyde, which is a major pheromone component of the olive fruit fly Prays oleae;
Δ_{Z9}-14:O-acetate, which is a major pheromone component of the fall armyworm Spodoptera frugiperda;
Δ_{E11}-14:O-acetate, which is a major pheromone component of the lightbrown apple moth E. postvittana;
Δ_{Z11}-14:O-acetate, which is a major pheromone component of the European corn borer O. nubilalis;
Δ_{Z7}-16-aldehyde, which is a minor pheromone component of the crambid stalkborer Diatraea considerata;
Δ_{Z7,Z11}-16:O-acetate, which is the major pheromone component of the pink bollworm Pectinophora gossypiella;
Δ_{Z9}-16:OH, which can be chemically oxidized into Δ_{Z9}-16-aldehyde [19]. Δ_{Z9}-16-aldehyde is the major pheromone component of the oriental tobacco budworm Helicoverpa assulta;
Δ_{Z11,Z13}-16:OH, which can be chemically oxidized into Δ_{Z11,Z13}-16-aldehyde [19]. Δ_{Z11,Z13}-16-aldehyde is the major pheromone component of the orange worm A. transitella.

Any of the yeast cells described herein above, for example in “Specific yeast cells”, can be employed in the present methods.

### Recovery

It may be desirable to recover the products obtained by the methods disclosed herein. Thus the present methods may further comprise a further step of recovering the desaturated fatty alcohol and/or the desaturated fatty acyl acetate produced by the present yeast cell.

In some embodiments, the method comprises a step of recovering the desaturated fatty alcohols. In a particular embodiment, the method comprises a step of recovering the desaturated fatty alcohols, including the desaturated fatty alcohols having a carbon chain length X′. In other embodiments, the method comprises a step of recovering the fatty acyl acetates, including the fatty acyl acetates of carbon chain length X′. In a particular embodiment, the method comprises a step of recovering the fatty acyl aldehydes, including the fatty acyl aldehydes having a carbon chain length X′.

Methods for recovering the products obtained by the present invention are known in the art and may comprise an extraction with a hydrophobic solvent such as decane, hexane or a vegetable oil.

The recovered products may be modified further, for example desaturated fatty alcohols may be converted to the corresponding desaturated fatty aldehydes as described herein above.

The recovered products, i.e. the desaturated fatty alcohols and/or desaturated fatty acyl acetates, may be formulated into a pheromone composition. The composition may further comprise one or more additional compounds such as a liquid or solid carrier or substrate. Fatty aldehydes obtained from said desaturated fatty alcohols may also be comprised in such compositions.

### Kit

Provided herein is a kit of parts for performing the present methods. The kit of parts may comprise a yeast cell “ready to use” as described herein, i.e. a yeast cell capable of producing a desaturated fatty alcohol, a desaturated fatty acyl acetate and/or a desaturated fatty aldehyde as described herein.

In another embodiment, the kit of parts comprises, in addition or alternatively to the above, nucleic acid constructs encoding the activities of interest to be introduced in the yeast cell. The nucleic acid construct may be provided as a plurality of nucleic acid constructs, such as a plurality of vectors, wherein each vector encodes one or several of the desired activities.

The kit of parts may comprise, in addition or alternatively to the above, sets of primers for introducing one or more mutations resulting in reduced activity of the one or more native acyl-CoA oxidases.

The kit of parts may also comprise template DNA and primers to be used in e.g. a PCR reaction to obtain nucleic acid constructs useful for introducing an acyl-CoA oxidase, a desaturase, an alcohol-forming fatty acyl-CoA reductase and optionally a dehydrogenase and/or an acetyltransferase and/or an aldehyde-forming fatty acyl-CoA reductase in a yeast cell, as described herein above. The kit may also comprise purified DNA which can be used directly for introducing an acyl-CoA oxidase, a desaturase, an alcohol-forming fatty acyl-CoA reductase and optionally a dehydrogenase and/or an acetyltransferase and/or an aldehyde-forming fatty acyl-CoA reductase in a yeast cell.

The kit of parts may optionally comprise the yeast cell to be modified, i.e. a yeast cell which has not yet been modified to perform the present methods.

In some embodiments, the kit of parts comprises all of the above.

### Pheromone Composition

The present disclosure thus provides compounds, in particular fatty alcohols and fatty acyl acetates, as well as derivatives thereof, and their use. In particular, the compounds obtainable using the present cells and methods are useful as components of pheromone compositions. Such pheromone compositions may be useful for integrated pest management. They can be used as is known in the art for e.g. mating disruption.

The desaturated fatty alcohols and desaturated fatty acyl acetates obtainable by the present methods or using the present yeast cells may be formulated in a pheromone composition.

Such pheromone compositions may be used as integrated pest management products, which can be used in a method of monitoring the presence of pest or in a method of disrupting the mating of pest. Thus is also provided herein the use of a desaturated fatty alcohol, a desaturated fatty acyl acetate and/or a desaturated fatty aldehyde obtainable by the present methods or produced by the present yeast cells, in a method of pest management.

Pheromone compositions as disclosed herein may be used as biopesticides. Such compositions can be sprayed or dispensed on a culture, in a field or in an orchard. They can also, as is known in the art, be soaked e.g. onto a rubber septa, or mixed with other components. This can result in mating disruption, thereby preventing pest reproduction, or it can be used in combination with a trapping device to entrap the pests. Non-limiting examples of pests against which the present pheromone compositions can be used are: cotton bollworm (Helicoverpa armigera), striped stemborer (Chilo suppressalis), diamond back moth (Plutella xylostella), cabbage moth (Mamestra brassicae), large cabbage-heart caterpillar (Crocidolomia binotalis), European corn stalk borer (Sesamia nonagrioides), currant clearwing (Synanthedon tipuliformis), artichoke plume moth (Platyptilia carduidactylal), Western bean cutworm (Striacosta albicosta), soybean looper (Chrysodeixis includes), grapevine moth (Lobesia botrana), oriental fruit moth (Grapholita molesta), codling moth (Cydia pomonella), grape berry moth (Paralobesia viteana), olive fruit fly (Prays oleae), fall armyworm (Spodoptera frugiperda), lightbrown apple moth (Epiphyas postvittana), European corn borer (Ostrinia nubilalis), crambid stalkborer (Diatraea considerata), pink bollworm (Pectinophora gossypiella), oriental tobacco budworm (Helicoverpa assulta), orange navelworm (Amyelois transitella). Accordingly, use of the present compositions on a culture can lead to increased crop yield, with substantially no environmental impact.

The relative amounts of desaturated fatty alcohols and desaturated fatty acyl acetates in the present pheromone compositions may vary depending on the nature of the crop and/or of the pest to be controlled; geographical variations may also exist. Determining the optimal relative amounts may thus require routine optimisation. The pheromone compositions may also comprise desaturated fatty aldehydes.

Examples of compositions used as repellents can be found in [27] for H. armigera, in [28] for C. suppressalis, in [29] for S. nonagrioides; in [30] for P. xylostella; in [31] for P. carduidactyla.

In some embodiments, the pheromone composition may further comprise one or more additional compounds such as a liquid or solid carrier or substrate. For example, suitable carriers or substrate include vegetable oils, refined mineral oils or fractions thereof, rubbers, plastics, silica, diatomaceous earth, wax matrix and cellulose powder.

The pheromone composition may be formulated as is known in the art. For example, it may be in the form of a solution, a gel, a powder. The pheromone composition may be formulated so that it can be easily dispensed, as is known in the art.

### EXAMPLES

### Example 1: Cloning and Strain Construction for Yarrowia Lipolytica

All heterologous genes were synthesized by GeneArt (Life Technologies) in codon optimized versions for Y. lipolytica. All the genes were amplified by PCR using Phusion U Hot Start DNA Polymerase (ThermoFisher) to obtain the fragments for cloning into yeast expression vectors. The primers are listed in Table 1 and the resulting DNA fragments (BioBricks) are listed in Table 2. The PCR products were separated on a 1%-agarose gel containing RedSafe™ (iNtRON Biotechnology). PCR products of the correct size were excised from the gel and purified using the Nucleospin Gel and PCR Clean-up kit (Macherey-Nagel).

Integrative yeast vectors with USER cassette were linearized with FastDigest SfaAl (ThermoFisher) for 2 hours at 37° C. and then nicked with Nb.Bsml (New England Biolabs) for 1 hour at 65° C. The resulting vectors containing sticky ends were separated by gel electrophoresis, excised from the gel, and gel-purified using the Nucleospin Gel and PCR Clean-up kit (Macherey-Nagel). The DNA fragments were cloned into the so prepared vectors by USER-cloning as described in [5]. The reaction was transformed into chemically competent E. coli DHalpha cells and the cells were plated on Lysogeny Broth (LB) agar plates with 100 mg/L ampicillin. The plates were incubated overnight at 37° C. and the resulting colonies were screened by colony PCR. The plasmids were purified from overnight E. coli liquid cultures and the correct cloning was confirmed by sequencing. The constructed vectors are listed in Table 3.

Yeast strains were constructed by transformation of DNA vectors as described in [5]. Integrative vectors were linearized with FastDigest Notl prior to transformation. When needed, helper vectors to promote the integration into specific genomic regions were co-transformed with the integrative plasmid or DNA repair fragments (Table 4). Strains were selected on yeast peptone dextrose (YPD) agar with appropriate antibiotics selection. Correct genotype was confirmed by colony PCR and when needed by sequencing. The resulting strains are listed in Table 5.

TABLE 1

Primers

| Primer name | Template | NCBI accession number | Hybridises at positions |
|---|---|---|---|
| PR-21648 | Yali0C | NC_006069 | 3195907..3195926 |
| PR-21649 | Yali0C | NC_006069 | 3195907..3195926 |
| PR-21650 | Yali0E | NC_006071 | 754578..754559 |
| PR-21651 | Yali0E | NC_006071 | 754559..754578 |
| PR-21652 | Yali0E | NC_006071 | 3897473..3897492 |
| PR-21653 | Yali0E | NC_006071 | 3897473..3897492 |
| PR-21654 | Yali0E | NC_006071 | 3265273..3265292 |
| PR-21655 | Yali0E | NC_006071 | 3265273..3265292 |
| PR-21656 | Yali0F | NC_006072 | 1763..1744 |
| PR-21657 | Yali0F | NC_006072 | 1451032..1451051 |
| PR-21658 | Yali0D | NC_006070 | 3293028..3293047 |
| PR-21659 | Yali0D | NC_006070 | 635..654 |
| PR-21660 | Yali0E | NC_006071 | 3896600..3896618 |
| PR-21661 | Yali0E | NC_006071 | - |
| PR-21662 | Yali0E | NC_006071 | - |
| PR-21663 | Yali0E | NC_006071 | 3899638..3899658 |
| PR-21664 | Yali0F | NC_006072 | 1448763..1448783 |
| PR-21665 | Yali0F | NC_006072 | 1449271..1449290 |
| PR-21666 | Yali0F | NC_006072 | 1451392..1451413 |
| PR-21667 | Yali0F | NC_006072 | 1451890..1451909 |
| PR-21668 | Yali0D | NC_006070 | 3291067..3291086 |
| PR-21669 | Yali0D | NC_006070 | 3291578..3291560 |
| PR-21670 | Yali0D | NC_006070 | 3293682..3293701 |
| PR-21671 | Yali0D | NC_006070 | 3294126..3294107 |
| PR-21672 | Yali0E | NC_006071 | 3266663..3266643 |
| PR-21673 | Yali0E | NC_006071 | - |
| PR-21674 | Yali0E | NC_006071 | - |
| PR-21675 | Yali0E | NC_006071 | 3263525..3263544 |
| PR-21676 | Yali0C | NC_006069 | 3195036..3195057 |
| PR-21677 | Yali0C | NC_006069 | 3195535..3195515 |
| PR-21678 | Yali0C | NC_006069 | 3197632..3197653 |
| PR-21679 | Yali0C | NC_006069 | 3198126..3198108 |
| PR-21680 | Yali0E | NC_006071 | 753726..753746 |
| PR-21681 | Yali0E | NC_006071 | 754225..754244 |
| PR-21682 | Yali0E | NC_006071 | 756316..756333 |
| PR-21683 | Yali0E | NC_006071 | 756810..756828 |
| PR-22825 (Yli_POX1_fw) | Yali0E | NC_006071 | 3897120..3897129 |
| PR-22826 (Yli_POX1_rv) | Yali0E | NC_006071 | 3899139..3899153 |
| PR-22827 (Yli_POX2_fw) | Yali0F | NC_006072 | 1449289..1449297 |
| PR-22828 (Yli_POX2_rv) | Yali0F | NC_006072 | 1451377..1451391 |
| PR-22829 (Yli_POX3_fw) | Yali0D | NC_006070 | 555907..555898 |
| PR-22830 (Yli_POX3_rv) | Yali0D | NC_006070 | 3291579..3291593 |
| PR-22831 (Yli_POX4_fw) | Yali0E | NC_006071 | 3266159..3266168 |
| PR-22832 (Yli_POX4_rv) | Yali0E | NC_006071 | 3264063..3264077 |
| PR-22833 (Yli_POX5_fw) | Yali0C | NC_006069 | 3488181..3488192 |
| PR-22834 | Yali0C | NC_006069 | 1534254..1534268 |
| (Yli_POX5_rv) | | | |
| PR-22835 (Yli_POX6_fw) | Yali0E | NC_006071 | 756305..756314 |
| PR-22836 (Yli_POX6_rv) | Yali0E | NC_006071 | 754245..754259 |
| PR-22837 (Ase_POX_fw) | SEQ ID NO:13 | - | 1..12 |
| PR-22838 (Ase_POX_rv) | SEQ ID NO:13 | - | 2074..2088 |
| PR-22839 (Ath_POX1_fw) | SEQ ID NO:15 | - | 1..12 |
| PR-22840 (Ath_POX1_rv) | SEQ ID NO:15 | - | 2029..2045 |
| PR-22841 (Ani_POX_fw) | SEQ ID NO:19 | - | 1..10 |
| PR-22842 (Ani_POX_rv) | SEQ ID NO:19 | - | 2090..2105 |
| PR-22843 (Cma_POX_fw) | SEQ ID NO:21 | - | 1..10 |
| PR-22844 (Cma_POX_rv) | SEQ ID NO:21 | - | 1975..1991 |
| PR-22845 (Hsa_POX1-2_fw) | SEQ ID NO:23 | - | 1..10 |
| PR-22846 (Hsa_POX1-2_rv) | SEQ ID NO:23 | - | 2017..2033 |
| PR-22847 (Pur_POX_fw) | SEQ ID NO:25 | - | |
| PR-22848 (Pur_POX_rv) | SEQ ID NO:25 | - | |
| PR-22849 (Rno_POX-2_fw) | SEQ ID NO:27 | - | |
| PR-22850 (Rno_POX-2_rv) | SEQ ID NO:27 | - | |
| PR-22851 (Ath_POX2_fw) | SEQ ID NO:17 | - | 1..10 |
| PR-22852 (Ath_POX2_rv) | SEQ ID NO:17 | - | 2113..2129 |
| PR-18928 (PrTEF1 <-_U1_fw) | Yali0C | NC_006069 | 1244239..124422 |
| PR-18975 (<-PrTef_fw) | Yali0C | NC_006069 | 1243860..1243877 |
| PR-10595 (PrTefYL _fw) | Yali0C | NC_006069 | 1244252..1244265 |
| PR-18489 | SEQ ID | - | 1599..1614 |
| (Dmd9_optYlip_G V2R) | NO:64 | - | |
| PR-21764 (Atrd11->_U2_rev) | SEQ ID NO:65 | - | 1491..1509 |
| PR-21737 (<-Cro_Z11_U1_fw) | SEQ ID NO:39 | - | 4..21 |
| PR-21738 (<-Cro_Z11_U1_rev) | SEQ ID NO:39 | - | 990..1008 |
| PR-21727 (<-Onu_11_U1_fw) | SEQ ID NO:41 | - | 4..21 |
| PR-21728 (<-Onu_11_U1_rev) | SEQ ID NO:41 | - | 971..990 |
| PR-21731 (<-Tpi_D13_U1_fw) | SEQ ID NO:43 | - | 4..24 |
| PR-21732 (<-Tpi_D13_U1_rev) | SEQ ID NO:43 | - | 1026..1044 |
| PR-21721 (Cpo_CRPQ->_U2_fw) | SEQ ID NO:49 | - | 4..21 |
| PR-21722 (Cpo_CRPQ->_U2_rev) | SEQ ID NO:49 | - | 1029..1047 |
| PR-21729 (<-EpoE11_U1_fw) | SEQ ID NO:51 | - | 4..24 |
| PR-21730 (<-EpoE11_U1_rev) | SEQ ID NO:51 | - | 981..999 |
| PR-21733 (<-SlsZ_E11_U1_fw) | SEQ ID NO:53 | - | 4..23 |
| PR-21734 (<-SlsZ_E11_U1_rev) | SEQ ID NO:53 | - | 999..1017 |
| PR-21739 (<-CpaE11_U1_fw) | SEQ ID NO:55 | - | 4..21 |
| PR-21740 (<-CpaE11_U1_rev) | SEQ ID NO:55 | - | 987..1005 |
| PR-16594 (Har_FAR_codopt YL_U2_fw) | SEQ ID NO:58 | - | 4..21 |
| PR-16595 (Har_FAR_codopt YL_U2_rev) | SEQ ID NO:58 | - | 1348..1368 |
| PR-18214 (PTEFintron_USE R_rv) | Yali0C | NC_006069 | 1243743..1243761 |
| PR-18930 (PrTEF1 <- | Yali0C | NC_006069 | 1244239..1244226 |
| _forfusion_U1_fw) | | | |
| PR-18977 (PrTef->_fw) | Yali0C | NC_006069 | |
| PR-22853 (Sce_OLE1_fw) | SEQ ID NO:29 | - | 4..18 |
| PR-22854 (Sce_OLE1_rv) | SEQ ID NO:29 | - | 1512..1533 |
| PR-22855 (Yli_OLE1_fw) | SEQ ID NO:31 | - | 4..18 |
| PR-22856 (Yli_OLE1_rv) | SEQ ID NO:31 | - | 1433..1449 |
| PR-22857 (Dme_D9_YlopIDT _fw) | SEQ ID NO:33 | - | 4..17 |
| PR-22858 (Dme_D9_YlopIDT _rv) | SEQ ID NO:33 | - | 1068..1086 |
| PR-22859 (Atr_D11_YlopIDT _fw) | SEQ ID NO:36 | - | 4..17 |
| PR-22860 (Atr_D11_YlopIDT _rv) | SEQ ID NO:36 | - | 963..981 |
| PR-22861 (Dpu_E9-14_fw) | SEQ ID NO:45 | - | 4..18 |
| PR-22862 (Dpu_E9-14_rv) | SEQ ID NO:45 | - | 1041..1059 |
| PR-22863 (Gmo_CPRQ_fw) | SEQ ID NO:47 | - | 4..18 |
| PR-22864 (Gmo_CPRQ_rv) | SEQ ID NO:47 | - | 1022..1041 |
| PR-22865 (Har_FAR_YlopID T_fw) | SEQ ID NO:57 | - | 4..18 |
| PR-22866 (Har_FAR_YlopID T_rv) | SEQ ID NO:57 | - | 1348..1368 |
| PR-22867 (Har_FAR_fw) | SEQ ID NO:58 | - | 4..17 |
| PR-22868 (Har_FAR_rv) | SEQ ID NO:58 | - | 1348..1368 |
| PR-22869 (Sc_ATF1_fw) | SEQ ID NO:60 | - | 4..18 |
| PR-22870 (Sc_ATF1_rv) | SEQ ID NO:60 | - | 1564..1578 |
| PR10595 | Yali0C | NC_006069 | 1244239..1244226 |
| PR10604 | pCfB3401 | | - |
| PR10607 | Yali0A | NC_006067 | 483924..483945; overhang: 1 |
| PR10655 | Yali0C | NC_006069 | - |
| PR10656 | Yali0C | NC_006069 | - |
| PR11110 | pCfB6681 | - | - |
| PR11111 | pCfB6681 | - | - |
| PR11138 | pCfB4132 | - | - |
| PR11139 | Yali0F | NC_006072 | - |
| PR14148 | Yali0E | NC_006071 | - |
| PR14149 | Yali0A | NC_006067 | - |
| PR14279 | Yali0C | NC_006069 | 163477..163466 |
| PR14581 | Yali0F | NC_006072 | 795904..795923 |
| PR14583 | Yali0F | NC_006072 | 796450..796469 |
| PR14584 | Yali0F | NC_006072 | 796943..796924 |
| PR14585 | Yali0C | NC_006069 | 568316..568337 |
| PR14586 | Yali0C | NC_006069 | 568817..568802 |
| PR14587 | Yali0C | NC_006069 | 568862..568881 |
| PR14588 | Yali0C | NC_006069 | - |
| PR15521 | Yali0C | NC_006069 | 1663140..1663158 |
| PR15522 | Yali0C | NC_006069 | - |
| PR15781 | Yali0A | NC_006067 | - |
| PR15788 | Yali0A | NC_006067 | - |
| PR15789 | Yali0F | NC_006072 | - |
| PR15930 | Yali0C | NC_006069 | - |
| PR18066 | SEQ ID NO:58 | - | 1..21 |
| PR18239 | Yali0C | NC_006069 | 568875..568856 |
| PR18240 | Yali0C | NC_006069 | 568856..568875 |
| PR18241 | Yali0D | NC_006070 | 2193232..2193214 |
| PR18242 | Yali0D | NC_006070 | 2193213..2193232 |
| PR18245 | Yali0E | NC_006071 | 1722566..1722585 |
| PR18246 | Yali0E | NC_006071 | 1722585..1722566 |
| PR18255 | Yali0E | NC_006071 | 2882052..2882071 |
| PR18256 | Yali0E | NC_006071 | 2882071..2882052 |
| PR18282 | Yali0E | NC_006071 | 1722589..1722608 |
| PR18284 | Yali0E | NC_006071 | 2882092..2882111 |
| PR18289 | Yali0D | NC_006070 | 2193222..2193242 |
| PR19018 | Yali0F | NC_006072 | 3236932..3236944 |
| PR20762 | Yali0B | NC_006068 | 2566663..2566682 |
| PR20763 | Yali0B | NC_006068 | - |
| PR20764 | Yali0B | NC_006068 | - |
| PR20765 | Yali0B | NC_006068 | 2567653..2567636 |
| PR21723 | SEQ ID NO:73 | - | 4..22 |
| PR21724 | SEQ ID NO:73 | - | 997..1014 |
| PR21755 | SEQ ID NO:71 | - | 1..19 |
| PR21756 | SEQ ID NO:71 | - | 1995..2013 |
| PR21757 | SEQ ID NO: 82 | - | 1..19 |
| PR21758 | SEQ ID NO: 82 | - | 2076..2094 |
| PR21759 | SEQ ID NO:84 | - | 1..18 |
| PR21760 | SEQ ID NO:84 | - | 2046..2064 |
| PR21767 | Yali0C | NC_006069 | 1663140..1663158 |
| PR21768 | Yali0C | NC_006069 | 1664141..1664123 |
| PR21769 | Yali0C | NC_006069 | 1663140..1663158 |
| PR21770 | Yali0C | NC_006069 | 1664141_1664123 |
| PR21771 | Yali0C | NC_006069 | - |
| PR21806 | SEQ ID NO: 86 | - | 2..21 |
| PR21807 | SEQ ID NO: 86 | - | 999..1017 |
| PR21925 | Yali0C | NC_006069 | - |
| PR22039 | Yali0F | NC_006072 | 796363..796382 |
| PR22040 | Yali0F | NC_006072 | 796382..796363 |
| PR22045 | Yali0F | NC_006072 | 796354..796335 |
| PR22075 | Yali0C | NC_006069 | - |
| PR22188 | Yali0E | NC_006069 | 1601119..1601100 |
| PR22191 | Yali0E | NC_006069 | 1600091..1600110 |
| PR22213 | Yali0C | NC_006069 | - |
| PR22295 | SEQ ID NO:98 | - | 1032..1047 |
| PR22776 | Yali0A | NC_006067 | 356505..356486 |
| PR22777 | Yali0A | NC_006067 | 356486..356505 |
| PR23004 | Yali0C | NC_006069 | 825834..825853 |
| PR23012 | SEQ ID NO:88 | - | 4..19 |
| PR23013 | SEQ ID NO:21 | - | 1100..1116 |
| PR23014 | SEQ ID NO:21 | - | 4..21 |
| PR23123 | Yali0E | NC_006069 | 3898880..3898860 |
| PR23124 | Yali0E | NC_006069 | 3898861..3898880 |
| PR23134 | Yali0C | NC_006069 | 405783..405802 |
| PR23135 | Yali0C | NC_006069 | 406298..406284 |
| PR23136 | Yali0C | NC_006069 | 405665..405647 |
| PR23137 | Yali0C | NC_006069 | 405163..405177 |
| PR23138 | Yali0E | NC_006069 | 2795545..2795556 |
| PR23139 | Yali0E | NC_006069 | 2796053..2796036 |
| PR23140 | Yali0E | NC_006069 | 2795473..2795459 |
| PR23141 | Yali0E | NC_006069 | 2268152..2268140 |
| PR23166 | Yali0E | NC_006069 | 2086416..2086402 |
| PR23167 | Yali0E | NC_006069 | 2085907..2085919 |
| PR23168 | Yali0E | NC_006069 | 2086480..2086497 |
| PR23169 | Yali0E | NC_006069 | 2087001..2086986 |
| PR23170 | Yali0D | NC_006070 | 1058703..1058686 |
| PR23171 | Yali0D | NC_006070 | 1058205..1058220 |
| PR23172 | Yali0D | NC_006070 | 1058776..1058791 |
| PR23173 | Yali0D | NC_006070 | 1059321..1059304 |
| PR23174 | Yali0C | NC_006069 | 405782..405763 |
| PR23175 | Yali0C | NC_006069 | 405763..405782 |
| PR23176 | Yali0E | NC_006071 | 2795508..2795527 |
| PR23177 | Yali0E | NC_006071 | 2795527..2795508 |
| PR23192 | Yali0D | NC_006070 | 1058729..1058710 |
| PR23193 | Yali0D | NC_006070 | 1058710..1058729 |
| PR23308 | Yali0E | NC_006071 | 3265514..3265533 |
| PR23309 | Yali0E | NC_006071 | 3265533..3265514 |
| PR23385 | Yali0D | NC_006070 | 3291828..3291809 |
| PR23386 | Yali0D | NC_006070 | 3291809..3291828 |
| PR23435 | SEQ ID NO:13 | - | 1..15 |
| PR23436 | SEQ ID NO:13 | - | 2752..2770 |
| PR23632 | SEQ ID NO:9 | - | 1..15 |
| PR23633 | SEQ ID NO:9 | - | 2752..2770 |
| PR23655 | SEQ ID NO:100 | - | 4..21 |
| PR23656 | SEQ ID NO:100 | - | 966..984 |
| PR23671 | Yali0F | NC_006072 | 1449403..1449422 |
| PR23672 | Yali0F | NC_006072 | 1449422..1449403 |
| PR23693 | SEQ ID NO:9 | - | 1373..1391 |
| PR23702 | SEQ ID NO:98 | - | 1032..1047 |
| PR23947 | SEQ ID NO:108 | - | 4..13 |
| PR23949 | SEQ ID NO:110 | - | 1360..1380 |
| PR23950 | SEQ ID NO:112 | - | 4..14 |
| PR23951 | SEQ ID NO:112 | - | 1326..1344 |
| PR23952 | SEQ ID NO:114 | - | 4..15 |
| PR23953 | SEQ ID NO:114 | - | 973..993 |

TABLE 2

DNA fragments (BioBricks) obtained by PCR using the indicated template and primers

| | | | | |
|---|---|---|---|---|
| Gene fragment name | Gene | Fw_primer | Rv_primer | Template DNA |
| BB2670 | Peroxisomal oxidase 5 from Y. lipolytica | PR-10607 | PR-15791 | BB1635 |
| | | | | BB1636 |
| | | | | PR-21648 |
| | | | | PR-21649* |
| BB2671 | Peroxisomal oxidase 6 from Y. lipolytica | PR-15790 | PR-10604 | BB1635 |
| | | | | BB1636 |
| | | | | PR-21650 |
| | | | | PR-21651* |
| BB2672 | Peroxisomal oxidase 1 from Y. lipolytica | PR-10607 | PR-15791 | BB1635 |
| | | | | BB1636 |
| | | | | PR-21652 |
| | | | | PR-21653* |
| BB2673 | Peroxisomal oxidase 4 from Y. lipolytica | PR-15790 | PR-10604 | BB1635 |
| | | | | BB1636 |
| | | | | PR-21654 |
| | | | | PR-21655* |
| BB2674 | Peroxisomal oxidase 2 from Y. lipolytica | PR-10607 | PR-15791 | BB1635 |
| | | | | BB1636 |
| | | | | PR-21656 |
| | | | | PR-21657* |
| BB2675 | Peroxisomal oxidase 3 from Y. lipolytica | PR-15790 | PR-10604 | BB1635 |
| | | | | BB1636 |
| | | | | PR-21658 |
| | | | | PR-21659* |
| BB8515 | Peroxisomal oxidase 1 from Y. lipolytica | PR-22825 | PR-22826 | Genomic DNA of Y. lipolytica |
| BB8516 | Peroxisomal oxidase 2 from Y. lipolytica | PR-22827 | PR-22828 | Genomic DNA of Y. lipolytica |
| BB8517 | Peroxisomal oxidase 3 from Y. lipolytica | PR-22829 | PR-22830 | Genomic DNA of Y. lipolytica |
| BB8518 | Peroxisomal oxidase 4 from Y. lipolytica | PR-22831 | PR-22832 | Genomic DNA of Y. lipolytica |
| BB8519 | Peroxisomal oxidase 5 from Y. lipolytica | PR-22833 | PR-22834 | Genomic DNA of Y. lipolytica |
| BB8520 | Peroxisomal oxidase 6 from Y. lipolytica | PR-22835 | PR-22836 | Genomic DNA of Y. lipolytica |
| BB8521 | Peroxisomal oxidase from Agrotis segetum | PR-22837 | PR-22838 | SEQ ID NO: 13 |
| BB8522 | Peroxisomal oxidase 1 from Arabidopsis thaliana | PR-22839 | PR-22840 | pBP8312 (Ath_POX1) |
| BB8523 | Peroxisomal oxidase from Aspergillus nidulans | PR-22841 | PR-22842 | SEQ ID NO: 19 |
| BB8524 | Peroxisomal oxidase from Cucurbita maxima | PR-22843 | PR-22844 | SEQ ID NO: 21 |
| BB8525 | Peroxisomal oxidase from Homo sapiens | PR-22845 | PR-22846 | SEQ ID NO: 23 |
| BB8526 | Peroxisomal oxidase from Paenarthro-bacter ureafaciens | PR-22847 | PR-22848 | SEQ ID NO: 25 |
| BB8527 | Peroxisomal oxidase 2 from Rattus norvegicus | PR-22849 | PR-22850 | SEQ ID NO: 27 |
| BB8528 | Peroxisomal oxidase 2 from Arabidopsis thaliana | PR-22851 | PR-22852 | SEQ ID NO: 15 |
| BB8302 | TEF1 promoter from Y. lipolytica | PR-18928 | PR-18975 | Genomic DNA of Y. lipolytica |
| BB8529 | Δ_{Z9}-desaturase from Saccharomyces cerevisiae | PR-22853 | PR-22854 | SEQ ID NO: 29 |
| BB8530 | Δ_{Z9}-desaturase from Y. lipolytica | PR-22855 | PR-22856 | Genomic DNA of Y. lipolytica |
| BB8531 | Δ_{Z9}-14-desaturase from D. melanogaster | PR-22857 | PR-22858 | SEQ ID NO: 33 |
| BB8251 | Δ_{Z9}-14-desaturase from D. melanogaster | PR-10595 | PR-18489 | SEQ ID NO: 34 |
| BB8532 | Δ_{Z11}-16-desaturase from A. transitella | PR-22859 | PR-22860 | SEQ ID NO: 36 |
| BB8248 | Δ_{Z11}-16- desaturase from A. transitella | PR-10595 | PR-21764 | SEQ ID NO: 38 |
| BB2700 | Δ_{Z11}-14- desaturase from Choristoneura rosaceana | PR-21737 | PR-21738 (<- | SEQ ID NO: 39 |
| BB2695 | Δ_{Z11}-14- desaturase from O. nubilalis | PR-21727 | PR-21728 | SEQ ID NO: 41 |
| BB2697 | Δ_{Z11}-14- desaturase from T. pityocampa | PR-21731 | PR-21732 | SEQ ID NO: 43 |
| BB8533 | Δ_{E9}-14- desaturase from D. punctatus | PR-22861 | PR-22862 | SEQ ID NO: 45 |
| BB8534 | Δ_{Z/E10}-14-desaturase from G. molesta | PR-22863 | PR-22864 | SEQ ID NO: 47 |
| BB2692 | Desaturase from C. pomonella | PR-21721 | PR-21722 | SEQ ID NO: 98 |
| BB2696 | Δ_{E11}-14-desaturase from E. postvittana | PR-21729 | PR-21730 | SEQ ID NO: 51 |
| BB2698 | Δ_{E11}-14-desaturase from S. littoralis | PR-21733 | PR-21734 | SEQ ID NO: 53 |
| BB2701 | Δ_{E11}-14-desaturase from C. parallela | PR-21739 | PR-21740 | SEQ ID NO: 55 |
| BB8535 | fatty acyl-CoA reductase from H. armigera | PR-22865 | PR-22866 | SEQ ID NO: 57 |
| BB8536 | fatty acyl-CoA reductase from H. armigera | PR-22867 | PR-22868 | SEQ ID NO: 58 |
| BB1740 | fatty acyl-CoA reductase from H. armigera | PR-16594 | PR-16595 | SEQ ID NO: 58 |
| BB8537 | acetyltransferase from S. cerevisiae | PR-22869 | PR-22870 | SEQ ID NO: 60 |
| BB2719 | TEFintron promoter from Y. lipolytica | PR-18928 | PR-18214 | Genomic DNA of Y. lipolytica |
| BB2093 | TEFintron promoter from Y. lipolytica | PR-10595 | PR-18214 | Genomic DNA of Y. lipolytica |
| BB2720 | TEFintron promoter from Y. lipolytica | PR-18930 (PrTEF1 <-_forfusion_U1 _fw) | PR-18214 (PTEFintron_U SER_rv) | Genomic DNA of Y. lipolytica |
| BB2726 | TEFintron promoter from Y. lipolytica | PR-18977 | PR-18214 | Genomic DNA of Y. lipolytica |
| BB01005 | Hyrogmycin -B-phoshotrans ferase | PR-11138 | PR-11139 | pCfB2195 [2] |
| BB01006 | Vector backbone | PR-10655 | PR-10656 | pCfB3405 [1] |
| BB1135 | Vector backbone | PR-11110 | PR-11111 | pCfB6681 [1] |
| BB1480 | non-coding region from Y. lipolytica genome | PR-14583 | PR-14584 | genomic DNA of Y. lipolytica |
| BB1481 | non-coding region from Y. lipolytica genome | PR-14585 | PR-14586 | genomic DNA of Y. lipolytica |
| BB1482 | non-coding region from Y. lipolytica genome | PR-14587 | PR-14588 | genomic DNA of Y. lipolytica |
| BB1558 | Promoter Exp from Y. lipolytica | PR-15521 | PR-15522 | genomic DNA of Y. lipolytica |
| BB1631 | Terminator PEX20 and terminator of LIP2 from Y. lipolytica | PR-14148 | PR-15781 | pCfB4586 [1] |
| BB1635 | tRNA from Y. lipolytica | PR-10607 | PR-15788 | pCfB4589 [1] |
| BB1636 | non-coding region from Y. lipolytica genome | PR-15789 | PR-10604 | pCfB4589 [1] |
| BB1688 | Promoter Tefintron from Y. lipolytica | PR-14279 | PR-15930 | genomic DNA of Y. lipolytica |
| BB2068 | Fatty acyl reductase from H. armigera | PR-18066 | PR-16595 | SEQ ID NO: 58 |
| BB2104 | Vector backbone | PR-18282 | PR-15781 | pCfB6367 [1] |
| BB2106 | Vector backbone | PR-18284 | PR-15781 | pCfB5251 [1] |
| BB2111 | Vector backbone | PR-18289 | PR-15781 | pCfB5582 [1] |
| BB2311 | Fatty acid synthase 2 from Y. lipolytica | PR-20762 | PR-20763 | genomic DNA of Y. lipolytica |
| BB2312 | Fatty acid synthase 2 from Y. lipolytica | PR-20764 | PR-20765 | genomic DNA of Y. lipolytica |
| BB2313 | Fatty acid synthase 2 from Y. lipolytica | PR-20762 | PR-20765 | BB2311, BB2312* |
| BB2646 | Peroxisomal oxidase 1 from Y. lipolytica | PR-21660 | PR-21661 | genomic DNA of Y. lipolytica |
| BB2647 | Peroxisomal oxidase 1 from Y. lipolytica | PR-21662 | PR-21663 | genomic DNA of Y. lipolytica |
| BB2648 | Peroxisomal oxidase 1 from Y. lipolytica | PR-21660 | PR-21663 | BB2646, BB2647* |
| BB2649 | Peroxisomal oxidase 2 from Y. lipolytica | PR-21664 | PR-21665 | genomic DNA of Y. lipolytica |
| BB2650 | Peroxisomal oxidase 2 from Y. lipolytica | PR-21666 | PR-21667 | genomic DNA of Y. lipolytica |
| BB2651 | Peroxisomal oxidase 2 from Y. lipolytica | PR-21664 | PR-21667 | BB2649, BB2650* |
| BB2652 | Peroxisomal oxidase 3 from Y. lipolytica | PR-21668 | PR-21669 | genomic DNA of Y. lipolytica |
| BB2653 | Peroxisomal oxidase 3 from Y. lipolytica | PR-21670 | PR-21671 | genomic DNA of Y. lipolytica |
| BB2654 | Peroxisomal oxidase 3 from Y. lipolytica | PR-21668 | PR-21671 | BB2652, BB2653* |
| BB2655 | Peroxisomal oxidase 4 from Y. lipolytica | PR-21672 | PR-21673 | genomic DNA of Y. lipolytica |
| BB2656 | Peroxisomal oxidase 4 from Y. lipolytica | PR-21674 | PR-21675 | genomic DNA of Y. lipolytica |
| BB2657 | Peroxisomal oxidase 4 from Y. lipolytica | PR-21672 | PR-21675 | BB2655, BB2656 |
| BB2658 | Peroxisomal oxidase 5 from Y. lipolytica | PR-21676 | PR-21677 | genomic DNA of Y. lipolytica |
| BB2659 | Peroxisomal oxidase 5 from Y. lipolytica | PR-21678 | PR-21679 | genomic DNA of Y. lipolytica |
| BB2660 | Peroxisomal oxidase 5 from Y. lipolytica | PR-21676 | PR-21679 | BB2658, BB2659* |
| BB2661 | Peroxisomal oxidase 6 from Y. lipolytica | PR-21680 | PR-21681 | genomic DNA of Y. lipolytica |
| BB2662 | Peroxisomal oxidase 6 from Y. lipolytica | PR-21682 | PR-21683 | genomic DNA of Y. lipolytica |
| BB2663 | Peroxisomal oxidase 6 from Y. lipolytica | PR-21680 | PR-21683 | BB2661, BB2662* |
| BB2693 | ΔZ11 desaturase from L. botrana | PR-21723 | PR-21724 | SEQ ID NO: 78 |
| BB2709 | Peroxisomal oxidase from L. botrana | PR-21755 | PR-21756 | SEQ ID NO: 80 |
| BB2710 | Peroxisomal oxidase from L. botrana | PR-21757 | PR-21758 | SEQ ID NO: 82 |
| BB2711 | Peroxisomal oxidase from L. botrana | PR-21759 | PR-21760 | SEQ ID NO: 84 |
| BB2721 | Promoter Exp from Y. lipolytica | PR-21767 | PR-21768 | genomic DNA of Y. lipolytica |
| BB2722 | Promoter Exp from Y. lipolytica | PR-21769 | PR-21770 | genomic DNA of Y. lipolytica |
| BB2723 | Promoter Exp from Y. lipolytica | PR-21771 | PR-15522 | genomic DNA of Y. lipolytica |
| BB8018 | Double promoter Exp/Tefintro n from Y. lipolytica | PR-18214 | PR-21925 | BB2720, BB2723* |
| BB8031 | non-coding region from Y. lipolytica genome | PR-14581 | PR-22045 | genomic DNA of Y. lipolytica |
| BB8141 | Promoter Yef3 from Y. lipolytica | PR-22191 | PR-22188 | genomic DNA of Y. lipolytica |
| BB8212 | Promoter Tefintron from Y. lipolytica and fatty acyl reductase of H. armigera | PR-10595 | PR-14149 | pBP7980 |
| BB8213 | Promoter Tefintron from Y. lipolytica and fatty acyl reductase of H. armigera | PR-22075 | PR-16595 | pBP7980 |
| BB8615 | Δ11 desaturase from L. botrana | PR-23012 | PR-23013 | SEQ ID NO: 88 |
| BB8616 | Δ9 desaturase from Chilo suppressalis | PR-23014 | PR-23013 | SEQ ID NO: 90 |
| BB8640 | Δ9 desaturase from Drosophila melanogast er | PR-19018 | PR-18930 | SEQ ID NO: 34 |
| BB8644 | Promoter GPD from Y. lipolytica | PR-23004 | PR-22213 | genomic DNA of Y. lipolytica |
| BB8663 | non-coding region from Y. lipolytica genome | PR-23135 | PR-23134 | genomic DNA of Y. lipolytica |
| BB8664 | non-coding region from Y. lipolytica genome | PR-23136 | PR-23137 | genomic DNA of Y. lipolytica |
| BB8665 | non-coding region from Y. lipolytica genome | PR-23139 | PR-23138 | genomic DNA of Y. lipolytica |
| BB8666 | non-coding region from Y. lipolytica genome | PR-23140 | PR-23141 | genomic DNA of Y. lipolytica |
| BB8679 | non-coding region from Y. lipolytica genome | PR-23167 | PR-23166 | genomic DNA of Y. lipolytica |
| BB8680 | non-coding region from Y. lipolytica genome | PR-23168 | PR-23169 | genomic DNA of Y. lipolytica |
| BB8681 | non-coding region from Y. lipolytica genome | PR-23171 | PR-23170 | genomic DNA of Y. lipolytica |
| BB8682 | non-coding region from Y. lipolytica genome | PR-23172 | PR-23173 | genomic DNA of Y. lipolytica |
| BB8715 | Vector backbone | PR-23170 | PR-23172 | pBP8566 [1] |
| BB8769 | Peroxisomal oxidase from Agrotis segetum | PR-23435 | PR-23436 | SEQ ID NO: 13 |
| BB8816 | Fatty acyl reductase from A. segetum | PR-23632 | PR-23633 | SEQ ID NO: 92 |
| BB8820 | Δ11 desaturase from Pectinophor a gossypiella | PR-23655 | PR-23656 | SEQ ID NO: 100 |
| BB8829 | Fatty acyl reductase from A. segetum | PR-23632 | PR-23693 | SEQ ID NO: 92 |
| BB8932 | desaturase from L. botrana | PR-23947 | PR-23949 | SEQ ID NO: 110 |
| BB8933 | desaturase from L. botrana | PR-23950 | PR-23951 | SEQ ID NO: 112 |
| BB8934 | desaturase from L. botrana | PR-23952 | PR-23953 | SEQ ID NO: 114 |

* The template for PCR reaction was obtained by mixing the indicating DNA fragments and treating them sequentially with USER-enzyme and then T4 DNA ligase.

TABLE 3

Integrative expression vectors

| | | |
|---|---|---|
| Integrative expression vector name | Parent vector | DNA fragments cloned into parent vector |
| pBP8339 (pintC_3-TPex20-Yli_POX1 {-PrTEF1- TLip2) | pCfB6371 | BB8302 ({-PrTEF1) |
| | | BB8515 ({-Yli_POX1) |
| pBP8340 (pintC_3-TPex20-Yli_POX2{-PrTEF1-TLip2) | pCfB6371 | BB8302 ({-PrTEF1) |
| | | BB8516 ({-Yli_POX2) |
| pBP8341 (pintC_3-TPex20-Yli_POX3{-PrTEF1-TLip2) | pCfB6371 | BB8302 ({-PrTEF1) |
| | | BB8517 ({-Yli_POX3) |
| pBP8342 (pintC_3-TPex20-Yli_POX4{-PrTEF1-TLip2) | pCfB6371 | BB8302 ({-PrTEF1) |
| | | BB8518 ({-Yli_POX4) |
| pBP8343 (pintC_3-TPex20-Yli_POX5{-PrTEF1-TLip2) | pCfB6371 | BB8302 ({-PrTEF1) |
| | | BB8519 ({-Yli_POX5) |
| pBP8344 (pintC_3-TPex20-Yli_POX6{-PrTEF1-TLip2) | pCfB6371 | BB8302 ({-PrTEF1) |
| | | BB8520 ({-Yli_POX6) |
| pBP8345 (pIntC_3-TPex20-Ase_POX{-PrTEF1-TLip2) | pCfB6371 | BB8302 ({-PrTEF1) |
| | | BB8521 ({-Ase_POX) |
| pBP8346 (pIntC_3-TPex20-Ath_POX1{-PrTEF1-TLip2) | pCfB6371 | BB8302 ({-PrTEF1) |
| | | BB8522 ({-Ath_POX1) |
| pBP8347 (pIntC_3-TPex20-Ani_POX{-PrTEF1-TLip2) | pCfB6371 | BB8302 ({-PrTEF1) |
| | | BB8523 ({-Ani_POX) |
| pBP8348 (pIntC_3-TPex20-Cma_POX{-PrTEF1-TLip2) | pCfB6371 | BB8302 ({-PrTEF1) |
| | | BB8524 ({-Cma_POX) |
| pBP8349 (pIntC_3-TPex20-Hsa_POX1-2{-PrTEF1-TLip2) | pCfB6371 | BB8302 ({-PrTEF1) |
| | | BB8525 ({-Hsa_POX1-2) |
| pBP8350 (pIntC_3-TPex20-Pur_POX{-PrTEF1-TLip2) | pCfB6371 | BB8302 ({-PrTEF1) |
| | | BB8526 ({-Pur_POX) |
| pBP8351 (pIntC_3-TPex20-Rno_POX-2{-PrTEF1-TLip2) | pCfB6371 | BB8302 ({-PrTEF1) |
| | | BB8527 ({-Rno_POX-2) |
| pBP8353 (pIntC_3-TPex20-Ath_POX2{-PrTEF1-TLip2) | pCfB6371 | BB8302 ({-PrTEF1) |
| | | BB8528 ({-Ath_POX2) |
| pBP8377 (pIntD_1-TPex20-Sce_OLE1 {-PrTEFintron-TLip2) | pCfB6684 | BB2719 ({-PrTefintron_USER) |
| | | BB8529 ({-Sce_OLE1) |
| pBP8378 (pIntD_1-TPex20-Yli_OLE1-{-PrTEFintron-TLip2) | pCfB6684 | BB2719 ({-PrTefintron_USER) |
| | | BB8530 ({-Yli_OLE1) |
| pBP8379 (pIntE_4-TPex20-Tefintron-}Dme_D9 YIopIDT-TLip2) | pCfB6679 | BB2093 (PrTEFintron_USER-}) |
| | | BB8531 (Dme_D9_YIopIDT-}) |
| pBP8132 (pIntE_4-TPex20-Tefintron-}Dmd9-TLip2) | pCfB6679 | BB8251 (Tefintron-Dmd9) |
| pBP8380 (plntE₋4-TPex20-Tefintron-}Atr_D11-YIopIDT -TLip2) | pCfB6679 | BB2093 (PrTEFintron_USER-}) |
| | | BB8532 (Atr_D11-YIopIDT-}) |
| pBP8131 (pIntE_4-TPex20-Tefintron-}Atrd11-TLip2) | pCfB6679 | BB8248 (Tefintron-Atrd11) |
| pBP7919 (pIntD_1-TPex20-Cro_Z11{-PrTEFintron-Tlip2) | pCfB6684 | BB2719 ({-PrTEFintron_USER) |
| | | BB2700 (<-Cro_Z11) |
| pBP7914 (pIntD_1-TPex20-Onu_11{-PrTEFintron-Tlip2) | pCfB6684 | BB2719 ({-PrTEFintron_USER) |
| | | BB2687 (<-Onu_11) |
| pBP7916 (pIntD_1-TPex20-Tpi_D13{-PrTEFintron-Tlip2) | pCfB6684 | BB2719 ({-PrTEFintron_USER) |
| | | BB2697 (<-Tpi_D13) |
| pBP8381 (pIntE_1-TPex20-Dpu_E9-14{-PrTEFintron-TLip2) | pCfB6677 | BB2719 ({-PrTefintron_USER) |
| | | BB8533 ({-Dpu_E9-14) |
| pBP8382 (pIntD_1-TPex20-Gmo_CPRQ{-PrTEFintron-TLip2) | pCfB6684 | BB2719 ({-PrTefintron_USER) |
| | | BB8534 ({-Gmo_CPRQ) |
| pBP7911 (pIntD_1-TPex20-PrTEFintron-}Cpo_CPRQ-Tlip2) | pCfB6684 | BB2093 (PrTEFintron_USER-}) |
| | | BB2692 (Cpo_CRPQ->) |
| pBP7915 (pIntD_1-TPex20-EpoE11{-PrTEFintron-Tlip2) | pCfB6684 | BB2719 ({-PrTefintron_USER) |
| | | BB2696 (<-EpoE11) |
| pBP7917 (pIntD_1-TPex20-SlsZE11{-PrTEFintron-Tlip2) | pCfB6684 | BB2719 ({-PrTefintron_USER) |
| | | BB2698 (<-SlsZ_E11) |
| pBP7920 (pIntD_1-TPex20-CpaE11{-PrTEFintron-Tlip2) | pCfB6684 | BB2719 ({-PrTefintron_USER) |
| | | BB2701 (<-CpaE11) |
| pBP8383 (pInte_2-TPex20-PrTefintron-}Har_FAR_YIopIDT-Tlip2) | pCfB6682 | BB2093 (PrTEFintron_USER-}) |
| | | BB8535 (Har_FAR_YIopIDT-}) |
| pBP8384 (plntC_2-TPex20-PrTefintron-}Har_FAR-Tlip2) | pCfB6682 | BB2093 (PrTEFintron_USER-}) |
| | | BB8536 (Har_FAR-}) |
| pBP8385 (pInte_2-TPex20-Sc_ATF1{-PrTefintron-PrTefintron-}Har_FAR-TLip2) | pCfB6682 | BB2720 ({-PrTefintron_USER_forfusion) BB8537 (TPex20-Atf1{-Tefintron) |
| | | BB2726 (PrTefintron_forfusion->) |
| | | BB8536 (Har_FAR-}) |
| pCfB6364 [3] | | |
| pCfB6371 | | BB1135 |
| | | BB1631 |
| | | BB1481 |
| | | BB1482 |
| pCfB6677 | | BB2104 |
| pCfB6679 | | BB2106 |
| pCfB6684 | | BB2111 |
| pBP7912 | pCfB6684 | BB2719 |
| | | BB2693 |
| pBP7914 | pCfB6684 | BB2719 |
| | | BB2695 |
| pBP7915 | pCfB6684 | BB2719 |
| | | BB2696 |
| pBP7917 | pCfB6684 | BB2719 |
| | | BB2698 |
| pBP7919 | pCfB6684 | BB2719 |
| | | BB2700 |
| pBP7920 | pCfB6684 | BB2719 |
| | | BB2701 |
| pBP7980 | pCfB6371 | BB1688 |
| | | BB1740 |
| pBP8009 | | BB1135 |
| | | BB1631 |
| | | BB8031 |
| | | BB1480 |
| pBP8071 | pCfB6679 | BB8212 |
| | | BB8213 |
| pBP8236 | pCfB6679 | BB1688 |
| | | BB1740 |
| pBP8340 | pCfB6371 | BB8516 |
| | | BB2721 |
| pBP8341 | pCfB6371 | BB8517 |
| | | BB2721 |
| pBP8343 | pCfB6371 | BB8519 |
| | | BB2721 |
| pBP8347 | pCfB6371 | BB8523 |
| | | BB2721 |
| pBP8348 | pCfB6371 | BB8524 |
| | | BB2721 |
| pBP8349 | pCfB6371 | BB8525 |
| | | BB2721 |
| pBP8350 | pCfB6371 | BB8526 |
| | | BB2721 |
| pBP8351 | pCfB6371 | BB8527 |
| | | BB2721 |
| pBP8377 | pCfB6684 | BB2719 |
| | | BB8529 |
| pBP8378 | pCfB6684 | BB2719 |
| | | BB8530 |
| pBP8400 | pCfB6371 | BB2709 |
| | | BB1558 |
| pBP8401 | pCfB6371 | BB2710 |
| | | BB1558 |
| pBP8577 | pCfB6684 | BB8132 |
| pBP8620 | | BB1135 |
| | | BB8682 |
| | | BB8681 |
| pBP8622 | | BB1135 |
| | | BB1631 |
| | | BB8664 |
| | | BB8663 |
| pBP8625 | pCfB3431 | BB1635 |
| | | BB1636 |
| | | PR23174 |
| | | PR23175 |
| pBP8627 | pBP8620 | BB8640 |
| | | BB2720 |
| | | BB8644 |
| | | BB2068 |
| pBP8660 | | BB1135 |
| | | BB8665 |
| | | BB1631 |
| | | BB8666 |
| pBP8662 | | BB1135 |
| | | BB8679 |
| | | BB8680 |
| | | BB1631 |
| pBP8754 | pBP8009 | BB2721 |
| | | BB8769 |
| pBP8782 | pBP8660 | BB2093 |
| | | BB8816 |
| pBP8789 | pBP8394 | BB2093 |
| | | BB8820 |
| pBP8819 | pCfB6684 | BB2093 |
| | | BB8615 |
| pBP8820 | pCfB6684 | BB2093 |
| | | BB8616 |
| pBP8875 | pBP8622 | BB2726 |
| | | BB8816 |
| | | BB2721 |
| | | BB8769 |
| pBP8878 | pBP8622 | BB2726 |
| | | BB8816 |
| | | BB8526 |
| | | BB2721 |
| pBP8879 | pBP8622 | BB8527 |
| | | BB2721 |
| | | BB2726 |
| | | BB8816 |
| pBP8880 | pBP8622 | BB2726 |
| | | BB8816 |
| | | BB8516 |
| | | BB2722 |
| pBP8882 | pBP8622 | BB8829 |
| | | BB8018 |
| | | BB2710 |
| pBP8883 | pBP8622 | BB8829 |
| | | BB8018 |
| | | BB2711 |
| pBP8977 | pBP8662 | BB2093 |
| | | BB8945 |

TABLE 4

Helper vectors

| | | | |
|---|---|---|---|
| gRNA vector name | Selection marker | Parent vector | DNA fragments cloned into parent vector |
| pBP7883 | Nat | pCfB3405 | BB2670 |
| | | | BB2671 |
| pBP7884 | Nat | pCfB3405 | BB2672 |
| | | | BB2673 |
| pBP7885 | Nat | pCfB3405 | BB2674 |
| | | | BB2675 |
| pCfB6627 | Nat | pCfB3405 | BB1635 |
| | | | BB1636 |
| | | | PR-18233 |
| | | | PR-18234 |
| pCfB6630 | Nat | pCfB3405 | BB1635 |
| | | | BB1636 |
| | | | PR-18239 |
| | | | PR-18240 |
| pCfB6631 | Nat | pCfB3405 | BB1635 |
| | | | BB1636 |
| | | | PR-18241 |
| | | | PR-18242 |
| pCfB6633 | Nat | pCfB3405 | BB1635 |
| | | | BB1636 |
| | | | PR-18245 |
| | | | PR-18246 |
| pCfB6638 | Nat | pCfB3405 | BB1635 |
| | | | BB1636 |
| | | | PR-18255 |
| | | | PR-18256 |
| pCfB3431 | HYG | | BB1006, BB1005 |
| pBP8033 | HYG | pCfB3431 | BB1635, BB1636, PR18241, PR18242 |
| pBP8161 | HYG | pCfB3431 | BB1635, BB1636, PR18255, PR18256 |
| pBP8535 | HYG | pCfB3431 | BB1635, PR23123, PR23124, BB1636 |
| pBP8568 | HYG | pCfB3431 | BB1635, BB1636, PR23176, PR23177 |
| pBP8576 | HYG | pCfB3431 | BB1635, BB1636, PR23192, PR23193 |
| pBP8656 | HYG | pCfB3431 | BB1635, BB1636, PR21658, PR21659 |
| pBP8658 | HYG | pCfB3431 | BB1635, BB1636, PR21650, PR21651 |
| pBP8802 | HYG | pBP8620 | BB2693, BB2720, BB8644, BB2068 |
| pBP8813 | HYG | pCfB3431 | BB1635, BB1636, PR23671, |
| | | | PR23672 |
| pCfB3405 [1] | NAT | | |
| pCfB7088 | NAT | pCfB3405 | BB1635, BB1636 |
| pBP8003 | NAT | pCfB3405 | BB1635, BB1636, PR22039, PR22040 |
| pBP8032 | NAT | pCfB3405 | BB1635, BB1636, PR18239, PR18240 |
| pBP8328 | NAT | pCfB3405 | BB1635, BB1636, PR22776, PR22777 |
| pBP8567 | NAT | pCfB3405 | BB1635, BB1636, PR23174, PR23175 |
| pBP8623 | NAT | pCfB3405 | BB1635, BB1636, PR23192, PR23193 |
| pBP8650 | NAT | pCfB3405 | BB1635, BB1636, PR23308, PR23309 |
| pBP8657 | NAT | pCfB3405 | BB1635, BB1636 |
| pBP8659 | NAT | pCfB3405 | BB1635, BB1636, PR21656, PR21657 |
| pBP8674 | NAT | pCfB3405 | BB1635, BB1636, PR23176, PR23177 |
| pBP8704 | NAT | pCfB3405 | BB1635, BB1636, PR23385, PR23386 |

TABLE 5

Yeast strains

| | | | |
|---|---|---|---|
| Strain name | Deleted intrinsic genes | Overexpressed gene(s) | Parent strain and (integrated vectors) |
| ST7986 (Δku70 Δhfd4 Δhfd1 PEX10 Δfao1 Δhfd2 Δhfd3 GPAT_100bpPr) | HFD1 (YALI0_F23793g) | - | ST6629 |
| | HFD2 (YALI0_E15400g) | | |
| | HFD3 (YALI0_A17875g) | | |
| | HFD4 (YALI0_B01298g) | | |
| | FAO1 (YALI0_B14014g) | | |
| ST7991 (Δku70 Δhfd4 Δhfd1 PEX10 Δfao1 Δhfd2 Δhfd3 GPAT_100bpPr Δpox5 Δpox6) | HFD1 (YALI0_F23793g) | - | ST7986 |
| | HFD2 (YALI0_E15400g) | | |
| | HFD3 (YALI0_A17875g) | | |
| | HFD4 (YALI0_B01298g) | | |
| | FAO1 (YALI0_B14014g) | | |
| | POX5 (YALI0_C23859g) | | |
| | POX6 (YALI0_E06567g) | | |
| ST8015 (Δku70Δhfd4 Δhfd1 PEX10 Δfao1 Δhfd2 Δhfd3 GPAT_100bpPr Δpox5 Δpox6 Δpox1 Δpox4) | HFD1 (YALI0_F23793g) | - | ST7991 |
| | HFD2 (YALI0_E15400g) | | |
| | HFD3 (YALI0_A17875g) | | |
| | HFD4 (YALI0_B01298g) | | |
| | FAO1 (YALI0_B14014g) | | |
| | POX5 (YALI0_C23859g) | | |
| | POX6 (YALI0_E06567g) | | |
| | POX1 (YALI0_E32835g) | | |
| | POX4 (YALI0_E27654g) | | |
| ST8016 (Δku70Δhfd4 Δhfd1 PEX10 Δfao1 Δhfd2 Δhfd3 GPAT_100bpPr Δpox1-6) | HFD1 (YALI0_F23793g) | - | ST8015 |
| | HFD2 (YALI0_E15400g) | | |
| | HFD3 (YALI0_A17875g) | | |
| | HFD4 (YALI0_B01298g) | | |
| | FAO1 (YALI0_B14014g) | | |
| | POX5 (YALI0_C23859g) | | |
| | POX6 (YALI0_E06567g) | | |
| | POX1 (YALI0_E32835g) | | |
| | POX4 (YALI0_E27654g) | | |
| | POX2 (YALI0_F10857g) | | |
| | POX3 (YALI0_D24750g) | | |
| ST8588 (Δku70Δhfd4 Δhfd1 PEX10 Δfao1 Δhfd2 Δhfd3 GPAT_100bpPr Δpox1-6 pIntC_3-Yli_POX1{-PrTEF1) | Reference: ST8016 | Yli_POX1 (SEQ ID NO.1) | ST8016 (pBP8339) |
| ST8589 (Δku70Δhfd4 Δhfd1 PEX10 Δfao1 Δhfd2 Δhfd3 GPAT_100bpPr Δpox1-6 pIntC_3-Yli_POX2{-PrTEF1) | Reference: ST8016 | Yli_POX2 (SEQ ID NO.3) | ST8016 (pBP8340) |
| ST8590 (Δku70Δhfd4 Δhfd1 PEX10 Δfao1 Δhfd2 Δhfd3 GPAT_100bpPr Δpox1-6 pIntC_3-Yli_POX3{-PrTEF1) | Reference: ST8016 | Yli_POX3 (SEQ ID NO.5) | ST8016 (pBP8341) |
| ST8591 (Δku70 Δhfd4 Δhfd1 PEX10 Δfao1 Δhfd2 Δhfd3 GPAT_100bpPr Δpox1-6 pIntC_3-Yli_POX4{-PrTEF1) | Reference: ST8016 | Yli_POX4 (SEQ ID NO.7) | ST8016 (pBP8342) |
| ST8592 (Δku70 Δhfd4 Δhfd1 PEX10 Δfao1 Δhfd2 Δhfd3 GPAT_100bpPr Δpox1-6 pIntC_3-Yli_POX5{-PrTEF1) | Reference: ST8016 | Yli_POX5 (SEQ ID NO.9) | ST8016 (pBP8343) |
| ST8593 (Δku70 Δhfd4 Δhfd1 PEX10 Δfao1 Δhfd2 Δhfd3 GPAT_100bpPr Δpox1-6 pIntC_3-Yli_POX6{-PrTEF1) | Reference: ST8016 | Yli_POX6 (SEQ ID NO.11) | ST8016 (pBP8344) |
| ST8594 (Δku70 Δhfd4 Δhfd1 PEX10 Δfao1 Δhfd2 Δhfd3 GPAT_100bpPr Δpox1-6 pIntC_3-Ase_POX{-PrTEF1) | Reference: ST8016 | Ase_POX (SEQ ID NO.13) | ST8016 (pBP8345) |
| ST8595 (Δku70 Δhfd4 Δhfd1 PEX10 Δfao1 Δhfd2 Δhfd3 GPAT_100bpPr Δpox1-6 pIntC_3-Ath_POX1{-PrTEF1) | Reference: ST8016 | Ath_POX1 (SEQ ID NO.15) | ST8016 (pBP8346) |
| ST8596 (Δku70 Δhfd4 Δhfd1 PEX10 Δfao1 Δhfd2 Δhfd3 GPAT_100bpPr Δpox1-6 pIntC_3-Ani_POX{-PrTEF1) | Reference: ST8016 | Ani_POX (SEQ ID NO.19) | ST8016 (pBP8347) |
| ST8597 (Δhfd4 Δhfd 1 PEX10 Δfao1 Δhfd2 Δhfd3 GPAT_100bpPr Δpox1-6 pIntC_3-Cma_POX{-PrTEF1) | Reference: ST8016 | Cma_POX (SEQ ID NO.21) | ST8016 (pBP8348) |
| ST8598 (Δku70 Δhfd4 Δhfd1 PEX10 Δfao1 Δhfd2 Δhfd3 GPAT_100bpPr Δpox1-6 pIntC_3-Hsa_POX1-2{-PrTEF1) | Reference: ST8016 | Hsa_POX1-2 (SEQ ID NO.23) | ST8016 (pBP8349) |
| ST8599 (Δku70 Δhfd4 Δhfd1 PEX10 Δfao1 Δhfd2 Δhfd3 GPAT_100bpPr Δpox1-6 pIntC_3-Pur_POX{-PrTEF1) | Reference: ST8016 | Pur_POX (SEQ ID NO.25) | ST8016 (pBP8350) |
| ST8600 (Δku70 Δhfd4 Δhfd1 PEX10 Δfao1 Δhfd2 Δhfd3 GPAT_100bpPr Δpox1-6 pIntC_3-Rno_POX-2{-PrTEF1) | Reference: ST8016 | Rno_POX-2 (SEQ ID NO.27) | ST8016 (pBP8351) |
| ST8601 (Δku70 Δhfd4 Δhfd1 PEX10 Δfao1 Δhfd2 Δhfd3 GPAT_100bpPr Δpox1-6 | Reference: ST8016 | Ath_POX2 (SEQ ID NO.17) | ST8016 (pBP8353) |
| pIntC-3-Ath_POX2{-PrTEF1) | | | |
| ST6029 | | D-serine deaminase | Wild type Y. lipolytica (pCfB6364) |
| ST6629 | HFD4 HFD1 PEX10 FAO1 HFD2 HFD3 GPAT_100bpPr | D-serine deaminase | |
| ST7982 | HFD4 HFD1 PEX10 FAO1 HFD2 HFD3 GPAT_100bpPr FAS2 (11220F) | | ST6629 (pCfB7088, BB2313) |
| ST8225 | Reference: ST7982 | HarFAR | ST7982 (pCfB6638, pBP8071) |
| ST8373 | Reference: ST7982 | HarFAR Lbo_PPTQ | ST8225 (pBP7912, pCfB6631) |
| ST8374 | Reference: ST7982 | HarFAROnu11 | ST8225 (pBP7914, pCfB6631) |
| ST8375 | Reference: ST7982 | HarFAR EpoE11 | ST8225 (pBP7915, pCfB6631) |
| ST8376 | Reference: ST7982 | HarFAR SlsZE11 | ST8225 (pBP7917, pCfB6631) |
| ST8377 | Reference: ST7982 | HarFAR CroZ11 | ST8225 (pBP7919, pCfB6631) |
| ST8378 | Reference: ST7982 | HarFAR CpaE11 | ST8225 (pBP7920, pCfB6631) |
| ST8680 | Reference: ST8616 | HarFAR | ST8616 (pBP8236, pBP8161) |
| ST8835 | Reference: ST8616 POX1 | | () |
| ST8915 | Reference: ST8616 | PEX10 | ST8835 (pBP8033, pBP8577) |
| ST8916 | Reference: ST8915 POX2 | | ST8915 (pBP8659, BB2651) |
| ST8982 | Reference: ST8915 POX2 POX5 | | ST8916 (pBP8657, BB2660) |
| ST8997 | Reference: ST8915 | | ST8982 |
| | POX2 POX5 POX6 | | (pBP8658, BB2663) |
| ST9008 | Reference: ST8915 POX2 POX5 POX6 POX4 | | ST8997 (pBP8650, BB2657) |
| ST9089 | Reference: ST8915 POX2 POX5 POX6 POX4 POX3 | | ST9008 (pBP8656, BB2654) |
| ST9138 | Reference: ST8915 POX2 POX5 POX6 POX4 POX3 POX1 | | ST9089 (pBP8535, BB2648) |
| ST9163 | POX5 | | ST6029 (pBP8657, BB2660) |
| ST9179 | POX5 POX3 | | ST9163 (BB2654, pBP8704) |
| ST9199 | Reference: ST9138 | POX2 | ST9138 (pCfB6630, pBP8340) |
| ST9200 | Reference: ST9138 | POX3 | ST9138 (pCfB6630, pBP8341) |
| ST9202 | Reference: ST9138 | POX5 | ST9138 (pCfB6630, pBP8343) |
| ST9206 | Reference: ST9138 | Ani_POX | ST9138 (pCfB6630, pBP8347) |
| ST9207 | Reference: ST9138 | Cma_POX | ST9138 (pCfB6630, pBP8348) |
| ST9208 | Reference: ST9138 | Hsa_POX1-2 | ST9138 (pCfB6630, pBP8349) |
| ST9209 | Reference: ST9138 | Pur_POX | ST9138 (pCfB6630, pBP8350) |
| ST9210 | Reference: ST9138 | Rno_POX-2 | ST9138 (pCfB6630, pBP8351) |
| ST9215 | POX5 POX3 POX1 | | ST9179 (BB2648, pBP8535) |
| ST9252 | POX5 POX3 POX1 POX4 | | ST9215 (BB2657, pBP8650) |
| ST9284 | Reference: ST9138 | Ase_POX | ST9138 |
| | | | (pBP8754, pBP8003) |
| ST9285 | Reference: ST9138 | SlitDes5 HarFAR | ST9138 (pBP8626, pBP8623) |
| ST9286 | Reference: ST9138 | POX2 SlitDes5 HarFAR | ST9199 (pBP8626, pBP8576) |
| ST9287 | Reference: ST9138 | POX3 SlitDes5 HarFAR | ST9200 (pBP8626, pBP8576) |
| ST9288 | Reference: ST9138 | POX5 SlitDes5 HarFAR | ST9202 (pBP8626, pBP8576) |
| ST9289 | Reference: ST9138 | Ani_POX SlitDes5 HarFAR | ST9206 (pBP8626, pBP8576) |
| ST9290 | Reference: ST9138 | Cma_POX SlitDes5 HarFAR | ST9207 (pBP8626, pBP8576) |
| ST9291 | Reference: ST9138 | Hsa_POX1-2 SlitDes5 HarFAR | ST9208 (pBP8626, pBP8576) |
| ST9292 | Reference: ST9138 | Pur_POX SlitDes5 HarFAR | ST9209 (pBP8626, pBP8576) |
| ST9293 | Reference: ST9138 | Rno_POX-2 SlitDes5 HarFAR | ST9210 (pBP8626, pBP8576) |
| ST9294 | Reference: ST9138 | Dmd9 HarFAR | ST9138 (pBP8627, pBP8623) |
| ST9296 | Reference: ST9138 | POX3 Dmd9 HarFAR | ST9200 (pBP8627, pBP8576) |
| ST9297 | Reference: ST9138 | POX5 Dmd9 HarFAR | ST9202 (pBP8627, pBP8576) |
| ST9298 | Reference: ST9138 | Ani_POX Dmd9 HarFAR | ST9206 (pBP8627, pBP8576) |
| ST9299 | Reference: ST9138 | Cma_POX Dmd9 HarFAR | ST9207 (pBP8627, pBP8576) |
| ST9300 | Reference: ST9138 | Hsa_POX1-2 Dmd9 HarFAR | ST9208 (pBP8627, pBP8576) |
| ST9301 | Reference: ST9138 | Pur_POX | ST9209 |
| | | Dmd9 HarFAR | (pBP8627, pBP8576) |
| ST9302 | Reference: ST9138 | Rno_POX-2 Dmd9 HarFAR | ST9210 (pBP8627, pBP8576) |
| ST9314 | Reference: ST9138 | Lbo_PPTQ HarFAR | ST9138 (pBP8802, pBP8623) |
| ST9315 | Reference: ST9138 | POX2 Lbo_PPTQ HarFAR | ST9199 (pBP8802, pBP8576) |
| ST9316 | Reference: ST9138 | POX3 Lbo_PPTQ HarFAR | ST9200 (pBP8802, pBP8576) |
| ST9317 | Reference: ST9138 | POX5 Lbo_PPTQ HarFAR | ST9202 (pBP8802, pBP8576) |
| ST9318 | Reference: ST9138 | Ani_POX Lbo_PPTQ HarFAR | ST9206 (pBP8802, pBP8576) |
| ST9319 | Reference: ST9138 | Cma_POX Lbo_PPTQ HarFAR | ST9207 (pBP8802, pBP8576) |
| ST9320 | Reference: ST9138 | Hsa_POX1-2 Lbo_PPTQ HarFAR | ST9208 (pBP8802, pBP8576) |
| ST9321 | Reference: ST9138 | Pur_POX Lbo_PPTQ HarFAR | ST9209 (pBP8802, pBP8576) |
| ST9322 | Reference: ST9138 | Rno_POX-2 Lbo_PPTQ HarFAR | ST9210 (pBP8802, pBP8576) |
| ST9328 | Reference: ST9138 | AsePOX SlitDes5 HarFAR | ST9284 (pBP8626, pBP8576) |
| ST9329 | Reference: ST9138 | Ase_POX Dmd9 HarFAR | ST9284 (pBP8627, pBP8576) |
| ST9330 | Reference: ST9138 | Ase_POX Lbo_PPTQ HarFAR | ST9284 (pBP8802, pBP8576) |
| ST9331 | POX5 POX3 POX1 POX4 POX2 | | ST9252 (BB2651, pBP8813) |
| ST9344 | Reference: ST9138 | SlitDes5 HarFAR Lbo31670 | ST9285 (pBP8400, pBP8032) |
| ST9345 | Reference: ST9138 | Lbo49554 | ST9285 |
| | | Slides5 HarFAR | (pBP8401, pBP8032) |
| ST9347 | Reference: ST9138 | Dmd9 HarFAR Lbo31670 | ST9294 (pBP8400, pBP8032) |
| ST9348 | Reference: ST9138 | Dmd9 HarFAR Lbo49554 | ST9294 (pBP8401, pBP8032) |
| ST9350 | Reference: ST9138 | Lbo_PPTQ HarFAR Lbo31670 | ST9314 (pBP8400, pBP8032) |
| ST9351 | Reference: ST9138 | Lbo_PPTQ HarFAR Lbo49554 | ST9314 (pBP8401, pBP8032) |
| ST9366 | Reference: ST8616 | HarFAR Lbo_KPSE | ST8680 (pBP8819, pCfB6631) |
| ST9367 | Reference: ST8616 | HarFAR Cpu_KPSE | ST8680 (pBP8820, pCfB6631) |
| ST9368 | Reference: ST8616 | HarFAR YlOle1 | ST8680 (pBP8378, pCfB6631) |
| ST9369 | Reference: ST8616 | HarFAR IntD_1-ScOle1 | ST8680 (pBP8377, pCfB6631) |
| ST9435 | Reference: ST9138 | PGDes8 | ST9138 (pBP8789, pBP8328) |
| ST9436 | Reference: ST9331 | SlitDes5 HarFAR | ST9331 (pBP8626, pBP8623) |
| ST9438 | Reference: ST9331 | Lbo_PPTQ HarFAR | ST9331 (pBP8802, pBP8623) |
| ST9443 | Reference: ST9331 | PGDes8 Pur_POX | ST9435 (pBP8032, pBP8350) |
| ST9444 | Reference: ST9331 | SlitDes5 HarFAR Ase_POX AseFAR | ST9436 (pBP8875, pBP8625) |
| ST9447 | Reference: ST9331 | SlitDes5 HarFAR Pur_POX AseFAR | ST9436 (pBP8878, pBP8625) |
| ST9448 | Reference: ST9331 | SlitDes5 HarFAR | ST9436 (pBP8879, |
| | | Rno_POX AseFAR | pBP8625) |
| ST9449 | Reference: ST9331 | SlitDes5 HarFAR Yli_POX AseFAR | ST9436 (pBP8880, pBP8625) |
| ST9451 | Reference: ST9331 | SlitDes5 HarFAR AseFAR Lbo49554 | ST9436 (pBP8882, pBP8625) |
| ST9452 | Reference: ST9331 | SlitDes5 HarFAR AseFAR Lbo49602 | ST9436 (pBP8883, pBP8625) |
| ST9462 | Reference: ST9331 | Lbo_PPTQ HarFAR Ase_POX AseFAR | ST9438 (pBP8875, pBP8625) |
| ST9465 | Reference: ST9331 | Lbo_PPTQ HarFAR Pur_POX AseFAR | ST9438 (pBP8878, pBP8625) |
| ST9466 | Reference: ST9331 | Lbo_PPTQ HarFAR Rno_POX AseFAR | ST9438 (pBP8879, pBP8625) |
| ST9467 | Reference: ST9331 | Lbo_PPTQ HarFAR Yli_POX AseFAR | ST9438 (pBP8880, pBP8625) |
| ST9469 | Reference: ST9331 | Lbo_PPTQ HarFAR AseFAR Lbo49554 | ST9438 (pBP8882, pBP8625) |
| ST9470 | Reference: ST9331 | Lbo_PPTQ HarFAR AseFAR Lbo49602 | ST9438 (pBP8883, pBP8625) |
| ST9500 | Reference: ST9331 | Pur_POX AseFAR | ST9331 (pBP8878, pBP8567) |
| ST9521 | Reference: ST9331 | AseFAR | ST9331 (pBP8782, pBP8674) |
| ST9522 | Reference: ST9331 | SlitDes5 HarFAR AseFAR | ST9436 (pBP8782, pBP8568) |
| ST9524 | Reference: ST9331 | Lbo_PPTQ | ST9438 |
| | | HarFAR AseFAR | (pBP8782, pBP8568) |
| ST9552 | Reference: ST9331 | SlitDes5 HarFAR AseFAR Ani_POX | ST9522 (pBP8347, pCfB6630) |
| ST9560 | Reference: ST9331 | SlitDes5 HarFAR AseFAR Lbo31670 | ST9522 (pBP8400, pCfB6630) |
| ST9561 | Reference: ST9331 | Lbo_PPTQ HarFAR AseFAR Ani_POX | ST9524 (pBP8347, pCfB6630) |
| ST9562 | Reference: ST9331 | Lbo_PPTQ HarFAR AseFAR Lbo31670 | ST9524 (pBP8400, pCfB6630) |
| ST9564 | Reference: ST9331 | SlitDes5 HarFAR AseFAR Cma_POX | ST9522 (pBP8348, pCfB6630) |
| ST9567 | Reference: ST9331 | Lbo_PPTQ HarFAR AseFAR Cma_POX | ST9524 (pBP8348, pCfB6630) |
| ST9640 | Reference: ST9138 | Lbo_PPTQ HarFAR Lbo31670 AseFAR | ST9350 (pBP8236, pBP8674) |

In strain ST6629 the open-reading frame of genes HFD4 (YALI0801298g), HFD3 (YALIOA17875), HFD2 (YALI0E15400) and HFD1 (YALI0F23793g), as well as nucleotides -1130 to -100 upstream of the coding sequence of GPAT (YALI0C00209g) were deleted. A premature Stop-codon and frame-shift was introduced in PEX10 (YALI0C01023g) and FAO1 (YALI0B14014g) resulting in non-functional genes.

Strain ST7982 is derived from ST6629 and additionally the amino acid 1220, isoleucine, of the fatty acyl synthase subunit 2 (YALI0B19382g) was replaced to phenylalanine. The amino acid change enables the cells to produce increased amounts of C14 fatty acids.

Strain ST8616 is derived from ST6629 and was additionally engineered to improve the supply of co-substrates for desaturases and to increase fatty acid metabolism.

### Example 2: Selection of Y. Lipolytica Strains With Desired Chain Shortening Specificity

The genes encoding peroxisomal oxidases are inactivated in Y. lipolytica (Δku70 Δhfd4 Δhfd1 Δpex10 Δfao1 Δhfd2 Δhfd3 GPAT_100bpPR) [3] and peroxisomal biogenesis factor 10 PEX10 is restored, resulting in strain ST8016. In this strain, various native and heterologous peroxisomal oxidases from diverse organisms (non-exhaustive list in Table 6) are expressed, resulting in strains ST8588-8601. The resulting strains are cultivated, and cell extracts are prepared. The activity of extracts on various substrates (C10-CoA, C12-CoA, C14-CoA, C16-CoA) is tested using enzymatic assay [6,7]. Alternatively, the strains are tested for ability to grow on fatty acids of specific lengths using spot assays on plates. If a strain has activity on C16-CoA and longer acyl-CoAs, but less or no activity on C10-C12-C14 acyl-CoA substrates, then this strain (C14-strain) is suitable for producing pheromones with carbon chain length 14. If a strain has activity on C14-CoA and longer acyl-CoAs, but less or no activity on C10-C12 substrates, then this strain (C12-strain) is suitable for producing pheromones with carbon chain length 12.

TABLE 6

Peroxisomal oxidases

| Peroxisomal oxidase (SEQ ID NO) | Source organism | UniProt ID | Reference |
|---|---|---|---|
| Ase_POX (14) | Agrotis segetum | A0A068FKE0 | [8] |
| Ath_POX1 (16) | Arabidopsis thaliana | O65202 | [9,10] |
| Ath_POX2 (18) | Arabidopsis thaliana | O65201 | [9,10] |
| Ani_POX (20) | Aspergillus nidulans | Q5AY78 | [11] |
| Cma_POX (22) | Cucurbita maxima | 064894 | [12] |
| Hsa_POX1-2 (24) | Homo sapiens | Q15067-2 | [13] |
| Pur_POX (26) | Paenarthrobacter ureafaciens | Q33DR0 | [14] |
| Pci_POX | Phascolarctos cinereus | Q8HYL8-1 | [15] |
| Rno_POX-2 (28) | Rattus norvegicus | P07872-2 | [16] |
| Yli_POX2 (4) | Yarrowia lipolytica | O74935 | [17,18] |

### Example 3: Cloning and Strain Construction for Saccharomyces Cerevisiae

All heterologous genes were synthesized by GeneArt (Life Technologies) in codon optimized versions for S. cerevisiae. All the genes were amplified by PCR using Phusion U Hot Start DNA Polymerase (ThermoFisher) to obtain the fragments for cloning into yeast expression vectors. The PCR products were separated on a 1%-agarose gel containing RedSafe™ (iNtRON Biotechnology). PCR products of the correct size were excised from the gel and purified using the Nucleospin Gel and PCR Clean-up kit (Macherey-Nagel).

Integrative yeast vectors with USER cassette were linearized with FastDigest SfaAl (ThermoFisher) for 2 hours at 37° C. and then nicked with Nb.Bsml (New England Biolabs) for 1 hour at 65° C. The resulting vectors containing sticky ends were separated by gel electrophoresis, excised from the gel, and gel-purified using the Nucleospin Gel and PCR Clean-up kit (Macherey-Nagel). The DNA fragments were cloned into the so prepared vectors by USER-cloning as described in [32,33]. The reaction was transformed into chemically competent E. coli DHalpha cells and the cells were plated on Lysogeny Broth (LB) agar plates with 100 mg/L ampicillin. The plates were incubated overnight at 37° C. and the resulting colonies were screened by colony PCR. The plasmids were purified from overnight E. coli liquid cultures and the correct cloning was confirmed by sequencing.

Yeast strains were constructed by transformation of DNA vectors as described in [33]. Integrative vectors were linearized with FastDigest Notl prior to transformation. When needed, helper vectors to promote the integration into specific genomic regions were co-transformed with the integrative plasmid or DNA repair fragments. Strains were selected on yeast peptone dextrose (YPD) agar with appropriate antibiotics selection. Correct genotype was confirmed by colony PCR and when needed by sequencing.

### Example 4: Selection of S. Cerevisiae Strains With Desired Chain Shortening Specificity

The gene POX1 (YGL205W) encoding peroxisomal oxidase is inactivated in S. cerevisiae. In this strain, various native and heterologous peroxisomal oxidases from diverse organisms (non-exhaustive list in Table 6) are expressed. The resulting strains are cultivated, and cell extracts are prepared. The activity of extracts on various substrates (C10-CoA, C12-CoA, C14-CoA, C16-CoA) is tested using enzymatic assay [6,7]. Alternatively, the strains are tested for ability to grow on fatty acids of specific lengths using spot assays on plates. If a strain has activity on C16-CoA and longer acyl-CoAs, but less or no activity on C10-C12-C14 acyl-CoA substrates, then this strain (C14-strain) is suitable for producing pheromones with carbon chain length 14. If a strain has activity on C14-CoA and longer acyl-CoAs, but less or no activity on C10-C12 substrates, then this strain (C12-strain) is suitable for producing pheromones with carbon chain length 12.

### Example 5: Production of Δ_{Z5}-12:OAc in Yeast

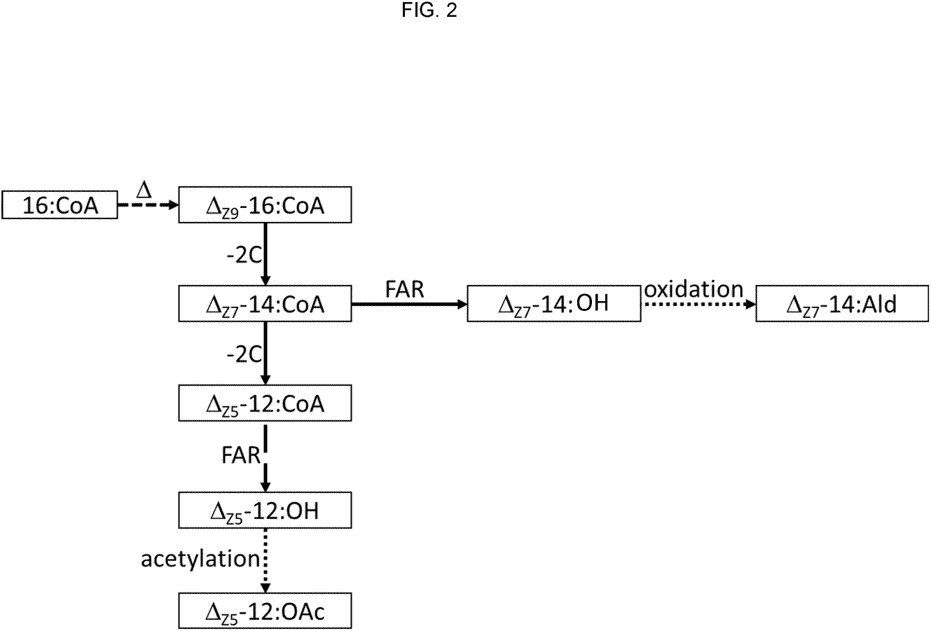

In a C12-strain of example 2 or example 4, the following genes are additionally expressed: Δ_{Z9}-desaturase from Saccharomyces cerevisiae (Sce_OLE1, SEQ ID NO: 30) or Y. lipolytica (Yli_OLE1, SEQ ID NO: 32), fatty acyl-CoA reductase from Helicoverpa armigera (Har_FAR, SEQ ID NO: 59), and acetyltransferase from S. cerevisiae (Sce_ATF1, SEQ ID NO: 61) (FIG. 2). The resulting strain is cultivated in growth medium. The fermentation broth samples are extracted with organic solvent and the extract is analyzed on GC-MS[3]. The production of Δ_{Z5}-12:OAc is detected. Δ_{z5}-12:OAc is a major pheromone component of the Western bean cutworm Striacosta albicosta and can be used to monitor or control this pest by mating disruption.

### Example 6: Production of Δ_{Z7}-12:OAc in Yeast

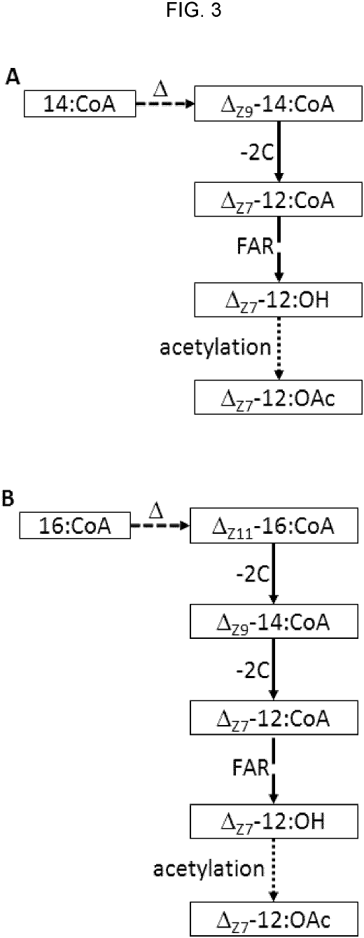

In a C12-strain of example 2 or example 4 the following genes are additionally expressed: Δ_{Z9}-14-desaturase from Drosophila melanogaster (Dme_D9, SEQ ID NO: 35), fatty acyl-CoA reductase from H. armigera (Har_FAR, SEQ ID NO: 59), and acetyltransferase from S. cerevisiae (Sce_ATF1, SEQ ID NO: 61) (FIG. 3A). Additionally, the FAS2 gene can be mutated to increase the formation of C14-CoA precursor. [4] The resulting strain is cultivated in growth medium. The fermentation broth samples are extracted with organic solvent and the extract is analyzed on GC-MS [3]. The production of Δ_{Z7}-12:OAc is detected. Δ_{Z7}-12:OAc is a major pheromone component of the soybean looper Chrysodeixis includens and can be used to monitor or control this pest by mating disruption.

### Example 7: Production of Δ_{Z7}-12:OAc in Yeast

In a C12-strain of example 2 or example 4 the following genes are additionally expressed: Δ_{Z11}-16-desaturase from Amyelois transitella (Atr_D11, SEQ ID NO: 38), fatty acyl-CoA reductase from H. armigera (Har_FAR, SEQ ID NO: 59), and acetyltransferase from S. cerevisiae (Sce_ATF1, SEQ ID NO: 61) (FIG. 3B). The resulting strain is cultivated in growth medium. The fermentation broth samples are extracted with organic solvent and the extract is analyzed on GC-MS. The production of Δ_{Z7}-12:OAc is detected. Δ_{Z7}-12:OAc is a major pheromone component of the soybean looper C. includens and can be used to monitor or control this pest by mating disruption.

### Example 8: Production of Δ_{E7}Δ_{Z9}-12:OAc in Yeast

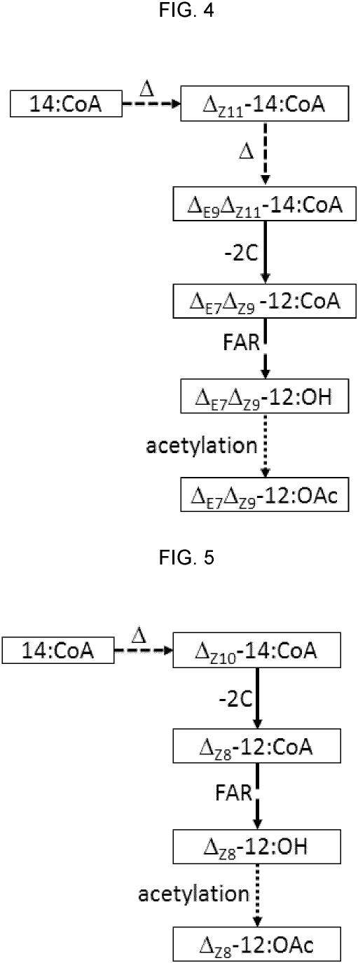

In a C12-strain of example 2 or example 4 the following genes are additionally expressed: Δ_{Z11}-14-desaturase from Choristoneura rosaceana (Cro_Z11, SEQ ID NO: 40), Ostrinia nubilalis (Onu_11, SEQ ID NO: 42) or Thaumetopoea pityocampa (Tpi_D13, SEQ ID NO: 44), Δ_{E9}-14-desaturase from Dendrolimus punctatus (Dpu_E9-14, SEQ ID NO: 46), fatty acyl-CoA reductase from H. armigera (Har_FAR, SEQ ID NO: 59), and acetyltransferase from S. cerevisiae (Sce_ATF1, SEQ ID NO: 61) (FIG. 4). Additionally, the FAS2 gene can be mutated to increase the formation of C14-CoA precursor. The resulting strain is cultivated in growth medium. The fermentation broth samples are extracted with organic solvent and the extract is analyzed on GC-MS. The production of Δ_{E7}Δ_{Z9}-12:OAc is detected. Δ_{E7}Δ_{Z9}-12:OAc is a major pheromone component of the grapevine moth Lobesia botrana and can be used to monitor or control this pest by mating disruption.

### Example 9: Production of Δ_{Z8}-12:OAc and Δ_{E8}-12:OAc in Yeast

In a C12-strain of example 2 or example 4 the following genes are additionally expressed: Δ_{Z/E10}-14-desaturase from Grapholita molesta (Gmo_CPRQ, SEQ ID NO: 48 or Gmo_KPSQ, SEQ ID NO: 96), fatty acyl-CoA reductase from H. armigera (Har_FAR, SEQ ID NO: 59), and acetyltransferase from S. cerevisiae (Sce_ATF1, SEQ ID NO: 61) (FIG. 5). Additionally, the FAS2 gene can be mutated to increase the formation of C14-CoA precursor. The resulting strain is cultivated in growth medium. The fermentation broth samples are extracted with organic solvent and the extract is analyzed on GC-MS. The production of Δ_{Z8}-12:OAc is detected, smaller amounts of Δ_{E8}-12:OAc are also detected. Δ_{Z8}-12:OAc is a major pheromone component and Δ_{E8}-12:OAc is a minor pheromone component of the oriental fruit moth G. molesta; these compounds can be used to monitor or control this pest by mating disruption.

### Example 10: Production of Δ_{E8}Δ_{E10}-C12:OH in Yeast

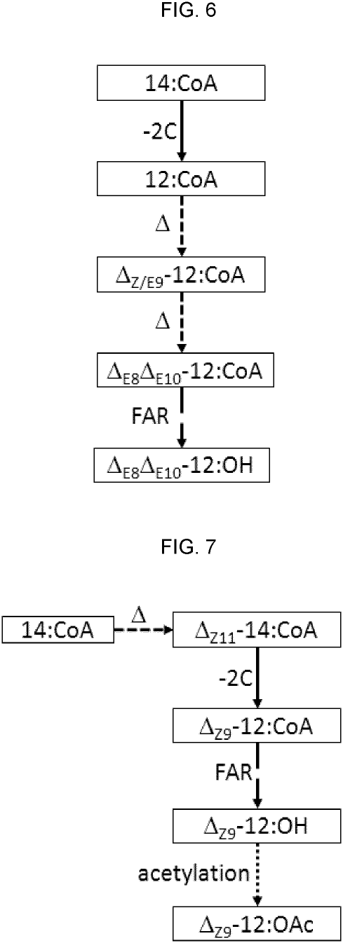

In a C12-strain of example 2 or example 4 the following genes are additionally expressed: a desaturase from Cydia pomonella (Cpo_CPRQ, SEQ ID NO: 50; Cpo_CPRQ + exon + tail1, SEQ ID NO: 129; Cpo_CPRQ + exon + tail2, SEQ ID NO: 131; Cpo_CPRQ - exon + tail 1, SEQ ID NO: 133; Cpo_CPRQ - exon + tail2, SEQ ID NO: 135, CPOM09289 + exon + tail 1, SEQ ID NO: 137; CPOM09289 + exon + tail2, SEQ ID NO: 139; CPOM09289 + exon - tail1, SEQ ID NO: 141; CPOM09289 + exon -tail 2, SEQ ID NO: 143; CPOM09289 - exon + tail1, SEQ ID NO: 145; CPOM09289 -exon + tail2, SEQ ID NO:147) and fatty acyl-CoA reductase from H. armigera (Har_FAR, SEQ ID NO: 59) (FIG. 6). Additionally, the FAS2 gene can be mutated to increase the formation of C14-CoA precursor. The resulting strain is cultivated in growth medium. The fermentation broth samples are extracted with organic solvent and the extract is analyzed on GC-MS. The production of Δ_{E8}Δ_{E10}-C12:OH is detected. Δ_{E8}Δ_{E10}-C12:OH is a major pheromone component of the codling moth C. pomonella and can be used to monitor or control this pest by mating disruption.

### Example 11: Production of Δ_{Z9}-12:OAc in Yeast

In a C12-strain of example 2 or example 4 the following genes are additionally expressed: Δ_{Z11}-14-desaturase from C. rosaceana (Cro_Z11, SEQ ID NO: 40), O. nubilalis (Onu_11, SEQ ID NO: 42) or T. pityocampa (Tpi_D13, SEQ ID NO: 44), fatty acyl-CoA reductase from H. armigera (Har_FAR, SEQ ID NO: 59), and acetyltransferase from S. cerevisiae (Sce_ATF1, SEQ ID NO: 61) (FIG. 7). Additionally, the FAS2 gene can be mutated to increase the formation of C14-CoA precursor. The resulting strain is cultivated in growth medium. The fermentation broth samples are extracted with organic solvent and the extract is analyzed on GC-MS. The production of Δ_{Z9}-12:OAc is detected. Δ_{Z9}-12:OAc is a major pheromone component of the grape berry moth Paralobesia viteana and can be used to monitor or control this pest by mating disruption.

### Example 12: Production of Δ_{Z7}-14:OH in Yeast

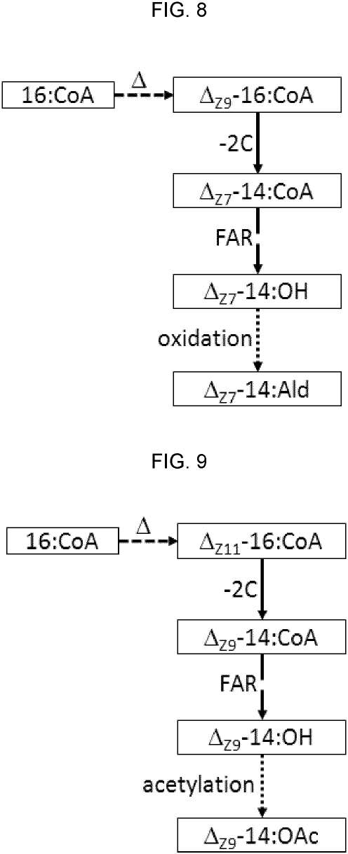

In a C14-strain of example 2 or example 4 the following genes are additionally expressed: Δ_{Z9}-desaturase from S. cerevisiae (Sce_OLE1, SEQ ID NO: 30) or Y. lipolytica (Yli_OLE1, SEQ ID NO: 32) and fatty acyl-CoA reductase from H. armigera (Har_FAR, SEQ ID NO: 59) (FIG. 8). The resulting strain is cultivated in growth medium. The fermentation broth samples are extracted with organic solvent and the extract is analyzed on GC-MS. The production of Δ_{Z7}-14:OH is detected. Δ_{Z7}-14:OH is chemically oxidized into Δ_{Z7}-14:AId [19]. Δ_{Z7}-14:AId is a major pheromone component of the olive fruit fly Prays oleae and can be used to monitor or control this pest by mating disruption.

### Example 13: Production of Δ_{Z9}-14:OAc in Yeast

In a C14-strain of example 2 or example 4 the following genes are additionally expressed: Δ_{Z11}-16-desaturase from A. transitella (Atr_D11, SEQ ID NO: 38), fatty acyl-CoA reductase from H. armigera (Har_FAR, SEQ ID NO: 59), and acetyltransferase from S. cerevisiae (Sce_ATF1, SEQ ID NO: 61) (FIG. 9). The resulting strain is cultivated in growth medium. The fermentation broth samples are extracted with organic solvent and the extract is analyzed on GC-MS. The production of Δ_{Z9}-14:OAc is detected. Δ_{Z9}-14:OAc is a major pheromone component of the fall armyworm Spodoptera frugiperda and can be used to monitor or control this pest by mating disruption.

### Example 14: Production of Δ_{E11}-14:OAc in Yeast

In a C14-strain of example 2 or example 4 the following genes are additionally expressed: Δ_{E11}-14-desaturase from O. nubilalis (Onu_11, SEQ ID NO: 42), from Epiphyas postvittana (Epo_E11, SEQ ID NO: 52), from Spodoptera littoralis (Sls_ZE11, SEQ ID NO: 54), from C. rosaceana (Cro_Z11, SEQ ID NO: 40) or from Choristoneura parallela (Cpa_E11, SEQ ID NO: 56), fatty acyl-CoA reductase from H. armigera (Har_FAR, SEQ ID NO: 59), and acetyltransferase from S. cerevisiae (Sce_ATF1, SEQ ID NO: 61) (FIG. 10). Additionally, the FAS2 gene can be mutated to increase the formation of C14-CoA precursor. The resulting strain is cultivated in growth medium. The fermentation broth samples are extracted with organic solvent and the extract is analyzed on GC-MS. The production of Δ_{E11}-14:OAc is detected. Δ_{E11}-14:OAc is a major pheromone component of the lightbrown apple moth E. postvittana and can be used to monitor or control this pests by mating disruption.

### Example 15: Production of Δ_{Z11}-14:OAc in Yeast

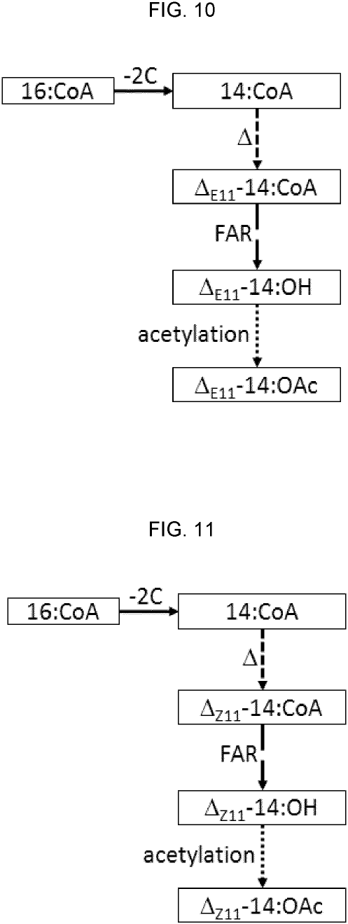

In a C14-strain of example 2 or example 4 the following genes are additionally expressed: Δ_{Z11}-14-desaturase from C. rosaceana (Cro_Z11, SEQ ID NO: 40), O. nubilalis (Onu_11, SEQ ID NO: 42) or T. pityocampa (Tpi_D13, SEQ ID NO: 44), fatty acyl-CoA reductase from H. armigera (Har_FAR, SEQ ID NO: 59), and acetyltransferase from S. cerevisiae (Sce_ATF1, SEQ ID NO: 61) (FIG. 11). Additionally, the FAS2 gene can be mutated to increase the formation of C14-CoA precursor. The resulting strain is cultivated in growth medium. The fermentation broth samples are extracted with organic solvent and the extract is analyzed on GC-MS. The production of Δ_{Z11}-14:OAc is detected. Δ_{Z11}-14:OAc is a major pheromone component of the European corn borer O. nubilalis and can be used to monitor or control this pest by mating disruption.

### Example 16: Production of Δ_{Z7}-16:OH in Yeast

In a C16-strain of example 2 or example 4 the following genes are additionally expressed: Δ_{Z9}-desaturase from S. cerevisiae (Sce_OLE1, SEQ ID NO: 30) or Y. lipolytica (Yli_OLE1, SEQ ID NO: 32) and fatty acyl-CoA reductase from H. armigera (Har_FAR, SEQ ID NO: 59) (FIG. 12). The resulting strain is cultivated in growth medium. The fermentation broth samples are extracted with organic solvent and the extract is analyzed on GC-MS. The production of Δ_{Z7}-16:OH is detected. Δ_{Z7}-16:OH is chemically oxidized into Δ_{Z7}-16:Ald [19]. Δ_{Z7}-16:Ald is a minor pheromone component of the crambid stalkborer Diatraea considerata and, together with Δ_{Z11}-16:Ald, can be used to monitor or control this pest by mating disruption.

### Example 17: Production of Δ_{Z7}Δ_{Z11}-16:OAc in Yeast

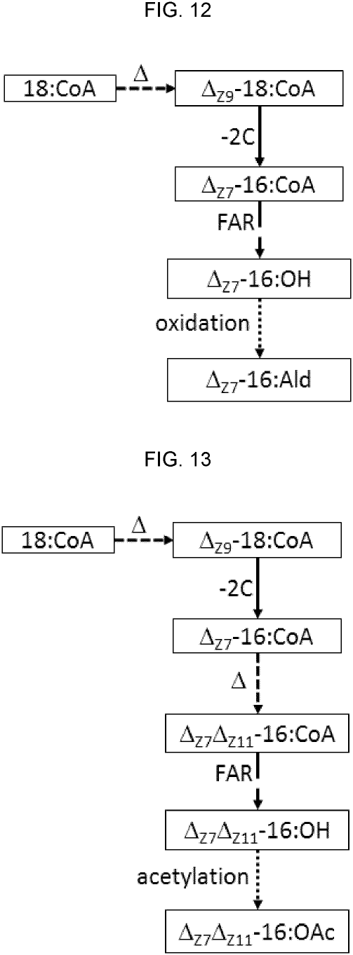

In a C16-strain of example 2 or example 4 the following genes are additionally expressed: Δ_{Z11}-16-desaturase from A. transitella (Atr_D11, SEQ ID NO: 38), fatty acyl-CoA reductase from H. armigera (Har_FAR, SEQ ID NO: 59), and acetyltransferase from S. cerevisiae (Sce_ATF1, SEQ ID NO: 61) (FIG. 13). Optionally, the native OLE1 gene can be overexpressed. The resulting strain is cultivated in growth medium, optionally oleic acid can be supplemented. The fermentation broth samples are extracted with organic solvent and the extract is analyzed on GC-MS. The production of Δ_{Z7}Δ_{Z11}-16:OAc is detected. Δ_{Z7}Δ_{Z11}-16:OAc is the major pheromone component of the pink bollworm Pectinophora gossypiella and can be used to monitor or control this pest by mating disruption.

### Example 18: Production of Δ_{Z9}-16:OH in Yeast

In a C16-strain of example 2 or example 4 the following genes are additionally expressed: Δ_{Z9}-desaturase from S. cerevisiae (Sce_OLE1, SEQ ID NO: 30) or Y. lipolytica (Yli_OLE1, SEQ ID NO: 32) and fatty acyl-CoA reductase from H. armigera (Har_FAR, SEQ ID NO: 59) (FIG. 14). The resulting strain is cultivated in growth medium. The fermentation broth samples are extracted with organic solvent and the extract is analyzed on GC-MS. The production of Δ_{Z9}-16:OH is detected. Δ_{Z9}-16:OH can be chemically oxidized into Δ_{Z9}-16:Ald [19]. Δ_{Z9}-16:Ald is the major pheromone component of the oriental tobacco budworm Helicoverpa assulta and can be used to monitor or control this pest by mating disruption.

Strains ST9366, ST9367, ST9368, and ST9369 express fatty acyl reductase from Helicoverpa armigera (Har_FAR, SEQ ID NO: 59) and the Δ_{Z9}-16 desaturases of Lobesia botrana (Lbo_KPSE, SEQ ID NO: 88), Chilo suppressalis (Csup_KPSE, SEQ ID NO: 90), Yarrowia lipolytica (YlOle1, SEQ ID NO: 31) and Saccharomyces cerevisiae (ScOle1, SEQ ID NO: 29), respectively, in the strain ST8616. Strain ST8680 serves as a control strain and only expresses the fatty acyl reductase from Helicoverpa armigera (Har_FAR, SEQ ID NO: 59) in strain ST8616.

TABLE 7

| Strain ID | Desaturase | Δ_{Z9}-16:OH (mg/L) |
|---|---|---|
| ST8680 | - | 214±15 |
| ST9366 | Lbo_KPSE | 179±14 |
| ST9367 | Csup_KPSE | 281±5 |
| ST9368 | YlOle1 | 202±13 |
| ST9369 | ScOle1 | 289±3 |

Table 7 shows the results of duplicate experiments. The strains expressing the Δ_{Z9}-16 desaturases Csup_KPSE and ScOLE1 showed an increased production of Δ_{Z9}-16:OH compared to the control strain ST8680. The Δ_{Z9}-16 desaturases Csup_KPSE and ScOLE1 can be used to increase the production of Δ_{Z7}-14:Me, the chain-shortened product of Δ_{Z9}-16:Me.

### Example 19: Production of Δ_{Z11}Δ_{Z13}-16:OH in Yeast

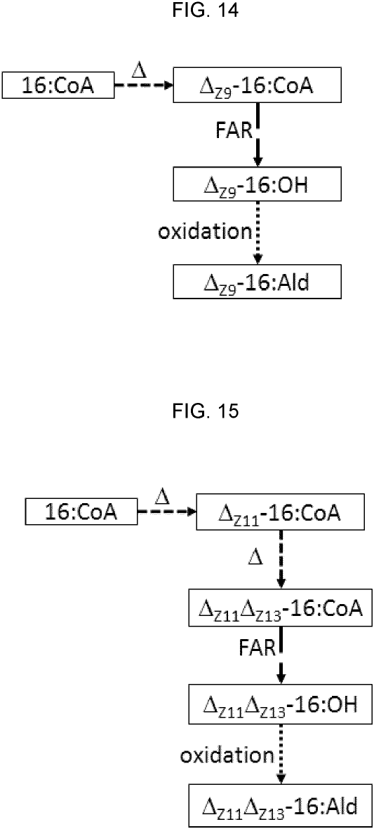

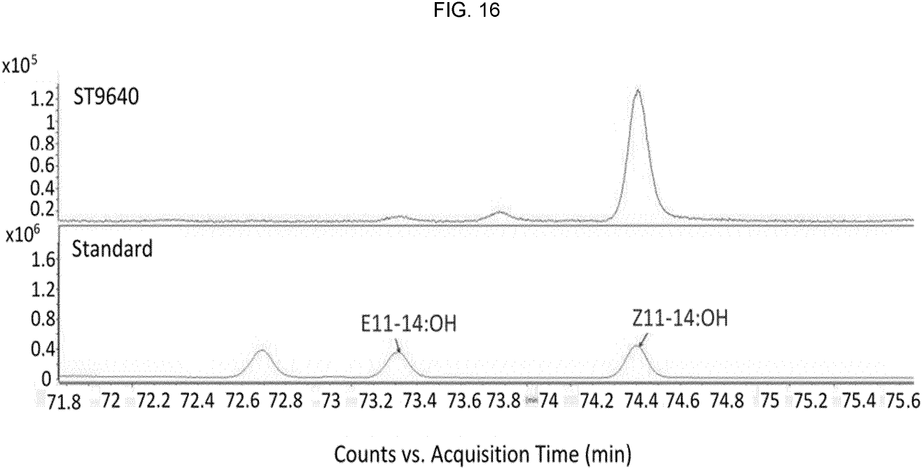

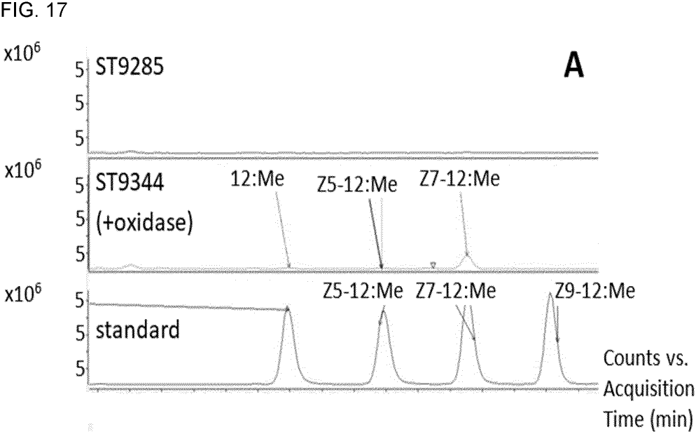

In a C16-strain of example 2 or example 4 the following genes are additionally expressed: Δ_{Z11}-16-desaturase from A. transitella (Atr_D11, SEQ ID NO: 38), Δ_{Z13}-16-desaturase from T. pityocampa (Tpi_D13, SEQ ID NO: 44), and fatty acyl-CoA reductase from H. armigera (Har_FAR, SEQ ID NO: 59) (FIG. 15). The resulting strain is cultivated in growth medium. The fermentation broth samples are extracted with organic solvent and the extract is analyzed on GC-MS. The production of Δ_{Z11}Δ_{Z13}-16:OH is detected. Δ_{Z11}Δ_{Z13}-16:OH is chemically oxidized into Δ_{Z11}Δ_{Z13}-16:Ald [19]. A_{Z11}A_{Z13}-16:Ald is the major pheromone component of the orange worm A. transitella and can be used to monitor or control this pest by mating disruption.

### Example 20: Cultivation of Yeast Cells and Sample Extraction and Analysis

Strains were inoculated from a YPD agar plate (10 g/L yeast extract, 10 g/L peptone, 20 g/L glucose, 15 g/L agar agar) to an initial OD₆₀₀ of 0.1-0.6 into 2.5 mL YPG medium (10 g/L yeast extract, 10 g/L peptone, 40 g/L glycerol) in 24 well-plates (EnzyScreen). The plates were incubated at 28° C., shaken at 300 rpm. After 22-24 h, the plates were centrifuged for 5 min at 4° C. and 3,000 xg. The supernatant was discarded, and the cells were resuspended in 1.25 mL production medium per well (50 g/L glycerol, 5 g/L yeast extract, 4 g/L KH₂PO₄, 1.5 g/L MgSO₄, 0.2 g/L NaCl, 0.265 g/L CaCl₂.2H₂O, 2 mL/L trace elements solution: 4.5 g/L CaCl₂.2H₂O, 4.5 g/L ZnSO₄.7H₂O, 3 g/L FeSO₄.7H₂O, 1 g/L H₃BO₃, 1 g/L MnCl₂.4H₂O, 0.4 g/L N Na₂MoO₄.2H₂O, 0.3 g/L CoC₁₂.6H₂O, 0.1 g/L CuSO₄.5H₂O, 0.1 g/L Kl, 15 g/L EDTA). In some cases, the medium was supplemented with 3.3 µl methyl myristate or 1.5 µL oleic acid. This supplementation is indicated where relevant in the results tables. The plate was incubated for 28 hours at 28° C., shaken at 300 rpm.

For analysis of the fatty acids, 1 mL of each vial was harvested by centrifugation for 5 min at 4° C. and 3,000 xg. Each pellet was extracted with 1000 µL 1 M HCl in methanol (anhydrous). The samples were vortexed for 20 sec and placed in the 80° C. water bath for 2 h. The samples were vortexed every 30 min for 10 sec. After cooling down of the samples to room temperature, 1000 µL of 1 M NaOH in methanol (anhydrous), 500 µL of NaCl saturated H₂O, 990 µL of hexane and 10 µL of 19:Me (2 mg/mL) as internal standard were added. The samples were vortexed and centrifuged for 5 min at 21° C. and 3,000 xg. The upper organic phase was analyzed via GC-MS.

For analysis of fatty alcohols, 1 ml of the broth was pelleted and extracted with 990 µL of ethyl acetate:ethanol (84:15) and 10 µL of 19:Me (2 mg/mL) as internal standard. The samples were vortexed for 20 sec and incubated for 1 h at room temperature, followed by 5 min of vortexing. 300 µL of H₂O was added to each sample. The samples were vortexed and centrifuged for 5 min at 21° C. and 3,000 xg. The upper organic phase was analyzed via gas chromatography-mass spectrometry (GC-MS).

GC-MS analyses were performed on an Agilent GC 7820A coupled to MS 5977B. The GC was equipped with a split/spitless injector and a DB-Fatwax UI column (30 m, 0.25 mm i.d. and 0.25 µm film).

The operation parameters were: 1 µl splitless injection, injector temperature 220° C. and constant flow 1 ml/min helium. Samples run either with an oven ramp a) 80° C. for 1 min, 15° C. /min to 210° C. for 7 min, 20° C./min to 230° C., b) 80° C. for 1 min, 1° C. /min to 145° C., 2° C. /min to 190° C. for 10 min, 20° C./min to 230° C. or c) 80° C. for 1 min, 1° C. /min to 145° C., 2° C. /min to 170° C., 20° C./min to 230° C.The MS was operated in electron impact mode (70 eV), scanning between m/z 30 and 350. Data were analysed by the Mass Hunter software B.08.00.

### Example 21: Production of Δ_{Z11}-14:OH in Yeast

Strains ST8373, ST8374, ST8375, ST8376, ST8377 and ST8378 express fatty acyl reductase from Helicoverpa armigera (Har_FAR, SEQ ID NO: 59) and the Δ_{Z11}-14 desaturases of Lobesia botrana (Lbo_PTTQ, SEQ ID NO: 78), Ostrinia nubilalis (Onu11, SEQ ID NO: 41), Epiphyas postvittana (EpoE11, SEQ ID NO: 51), Spodoptera littoralis (SlsZE11, SEQ ID NO: 53), Choristoneura rosaceana (CroZ11, SEQ ID: 39) and Choristoneura parallela (CpaE11, SEQ ID NO:55), respectively, in strain ST7982 optimized for C14 fatty acid production. Strain ST8225 serves as a control strain and only expresses the fatty acyl reductase from Helicoverpa armigera (Har_FAR, SEQ ID NO: 59). The samples were separated with the oven ramp a) described in example 20.

TABLE 8

production of Δ_{Z11}-14:OH production in yeast

| Strain ID | Desaturase | Δ_{Z11}-14:OΗ (mg/L) |
|---|---|---|
| ST8225 | - | 0±0 |
| ST8373 | Lbo_PPTQ | 6.2±1.3 |
| ST8374 | Onu11 | 0±0 |
| ST8375 | EpoE11 | 0±0 |
| ST8376 | SlsZE11 | 0.2±0.1 |
| ST8377 | CroZ11 | 0.6±0 |
| ST8378 | CpaE11 | 0±0 |

Strains ST8373, ST8376 and ST8377 expressing the Δ_{Z11}-14 desaturases of Lobesia botrana, Spodoptera littoralis and Choristoneura rosaceana, respectively showed production of Δ_{Z11}-14:OH. The Δ_{Z11}-14 desaturases of Lobesia botrana (Lbo_PPTQ) produces the highest amount of Δ_{Z11}-14:OH.

Strain ST9640 expressing fatty acyl reductase from Helicoverpa armigera (Har_FAR, SEQ ID NO: 59) and the Δ_{Z11}-14 desaturases of Lobesia botrana (Lbo_PTTQ, SEQ ID NO: 78) was cultivated and the fatty alcohols were extracted. When the extracts were separated with a long GC method (oven ramp c)) Δ_{Z11}-14:OH but also Δ_{E11}-14:OH could be detected (FIG. 16). Both, retention time and spectrum are matching the reference compounds.

Δ_{Z11}-14:OH can be converted into the corresponding aldehyde or acetate. Δ_{Z11}-14:Ac is the pheromone of Ostrinia nubilalis.

### Example 22: Yeast Strains With Engineered Beta-Oxidation for Chain Shortening of Fatty Acids

All strains in this example are derived from ST9331 which bears deletion of the native peroxisomal oxidases POX1-5.

ST9524 expresses fatty acyl reductase from Helicoverpa armigera (Har_FAR, SEQ ID NO: 59), the fatty acyl reductase of Agrotis segetum (AseFAR, SEQ ID NO: 92) and the Δ_{Z11}-14 desaturase from Lobesia botrana (Lbo_PTTQ, SEQ ID NO: 86), but no peroxisomal oxidase. Strains ST9462, ST9561, ST9567, ST9465, ST9466, ST9467, ST9562, ST9469, and ST9470 are derived from strain ST9524, and additionally express different peroxisomal oxidases. Cultures of strains expressing the Lbo_PTTQ desaturase were supplemented with methyl myristate.

Strain ST9521 only expressed the fatty acyl reductase of Agrotis segetum (AseFAR, SEQ ID NO: 92) but no peroxisomal oxidase. Strain ST9500 also expresses fatty acyl reductase of Agrotis segetum (Ase_FAR, SEQ ID NO: 92) and additionally the peroxisomal oxidase of Paenarthrobacter ureafaciens (Pur_POX, SEQ ID NO: 25). The samples were separated with the oven ramp program a) as described in example 20.

Both retention times and spectra are matching the reference compounds.

TABLE 9

| Strain ID | Desaturase | Oxidase | Δ_{Z7}-16:Me (mg/L) | Δ_{Z9}-16:Me (mg/L) |
|---|---|---|---|---|
| ST9524 | Lbo_PPTQ | | 0.1 | 46.1 |
| ST9462 | Lbo_PPTQ | AsePox | 0 | 58.3 |
| ST9561 | Lbo_PPTQ | AniPox | 2.3 | 70.9 |
| ST9567 | Lbo_PPTQ | CmaPOX | 1.9 | 45.9 |
| ST9465 | Lbo_PPTQ | PurPOX | 0.9 | 58.3 |
| ST9466 | Lbo_PPTQ | RnoPOX-2 | 0.8 | 50.9 |
| ST9467 | Lbo_PPTQ | YliPOX2 | 1.8 | 62.8 |
| ST9562 | Lbo_PPTQ | Lbo31670 | 2.9 | 49.2 |
| ST9469 | Lbo_PPTQ | Lbo49554 | 2.3 | 47.6 |
| ST9470 | Lbo_PPTQ | Lbo49602 | 1.9 | 52.8 |
| ST9521 | - | - | 1.3 | 69.1 |
| ST9500 | - | PurPOX | 0.9 | 53.3 |
| ST9521* | - | - | 4.7 | 47.7 |
| ST9500* | - | PurPOX | 8 | 43.5 |

* supplemented with oleic acid

Increased amounts of Δ_{Z7}-16:methyl ester, the chain-shortened product of Δ_{Z9}-18:Me, could be detected in strains expressing AniPOX (SEQ ID NO: 20), CmaPOX (SEQ ID NO: 22), PurPOX (SEQ ID NO: 26), RnoPOX-2 (SEQ ID NO: 28), YliPOX2 (SEQ ID NO: 4), Lbo31670 (SEQ ID NO: 81), Lbo49554 (SEQ ID NO: 83), and Lbo49602 (SEQ ID NO: 85). The production of Δ_{Z7}-16:Me in the strains not expressing any heterologous oxidase most likely originates from the oxidase activity of the native Y. lipolytica POX6.

Both retention times and spectra are matching the reference compounds.

### Example 23: Improved Production of Unsaturated Fatty Acid Methyl Esters via Chain Shortening

All strains in this example are derived from ST9138 which bears deletions of all Y. lipolytica native peroxisomal oxidases POX1-6. Strains ST9285, ST9294 and ST9314 express fatty acyl reductase from Helicoverpa armigera (Har_FAR, SEQ ID NO: 59) and the Δ_{Z11}-16 desaturase from Spodoptera litura (Slitdes5, SEQ ID NO: 86), the Δ_{Z9}-14 desaturase from Drosophila melanogaster (Dmd9, SEQ ID NO: 33) and Δ_{Z11}-14 desaturase from Lobesia botrana (LboPPTQ, SEQ ID NO: 78), respectively. These three strains were then combined with various peroxisomal oxidases as indicated in Table 10. Cultures of strains expressing Dmd9 or Lbo_PPTQ were supplemented methyl myristate. The samples were separated with the oven ramp program a) as described in example 20.

TABLE 10

| Strain ID | Heterologous desaturase | Oxidase | 12:Me (mg/L) | Δ_{Z5}- 12:Me (mg/L) | Δ_{Z7}- 12:Me (mg/L) | Δ_{Z9}-12:Me (mg/L) |
|---|---|---|---|---|---|---|
| ST9285 | Slitdes5 | - | 0.18 | ND | 0.00 | ND |
| ST9286 | Slitdes5 | POX2 | 0.03 | ND | 0.00 | ND |
| ST9287 | Slitdes5 | POX3 | 0.02 | ND | 0.02 | ND |
| ST9288 | Slitdes5 | POX5 | 0.03 | ND | 0.01 | ND |
| ST9289 | Slitdes5 | Ani_POX | 0.00 | ND | 0.00 | ND |
| ST9290 | Slitdes5 | Cma_POX | 0.10 | ND | 0.10 | ND |
| ST9285 | Slitdes5 | - | 0.01 | ND | 0.01 | ND |
| ST9291 | Slitdes5 | Hsa_POX1-2 | 0.00 | ND | 0.00 | ND |
| ST9292 | Slitdes5 | Pur_POX | 0.00 | ND | 0.00 | ND |
| ST9293 | Slitdes5 | Rno_POX-2 | 0.01 | ND | 0.01 | ND |

| Strain ID | Heterologous desaturase | Oxidase | 12:Me (mg/L) | Δ_{Z5}- 12:Me (mg/L) | Δ_{Z7}- 12:Me (mg/L) | Δ_{Z9}-12:Me (mg/L) |
|---|---|---|---|---|---|---|
| ST9285 | Slitdes5 | - | 0.11 | 0.00 | 0.00 | 0.07 |
| ST9328 | Slitdes5 | Ase_POX_ GeneArt | 0.10 | 0.00 | 0.00 | 0.07 |
| ST9344 | Slitdes5 | Lbo31670 | 0.09 | 0.00 | 0.20 | 0.00 |
| ST9345 | Slitdes5 | Lbo49554 | 0.10 | 0.02 | 0.07 | 0.00 |
| ST9294 | Dmd9 | - | 0.83 | 0.00 | 0.16 | 0.00 |
| ST9296 | Dmd9 | POX3 | 0.18 | 0.00 | 0.03 | 0.00 |
| ST9297 | Dmd9 | POX5 | 0.33 | 0.00 | 0.06 | 0.00 |
| ST9298 | Dmd9 | Ani_POX | 0.58 | 0.00 | 0.10 | 0.00 |
| ST9299 | Dmd9 | Cma_POX | 2.34 | 0.26 | 3.43 | 0.00 |
| ST9300 | Dmd9 | Hsa_POX1-2 | 1.81 | 0.03 | 1.46 | 0.00 |
| ST9301 | Dmd9 | Pur_POX | 1.05 | 0.27 | 0.63 | 0.00 |
| ST9302 | Dmd9 | Rno_POX-2 | 3.17 | 0.38 | 2.88 | 0.00 |
| ST9329 | Dmd9 | Ase_POX_ GeneArt | 0.79 | 0.00 | 0.22 | 0.00 |
| ST9347 | Dmd9 | Lbo31670 | 3.44 | 0.14 | 4.85 | 0.00 |
| ST9348 | Dmd9 | Lbo49554 | 1.98 | 0.18 | 0.93 | 0.00 |
| ST9314 | Lbo_PPTQ | - | 0.54 | 0.00 | 0.00 | 0.14 |
| ST9315 | Lbo_PPTQ | POX2 | 0.39 | 0.00 | 0.00 | 0.00 |
| ST9316 | Lbo_PPTQ | POX3 | 0.51 | 0.00 | 0.00 | 0.00 |
| ST9317 | Lbo_PPTQ | POX5 | 0.85 | 0.00 | 0.00 | 0.00 |
| ST9318 | Lbo_PPTQ | Ani_POX | 0.47 | 0.00 | 0.00 | 0.00 |
| ST9319 | Lbo_PPTQ | Cma_POX | 1.30 | 0.16 | 0.06 | 1.03 |
| ST9320 | Lbo_PPTQ | Hsa_POX1-2 | 2.61 | 0.07 | 0.05 | 2.34 |
| ST9321 | Lbo_PPTQ | Pur_POX | 2.25 | 0.88 | 0.05 | 0.58 |
| ST9322 | Lbo_PPTQ | Rno_POX-2 | 2.25 | 0.38 | 0.06 | 1.43 |
| ST9330 | Lbo_PPTQ | Ase_POX_ GeneArt | 0.52 | 0.00 | 0.00 | 0.23 |
| ST9350 | Lbo_PPTQ | Lbo31670 | 1.70 | 0.13 | 0.07 | 1.68 |
| ST9351 | Lbo_PPTQ | Lbo49554 | 1.86 | 0.12 | 0.03 | 1.16 |

| Strain ID | 14:Me (mg/L) | Δ_{Z5}-14:Me (mg/L) | Δ_{Z7}-14:Me (mg/L) | Δ_{Ζ9}-14:Μe (mg/L) | Δ_{Z11}-14:Μe (mg/L) |
|---|---|---|---|---|---|
| ST9285 | 0.12 | ND | ND | 0.32 | ND |
| ST9286 | 0.03 | ND | ND | 0.12 | ND |
| ST9287 | 0.03 | ND | ND | 0.12 | ND |
| ST9288 | 0.03 | ND | ND | 0.24 | ND |
| ST9289 | 0.07 | ND | ND | 0.25 | ND |
| ST9290 | 0.08 | ND | ND | 1.77 | ND |
| ST9285 | 0.10 | ND | ND | 0.31 | ND |
| ST9291 | 0.09 | ND | ND | 1.33 | ND |
| ST9292 | 0.08 | ND | ND | 0.29 | ND |
| ST9293 | 0.06 | ND | ND | 1.65 | ND |

| Strain ID | 14:Me (mg/L) | Δ_{Z5}-14:Me (mg/L) | Δ_{Z7}-14:Me (mg/L) | Δ_{Ζ9}-14:Μe (mg/L) | Δ_{Z11}-14:Μe (mg/L) |
|---|---|---|---|---|---|
| ST9285 | 0.58 | 0.00 | 0.00 | 0.00 | 0.08 |
| ST9328 | 0.47 | 0.00 | 0.00 | 0.00 | 0.07 |
| ST9344 | 0.15 | 0.01 | 0.00 | 0.00 | 0.00 |
| ST9345 | 0.16 | 0.01 | 0.00 | 0.00 | 0.00 |
| ST9294 | 157.66 | 0.04 | 0.00 | 0.00 | 0.00 |
| ST9296 | 86.89 | 0.01 | 0.01 | 0.00 | 0.00 |
| ST9297 | 110.66 | 0.00 | 0.02 | 0.00 | 0.00 |
| ST9298 | 137.14 | 0.05 | 0.02 | 0.00 | 0.00 |
| ST9299 | 163.67 | 0.25 | 0.46 | 0.00 | 0.00 |
| ST9300 | 203.94 | 0.07 | 0.12 | 0.00 | 0.00 |
| ST9301 | 133.32 | 0.50 | 0.08 | 0.00 | 0.00 |
| ST9302 | 188.46 | 0.41 | 0.33 | 0.00 | 0.00 |
| ST9329 | 162.44 | 0.03 | 0.00 | 0.00 | 0.00 |
| ST9347 | 258.42 | 0.11 | 0.22 | 0.00 | 0.00 |
| ST9348 | 105.76 | 0.32 | 0.07 | 0.00 | 0.00 |
| ST9314 | 139.62 | 0.02 | 0.00 | 0.00 | 5.34 |
| ST9315 | 185.52 | 0.05 | 0.05 | 0.00 | 1.64 |
| ST9316 | 164.17 | 0.02 | 0.03 | 0.00 | 2.70 |
| ST9317 | 209.79 | 0.03 | 0.04 | 0.00 | 2.61 |
| ST9318 | 87.67 | 0.03 | 0.01 | 0.00 | 2.18 |
| ST9319 | 184.65 | 0.20 | 0.32 | 0.00 | 6.34 |
| ST9320 | 290.24 | 0.10 | 0.26 | 0.00 | 11.62 |
| ST9321 | 162.06 | 1.24 | 0.26 | 0.00 | 5.69 |
| ST9322 | 191.41 | 0.44 | 0.35 | 0.00 | 6.67 |
| ST9330 | 188.85 | 0.03 | 0.00 | 0.00 | 10.56 |
| ST9350 | 186.19 | 0.07 | 0.16 | 0.00 | 4.99 |
| ST9351 | 95.88 | 0.10 | 0.10 | 0.00 | 6.68 |

| Strain ID | 16:Me (mg/L) | Δ_{Z7}-16:Me (mg/L) | Δ_{Z9}-16:OH | Δ_{Z11}- 16:Me (mg/L) | Δ_{Z9}-18:Me (mg/L) |
|---|---|---|---|---|---|
| ST9285 | 10.85 | 0.00 | 5.86 | 145.41 | 157.36 |
| ST9286 | 7.28 | 8.82 | 1.79 | 59.63 | 131.52 |
| ST9287 | 7.68 | 10.41 | 2.42 | 75.43 | 160.20 |
| ST9288 | 7.30 | 8.05 | 3.30 | 67.85 | 144.44 |
| ST9289 | 10.40 | 4.43 | 6.05 | 107.13 | 178.91 |
| ST9290 | 11.25 | 8.37 | 6.51 | 120.22 | 146.73 |
| ST9285 | 9.18 | 0.00 | 5.65 | 87.27 | 92.32 |
| ST9291 | 11.14 | 2.77 | 6.88 | 113.37 | 147.48 |
| ST9292 | 10.38 | 13.73 | 4.81 | 91.29 | 176.77 |
| ST9293 | 10.61 | 7.87 | 7.19 | 119.92 | 204.17 |

| Strain ID | 16:Me (mg/L) | Δ_{Z7}-16:Me (mg/L) | Δ_{Z9}-16:OH | Δ_{Z11}- 16:Me (mg/L) | Δ_{Z9}-18:Me (mg/L) |
|---|---|---|---|---|---|
| ST9285 | 11.37 | 0.00 | 5.25 | 102.33 | 107.75 |
| ST9328 | 9.11 | 0.00 | 4.42 | 88.45 | 95.17 |
| ST9344 | 4.55 | 1.82 | 1.46 | 45.19 | 83.45 |
| ST9345 | 4.35 | 1.42 | 1.39 | 44.07 | 69.07 |
| ST9294 | 49.45 | 0.00 | 38.02 | 9.97 | 91.03 |
| ST9296 | 39.82 | 14.73 | 31.84 | 5.76 | 110.33 |
| ST9297 | 43.16 | 11.97 | 27.68 | 3.98 | 100.94 |
| ST9298 | 55.48 | 6.75 | 44.86 | 8.71 | 129.11 |
| ST9299 | 56.42 | 3.90 | 44.40 | 9.36 | 110.51 |
| ST9300 | 41.41 | 0.93 | 34.68 | 8.14 | 91.03 |
| ST9301 | 33.85 | 3.56 | 27.95 | 4.52 | 74.90 |
| ST9302 | 30.80 | 3.43 | 27.85 | 6.55 | 78.06 |
| ST9329 | 54.21 | 0.00 | 36.92 | 7.52 | 90.75 |
| ST9347 | 44.31 | 1.95 | 36.95 | 7.97 | 114.67 |
| ST9348 | 41.61 | 6.85 | 30.91 | 5.85 | 100.61 |
| ST9314 | 41.47 | 0.00 | 29.74 | 0.00 | 69.47 |
| ST9315 | 42.97 | 13.32 | 25.98 | 0.00 | 103.05 |
| ST9316 | 51.01 | 15.90 | 37.73 | 0.00 | 123.91 |
| ST9317 | 52.00 | 16.55 | 39.73 | 0.00 | 125.39 |
| ST9318 | 40.77 | 4.14 | 35.09 | 0.00 | 89.35 |
| ST9319 | 49.94 | 2.68 | 42.30 | 0.00 | 99.29 |
| ST9320 | 57.96 | 1.48 | 47.66 | 0.00 | 116.77 |
| ST9321 | 58.14 | 7.64 | 47.40 | 0.00 | 125.40 |
| ST9322 | 51.55 | 4.71 | 44.44 | 0.00 | 120.42 |
| ST9330 | 63.99 | 0.00 | 42.92 | 0.00 | 102.99 |
| ST9350 | 47.22 | 2.05 | 38.52 | 38.41 | 111.42 |
| ST9351 | 43.19 | 1.58 | 32.98 | 17.89 | 96.42 |

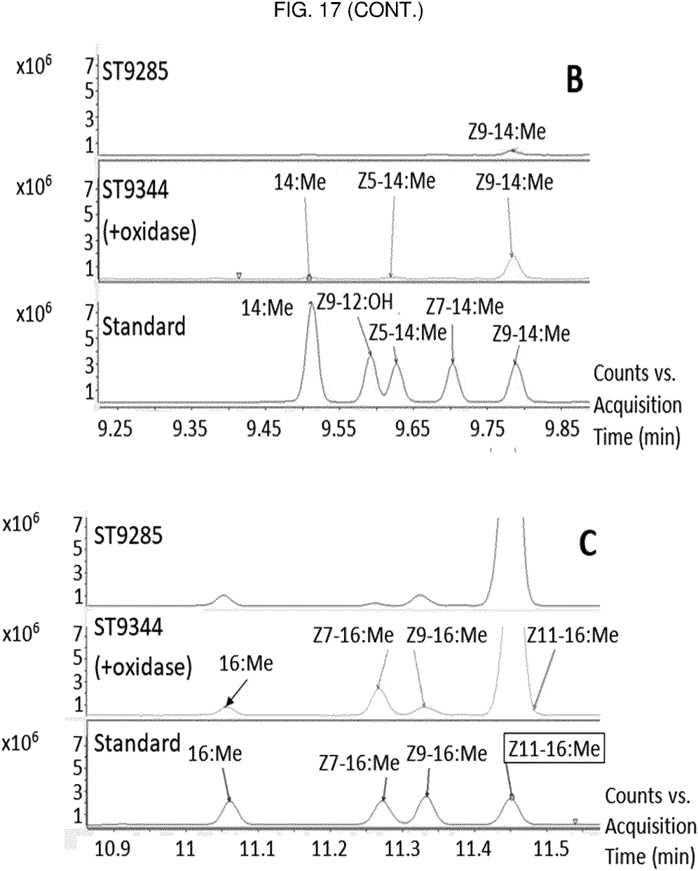

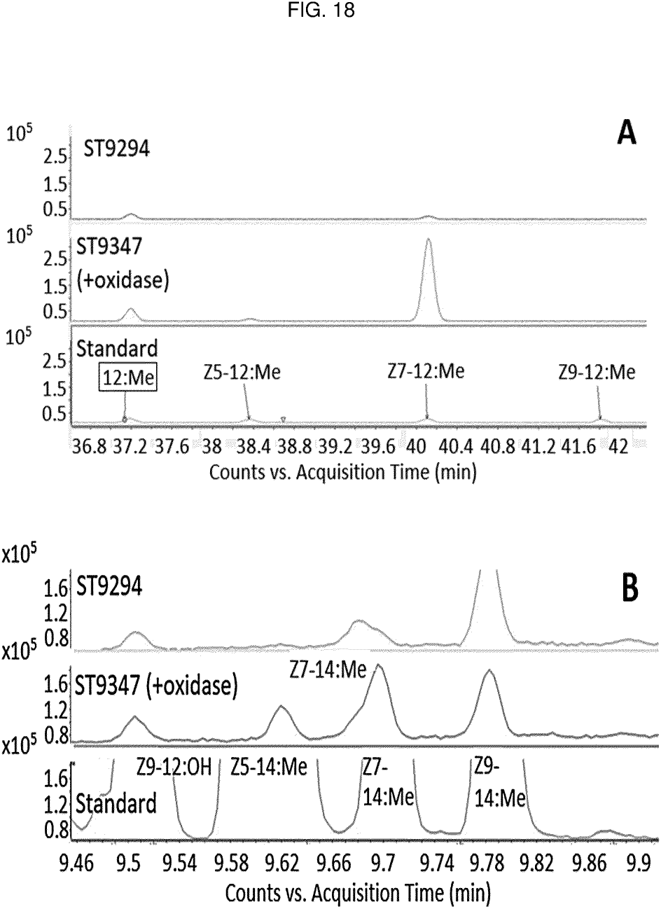

Several chain-shortened fatty acids could be detected. In strains combining the expression of Δ_{Z11}-16 desaturase Slitdes5 (SEQ ID NO: 87) and peroxisomal oxidases Cma_POX (SEQ ID NO: 22), Hsa_POX1-2 (SEQ ID NO: 24) and RnoPOX-2 increased production of Δ_{Z9}-14:Me, and in strains expressing Slitdes5 and peroxisomal oxidases POX3, POX5, Cma_POX and RnoPOX-2 production of Δ_{Z7}-12:Me, respectively, could be detected (FIGS. 17A and B).

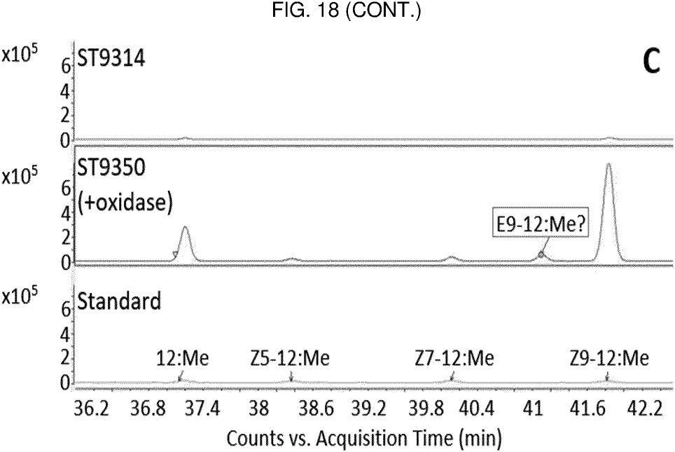

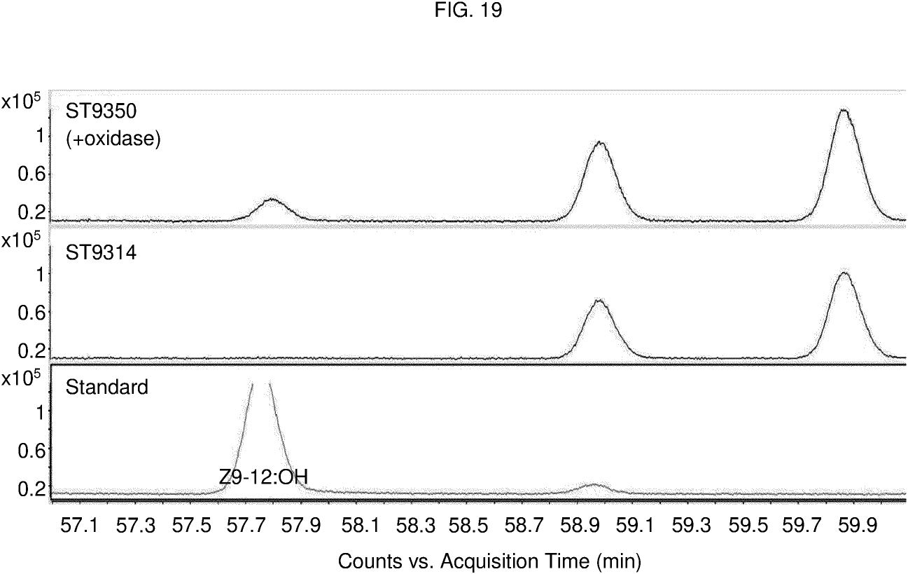

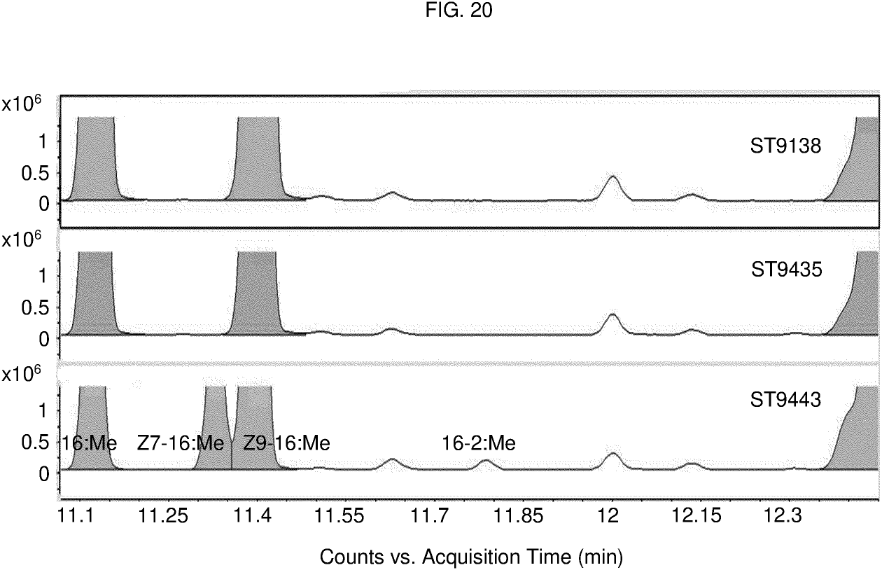

In strains combining the expression of Δ_{Z9}-14 desaturase Dmd9 and peroxisomal oxidases Cma_POX, Hsa_POX1-2, RnoPOX-2, Lbo31670 (SEQ ID NO: 81) and Lbo49554 (SEQ ID NO: 83), respectively, increased production of Δ_{Z7}-12:Me could be detected (FIG. 18A).

In strains combining the expression of Δ_{Z11}-14 desaturase Lbo_PTTQ and peroxisomal oxidases Cma_POX, Hsa_POX1-2, PurPOX (SEQ ID NO: 26), RnaPOX-2, Ase_POX (SEQ ID NO: 14), Lbo31670 and Lbo49554, respectively, increased production of Δ_{Z9}-12:Me could be detected (FIG. 18C). The additional monounsaturated methyl dodecenoate is probably Δ_{E9}-12:Me originating from the chain-shortening of Δ_{E11}-14:Me produced by the Δ_{Z11}-14 desaturase Lbo_PPTQ.

The chain-shortened product of the natively produced Δ_{Z9}-18:CoA Δ_{Z7}-16:CoA could be detected in strains expressing the peroxisomal oxidases Ani_POX, Cma_POX, Hsa_POX1-2, PurPOX, Rno_POX-2, Lbo31670, Lbo49554 and the native Y. lipolytica oxidases POX2, POX3 and POX5 (FIG. 17C).

The chain-shortened product of the natively produced Δ_{Z9}-16:CoA Δ_{Z7}-14:CoA and Δ_{Z5}-12:Me could be detected in strains expressing the peroxisomal oxidases Cma_POX, Hsa_POX1-2, PurPOX, Rno_POX-2, Lbo31670 and Lbo49554 (FIGS. 18A and B).

Both retention times and spectra are matching the reference compounds.

### Example 24: Production of ΔZ9-12:OH in Yeast

Strain ST9640 is derived from ST9138 and bears deletions of all Y. lipolytica native peroxisomal oxidases POX1-6. Strain ST9640 additionally expresses fatty acyl reductase from Helicoverpa armigera (Har_FAR, SEQ ID NO: 59), the fatty acyl reductase of Agrotis segetum (AseFAR, SEQ ID NO: 92) and the Δ_{Z11}-14 desaturase from Lobesia botrana (Lbo_PTTQ, SEQ ID NO: 86) and the peroxisomal oxidase Lbo31670 from Lobesia botrana (Lbo31670, SEQ ID NO: 80).

Production of Δ_{Z9}-12:OH could be detected in strain ST9350 (FIG. 19). Both, retention time and spectrum is matching the reference compound. Δ_{Z9}-12:OH can be chemically acetylated into Δ_{Z9}-12:OAc. Δ_{Z9}-12:OAc is a major pheromone component of the grape berry moth Paralobesia viteana and can be used to monitor or control this pest by mating disruption.

### Example 25: Production of Δ_{Z7}, Δ_{Z/E11}-16:Me and Δ_{Z7}, Δ_{Z/E11}-16:OH

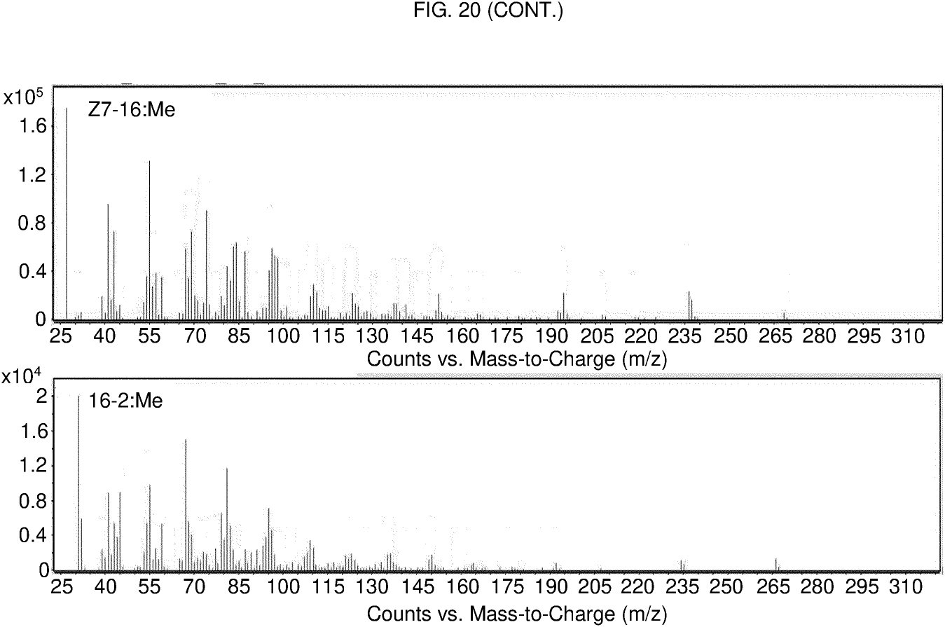

Strain ST9435 is derived from ST9138 and bears deletions of all Y. lipolytica native peroxisomal oxidases POX1-6. Strain ST9435 additionally expresses the desaturase PGDes8 from Pectinophora gossypiella (SEQ ID NO: 100). Strain ST9443 is derived from ST9435 and additionally expresses the peroxisomal oxidase from P. ureafaciens (Pur_POX, SEQ ID NO: 25). When separated by GC-MS (oven ramp program a) the extracts of all three strains contain peaks which correspond to saturated methyl palmitate and one monounsaturated hexadecenoic acid methyl ester, presumably the natively occurring Δ_{Z9}-hexadecenoic acid methyl ester. The extract of ST9443 contained two additional compounds compared to ST9435 eluting at 11.32 minutes and 11.8 minutes (FIG. 20). The mass spectrum of the 11.32-minutes-eluent matches with the spectrum of a monounsaturated hexadecenoic acid methyl ester and the 11.8-minute-eluent matches with a double unsaturated hexadecenoic acid methyl ester, respectively (FIG. 20). The second monounsaturated hexadecenoic acid methyl ester might be Δ_{Z7}-hexadecenoic acid methyl ester arising from peroxisomal chain shortening of oleic acid by Pur_POX peroxisomal oxidase. The double unsaturated hexadecenoic acid methyl ester might be Δ_{Z7}, Δ_{Z11}-16:Me and/or Δ_{Z7}, Δ_{E11}-16:Me as it does not occur e.g. in strain ST9292 which only expresses the peroxisomal oxidase Pur_POX but not the desaturase PGDes8 of Pectinophora gossypiella.

Sequences

| SEQ ID NO: | | | | |
|---|---|---|---|---|
| 1 | DNA | Yarrowia lipolytica | Peroxisomal oxidase 1 | Yli_POX1 (YALI0_E32835g) |
| 2 | protein | Yarrowia lipolytica | Peroxisomal oxidase 1 | Yli_POX1 (XP_504703) |
| 3 | DNA | Yarrowia lipolytica | Peroxisomal oxidase 2 | Yli_POX2 (YALI0_F10857g) |
| 4 | protein | Yarrowia lipolytica | Peroxisomal oxidase 2 | Yli_POX2 (XP_505264) |
| 5 | DNA | Yarrowia lipolytica | Peroxisomal oxidase 3 | Yli_POX3 (YALI0_D24750g |
| 6 | protein | Yarrowia lipolytica | Peroxisomal oxidase 3 | Yli_POX3 (XP_503244 |
| 7 | DNA | Yarrowia lipolytica | Peroxisomal oxidase 4 | Yli_POX4 (YALI0_E27654g |
| 8 | protein | Yarrowia lipolytica | Peroxisomal oxidase 4 | Yli_POX4 (XP_504475) |
| 9 | DNA | Yarrowia lipolytica | Peroxisomal oxidase 5 | Yli_POX5 (YALI0_C23859g) |
| 10 | protein | Yarrowia lipolytica | Peroxisomal oxidase 5 | Yli_POX5 (XP_502199) |
| 11 | DNA | Yarrowia lipolytica | Peroxisomal oxidase 6 | Yli_POX6 (YALI0_E06567g) |
| 12 | protein | Yarrowia lipolytica | Peroxisomal oxidase 6 | Yli_POX6 (XP_503632) |
| 13 | mRNA-coding sequence, codon-optimized for Y. lipolytica | Agrotis segetum | Peroxisomal oxidase | Ase_POX |
| 14 | protein | Agrotis segetum | Peroxisomal oxidase | Ase_POX |
| 15 | mRNA-coding sequence, codon-optimized for Y. lipolytica | Arabidopsis thaliana | Peroxisomal oxidase 1 | Ath_POX1 |
| 16 | protein | Arabidopsis thaliana | Peroxisomal oxidase 1 | Ath_POX1 |
| 17 | mRNA-coding sequence, codon-optimized for Y. lipolytica | Arabidopsis thaliana | Peroxisomal oxidase 2 | Ath_POX2 |
| 18 | protein | Arabidopsis thaliana | Peroxisomal oxidase 2 | Ath_POX2 |
| 19 | mRNA-coding sequence, codon-optimized for Y. lipolytica | Aspergillus nidulans | Peroxisomal oxidase | Ani_POX |
| 20 | protein | Aspergillus nidulans | Peroxisomal oxidase | Ani_POX |
| 21 | mRNA-coding sequence, codon-optimized for Y. lipolytica | Cucurbita maxima | Peroxisomal oxidase | Cma_POX |
| 22 | protein | Cucurbita maxima | Peroxisomal oxidase | Cma_POX |
| 23 | mRNA-coding sequence, codon-optimized for Y. lipolytica | Homo sapiens | Peroxisomal oxidase | Hsa_POX1-2 |
| 24 | protein | Homo sapiens | Peroxisomal oxidase | Hsa_POX1-2 |
| 25 | mRNA-coding sequence, codon-optimized for Y. lipolytica | Paenarthrobac ter ureafaciens | Peroxisomal oxidase | Pur_POX |
| 26 | protein | Paenarthrobac ter ureafaciens | Peroxisomal oxidase | Pur_POX |
| 27 | mRNA-coding sequence, codon-optimized for Y. lipolytica | Rattus norvegicus | Peroxisomal oxidase | Rno_POX-2 |
| 28 | protein | Rattus norvegicus | Peroxisomal oxidase | Rno_POX-2 |
| 29 | mRNA-coding sequence, codon-optimized for Y. lipolytica | Saccharomyces cerevisiae | Δ_{Z9}-desaturase | Sce_OLE1 |
| 30 | protein | S. cerevisiae | Δ_{Z9}-desaturase | Sce_OLE1 |
| 31 | mRNA-coding sequence | Y. lipolytica | Δ_{Z9}-desaturase | Yli_OLE1 (YALI0C05951g) |
| 32 | protein | Y. lipolytica | Δ_{Z9}-desaturase | Yli_OLE1 (XP_501496) |
| 33 | mRNA-coding sequence, codon-optimized for Y. lipolytica using IDT tools | Drosophila melanogaster | Δ_{Z9}-14-desaturase | Dme_D9_YIopIDT |
| 34 | mRNA-coding sequence, codon-optimized for Y. lipolytica | Drosophila melanogaster | Δ_{Z9}-14-desaturase | Dme_D9 |
| 35 | Protein | Drosophila melanogaster | Δ_{Z9}-14-desaturase | Dme_D9 |
| 36 | mRNA-coding sequence, codon-optimized for Y. lipolytica using IDT tools | Amyelois transitella | Δ_{Z11}-16-desaturase | Atr_D11_YIopIDT |
| 37 | mRNA-coding sequence, codon-optimized for Y. lipolytica using GeneArt | Amyelois transitella | Δ_{Z11}-16-desaturase | Atr_D11 |
| 38 | protein | Amyelois transitella | Δ_{Z11}-16-desaturase | Atr_D11 |
| 39 | mRNA-coding sequence, codon-optimized for Y. lipolytica | Choristoneura rosaceana | Δ_{Z11}-14-desaturase | Cro_Z11 |
| 40 | protein | Choristoneura rosaceana | Δ_{Z11}-14-desaturase | Cro_Z11 |
| 41 | mRNA-coding sequence, codon-optimized for Y. lipolytica | Ostrinia nubilalis | Δ_{Z11}-14-desaturase | Onu_11 |
| 42 | protein | Ostrinia nubilalis | Δ_{Z11}-14-desaturase | Onu_11 |
| 43 | mRNA-coding sequence, codon-optimized for Y. lipolytica | Thaumetopoea pityocampa | Δ_{Z11}-14-desaturase | Tpi_D13 |
| 44 | protein | Thaumetopoea pityocampa | Δ_{Z11}-14-desaturase | Tpi_D13 |
| 45 | mRNA-coding sequence, codon-optimized for Y. lipolytica | Dendrophilus punctatus | Δ_{E9}-14-desaturase | Dpu_E9-14 |
| 46 | protein | Dendrophiluspunctatus | Δ_{E9}-14-desaturase | Dpu_E9-14 |
| 47 | mRNA-coding sequence, codon-optimized for Y. lipolytica | Grapholita molesta | Δ_{Z/E10}-14-desaturase | Gmo_CPRQ |
| 48 | protein | Grapholita molesta | Δ_{Z/E10}-14-desaturase | Gmo_CPRQ |
| 49 | mRNA-coding sequence, codon-optimized for Y. lipolytica | Cydia pomonella | desaturase | Cpo_CPRQ |
| 50 | protein | Cydia pomonella | desaturase | Cpo_CPRQ |
| 51 | mRNA-coding sequence, codon-optimized for Y. lipolytica | Epiphyas postvittana | desaturase | Epo_E11 |
| 52 | protein | Epiphyas postvittana | desaturase | Epo_E11 |
| 53 | mRNA-coding sequence, codon-optimized for Y. lipolytica | Spodoptera littoralis | desaturase | Sls_ZE11 |
| 54 | protein | Spodoptera littoralis | desaturase | Sls_ZE11 |
| 55 | mRNA-coding sequence, codon-optimized for Y. lipolytica | Choristoneura parallela | desaturase | Cpa_E11 |
| 56 | protein | Choristoneura parallela | desaturase | Cpa_E11 |
| 57 | mRNA-coding sequence, codon-optimized for Y. lipolytica using IDT tools | H. armigera | fatty acyl-CoA reductase | Har_FAR |
| 58 | mRNA-coding sequence, codon-optimized for Y. lipolytica using GeneArt | H. armigera | fatty acyl-CoA reductase | Har_FAR |
| 59 | protein | H. armigera | fatty acyl-CoA reductase | Har_FAR |
| 60 | mRNA-coding sequence, codon-optimized for Y. lipolytica | S. cerevisiae | acetyltransferase | Sce_ATF1 |
| 61 | protein | S. cerevisiae | acetyltransferase | Sce_ATF1 |
| 62 | DNA | Y. lipolytica | TEF1 promoter | |
| 63 | | Y. lipolytica | TEF1intron promoter | |
| 64 | DNA | | BB8251 | Tefintron-Dmd9 |
| 65 | DNA | | BB8248 | Tefintron-Atrd11 |
| 66 | DNA | Y. lipolytica | peroxisome biogenesis factor pex10 | PEX10 |
| 67 | Protein | Y. lipolytica | peroxisome biogenesis factor pex10 | PEX10 |
| 68 | DNA | Y. lipolytica | Fatty acid synthase 1 | FAS1 |
| 69 | Protein | Y. lipolytica | Fatty acid synthase 1 | FAS1 |
| 70 | DNA | Y. lipolytica | Fatty acid synthase 2 | FAS2 |
| 71 | Protein | Y. lipolytica | Fatty acid synthase 2 | FAS2 |
| 72 | S. cerevisiae-codon-optimised nucleotide sequence; mRNA-coding sequence | H. subflexa | Fatty acyl-CoA reductase | Hs_FAR |
| 73 | Protein | H. subflexa | Fatty acyl-CoA reductase | Hs_FAR |
| 74 | DNA | Helicoverpa assulta | Fatty acyl-CoA reductase | Has_FAR |
| 75 | Protein | Helicoverpa assulta | Fatty acyl-CoA reductase | Has_FAR |
| 76 | DNA | Bicyclus anynana | Fatty acyl-CoA reductase | Ban_FAR |
| 77 | Protein | Bicyclus anynana | Fatty acyl-CoA reductase | Ban_FAR |
| 78 | mRNA-coding sequence, codon-optimized for Y. lipolytica | Lobesia botrana | desaturase | Lbo_PTTQ |
| 79 | Protein | Lobesia botrana | desaturase | Lbo_PTTQ |
| 80 | mRNA-coding sequence, codon-optimized for Y. lipolytica | Lobesia botrana | Peroxisomal oxidase | Lbo31670 |
| 81 | Protein | Lobesia botrana | Peroxisomal oxidase | Lbo31670 |
| 82 | mRNA-coding sequence, codon-optimized for Y. lipolytica | Lobesia botrana | Peroxisomal oxidase | Lbo49554 |
| 83 | Protein | Lobesia botrana | Peroxisomal oxidase | Lbo49554 |
| 84 | mRNA-coding sequence, codon-optimized for Y. lipolytica | Lobesia botrana | Peroxisomal oxidase | Lbo49602 |
| 85 | Protein | Lobesia botrana | Peroxisomal oxidase | Lbo49602 |
| 86 | mRNA-coding sequence, codon-optimized for Y. lipolytica | Spodoptera litura | desaturase | Slitdes5 |
| 87 | Protein | Spodoptera litura | desaturase | Slitdes5 |
| 88 | mRNA-coding sequence, codon- | Lobesia botrana | desaturase | Lbo_KPSE |
| | optimized for Y. lipolytica | | | |
| 89 | Protein | Lobesia botrana | desaturase | Lbo_KPSE |
| 90 | mRNA-coding sequence, codon-optimized for Y. lipolytica | Chilo suppressalis | desaturase | Csup_KPSE |
| 91 | Protein | Chilo suppressalis | desaturase | Csup_KPSE |
| 92 | mRNA-coding sequence, codon-optimized for Y. lipolytica | Agrotis segetum | Fatty acyl-CoA reductase | AseFAR |
| 93 | Protein | Agrotis segetum | Fatty acyl-CoA reductase | AseFAR |
| 94 | DNA | Artificial | GTGCAGGUGCCACAAT GACCGAGG | PR-22847 |
| 95 | DNA | Artificial | CGTGCGAUCTACAGCT TCGAAGAAG | PR-22848 |
| 96 | mRNA-coding sequence, codon-optimized | Grapholita molesta | desaturase | Gmo_KPSQ |
| 97 | protein | Grapholita molesta | desaturase | Gmo_KPSQ |
| 98 | mRNA-coding sequence, codon-optimized | Cydia pomonella | desaturase | Cpo_CPRQ |
| 99 | protein | Cydia pomonella | desaturase | Cpo_CPRQ |
| 100 | mRNA-coding sequence, codon-optimized | Pectinophora gossypiella | desaturase | PGDes8 |
| 101 | protein | Pectinophora gossypiella | desaturase | PGDes8 |
| 102 | DNA | Artificial | Primer sequence | PR-21661 |
| 103 | DNA | Artificial | Primer sequence | PR-21662 |
| 104 | DNA | Artificial | Primer sequence | PR-21673 |
| 105 | DNA | Artificial | Primer sequence | PR-21674 |
| 106 | DNA | Artificial | Primer sequence | PR-18977 (PrTef->_fw) |
| 107 | DNA | Artificial | Primer sequence | PR10604 |
| 108 | DNA | Artificial | Primer sequence | PR10655 |
| 109 | DNA | Artificial | Primer sequence | PR10656 |
| 110 | DNA | Artificial | Primer sequence | PR11110 |
| 111 | DNA | Artificial | Primer sequence | PR11111 |
| 112 | DNA | Artificial | Primer sequence | PR11138 |
| 113 | DNA | Artificial | Primer sequence | PR11139 |
| 114 | DNA | Artificial | Primer sequence | PR14148 |
| 115 | DNA | Artificial | Primer sequence | PR14149 |
| 116 | DNA | Artificial | Primer sequence | PR14588 |
| 117 | DNA | Artificial | Primer sequence | PR15522 |
| 118 | DNA | Artificial | Primer sequence | PR15781 |
| 119 | DNA | Artificial | Primer sequence | PR15788 |
| 120 | DNA | Artificial | Primer sequence | PR15789 |
| 121 | DNA | Artificial | Primer sequence | PR15930 |
| 122 | DNA | Artificial | Primer sequence | PR20763 |
| 123 | DNA | Artificial | Primer sequence | PR20764 |
| 124 | DNA | Artificial | Primer sequence | PR21771 |
| 125 | DNA | Artificial | Primer sequence | PR21925 |
| 126 | DNA | Artificial | Primer sequence | PR22075 |
| 127 | DNA | Artificial | Primer sequence | PR22213 |
| 128 | mRNA-coding sequence, codon-optimized for Y. lipolytica | Cydia pomonella | desaturase | Cpo_CPRQ + exon + tail 1 |
| 129 | protein | Cydia pomonella | desaturase | Cpo_CPRQ + exon + tail 1 |
| 130 | mRNA-coding sequence, codon-optimized for Y. lipolytica | Cydia pomonella | desaturase | Cpo_CPRQ + exon + tail 2 |
| 131 | protein | Cydia pomonella | desaturase | Cpo_CPRQ + exon + tail 2 |
| 132 | mRNA-coding sequence, codon-optimized for Y. lipolytica | Cydia pomonella | desaturase | Cpo_CPRQ - exon + tail 1 |
| 133 | protein | Cydia pomonella | desaturase | Cpo_CPRQ - exon + tail 1 |
| 134 | mRNA-coding sequence, codon-optimized for Y. lipolytica | Cydia pomonella | desaturase | Cpo_CPRQ - exon + tail 2 |
| 135 | protein | Cydia pomonella | desaturase | Cpo_CPRQ -exon + tail 2 |
| 136 | mRNA-coding sequence, codon-optimized for Y. lipolytica | Cydia pomonella | desaturase | CPOM09289 + exon + tail 1 |
| 137 | protein | Cydia pomonella | desaturase | CPOM09289 + exon + tail 1 |
| 138 | mRNA-coding sequence, codon-optimized for Y. lipolytica | Cydia pomonella | desaturase | CPOM09289 + exon + tail 2 |
| 139 | Protein | Cydia pomonella | desaturase | CPOM09289 + exon + tail 2 |
| 140 | mRNA-coding sequence, codon-optimized for Y. lipolytica | Cydia pomonella | desaturase | CPOM09289 + exon ― tail 1 |
| 141 | protein | Cydia pomonella | desaturase | CPOM09289 + exon ― tail 1 |
| 142 | mRNA-coding sequence, codon-optimized for Y. lipolytica | Cydia pomonella | desaturase | CPOM09289 + exon - tail 2 |
| 143 | protein | Cydia pomonella | desaturase | CPOM09289 + exon ― tail 2 |
| 144 | mRNA-coding sequence, codon-optimized for Y. lipolytica | Cydia pomonella | desaturase | CPOM09289 - exon + tail 1 |
| 145 | protein | Cydia pomonella | desaturase | CPOM09289 - exon + tail 1 |
| 146 | mRNA-coding sequence, codon-optimized for Y. lipolytica | Cydia pomonella | desaturase | CPOM09289 - exon + tail 2 |
| 147 | protein | Cydia pomonella | desaturase | CPOM09289 - exon + tail 2 |

### REFERENCES

Böröczky K. Pheromone Communication in Moths: Evolution, Behavior, and Application. Am Entomol 2017;63:260-1. doi:10.1093/ae/tmx069.

Borodina I, Holkenbrink C, Dam M, Löfstedt C, Ding B, Wang H. Method for production of moth pheromones in yeast, 2016.

Borodina I, Holkenbrink C, Dam M, Löfstedt C. Methods for producing fatty alcohols and derivatives thereof in yeast, 2018.

Borodina I, Holkenbrink C, Dam M, Löfstedt C, Ding B, Wang H-L. Production of desaturated fatty alcohols and desaturated fatty acyl acetates in yeast., 2018.

Holkenbrink C, Dam MI, Kildegaard KR, Beder J, Dahlin J, Belda DD, et al. EasyCloneYALI: CRISPR/Cas9-Based Synthetic Toolbox for Engineering of the Yeast Yarrowia lipolytica. Biotechnol J n.d.;0:1700543. doi:10.1002/biot.201700543.

Wang HJ, Le Dall M-T, Waché Y, Laroche C, Belin J-M, Gaillardin C, et al. Evaluation of acyl coenzyme A oxidase (Aox) isozyme function in the n-alkane-assimilating yeast Yarrowia lipolytica. J Bacteriol 1999; 181:5140-8. doi:10.1097/CHI.0b013e3181af8216.

Shimizu S, Yasui K, Yoshiki T, Yamada H. Acyl-CoA oxidase from Candida tropicalis. Biochem Biophys Res Commun 1979;91:108-13.

Ding B-J, Löfstedt C. Analysis of the Agrotis segetum pheromone gland transcriptome in the light of sex pheromone biosynthesis. BMC Genomics 2015;16:711. doi:10.1186/s12864-015-1909-2.

Hooks MA, Kellas F, Graham IA. Long-chain acyl-CoA oxidases of Arabidopsis. Plant J 1999;20:1-13. doi:10.1046/j.1365-313X.1999.00559.x.

Khan BR, Adham AR, Zolman BK. Peroxisomal Acyl-CoA oxidase 4 activity differs between Arabidopsis accessions. Plant Mol Biol 2012;78:45-58. doi:10.1007/s11103-011-9843-4.

Reiser K, Davis MA, Hynes MJ. AoxA is a major peroxisomal long chain fatty acyl-CoA oxidase required for β-oxidation in A. nidulans. Curr Genet 2010;56:139-50. doi:10.1007/s00294-009-0286-2.

Hayashi H, Bellis LD, Yamaguchi K, Kato A, Hayashi M, Nishimura M. Molecular Characterization of a Glyoxysomal Long Chain Acyl-CoA Oxidase That Is Synthesized as a Precursor of Higher Molecular Mass in Pumpkin. J Biol Chem 1998;273:8301-7. doi:10.1074/jbc.273.14.8301.

Oaxaca-Castillo D, Andreoletti P, Vluggens A, Yu S, van Veldhoven PP, Reddy JK, et al. Biochemical characterization of two functional human liver acyl-CoA oxidase isoforms 1a and 1b encoded by a single gene. Biochem Biophys Res Commun 2007;360:314-9. doi:10.1016/j.bbrc.2007.06.059.

Bakke M, Setoyama C, Miura R, Kajiyama N. N-Ethylmaleimide-resistant acyl-coenzyme A oxidase from Arthrobacter ureafaciens NBRC 12140: Molecular cloning, gene expression and characterization of the recombinant enzyme. Biochim Biophys Acta BBA - Proteins Proteomics 2007;1774:65-71. doi: 10.1016/j.bbapap.2006.10.008.

Ngo SNT, McKinnon RA, Stupans I. Identification and cloning of two forms of liver peroxisomal fatty Acyl CoA Oxidase from the koala (Phascolarctos cinereus). Gene 2003;309:91-9. doi:10.1016/S0378-1119(03)00491-8.

Miyazawa S, Hayashi H, Hijikata M, Ishii N, Furuta S, Kagamiyama H, et al. Complete nucleotide sequence of cDNA and predicted amino acid sequence of rat acyl-CoA oxidase. J Biol Chem 1987;262:8131-7.

Luo Y-S, Nicaud J-M, Van Veldhoven PP, Chardot T. The acyl-CoA oxidases from the yeast Yarrowia lipolytica: characterization of Aox2p. Arch Biochem Biophys 2002;407:32-8. doi:10.1016/S0003-9861(02)00466-6.

Wang H, Dall M-TL, Waché Y, Laroche C, Belin J-M, Nicaud J-M. Cloning, sequencing, and characterization of five genes coding for Acyl-CoA oxidase isozymes in the yeast Yarrowia lipolytica. Cell Biochem Biophys 1999;31:165-74. doi:10.1007/BF02738170.

Corberán VC, González-Pérez ME, Martínez-González S, Gómez-Avilés A. Green oxidation of fatty alcohols: challenges and opportunities. Appl Catal Gen 2014;474:211-23. doi:10.1016/j.apcata.2013.09.040.

Yadav, Reddy, Basak, Narsaiah, 2004. Recyclable 2nd generation ionic liquids as green solvents for the oxidation of alcohols with hypervalent iodine reagents, Tetrahedron, 60, 2131-2135.

Meyer, Schreiber, 1994. Acceleration of the Dess-Martin oxidation by water J. Org. Chem., 59, 7549-7552.

Steves J.E. and Stahl S.S., 2013. Copper(I)/ABNO-catalyzed aerobic alcohol oxidation: alleviating steric and electronic constraints of Cu/TEMPO catalyst systems. J. Am. Chem. Soc., 135, 15742-15745.

Okada, Asawa, Sugiyama, Kirihara, Iwai, Kimura, 2014. Sodium hypochlorite pentahydrate (NaOCl·5H2O) crystals as an extraordinary oxidant for primary and secondary alcohols. Synlett, 25, 596-598.

Tamura, Aoyama, Takido, Kodomari, 2012. Novel [4-Hydroxy-TEMPO + NaCl]/SiO2 as a reusable catalyst for aerobic oxidation of alcohols to carbonyls. Synlett, 23, 1397-1407.

Li, Zhang, 2009. An environmentally benign TEMPO-catalyzed efficient alcohol oxidation system with a recyclable hypervalent iodine(III) reagent andilts facile preparation. Synthesis, 1163-1169a.

Ferrell, Yao, 1972. Reductive and oxidative synthesis of saturated and unsaturated fatty aldehydes, J Lipid Res. 13(1):23-6.).

Kehat, Dunkelblum, 1993. Sex Pheromones: achievements in monitoring and mating disruption of cotton pests in Israel, Achieves of Insect Biochemistry and Physiology. 22:425-431.

Alfaro, Navarro-Llopis, Primo, 2009. Optimization of pheromone dispenser density for managing the rice striped stem borer, Chilo suppressalis (Walker), by mating disruption. Crop Protection. 28:567-572.

Eizaguirre, Sans, López, Albajes. 2002. Effects of mating disruption against the Mediterranean corn borer, Sesamia nonagrioides, on the European corn borer Ostrinia nubilalis. Use of pheromones and other semiochemicals in integrated production IOBC wprs Bulletin.

Wu, Zhang, Yao, Xu, Wang and Zhang, 2012. Management of diamondback moth, Plutella xylostella (Lepidoptera: Plutellidae) by mating disruption. Insect Science 19 (6), 643-648.

Bari, 2003. Development of pheromone mating disruption strategies for the suppression of the artichoke plume moth in artichokes grown on the central coast of California. ISHS Acta Horticulturae 660: V International Congress on Artichoke. doi: 10.17660/ActaHortic.2004.660.80

Jensen, Strucko, Kildegaard, David, Maury, Mortensen, Nielsen and Borodina, 2014. EasyClone: method for iterative chromosomal integration of multiple genes Saccharomyces cerevisiae, FEMS Yeast Research, Volume 14, Issue 2, 238-248,

Stovicek, Borja, Forster and Borodina, 2015. EasyClone 2.0: expanded toolkit of integrative vectors for stable gene expression in industrial Saccharomyces cerevisiae strains. J Ind Microbiol Biotechnol 42, 1519-1531 (2015). https://doi.org/10.1007/s10295-015-1684-8

### Items

1. A yeast cell capable of producing a desaturated fatty alcohol and optionally a desaturated fatty acyl acetate and/or a desaturated fatty aldehyde, said yeast cell:
i) having one or more mutations resulting in reduced activity of one or more native acyl-CoA oxidases; and
ii) expressing at least one first group of enzymes comprising at least one acyl-CoA oxidase capable of oxidising a fatty acyl-CoA, wherein the first group of enzymes is capable of shortening a fatty acyl-CoA of a first carbon chain length X to a shortened fatty acyl-CoA having a second carbon chain length X′, wherein X′ ≤ X-2; and
iii) expressing at least one heterologous desaturase capable of introducing at least one double bond in said fatty acyl-CoA and/or in said shortened fatty acyl-CoA; and
iv) expressing at least one heterologous fatty acyl-CoA reductase, capable of converting at least part of said desaturated fatty acyl-CoA to a desaturated fatty alcohol; and
v) optionally expressing at least one acetyltransferase capable of converting at least part of said desaturated fatty alcohol to a desaturated fatty acyl acetate, and/or at least one alcohol dehydrogenase and/or fatty alcohol oxidase capable of converting at least part of said desaturated fatty alcohol to a desaturated fatty aldehyde.

2. The yeast cell according to item 1, wherein

$$\text{X′ = X-2, X′ = X-4 or X′ = X-6} \text{.}$$

3. The yeast cell according to any one of the preceding items, wherein the native acyl-CoA oxidase and/or the heterologous acyl-CoA oxidase is a peroxisomal acyl-CoA oxidase.

4. The yeast cell according to any one of the preceding items, wherein the one or more mutations resulting in reduced activity of one or more native acyl-CoA oxidases results in partial or total loss of activity of the one or more native acyl-CoA oxidases.

5. The yeast cell according to any one of the preceding items, wherein the reduced activity of the one or more native acyl-CoA oxidases is a reduced activity on acyl-CoAs having a carbon chain length smaller than X, such as smaller than X′.

6. The yeast cell according to any one of the preceding items, wherein the first group of enzymes has greater activity towards fatty acyl-CoAs of carbon chain length greater than X′ than towards fatty acyl-CoAs of carbon chain length equal to or smaller than X′.

7. The yeast cell according to any one of the preceding items, wherein the at least one acyl-CoA oxidase of the first group of enzymes of step ii) is a native acyl-CoA oxidase or a heterologous acyl-CoA oxidase.

8. The yeast cell according to any one of the preceding items, wherein the at least one acyl-CoA oxidase of the first group of enzymes of step ii) is a native acyl-CoA oxidase, which is overexpressed compared to a reference yeast strain not expressing said at least one first group of enzymes.

9. The yeast cell according to any one of the preceding items, wherein the at least one acyl-CoA oxidase of the first group of enzymes of step ii) is a heterologous acyl-CoA oxidase.

10. The yeast cell according to any one of the preceding items, wherein the at least one first group of enzymes comprises an acyl-CoA oxidase derived from a prokaryotic organism or from a eukaryotic organism.

11. The yeast cell according to any one of the preceding items, wherein the at least one acyl-CoA oxidase of the first group of enzymes is an acyl-CoA oxidase derived from a yeast, a fungus, an insect, a mammalian, a bird or a plant, such as the at least one acyl-CoA oxidase of the first group of enzymes is derived from a yeast, a fungus, an insect, a mammalian, a bird or a plant.

12. The yeast cell according to any one of the preceding items, wherein the at least one first group of enzymes comprises an acyl-CoA oxidase derived from an organism of a genus selected from Yarrowia, Agrotis, Arabidopsis, Aspergillus, Cucurbita, Homo, Paenarthrobacter, Lobesia or Rattus.

13. The yeast cell according to any one of the preceding items, wherein the at least one first group of enzymes comprises an acyl-CoA oxidase derived from Yarrowia lipolytica, Agrotis segetum, Arabidopsis thaliana, Aspergillus nidulans, Cucurbita maxima, Homo sapiens, Paenarthrobacter ureafaciens, Lobesia botrana or Rattus norvegicus.

14. The yeast cell according to any one of the preceding items, wherein the at least one acyl-CoA oxidase of the first group of enzymes is an acyl-CoA oxidase selected from the group consisting of Yli_POX1 (SEQ ID NO: 2), Yli_POX2 (SEQ ID NO: 4), Yli_POX3 (SEQ ID NO: 6), Yli_POX4 (SEQ ID NO: 8), Yli_POX5 (SEQ ID NO: 10), Yli_POX6 (SEQ ID NO: 12), Ase_POX (SEQ ID NO: 14), Ath_POX1 (SEQ ID NO: 16), Ath_POX2 (SEQ ID NO: 18), Ani_POX (SEQ ID NO: 20), Cma_POX (SEQ ID NO: 22), Hsa_POX1-2 (SEQ ID NO: 24), Pur_POX (SEQ ID NO: 26), Lbo31670 (SEQ ID NO: 81), Lbo49554 (SEQ ID NO: 83), Lbo49602 (SEQ ID NO: 85) and Rno_POX2 (SEQ ID NO: 28), or a functional variant thereof having at least 60% homology thereto, such as at least 65%, such as at least 70%, such as at least 75%, such as at least 80%, such as at least 81%, such as at least 82%, such as at least 83%, such as at least 84%, such as at least 85%, such as at least 86%, such as at least 87%, such as at least 88%, such as at least 89%, such as at least 90%, such as at least 91%, such as at least 92%, such as at least 93%, such as at least 94%, such as at least 95%, such as at least 96%, such as at least 97%, such as at least 98%, such as at least 99% homology thereto, preferably wherein the at least one acyl-CoA oxidase of the first group of enzymes is an acyl-CoA oxidase selected from the group consisting of Yli_POX2 (SEQ ID NO: 4), Ani_POX (SEQ ID NO: 20), Cma_POX (SEQ ID NO: 22), Pur_POX (SEQ ID NO: 26), Lbo31670 (SEQ ID NO: 81), Lbo49554 (SEQ ID NO: 83), Lbo49602 (SEQ ID NO: 85) and Rno_POX2 (SEQ ID NO: 28).

15. The yeast cell according to any one of the preceding items, wherein X is 8, 9, 10, 11, 12, 13, 14, 15, 16, 17, 18, 19, 20, 21 or 22.

16. The yeast cell according to any one of the preceding items, wherein the yeast is of a genus selected from Saccharomyces, Pichia, Yarrowia, Kluyveromyces, Candida, Rhodotorula, Rhodosporidium, Cryptococcus, Trichosporon and Lipomyces, preferably the genus is Saccharomyces or Yarrowia, most preferably the genus is Yarrowia.

17. The yeast cell according to any one of the preceding items, wherein the yeast is of a species selected from Saccharomyces cerevisiae, Pichia pastoris, Kluyveromyces marxianus, Cryptococcus albidus, Lipomyces lipofera, Lipomyces starkeyi, Rhodosporidium toruloides, Rhodotorula glutinis, Trichosporon pullulan and Yarrowia lipolytica, preferably the yeast cell is a Saccharomyces cerevisiae cell or a Yarrowia lipolytica cell.

18. The yeast cell according to any one of the preceding items, wherein the at least one heterologous desaturase is selected from the group consisting of a Δ3 desaturase, a Δ5 desaturase, a Δ6 desaturase, a Δ7 desaturase, a Δ8 desaturase, a Δ9 desaturase, a Δ10 desaturase, a Δ11 desaturase, a Δ12 desaturase, a Δ13 desaturase and a Δ14 desaturase.

19. The yeast cell according to any one of the preceding items, wherein the desaturase is derived from a yeast such as Saccharomyces or Yarrowia, such as Saccharomyces cerevisiae or Yarrowia lipolytica, or from an insect, such as from the Diptera, the Coleoptera, or the Lepidoptera order, such as of the genus Amyelois, Choristoneura, Helicoverpa, Drosophila, Ostrinia, Thaumetopoea, Dendrophilus, Grapholita, Cydia, Epiphyas, Lobesia, Chilo, Pectinophora or Spodoptera, such as Drosophila melanogaster, Amyelois transitella, Helicoverpa assulta, Helicoverpa armigera, Choristoneura rosaceana, Ostrinia nubilalis, Thaumetopoea pityocampa, Dendrophilus punctatus, Grapholita molesta, Cydia pomonella, Epiphyas postvittana, Spodoptera littoralis, Spodoptera litura, Lobesia botrana, Chilo suppressalis, Pectinophora gossypiella or Choristoneura parallela.

20. The yeast cell according to any one of the preceding items, wherein the desaturase is a Δ_{Z9}-desaturase such as Sce_OLE1 (SEQ ID NO: 30), Yli_OLE1 (SEQ ID NO: 32) or Dme_D9 (SEQ ID NO: 34), a Δ_{Z11}-desaturase such as Atr_D11 (SEQ ID NO: 38), Cro_Z11 (SEQ ID NO: 40), Onu_11 (SEQ ID NO: 42), Tpi_D13 (SEQ ID NO: 44), a Δ_{E9}-desaturase such as Dpu_E9-14 (SEQ ID NO: 46), a Δ_{Z/E10}-desaturase such as Gmo_CPRQ (SEQ ID NO: 48) or Gmp_KPSQ (SEQ ID NO: 96), or a desaturase such as Epo_E11 (SEQ ID NO: 52), Sls_ZE11 (SEQ ID NO: 54), Cpa_E11 (SEQ ID NO: 56), Lbo_PTTQ (SEQ ID NO: 79), Slitdes5 (SEQ ID NO: 87), Lbo_KPSE (SEQ ID NO: 89), Csup_KPSE (SEQ ID NO: 91), Cpo_CPRQ (SEQ ID NO: 50, SEQ ID NO: 129, SEQ ID NO: 131, SEQ ID NO: 133, SEQ ID NO: 135, SEQ ID NO: 137, SEQ ID NO: 139, SEQ ID NO: 141, SEQ ID NO: 143 or SEQ ID NO: 145) or PGDes8 (SEQ ID NO: 101), or a functional variant thereof having at least 60% homology thereto, such as at least 65%, such as at least 70%, such as at least 75%, such as at least 80%, such as at least 81%, such as at least 82%, such as at least 83%, such as at least 84%, such as at least 85%, such as at least 86%, such as at least 87%, such as at least 88%, such as at least 89%, such as at least 90%, such as at least 91%, such as at least 92%, such as at least 93%, such as at least 94%, such as at least 95%, such as at least 96%, such as at least 97%, such as at least 98%, such as at least 99% homology thereto, preferably wherein the desaturase is selected from Sce_OLE1 (SEQ ID NO: 30), Csup_KPSE (SEQ ID NO: 91), Lbo_PTTQ (SEQ ID NO: 79), Sls_ZE11 (SEQ ID NO: 54), Cro_Z11 (SEQ ID NO: 40), Slitdes5 (SEQ ID NO: 87) and Dme_D9 (SEQ ID NO: 34).

21. The yeast cell according to any one of the preceding items, wherein the fatty acyl-CoA reductase is derived from an insect such as an insect of the Lepidoptera order, such as of the genus Helicoverpa, Agrotis, Heliothis or Bicyclus.

22. The yeast cell according to any one of the preceding items, wherein the fatty acyl-CoA reductase is a fatty acyl-CoA reductase native to Helicoverpa armigera, Helicoverpa assulta, Agrotis segetum, Heliothis subflexa, Bicyclus anynana, or a functional variant thereof.

23. The yeast cell according to any one of the preceding items, wherein the fatty acyl-CoA reductase is selected from the group consisting of a fatty acyl-CoA reductase having at least 80% homology to Har_FAR (SEQ ID NO: 59), Has_FAR (SEQ ID NO: 75), Ban_FAR (SEQ ID NO: 77), AseFAR (SEQ ID NO: 93) or Hs_FAR (SEQ ID NO: 73).

24. The yeast cell according to any one of the preceding items, wherein the acetyltransferase is Sce_ATF1 as set forth in SEQ ID NO: 61 or a functional variant thereof having at least 60% homology thereto, such as at least 65%, such as at least 70%, such as at least 75%, such as at least 80%, such as at least 81%, such as at least 82%, such as at least 83%, such as at least 84%, such as at least 85%, such as at least 86%, such as at least 87%, such as at least 88%, such as at least 89%, such as at least 90%, such as at least 91%, such as at least 92%, such as at least 93%, such as at least 94%, such as at least 95%, such as at least 96%, such as at least 97%, such as at least 98%, such as at least 99% homology thereto.

25. The yeast cell according to any one of the preceding items, wherein the acetyltransferase is overexpressed compared to a wild type yeast cell.

26. The yeast cell according to any one of the preceding items, further comprising a mutation in one or more genes encoding enzymes capable of degrading or synthesising fatty acyl-CoAs, such as a mutation in one or more subunits of the fatty acyl-CoA synthetase complex, wherein the mutation yields a modified enzyme having a changed product profile such as reduced capability to degrade fatty acyl-CoAs or increased capability to synthesise fatty acyl-CoAs compared to an unmodified enzyme.

27. The yeast cell according to item 26, wherein the yeast cell is a Yarrowia lipolytica cell and the mutation is in the gene encoding FAS1 (SEQ ID NO: 69) and/or FAS2 (SEQ ID NO: 71) and yields a modified FAS1 and/or FAS2 having reduced capability to degrade fatty acyl-CoAs or increased capability to synthesise fatty acyl-CoAs compared to an unmodified FAS1 and/or FAS2, respectively.

28. The yeast cell according to any one of the preceding items, wherein the genes encoding the at least one acyl-CoA oxidase of the first group of enzymes, the at least one desaturase, the at least one fatty acyl-CoA reductase, or the at least one acetyltransferase and/or alcohol dehydrogenase are independently comprised within the genome of said yeast cell or within one or more vector comprised within said yeast cell.

29. The yeast cell according to any one of the preceding items, wherein at least one of the genes encoding the at least one acyl-CoA oxidase of the first group of enzymes, the at least one desaturase, the at least one fatty acyl-CoA reductase, or the at least one acetyltransferase and/or alcohol dehydrogenase is present in high copy number.

30. The yeast cell according to any one of the preceding items, wherein at least one of the genes encoding at least one acyl-CoA oxidase of the first group of enzymes, the at least one desaturase, the at least one fatty acyl-CoA reductase, or the at least one acetyltransferase and/or alcohol dehydrogenase is under the control of an inducible promoter.

31. The yeast cell according to any one of the preceding items, wherein at least one of the genes encoding at least one acyl-CoA oxidase of the first group of enzymes, the at least one desaturase, the at least one fatty acyl-CoA reductase, or the at least one acetyltransferase and/or alcohol dehydrogenase is codon-optimised for said yeast cell.

32. The yeast cell according to any one of the preceding items, wherein the yeast cell further comprises one or more mutations resulting in partial or total loss of activity of one or more of: HFD1, HFD2, HFD3, HFD4, FAO1, POX1, POX2, POX3, POX4, POX5, POX6, GPAT and PEX10.

33. A method for producing a desaturated fatty alcohol and optionally a desaturated fatty acyl acetate and/or a fatty aldehyde of carbon chain length X′, comprising the steps of providing a yeast cell capable of converting a fatty acyl-CoA of a first carbon chain length X to a desaturated fatty alcohol and optionally a desaturated fatty acyl acetate and/or a fatty aldehyde of carbon chain length X′ and incubating said yeast cell in a medium, wherein the yeast cell is as defined in any one of the preceding items and wherein X′ ≤ X-2.

34. The method according to item 33, wherein X′ = X-2, X′ = X-4 or X′ = X-6.

35. The method according to any one of items 33 to 34, wherein the desaturated fatty acyl acetate and/or fatty aldehyde is obtained by expression of an acetyltransferase or by chemical conversion.

36. The method according to any one of items 33 to 35, wherein the method yields said desaturated fatty alcohol, and optionally said desaturated fatty acyl acetate and/or desaturated fatty aldehyde with a titre of at least 1 mg/L, such as at least 1.5 mg/L, such as at least 5 mg/L, such as at least 10 mg/L, such as at least 25 mg/L, such as at least 50 mg/L, such as at least 100 mg/L, such as at least 250 mg/L, such as at least 500 mg/L, such as at least 750 mg/L, such as at least 1 g/L, such as at least 2 g/L, such as at least 3 g/L, such as at least 4 g/L, such as at least 5 g/L, such as at least 6 g/L, such as at least 7 g/L, such as at least 8 g/L, such as at least 9 g/L, such as at least 10 g/L, such as at least 11 g/L, such as at least 12 g/L, such as at least 13 g/L, such as at least 14 g/L, such as at least 15 g/L, such as at least 16 g/L, such as at least 17 g/L, such as at least 18 g/L, such as at least 19 g/L, such as at least 20 g/L, such as at least 25 g/L, such as at least 30 g/L, such as at least 35 g/L, such as at least 40 g/L, such as at least 45 g/L, such as at least 50 g/L, or more.

37. The method according to any one of items 33 to 36, further comprising the step of recovering said desaturated fatty alcohol and/or desaturated fatty acyl acetate.

38. The method according to any one of items 33 to 37, wherein the method yields fatty alcohols with a total titre of at least 1 mg/L, such as at least 5 mg/L, such as at least 10 mg/L, such as at least 25 mg/L, such as at least 50 mg/L, such as at least 100 mg/L, such as at least 250 mg/L, such as at least 500 mg/L, such as at least 1 g/L, such as at least 2 g/L, such as at least 3 g/L, such as at least 4 g/L, such as at least 5 g/L, such as at least 6 g/L, such as at least 7 g/L, such as at least 8 g/L, such as at least 9 g/L, such as at least 10 g/L, wherein the total titre is the sum of the titre of desaturated fatty alcohols and the titre of saturated fatty alcohols.

39. The method according to any one of items 33 to 38, wherein the total desaturated fatty alcohols produced represent at least 5% of the total fatty alcohols produced by the cell, such as at least 10%, such as at least 20%, such as at least 30%, such as at least 40%, such as at least 50%, such as at least 60%, such as at least 70%, such as at least 80%, such as at least 90% of the total fatty alcohols produced by the cell, wherein the total fatty alcohols produced by the cell are the sum of the saturated fatty alcohols produced by the cell and the desaturated fatty alcohols produced by the cell.

40. The method according to any one of items 33 to 39, wherein the desaturated fatty alcohol of carbon chain length X′ produced by the cell represents at least 2% of the total desaturated fatty alcohols produced by the cell, such as at least 5%, such as at least 6%, such as at least 7%, such as at least 8%, such as at least 9%, such as at least 10%, such as at least 15%, such as at least 20%, such as at least 30%, such as at least 40%, such as at least 50%, such as at least 60%, such as at least 70%, such as at least 80%, such as at least 90% of the total desaturated fatty alcohols produced by the cell.

41. The method according to any one of items 33 to 40, wherein the desaturated fatty alcohol of carbon chain length X′ produced by the cell represents at least 5% of the total fatty alcohols of chain length X′ produced by the cell, such as at least 10%, such as at least 15%, such as at least 20%, such as at least 30%, such as at least 40%, such as at least 50%, such as at least 60%, such as at least 70%, such as at least 80%, such as at least 90% of the total fatty alcohols of carbon chain length X′ produced by the cell, wherein the total fatty alcohols of carbon chain length X′ is the sum of desaturated and saturated fatty alcohol of carbon chain length X′..

42. The method according to any one of items 33 to 41, further comprising the step of recovering the desaturated fatty alcohol, the fatty acyl acetate and/or the aldehyde.

43. The method according to item 42, further comprising the step of formulating the recovered desaturated fatty alcohol and/or desaturated fatty acyl acetate and/or desaturated fatty aldehyde into a pheromone composition.

44. The method according to item 43, wherein the step of formulating the recovered desaturated fatty alcohol and/or desaturated fatty acyl acetate and/or desaturated fatty aldehyde into a pheromone composition further comprises adding one or more additional compounds such as a liquid or solid carrier or substrate to the pheromone composition.

45. A nucleic acid construct for modifying a yeast cell, said construct comprising at least one first group of polynucleotides encoding at least one first group of enzymes comprising at least one acyl-CoA oxidase capable of oxidising a fatty acyl-CoA, wherein the first group of enzymes is capable of shortening a fatty acyl-CoA of a first carbon chain length X to a shortened fatty acyl-CoA having a second carbon chain length X′, wherein X′ ≤ X-2, preferably wherein said acyl-CoA oxidase is as defined in any one of the preceding items.

46. The nucleic acid construct according to item 45, further comprising a second polynucleotide encoding at least one heterologous desaturase capable of introducing at least one double bond in said shortened fatty acyl-CoA, preferably wherein said heterologous desaturase is as defined in any one of the preceding items.

47. The nucleic acid construct according to any one of items 45 to 46, further comprising a third polynucleotide encoding at least one heterologous fatty acyl-CoA reductase (FAR), capable of converting at least part of a desaturated fatty acyl-CoA to a desaturated fatty alcohol, preferably wherein said heterologous FAR is as defined in any one of the preceding items.

48. The nucleic acid construct according to any one of items 45 to 47, further comprising a fourth polynucleotide encoding an acetyltransferase capable of converting at least part of a desaturated fatty alcohol to a desaturated fatty acyl acetate, preferably wherein said acetyltransferase is as defined in any one of the preceding items.

49. The nucleic acid construct according to any one of items 45 to 48, further comprising a fifth polynucleotide encoding at least one alcohol dehydrogenase and/or fatty alcohol oxidase capable of converting at least part of a desaturated fatty alcohol to a desaturated fatty aldehyde, preferably wherein said alcohol dehydrogenase is as defined in any one of the preceding items.

50. The nucleic acid construct according to any one of items 45 to 49, wherein the first group of polynucleotides comprises or consists of a nucleic acid encoding the acyl-CoA oxidase which is selected from SEQ ID NO: 1, SEQ ID NO: 3, SEQ ID NO: 5, SEQ ID NO: 7, SEQ ID NO: 9, SEQ ID NO: 11, SEQ ID NO: 13, SEQ ID NO: 15, SEQ ID NO: 17, SEQ ID NO: 19, SEQ ID NO: 21, SEQ ID NO: 23, SEQ ID NO: 25, SEQ ID NO: 27, SEQ ID NO: 80, SEQ ID NO: 82, SEQ ID NO: 84 or homologues thereof having at least 60% homology thereto, such as at least 65%, such as at least 70%, such as at least 75%, such as at least 80%, such as at least 81%, such as at least 82%, such as at least 83%, such as at least 84%, such as at least 85%, such as at least 86%, such as at least 87%, such as at least 88%, such as at least 89%, such as at least 90%, such as at least 91%, such as at least 92%, such as at least 93%, such as at least 94%, such as at least 95%, such as at least 96%, such as at least 97%, such as at least 98%, such as at least 99% homology thereto, preferably the nucleic acid is selected from SEQ ID NO: 3, SEQ ID NO: 19, SEQ ID NO: 21, SEQ ID NO: 25, SEQ ID NO: 80, SEQ ID NO: 82, SEQ ID NO: 84 and SEQ ID NO: 27.

51. The nucleic acid construct according to any one of items 45 to 50, wherein the second polynucleotide comprises or consists of a nucleic acid having at least 60% homology to SEQ ID NO: 36, SEQ ID NO: 37, SEQ ID NO: 39, SEQ ID NO: 55, SEQ ID NO: 41, SEQ ID NO: 43, SEQ ID NO: 34, SEQ ID NO: 33, SEQ ID NO: 29, SEQ ID NO: 31, SEQ ID NO: 45, SEQ ID NO: 47, SEQ ID NO: 49, SEQ ID NO: 51, SEQ ID NO: 53, SEQ ID NO: 55, SEQ ID NO: 78, SEQ ID NO: 86, SEQ ID NO: 88, SEQ ID NO: 90, SEQ ID NO: 98 or SEQ ID NO: 100, such as at least 61% homology, such as at least 62% homology, such as at least 63% homology, such as at least 64% homology, such as at least 65% homology, such as at least 66% homology, such as at least 67% homology, such as at least 68% homology, such as at least 69% homology, such as at least 70% homology, such as at least 71% homology, such as at least 72%, such as at least 73%, such as at least 74%, such as at least 75%, such as at least 76%, such as at least 77%, such as at least 78%, such as at least 79%, such as at least 80%, such as at least 81%, such as at least 82%, such as at least 83%, such as at least 84%, such as at least 85%, such as at least 86%, such as at least 87%, such as at least 88%, such as at least 89%, such as at least 90%, such as at least 91%, such as at least 92%, such as at least 93%, such as at least 94%, such as at least 95%, such as at least 96%, such as at least 97%, such as at least 98%, such as at least 99% homology to SEQ ID NO: 36, SEQ ID NO: 37, SEQ ID NO: 39, SEQ ID NO: 55, SEQ ID NO: 41, SEQ ID NO: 43, SEQ ID NO: 34, SEQ ID NO: 33, SEQ ID NO: 29, SEQ ID NO: 31, SEQ ID NO: 45, SEQ ID NO: 47, SEQ ID NO: 49, SEQ ID NO: 51, SEQ ID NO: 53, SEQ ID NO: 55, SEQ ID NO: 78, SEQ ID NO: 86, SEQ ID NO: 88, SEQ ID NO: 90, SEQ ID NO: 98 or SEQ ID NO: 100.

52. The nucleic acid construct according to any one of items 45 to 51, wherein the third polynucleotide comprises or consists of a nucleic acid having at least 60% homology to SEQ ID NO: 55, SEQ ID NO: 74, SEQ ID NO: 72, SEQ ID NO: 76, or SEQ ID NO: 92, such as at least 61% homology, such as at least 62% homology, such as at least 63% homology, such as at least 64% homology, such as at least 65% homology, such as at least 66% homology, such as at least 67% homology, such as at least 68% homology, such as at least 69% homology, such as at least 70% homology, such as at least 71% homology, such as at least 72%, such as at least 73%, such as at least 74%, such as at least 75%, such as at least 76%, such as at least 77%, such as at least 78%, such as at least 79%, such as at least 80%, such as at least 81%, such as at least 82%, such as at least 83%, such as at least 84%, such as at least 85%, such as at least 86%, such as at least 87%, such as at least 88%, such as at least 89%, such as at least 90%, such as at least 91%, such as at least 92%, such as at least 93%, such as at least 94%, such as at least 95%, such as at least 96%, such as at least 97%, such as at least 98%, such as at least 99%, such as 100% homology to SEQ ID NO: 55, SEQ ID NO: 74, SEQ ID NO: 72, SEQ ID NO: 76, or SEQ ID NO: 92.

53. The nucleic acid construct according to any one of items 45 to 52, wherein the fourth polynucleotide comprises or consists of a nucleic acid having at least 60% homology to SEQ ID NO: 60, such as at least 61% homology, such as at least 62% homology, such as at least 63% homology, such as at least 64% homology, such as at least 65% homology, such as at least 66% homology, such as at least 67% homology, such as at least 68% homology, such as at least 69% homology, such as at least 70% homology, such as at least 71% homology, such as at least 72%, such as at least 73%, such as at least 74%, such as at least 75%, such as at least 76%, such as at least 77%, such as at least 78%, such as at least 79%, such as at least 80%, such as at least 81%, such as at least 82%, such as at least 83%, such as at least 84%, such as at least 85%, such as at least 86%, such as at least 87%, such as at least 88%, such as at least 89%, such as at least 90%, such as at least 91%, such as at least 92%, such as at least 93%, such as at least 94%, such as at least 95%, such as at least 96%, such as at least 97%, such as at least 98%, such as at least 99%, such as 100% homology to SEQ ID NO: 60.

54. A kit of parts comprising:
a) The yeast cell according to any one of items 1 to 32; and/or
b) The nucleic acid construct according to any one of items 45 to 53; and/or
c) A set of primers for introducing one or more mutations resulting in reduced activity of one or more acyl-CoA oxidases; and optionally the yeast cell to be modified.

55. A desaturated fatty alcohol, a desaturated fatty acyl acetate or a desaturated fatty aldehyde obtainable by the method according to any one of items 33 to 44.

56. Use of a desaturated fatty alcohol, a desaturated fatty acyl acetate or a desaturated fatty aldehyde according to item 55.

57. Use of a desaturated fatty alcohol, a desaturated fatty acyl acetate or a desaturated fatty aldehyde according to item 54 in a method of pest management.

1. A yeast cell capable of producing a compound selected from the group consisting of Z7-12:CoA, Z7-14:CoA, Z7-16:CoA, Z9-12:CoA, Z7-12:OH, Z7-14:OH, Z7-16:OH and Z9-12:OH, said yeast cell:
i) having one or more mutations resulting in reduced activity of one or more native acyl-CoA oxidases; and
ii) expressing at least one first group of enzymes comprising at least one acyl-CoA oxidase capable of oxidising a fatty acyl-CoA, wherein the first group of enzymes is capable of shortening a fatty acyl-CoA of a first carbon chain length X to a shortened fatty acyl-CoA having a second carbon chain length X′, wherein X′ ≤ X-2; and
iii) expressing at least one heterologous desaturase capable of introducing at least one double bond in said fatty acyl-CoA and/or in said shortened fatty acyl-CoA; and
iv) expressing at least one heterologous fatty acyl-CoA reductase, capable of converting at least part of said desaturated fatty acyl-CoA to a desaturated fatty alcohol .

2. The yeast cell according to claim 1, wherein X′ = X-2, X′ = X-4 or X′ = X-6.

3-6. (canceled)

7. The yeast cell according to claim 1, wherein the at least one acyl-CoA oxidase of the first group of enzymes of step ii) is a native acyl-CoA oxidase or a heterologous acyl-CoA oxidase.

8-10. (canceled)

11. The yeast cell according to claim 1, wherein the at least one acyl-CoA oxidase of the first group of enzymes is an acyl-CoA oxidase derived from a yeast, a fungus, an insect, a mammalian, a bird or a plant .

12. The yeast cell according to claim 1, wherein the at least one first group of enzymes comprises an acyl-CoA oxidase derived from an organism of a genus selected from Yarrowia, Agrotis, Arabidopsis, Aspergillus, Cucurbita, Homo, Paenarthrobacter, Lobesia or Rattus.

13. The yeast cell according to claim 1, wherein the at least one first group of enzymes comprises an acyl-CoA oxidase derived from Yarrowia lipolytica, Agrotis segetum, Arabidopsis thaliana, Aspergillus nidulans, Cucurbita maxima, Homo sapiens, Paenarthrobacter ureafaciens, Lobesia botrana or Rattus norvegicus.

14. The yeast cell according to claim 1, wherein the at least one acyl-CoA oxidase of the first group of enzymes is an acyl-CoA oxidase selected from the group consisting of Yli_POX1 (SEQ ID NO: 2), Yli_POX2 (SEQ ID NO: 4), Yli_POX3 (SEQ ID NO: 6), Yli_POX4 (SEQ ID NO: 8), Yli_POX5 (SEQ ID NO: 10), Yli_POX6 (SEQ ID NO: 12), Ase POX (SEQ ID NO: 14), Ath_POX1 (SEQ ID NO: 16), Ath_POX2 (SEQ ID NO: 18), Ani_POX (SEQ ID NO: 20), Cma_POX (SEQ ID NO: 22), Hsa_POX1-2 (SEQ ID NO: 24), Pur_POX (SEQ ID NO: 26), Lbo31670 (SEQ ID NO: 81), Lbo49554 (SEQ ID NO: 83), Lbo49602 (SEQ ID NO: 85) and Rno_POX2 (SEQ ID NO: 28), or a functional variant thereof having at least 60% homology thereto .

15. (canceled)

16. The yeast cell according to claims 1, wherein the yeast is of a genus selected from Saccharomyces, Pichia, Yarrowia, Kluyveromyces, Candida, Rhodotorula, Rhodosporidium, Cryptococcus, Trichosporon and Lipomyces .

17. The yeast cell according to claim 1, wherein the yeast is of a species selected from Saccharomyces cerevisiae, Pichia pastoris, Kluyveromyces marxianus, Cryptococcus albidus, Lipomyces lipofera, Lipomyces starkeyi, Rhodosporidium toruloides, Rhodotorula glutinis, Trichosporon pullulan and Yarrowia lipolytica .

18-19. (canceled)

20. The yeast cell according to claims 1, wherein the desaturase is a Δ_{Z9}-desaturase selected from the group consisting of Sce_OLE1 (SEQ ID NO: 30), Yli_OLE1 (SEQ ID NO: 32) and Dme_D9 (SEQ ID NO: 34), a Δ_{Z11}-desaturase selected from the group consisting of Atr_D11 (SEQ ID NO: 38), Cro_Z11 (SEQ ID NO: 40), Onu_11 (SEQ ID NO: 42), and Tpi_D13 (SEQ ID NO: 44), a Δ_{E9}-desaturase in the form of Dpu_E9-14 (SEQ ID NO: 46), a Δ_{Z/El0}-desaturase selected from the group consisting of Gmo_CPRQ (SEQ ID NO: 48) and Gmo_KPSQ (SEQ ID NO: 96), or a desaturase selected from the group consisting of Epo_E11 (SEQ ID NO: 52), Sls_ZE11 (SEQ ID NO: 54), Cpa_E11 (SEQ ID NO: 56), Lbo_PTTQ (SEQ ID NO: 79), Slitdes5 (SEQ ID NO: 87), Lbo_KPSE (SEQ ID NO: 89), Csup_KPSE (SEQ ID NO: 91), Cpo_CPRQ (SEQ ID NO: 50, SEQ ID NO: 129, SEQ ID NO: 131, SEQ ID NO: 133, SEQ ID NO: 135, SEQ ID NO: 137, SEQ ID NO: 139, SEQ ID NO: 141, SEQ ID NO: 143 or SEQ ID NO: 145) and PGDes8 (SEQ ID NO: 101), or a functional variant thereof having at least 60% homology .

21. The yeast cell according to claims 1, wherein the fatty acyl-CoA reductase is derived from an insect .

22. (canceled)

23. The yeast cell according to claim 1, wherein the fatty acyl-CoA reductase is selected from the group consisting of a fatty acyl-CoA reductase having at least 80% homology to Har_FAR (SEQ ID NO: 59), Has_FAR (SEQ ID NO: 75), Ban_FAR (SEQ ID NO: 77), AseFAR (SEQ ID NO: 93) and Hs_FAR (SEQ ID NO: 73) .

24-32. (canceled)

33. A method for producing a compound selected from the group consisting of Z7-12:CoA, Z7-14:CoA, Z7-16:CoA, Z9-12:CoA, Z7-12:OH, Z7-14:OH, Z7-16:OH and Z9-12:OH, comprising the steps of providing a yeast cell capable of converting at least part of a fatty acyl-CoA of a first carbon chain length X to a desaturated fatty alcohol and incubating said yeast cell in a medium, wherein the yeast cell is as defined in claims 1 and wherein X′ ≤ X-2.

34. The method according to claim 0, wherein X′ = X-2, X′ = X-4 or X′ = X-6.

35. (canceled)

36. The method according to claim 0, wherein the method yields said compound selected from the group consisting of Z7-12:CoA, Z7-14:CoA, Z7-16:CoA, Z9-12:CoA, Z7-12:OH, Z7-14:OH, Z7-16:OH and Z9-12:OH .

37-41. (canceled)

42. The method according to claim 0 further comprising the step of recovering the compound selected from the group consisting of Z7-12:OH, Z7-14:OH, Z7-16:OH and Z9-12:OH .

43. The method according to claim 0, further comprising the step of formulating the recovered compound selected from the group consisting of Z7-12:OH, Z7-14:OH, Z7-16:OH and Z9-12:OH into a pheromone composition.

44. The method according to claim 0, wherein the step of formulating the recovered compound selected from the group consisting of Z7-12:OH, Z7-14:OH, Z7-16:OH and Z9-12:OH into a pheromone composition further comprises adding one or more additional compounds selected from a liquid or solid carrier or substrate to the pheromone composition.

45. A nucleic acid construct for modifying a yeast cell, said construct comprising at least one first group of polynucleotides encoding at least one first group of enzymes comprising at least one acyl-CoA oxidase capable of oxidising a fatty acyl-CoA, wherein the first group of enzymes is capable of shortening a fatty acyl-CoA of a first carbon chain length X to a shortened fatty acyl-CoA having a second carbon chain length X′, wherein X′ ≤ X-2, and further comprising a second polynucleotide encoding at least one heterologous desaturase capable of introducing at least one double bond in said shortened fatty acyl-CoA.

46-49. (canceled)

50. The nucleic acid construct according to claim 0, wherein the first group of polynucleotides comprises or consists of a nucleic acid encoding the acyl-CoA oxidase which is selected from SEQ ID NO: 1, SEQ ID NO: 3, SEQ ID NO: 5, SEQ ID NO: 7, SEQ ID NO: 9, SEQ ID NO: 11, SEQ ID NO: 13, SEQ ID NO: 15, SEQ ID NO: 17, SEQ ID NO: 19, SEQ ID NO: 21, SEQ ID NO: 23, SEQ ID NO: 25, SEQ ID NO: 27, SEQ ID NO: 80, SEQ ID NO: 82, SEQ ID NO: 84 or homologues thereof having at least 60% homology thereto .

51-57. (canceled)
